(19) United States
(12) Patent Application Publication
(10) Pub. No.: US 2023/0345947 A1
(43) Pub. Date: Nov. 2, 2023

(54) RNA-BASED CONTROL OF LEPIDOPTERAN PESTS

(71) Applicant: GREENLIGHT BIOSCIENCES, INC., Lexington, MA (US)

(72) Inventors: Kenneth E. NARVA, Medford, MA (US); Krishnakumar SRIDHARAN, Medford, MA (US); Upendra Kumar DEVISETTY, Medford, MA (US); Thais BARROS RODRIGUES, Medford, MA (US); Cris OPPERT, Medford, MA (US); Suresh DESAI, Medford, MA (US); Sambit Kumar MISHRA, Medford, MA (US); Evelien VAN EKERT, Medford, MA (US)

(21) Appl. No.: 17/925,204

(22) PCT Filed: May 13, 2021
(86) PCT No.: PCT/US2021/032334

§ 371 Date: Nov. 14, 2022

Related U.S. Application Data
(60) Provisional application No. 63/024,133, filed on May 13, 2020.
(60) Provisional application No. 63/106,614, filed on Oct. 28, 2020.

(51) Int. Cl. A01N 63/60; C12N 15/113; C12N 15/82; C12P 19/34; A01P 7/04

(52) U.S. Cl. CPC A01N 63/60; C12N 15/113; C12N 15/8286; C12P 19/34; A01P 7/04; C12N 2310/14

(57) ABSTRACT

Disclosed herein are compositions and methods of controlling pests that involves administering RNA interfering agents. Particularly exemplified is the control of Lepidopteran species such as diamondback moths and fall armyworms. Also disclosed is the use of double-stranded RNA molecules to target certain genes in Lepidopteran species.

### RELATED APPLICATIONS

This application claims the benefit under 35 U.S.C. (s) 119(e) of U.S. provisional application No. 63/024,133 and U.S. provisional application No. 63/106,614, which are incorporated by reference herein in their entireties.

### BACKGROUND

Crops are often the target of insect attacks. Globally, farmers lose 30 to 40 percent of their crops due to pests and diseases, according to the UN Food and Agricultural Organization. Crop maintenance and crop health are essential for yield and quality of produce, which ultimately require long-term strategies for the minimization of pest and disease occurrence. The annual costs of controlling crop pests (e.g., Lepidoptera, Diptera, Coleoptera, Hemiptera, and others) are estimated to be in the tens of millions of dollars, with projected annual costs of crop loss reaching billions of dollars if left uncontrolled.

While chemical pesticides have been one solution for eradicating pest infestations, alternative, more environmentally safe, solutions are needed. Chemical pesticides are harmful to the environment and may lack specificity or selectivity which ultimately results in non-target effects. Additionally, given the slow metabolism of chemical pesticides and the likelihood of chemical pesticides to accumulate, resistance is likely to occur. Thus, there has been a long-felt need for more environmentally friendly methods for controlling or eradicating insect infestations which are more selective, environmentally safe, and biodegradable.

### SUMMARY

The present embodiments are related to control of Lepidopteran pests, especially those that are economically or agriculturally important pests. In various embodiments, the Lepidopteran pest is at least one selected from the group consisting of Spodoptera spp (such as S. frugiperda, a.k.a., fall armyworm)) and Plutella spp (such as P. xylostella a.k.a., diamondback moth)).

The compositions and methods described herein include recombinant polynucleotide molecules, such as recombinant DNA constructs for making transgenic plants resistant to infestation by Lepidopteran pests, and single- or double-stranded DNA or RNA molecules, referred to herein as “triggers”, that are useful for controlling or preventing infestation of a plant by that Lepidopteran pest. In some embodiments, polynucleotide triggers are provided as topically applied agents for controlling or preventing infestation of a plant by a Lepidopteran pest. In some embodiments, crop plants with improved resistance to infestation by Lepidopteran pest, such as transgenic crop plants (including seeds or propagatable parts such as tubers) expressing a polynucleotide trigger are provided. In some embodiments, crop plants (including seeds or propagatable parts such as tubers) that have been topically treated with a composition comprising a polynucleotide trigger (e.g., crop plants that have been sprayed with a solution of dsRNA molecules) are provided. Also provided are polynucleotide-containing compositions that are topically applied to a Lepidopteran pest or to a plant, plant part, or seed to be protected from infestation by a Lepidopteran pest.

Several embodiments relate to suppression of a target gene in a Lepidopteran pest by a polynucleotide trigger. Some embodiments relate to methods for selecting Lepidopteran target genes that may be effective targets for RNAi-mediated control of a Lepidopteran pest. In some embodiments, target genes selected for RNAi-mediated suppression are genes that are non-repetitive and non-redundant in a Lepidopteran pest genome, or that have low nucleotide diversity, or that are evolutionarily or functionally constrained to have a more synonymous (Kₛ) than nonsynonymous (Kₐ) nucleotide changes. Provided herein are nucleotide sequences referred to herein as the “Target Gene Sequences Group”, which consists of SEQ ID NOs: 1-114, 229-361, 495-511; 530-535, 542-822, 1104-1333, and 1564-1623. Also provided are nucleotide sequences referred to herein as the “Trigger Sequences Group”, which consists of SEQ ID NOs: 115-228, 362-494, 512-529; 536-541, 823-1103, 1334-1563, and 1624-1683. The SEQ ID NOs relate to the sequence provided in SEQ ID listing provided herewith.

In one aspect, a method for controlling a Lepidopteran pest infestation of a plant comprising contacting the Lepidopteran pest with a polynucleotide comprising at least one segment of 18 or more contiguous nucleotides with a sequence of about 95% to about 100% identity (e.g., a segment of 21 contiguous nucleotides with a sequence of 100% identity would be included) with a corresponding fragment of a DNA having a sequence selected from the Target Gene Sequences Group or the Trigger Sequences Group, or the DNA complement thereof. In an embodiment, the method for controlling a Lepidopteran pest infestation of a plant comprises contacting the Lepidopteran pest with a polynucleotide comprising a nucleotide sequence that is complementary to at least 18 contiguous nucleotides of a target gene having a nucleotide sequence selected from the group consisting of: SEQ ID NOs: 2, 5, 7, 9, 13, 21, 26, 27, 29, 36, 37, 39, 44, 47, 87, 95, 99, 103, 301, 309, 318, 319, 320, 321, 323, 324, 328, 341, 343, 346, 356, 358, 359, 503, 504, 505, 506, 509, 510, 530, 532, 533, 542, 545, 546, 547, 549, 551, 552, 553, 559, 560, 561, 563, 564, 570, 572, 573, 581, 593, 596, 602, 611, 612, 620, 665, 666, 672, 717, 730, 731, 736, 737, 738, 740, 741, 742, 743, 749, 757, 760, 761, 763, 766, 808, 809, 810, 814, 815, 817, 818, 821, 822, 1104, 1105, 1106, 1111, 1113, 1116, 1121, 1123, 1124, 1132, 1133, 1147, 1156, 1157, 1163, 1166, 1187, 1190, 1192, 1195, 1196, 1216, 1217, 1240, 1243, 1251, 1263, 1264, 1265, 1270, 1275, 1279, 1283, 1290, 1308, 1310, 1316, 1318, 1320, 1564, 1565, 1569, 1622, and 1623, or an RNA transcribed from the target gene. In some embodiments, the polynucleotide is double-stranded RNA. In some embodiments, the polynucleotide comprises one or more nucleotide sequences selected from the Trigger Sequences Group. In some embodiments, the contacting with a polynucleotide is achieved by topical application of the polynucleotide, or of a composition or solution containing the polynucleotide (e.g., by spraying or dusting or soaking), directly to the Lepidopteran pest or to a surface or matrix (e.g., a plant or soil) contacted by the Lepidopteran pest. In some embodiments, the contacting with a polynucleotide is achieved by providing a polynucleotide that is ingested by the Lepidopteran pest. In some embodiments, the contacting with a polynucleotide is achieved by providing a transgenic plant that expresses to the Lepidopteran pest.

Several embodiments relate to a method for controlling a Lepidopteran pest infestation of a plant by providing in the diet of a Lepidopteran pest an agent comprising a polynucleotide having at least one segment of 18 or more contiguous nucleotides with a sequence of about 95% to about 100% identity (e.g., a segment of 21 contiguous nucleotides with a sequence of 100% identity) with a corresponding fragment of a DNA having a sequence selected from the Target Gene Sequences Group, the Trigger Sequences Group, or the DNA complement of any thereof, and wherein the agent functions upon ingestion by the Lepidopteran pest to inhibit a biological function within the Lepidopteran pest thereby controlling infestation by the Lepidopteran pest. In an embodiment, the method for controlling a Lepidopteran pest infestation of a plant comprises providing in the diet of the Lepidopteran pest a polynucleotide comprising a nucleotide sequence that is complementary to at least 18 contiguous nucleotides of a target gene having a nucleotide sequence selected from the group consisting of: SEQ ID NOs: 2, 5, 7, 9, 13, 21, 26, 27, 29, 36, 37, 39, 44, 47, 87, 95, 99, 103, 301, 309, 318, 319, 320, 321, 323, 324, 328, 341, 343, 346, 356, 358, 359, 503, 504, 505, 506, 509, 510, 530, 532, 533, 542, 545, 546, 547, 549, 551, 552, 553, 559, 560, 561, 563, 564, 570, 572, 573, 581, 593, 596, 602, 611, 612, 620, 665, 666, 672, 717, 730, 731, 736, 737, 738, 740, 741, 742, 743, 749, 757, 760, 761, 763, 766, 808, 809, 810, 814, 815, 817, 818, 821, 822, 1104, 1105, 1106, 1111, 1113, 1116, 1121, 1123, 1124, 1132, 1133, 1147, 1156, 1157, 1163, 1166, 1187, 1190, 1192, 1195, 1196, 1216, 1217, 1240, 1243, 1251, 1263, 1264, 1265, 1270, 1275, 1279, 1283, 1290, 1308, 1310, 1316, 1318, 1320, 1564, 1565, 1569, 1622, and 1623, or an RNA transcribed from the target gene. In some embodiments, the polynucleotide comprises one or more nucleotide sequences selected from the Trigger Sequences Group. In some embodiments, the polynucleotide is double-stranded RNA. In some embodiments, the agent containing the polynucleotide is formulated for application to fields of crop plants, e.g., in sprayable solutions or emulsions, tank mixes, or powders. In some embodiments, the agent is biologically produced, e.g., in the form of a microbial fermentation product or expressed in a transgenic plant cell.

In another aspect, a method of causing mortality or stunting in Lepidopteran pest larvae is provided. In some embodiments, at least one RNA comprising at least one silencing element is provided in the diet of a Lepidopteran pest larvae wherein ingestion of the RNA by the Lepidopteran pest larvae results in mortality or stunting in the Lepidopteran pest larvae. In some embodiments, the silencing element is essentially identical or essentially complementary to a fragment of a target gene sequence of the Lepidopteran pest larvae, wherein the target gene is selected from the group consisting of the genes in the Target Gene Sequences Group. In an embodiment, the method of causing mortality or stunting in larvae of the Lepidopteran pest comprises providing in the diet of the larvae at least one polynucleotide comprising at least one silencing element comprising at least 18, 19, 20, or 21 contiguous nucleotides that are complementary to a target gene having a nucleotide sequence selected from the Target Gene Sequences Group. In a specific embodiment, the target gene is selected from the group consisting of: SEQ ID NOs: 2, 5, 7, 9, 13, 21, 26, 27, 29, 36, 37, 39, 44, 47, 87, 95, 99, 103, 301, 309, 318, 319, 320, 321, 323, 324, 328, 341, 343, 346, 356, 358, 359, 503, 504, 505, 506, 509, 510, 530, 532, 533, 542, 545, 546, 547, 549, 551, 552, 553, 559, 560, 561, 563, 564, 570, 572, 573, 581, 593, 596, 602, 611, 612, 620, 665, 666, 672, 717, 730, 731, 736, 737, 738, 740, 741, 742, 743, 749, 757, 760, 761, 763, 766, 808, 809, 810, 814, 815, 817, 818, 821, 822, 1104, 1105, 1106, 1111, 1113, 1116, 1121, 1123, 1124, 1132, 1133, 1147, 1156, 1157, 1163, 1166, 1187, 1190, 1192, 1195, 1196, 1216, 1217, 1240, 1243, 1251, 1263, 1264, 1265, 1270, 1275, 1279, 1283, 1290, 1308, 1310, 1316, 1318, 1320, 1564, 1565, 1569, 1622, and 1623, or an RNA transcribed from the target gene. In some embodiments, the silencing element comprises one or more nucleotide sequences selected from the Trigger Sequences Group. In some embodiments, the polynucleotide is double-stranded RNA. Some embodiments relate to a method of causing mortality or lower fecundity in Lepidopteran pest comprising providing in the diet of Lepidopteran pest at least one RNA comprising at least one silencing element essentially identical or essentially complementary to a fragment of a target gene sequence of the Lepidopteran pest larvae wherein ingestion of the RNA by the Lepidopteran pest results in mortality or lower fecundity in the Lepidopteran pest. In some embodiments, the target gene is selected from the group consisting of the genes in the Target Gene Sequences Group. In some embodiments, the method causes a decrease in metamorphosis rate or a decrease in feeding activity. In some embodiments, the method is useful for providing plants having increased resistance to infestation by Lepidopteran pest.

Several embodiments relate to a method of providing a plant having improved resistance to a Lepidopteran pest infestation comprising topically applying to the plant a composition comprising at least one polynucleotide having at least one segment of 18 or more contiguous nucleotides with a sequence of about 95% to about 100% identity (e.g., a segment of 21 contiguous nucleotides with a sequence of 100% identity) with a corresponding fragment of a DNA having a sequence selected from the Target Gene Sequences Group, the Trigger Sequences Group, or the DNA complement of any thereof. In an embodiment, the method of providing a plant having improved resistance to a Lepidopteran pest infestation comprises topically applying to the plant a composition comprising at least one polynucleotide comprising a nucleotide sequence that is complementary to at least 18 contiguous nucleotides of a target gene having a nucleotide sequence selected from the group consisting of: SEQ ID NOs: 2, 5, 7, 9, 13, 21, 26, 27, 29, 36, 37, 39, 44, 47, 87, 95, 99, 103, 301, 309, 318, 319, 320, 321, 323, 324, 328, 341, 343, 346, 356, 358, 359, 503, 504, 505, 506, 509, 510, 530, 532, 533, 542, 545, 546, 547, 549, 551, 552, 553, 559, 560, 561, 563, 564, 570, 572, 573, 581, 593, 596, 602, 611, 612, 620, 665, 666, 672, 717, 730, 731, 736, 737, 738, 740, 741, 742, 743, 749, 757, 760, 761, 763, 766, 808, 809, 810, 814, 815, 817, 818, 821, 822, 1104, 1105, 1106, 1111, 1113, 1116, 1121, 1123, 1124, 1132, 1133, 1147, 1156, 1157, 1163, 1166, 1187, 1190, 1192, 1195, 1196, 1216, 1217, 1240, 1243, 1251, 1263, 1264, 1265, 1270, 1275, 1279, 1283, 1290, 1308, 1310, 1316, 1318, 1320, 1564, 1565, 1569, 1622, and 1623, or an RNA transcribed from the target gene. In an embodiment, the method of providing a plant having improved resistance to a Lepidopteran pest infestation comprises topically applying to the plant a composition comprising at least one polynucleotide in a manner such that an effective amount of the polynucleotide is ingested by a Lepidopteran pest feeding on the plant, the polynucleotide comprising at least 18 contiguous nucleotides that are complementary to a target gene having a nucleotide sequence selected from the group consisting of: SEQ ID NOs: 2, 5, 7, 9, 13, 21, 26, 27, 29, 36, 37, 39, 44, 47, 87, 95, 99, 103, 301, 309, 318, 319, 320, 321, 323, 324, 328, 341, 343, 346, 356, 358, 359, 503, 504, 505, 506, 509, 510, 530, 532, 533, 542, 545, 546, 547, 549, 551, 552, 553, 559, 560, 561, 563, 564, 570, 572, 573, 581, 593, 596, 602, 611, 612, 620, 665, 666, 672, 717, 730, 731, 736, 737, 738, 740, 741, 742, 743, 749, 757, 760, 761, 763, 766, 808, 809, 810, 814, 815, 817, 818, 821, 822, 1104, 1105, 1106, 1111, 1113, 1116, 1121, 1123, 1124, 1132, 1133, 1147, 1156, 1157, 1163, 1166, 1187, 1190, 1192, 1195, 1196, 1216, 1217, 1240, 1243, 1251, 1263, 1264, 1265, 1270, 1275, 1279, 1283, 1290, 1308, 1310, 1316, 1318, 1320, 1564, 1565, 1569, 1622, and 1623, or an RNA transcribed from the target gene. In some embodiments, the polynucleotide comprises one or more nucleotide sequences selected from the Trigger Sequences Group. In some embodiments, the polynucleotide is double-stranded RNA. Several embodiments relate to compositions comprising the polynucleotide, formulated for application to fields of crop plants, e.g., in sprayable solutions or emulsions, tank mixes, or powders.

Several embodiments relate to an insecticidal composition for controlling a Lepidopteran pests comprising an insecticidally effective amount of at least one polynucleotide molecule comprising at least one segment of 18 or more contiguous nucleotides that are essentially identical or complementary (e.g., a segment of 21 contiguous nucleotides with a sequence of 100% identity or complementarity) with the corresponding fragment of DNA having a sequence selected from the Target Gene Sequences Group, the Trigger Sequences Group, or the DNA complement of any thereof. In some embodiments, the polynucleotide molecule comprises at least 18 contiguous nucleotides that are complementary to a target gene having a nucleotide sequence selected from the group consisting of: SEQ ID NOs: 2, 5, 7, 9, 13, 21, 26, 27, 29, 36, 37, 39, 44, 47, 87, 95, 99, 103, 301, 309, 318, 319, 320, 321, 323, 324, 328, 341, 343, 346, 356, 358, 359, 503, 504, 505, 506, 509, 510, 530, 532, 533, 542, 545, 546, 547, 549, 551, 552, 553, 559, 560, 561, 563, 564, 570, 572, 573, 581, 593, 596, 602, 611, 612, 620, 665, 666, 672, 717, 730, 731, 736, 737, 738, 740, 741, 742, 743, 749, 757, 760, 761, 763, 766, 808, 809, 810, 814, 815, 817, 818, 821, 822, 1104, 1105, 1106, 1111, 1113, 1116, 1121, 1123, 1124, 1132, 1133, 1147, 1156, 1157, 1163, 1166, 1187, 1190, 1192, 1195, 1196, 1216, 1217, 1240, 1243, 1251, 1263, 1264, 1265, 1270, 1275, 1279, 1283, 1290, 1308, 1310, 1316, 1318, 1320, 1564, 1565, 1569, 1622, and 1623, or an RNA transcribed from the target gene. In some embodiments, the polynucleotide comprises one or more nucleotide sequences selected from the Trigger Sequences Group. In some embodiments, the polynucleotide molecule is a recombinant polynucleotide. In some embodiments, the polynucleotide molecule is RNA. In some embodiments, the polynucleotide molecule is double-stranded RNA. Related embodiments include insecticidal compositions comprising the polynucleotide molecule formulated for application to fields of crop plants, e.g., in sprayable solutions or emulsions, tank mixes, or powders, and optionally comprising one or more additional components, such as a carrier agent, a surfactant, a cationic lipid, an organosilicone, an organosilicone surfactant, a polynucleotide herbicidal molecule, a non-polynucleotide herbicidal molecule, a non-polynucleotide pesticide, a safeners, and an insect growth regulator.

Several embodiments relate to a method of providing a plant having improved resistance to a Lepidopteran pest infestation comprising expressing in the plant at least one polynucleotide comprising at least one segment of 18 or more contiguous nucleotides that are essentially identical or complementary to (e.g., a segment of 21 contiguous nucleotides with a sequence of 100% identity or complementarity with) the corresponding fragment of DNA having a sequence selected from the Target Gene Sequences Group, the Trigger Sequences Group, or the DNA complement of any thereof. In some embodiments, the polynucleotide comprises one or more nucleotide sequences selected from the Trigger Sequences Group. In some embodiments, the polynucleotide is double-stranded RNA.

Several embodiments relate to a recombinant DNA construct comprising a heterologous promoter operably linked to a DNA element comprising at least one segment of 18 or more contiguous nucleotides with a sequence of about 95% to about 100% identity (e.g., a segment of 21 contiguous nucleotides with a sequence of 100% identity) with the corresponding fragment of DNA having a sequence selected from the Target Gene Sequences Group, the Trigger Sequences Group, or the DNA complement of any thereof. In some embodiments, the DNA element encodes a double-stranded RNA. In some embodiments, the double-stranded RNA comprises one or more nucleotide sequences selected from the Trigger Sequences Group. Related embodiments include a plant chromosome or a plastid or a recombinant plant virus vector or a recombinant baculovirus vector comprising the recombinant DNA construct, or comprising the DNA element without the heterologous promoter.

Several embodiments relate to a transgenic crop plant cell having in its genome a recombinant DNA encoding RNA that suppresses expression of a target gene in a Lepidopteran pest that contacts or ingests the RNA, wherein the RNA comprises at least one silencing element having at least one segment of 18 or more contiguous nucleotides complementary to a fragment of a target gene. In some embodiments, the target gene is selected from the Target Gene Sequences Group. A specific embodiment is a transgenic crop plant cell having in its genome a recombinant DNA encoding RNA for silencing one or more target genes selected from the Target Gene Sequences Group. In some embodiments, the RNA comprises one or more nucleotide sequences selected from the Trigger Sequences Group.

Several embodiments relate to an isolated recombinant RNA molecule that causes mortality or stunting of growth in a Lepidopteran pest when ingested or contacted by the Lepidopteran pest, wherein the recombinant RNA molecule comprises at least one segment of 18 or more contiguous nucleotides that are essentially complementary to (e.g., a segment of 21 contiguous nucleotides with a sequence of 100% complementarity with) the corresponding fragment of DNA having a sequence selected from the Target Gene Sequences Group, the Trigger Sequences Group, or the DNA complement of any thereof. In some embodiments, the recombinant RNA molecule is double-stranded RNA. Specific embodiments include an isolated recombinant double-stranded RNA molecule with a strand having a sequence selected from the group consisting of SEQ ID NOs: 115, 116, 119, 121, 123, 127, 135, 140, 141, 143, 150, 151, 153, 158, 161, 201, 209, 213, 217, 434, 442, 451, 452, 453, 454, 456, 457, 461, 474, 476, 479, 489, 491, 492, 521, 522, 523, 524, 527, 528, 536, 538, 539, 823, 826, 827, 828, 830, 832, 833, 834, 840, 841, 842, 844, 845, 851, 853, 854, 862, 874, 877, 883, 892, 893, 901, 946, 947, 953, 998, 1011, 1012, 1017, 1018, 1019, 1021, 1022, 1023, 1024, 1030, 1038, 1041, 1042, 1044, 1047, 1089, 1090, 1091, 1095, 1096, 1098, 1099, 1102, 1103, 1334, 1335, 1336, 1341, 1343, 1346, 1351, 1353, 1354, 1362, 1363, 1377, 1386, 1387, 1393, 1396, 1417, 1420, 1422, 1425, 1426, 1446, 1447, 1470, 1473, 1481, 1493, 1494, 1495, 1500, 1505, 1509, 1513, 1520, 1538, 1540, 1546, 1548, 1550, 1624, 1625, 1629, 1682, or 1683, or a combination thereof. Another embodiment pertains to an isolated recombinant double-stranded RNA molecule with a strand having a sequence selected from the group consisting of SEQ ID NOs: 434, 442, 451, 452, 453, 454, 456, 457, 461, 474, 476, 479, 489, 491, 492, 521, 522, 523, 524, 527, 528, 536, 538 and 539, or a combination thereof.

Several embodiments relate to a method of providing a plant having improved resistance to a Lepidopteran pest infestation comprising providing to the plant at least one polynucleotide comprising at least one segment of 18 or more contiguous nucleotides that are essentially identical or complementary to (e.g., a segment of 21 contiguous nucleotides with a sequence of 100% identity or complementarity with) the corresponding fragment of a target gene selected from the Target Gene Sequences Group. In an embodiment, the method of providing a plant having improved resistance to a Lepidopteran pest infestation comprises providing to the plant at least one polynucleotide comprising at least one segment that is identical or complementary to at least 18 contiguous nucleotides of a target gene or an RNA transcribed from the target gene, wherein the target gene is selected from the group consisting of: the genes identified in the Target Gene Sequences Group. In some embodiments, the polynucleotide comprises one or more nucleotide sequences selected from the Trigger Sequences Group. In some embodiments, the polynucleotide is double-stranded RNA.

Several embodiments relate to a method for controlling a Lepidopteran pest infestation of a plant comprising contacting the Lepidopteran pest with a polynucleotide comprising at least one segment of 18 or more contiguous nucleotides that are essentially identical or complementary to (e.g., a segment of 21 contiguous nucleotides with a sequence of 100% identity or complementarity with) the corresponding fragment of equivalent length of a DNA of a target gene selected from the Target Gene Sequences Group. In some embodiments, the polynucleotide is double-stranded RNA. Several embodiments relate to a method of selecting target genes for RNAi-mediated silencing from a plant genome or from an animal genome (e.g. insects and arthropods). In various embodiments, the method provides a subset of target genes that are present in single- or low-copy-number (non-repetitive and non-redundant) in a particular genome, or that have low nucleotide diversity, or that have a ratio of synonymous (Kₛ) to nonsynonymous (Kₐ) nucleotide changes where Kₛ>>Kₐ.

Several embodiments relate to man-made compositions comprising at least one polynucleotide as described herein. In some embodiments, formulations useful for topical application to a plant or substance in need of protection from a Lepidopteran pest infestation are provided. In some embodiments, recombinant constructs and vectors useful for making transgenic crop plant cells and transgenic crop plants are provided. In some embodiments, formulations and coatings useful for treating crop plants, crop plant seeds or propagatable parts such as tubers are provided. In some embodiments, commodity products and foodstuffs produced from such crop plants, seeds, or propagatable parts treated with or containing a polynucleotide as described herein (especially commodity products and foodstuffs having a detectable amount of a polynucleotide as described herein) are provided. Several embodiments relate to polyclonal or monoclonal antibodies that bind a protein encoded by a sequence or a fragment of a sequence selected from the Target Gene Sequences Group. Another aspect relates to polyclonal or monoclonal antibodies that bind a protein encoded by a sequence or a fragment of a sequence selected from the Trigger Sequences Group, or the complement thereof. Such antibodies are made by routine methods as known to one of ordinary skill in the art.

In the various embodiments described herein, the plant can be any plant that is subject to infestation by a Lepidopteran pest. Of particular interest are embodiments wherein the plant is a [name families of plants]. Examples include a plant selected from the group consisting of [name specific crops plants]. Embodiments include those wherein the plant is an ungerminated crop plant seed, a crop plant in a vegetative stage, or a crop plant in a reproductive stage.

Other aspects and specific embodiments of this invention are disclosed in the following detailed description.

### DETAILED DESCRIPTION

### I. Definitions

Unless defined otherwise, all technical and scientific terms used have the same meaning as commonly understood by one of ordinary skill in the art to which this invention belongs. Where a term is provided in the singular, the inventors also contemplate aspects of the invention described by the plural of that term. Where there are discrepancies in terms and definitions used in references that are incorporated by reference, the terms used in this application shall have the definitions given herein. Other technical terms used have their ordinary meaning in the art in which they are used, as exemplified by various art-specific dictionaries, for example, “The American Heritage® Science Dictionary” (Editors of the American Heritage Dictionaries, 2011, Houghton Mifflin Harcourt, Boston and New York), the “McGraw-Hill Dictionary of Scientific and Technical Terms” (6^{th} edition, 2002, McGraw-Hill, New York), or the “Oxford Dictionary of Biology” (6^{th} edition, 2008, Oxford University Press, Oxford and New York). The inventors do not intend to be limited to a mechanism or mode of action. Reference thereto is provided for illustrative purposes only.

Unless otherwise stated, nucleic acid sequences in the text of this specification are given, when read from left to right, in the 5′ to 3′ direction. One of skill in the art would be aware that a given DNA sequence is understood to define a corresponding RNA sequence which is identical to the DNA sequence except for replacement of the thymine (T) nucleotides of the DNA with uracil (U) nucleotides. Thus, providing a specific DNA sequence is understood to define the exact RNA equivalent and the term “identity” or “essentially identical” in reference to a DNA sequence includes an RNA sequence meeting these criteria except that thymine nucleotides are replaced with uracil nucleotides. A given first polynucleotide sequence, whether DNA or RNA, further defines the sequence of its exact complement (which can be DNA or RNA), a second polynucleotide that hybridizes perfectly to the first polynucleotide by forming Watson-Crick base-pairs. For DNA:DNA duplexes (hybridized strands), base-pairs are adenine:thymine or guanine:cytosine; for DNA:RNA duplexes, base-pairs are adenine:uracil or guanine:cytosine. Thus, the nucleotide sequence of a blunt-ended double-stranded polynucleotide that is perfectly hybridized (where there is “100% complementarity” between the strands or where the strands are “complementary”) is unambiguously defined by providing the nucleotide sequence of one strand, whether given as DNA or RNA. By “essentially identical” or “essentially complementary” to a target gene or a fragment of a target gene is meant that a polynucleotide strand (or at least one strand of a double-stranded polynucleotide) is designed to hybridize (generally under physiological conditions such as those found in a living plant or animal cell) to a target gene or to a fragment of a target gene or to the transcript of the target gene or the fragment of a target gene; one of skill in the art would understand that such hybridization does not necessarily require 100% sequence identity or complementarity. A first nucleic acid sequence is “operably” connected or “linked” with a second nucleic acid sequence when the first nucleic acid sequence is placed in a functional relationship with the second nucleic acid sequence. For example, a promoter sequence is “operably linked” to a DNA if the promoter provides for transcription or expression of the DNA. Generally, operably linked DNA sequences are contiguous.

The term “polynucleotide” commonly refers to a DNA or RNA molecule containing multiple nucleotides and generally refers both to “oligonucleotides” (a polynucleotide molecule of 18-25 nucleotides in length) and longer polynucleotides of 26 or more nucleotides. Polynucleotides also include molecules containing multiple nucleotides including non-canonical nucleotides or chemically modified nucleotides as commonly practiced in the art; see, e.g., chemical modifications disclosed in the technical manual “RNA Interference (RNAi) and DsiRNAs”, 2011 (Integrated DNA Technologies Coralville, Iowa). Generally, polynucleotides as described herein, whether DNA or RNA or both, and whether single- or double-stranded, include at least one segment of 18 or more contiguous nucleotides (or, in the case of double-stranded polynucleotides, at least 18 contiguous base-pairs) that are essentially identical or complementary to a fragment of equivalent size of the DNA of a target gene or the target gene's RNA transcript. Throughout this disclosure, “at least 18 contiguous” means “from about 18 to about 10,000, including every whole number point in between”. Thus, embodiments of this invention include oligonucleotides having a length of 18-25 nucleotides (18-mers, 19-mers, 20-mers, 21-mers, 22-mers, 23-mers, 24-mers, or 25-mers), or medium-length polynucleotides having a length of 26 or more nucleotides (polynucleotides of 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, about 65, about 70, about 75, about 80, about 85, about 90, about 95, about 100, about 110, about 120, about 130, about 140, about 150, about 160, about 170, about 180, about 190, about 200, about 210, about 220, about 230, about 240, about 250, about 260, about 270, about 280, about 290, or about 300 nucleotides), or long polynucleotides having a length greater than about 300 nucleotides (e.g., polynucleotides of between about 300 to about 400 nucleotides, between about 400 to about 500 nucleotides, between about 500 to about 600 nucleotides, between about 600 to about 700 nucleotides, between about 700 to about 800 nucleotides, between about 800 to about 900 nucleotides, between about 900 to about 1000 nucleotides, between about 300 to about 500 nucleotides, between about 300 to about 600 nucleotides, between about 300 to about 700 nucleotides, between about 300 to about 800 nucleotides, between about 300 to about 900 nucleotides, or about 1000 nucleotides in length, or even greater than about 1000 nucleotides in length, for example up to the entire length of a target gene including coding or non-coding or both coding and non-coding portions of the target gene). Where a polynucleotide is double-stranded, its length can be similarly described in terms of base pairs.

The polynucleotides described herein can be single-stranded (ss) or double-stranded (ds). “Double-stranded” refers to the base-pairing that occurs between sufficiently complementary, anti-parallel nucleic acid strands to form a double-stranded nucleic acid structure, generally under physiologically relevant conditions. Embodiments include those wherein the polynucleotide is selected from the group consisting of sense single-stranded DNA (ssDNA), sense single-stranded RNA (ssRNA), double-stranded RNA (dsRNA), double-stranded DNA (dsDNA), a double-stranded DNA/RNA hybrid, anti-sense ssDNA, or anti-sense ssRNA; a mixture of polynucleotides of any of these types can be used. In some embodiments, the polynucleotide is double-stranded RNA of a length greater than that which is typical of naturally occurring regulatory small RNAs (such as endogenously produced siRNAs and mature miRNAs). In some embodiments, the polynucleotide is double-stranded RNA of at least about 30 contiguous base-pairs in length. In some embodiments, the polynucleotide is double-stranded RNA with a length of between about 50 to about 500 base-pairs. In some embodiments, the polynucleotide can include components other than standard ribonucleotides, e.g., an embodiment is an RNA that comprises terminal deoxyribonucleotides.

Embodiments provide protection for crop plants against Lepidopteran pests. The crop plants include, but are not limited to, grain crop plants (such as wheat, oat, barley, maize, rye, triticale, rice, millet, sorghum, quinoa, amaranth, and buckwheat); forage crop plants (such as forage grasses and forage dicots including alfalfa, vetch, clover, and the like); oilseed crop plants (such as cotton, safflower, sunflower, soybean, canola, rapeseed, flax, peanuts, and oil palm); tree nuts (such as walnut, cashew, hazelnut, pecan, almond, and the like); sugarcane, coconut, date palm, olive, sugarbeet, tea, and coffee; wood- or pulp-producing trees; vegetable crop plants such as legumes (for example, beans, peas, lentils, alfalfa, peanut), lettuce, asparagus, artichoke, celery, carrot, radish, the brassicas (for example, cabbages, kales, mustards, and other leafy brassicas, broccoli, cauliflower, Brussels sprouts, turnip, kohlrabi), edible cucurbits (for example, cucumbers, melons, summer squashes, winter squashes), edible alliums (for example, onions, garlic, leeks, shallots, chives), edible members of the Solanaceae (for example, tomatoes, eggplants, potatoes, peppers, groundcherries), and edible members of the Chenopodiaceae (for example, beet, chard, spinach, quinoa, amaranth); fruit crop plants such as apple, pear, citrus fruits (for example, orange, lime, lemon, grapefruit, and others), stone fruits (for example, apricot, peach, plum, nectarine), banana, pineapple, grape, kiwifruit, papaya, avocado, and berries.

In various embodiments, the polynucleotide described herein comprise naturally occurring nucleotides, such as those which occur in DNA and RNA. In certain embodiments, the polynucleotide is a combination of ribonucleotides and deoxyribonucleotides, for example, synthetic polynucleotides consisting mainly of ribonucleotides but with one or more terminal deoxyribonucleotides or one or more terminal dideoxyribonucleotides or synthetic polynucleotides consisting mainly of deoxyribonucleotides but with one or more terminal dideoxyribonucleotides. In certain embodiments, the polynucleotide comprises non-canonical nucleotides such as inosine, thiouridine, or pseudouridine. In certain embodiments, the polynucleotide comprises chemically modified nucleotides. Examples of chemically modified oligonucleotides or polynucleotides are well known in the art; see, for example, U.S. Patent Publication 2011/0171287, U.S. Patent Publication 2011/0171176, U.S. Patent Publication 2011/0152353, U.S. Patent Publication 2011/0152346, and U.S. Patent Publication 2011/0160082, which are herein incorporated by reference. Illustrative examples include, but are not limited to, the naturally occurring phosphodiester backbone of an oligonucleotide or polynucleotide which can be partially or completely modified with phosphorothioate, phosphorodithioate, or methylphosphonate internucleotide linkage modifications, modified nucleoside bases or modified sugars can be used in oligonucleotide or polynucleotide synthesis, and oligonucleotides or polynucleotides can be labeled with a fluorescent moiety (e.g., fluorescein or rhodamine) or other label (e.g., biotin).

Several embodiments relate to a polynucleotide comprising at least one segment of 18 or more contiguous nucleotides with a sequence of about 95% to about 100% identity with a fragment of equivalent length of a DNA or target gene having a sequence selected from the Target Gene Sequences Group or the Trigger Sequences Group, or a RNA transcript of any thereof, or the DNA or RNA complement of any of the foregoing. In some embodiments, the contiguous nucleotides number at least 18, e.g., between 18-24, or between 18-28, or between 20-30, or between 20-50, or between 20-100, or between 50-100, or between 50-500, or between 100-250, or between 100-500, or between 200-1000, or between 500-2000, or even greater. In some embodiments, the contiguous nucleotides number more than 18, e.g., 19, 20, 21, 22, 23, 24, 25, 26, 27, 28, 29, 30, or greater than 30, e.g., at least about 35, at least about 40, at least about 45, at least about 50, at least about 55, at least about 60, at least about 65, at least about 70, at least about 75, at least about 80, at least about 85, at least about 90, at least about 95, at least about 100, at least about 110, at least about 120, at least about 130, at least about 140, at least about 150, at least about 160, at least about 170, at least about 180, at least about 190, at least about 200, at least about 210, at least about 220, at least about 230, at least about 240, at least about 250, at least about 260, at least about 270, at least about 280, at least about 290, at least about 300, at least about 350, at least about 400, at least about 450, at least about 500, or greater than 500 contiguous nucleotides. In some embodiments, the polynucleotide comprises at least one segment of at least 18, 19, 20, or 21 (reference to at least 18, 19, 20 or 21 as used throughout is intended to mean that any of these lower limits of the group can be individualized) contiguous nucleotides with a sequence of 100% identity with a fragment of equivalent length of a DNA or target gene having a sequence selected from the Target Gene Sequences Group or the Trigger Sequences Group, or the DNA or RNA complement of any of the foregoing. In some embodiments, the polynucleotide is a double-stranded nucleic acid (e.g., dsRNA) with one strand comprising at least one segment of at least 18, 19, 20, or 21 contiguous nucleotides with 100% identity with a fragment of equivalent length of a DNA or target gene having a sequence selected from the Target Gene Sequences Group or the Trigger Sequences Group, or the DNA or RNA complement of any of the foregoing; expressed as base-pairs, such a double-stranded nucleic acid comprises at least one segment of at least 18 contiguous, perfectly matched base-pairs which correspond to a fragment of equivalent length of a DNA or target gene having a sequence selected from the Target Gene Sequences Group or the Trigger Sequences Group, or the DNA or RNA complement of any of the foregoing. In some embodiments, each segment contained in the polynucleotide is of a length greater than that which is typical of naturally occurring regulatory small RNAs, for example, each segment is at least about 30 contiguous nucleotides (or base-pairs) in length. In some embodiments, the total length of the polynucleotide, or the length of each segment contained in the polynucleotide, is less than the total length of the DNA or target gene having a sequence selected from the Target Gene Sequences Group or the Trigger Sequences Group. In some embodiments, the total length of the polynucleotide is between about 50 to about 500 nucleotides (for single-stranded polynucleotides) or base-pairs (for double-stranded polynucleotides). In some embodiments, the polynucleotide is a dsRNA of between about 100 to about 500 base-pairs, such as a dsRNA of the length of any of the dsRNA triggers disclosed in Tables 1A, 1B and 1C. Embodiments include those in which the polynucleotide expressed in the plant is an RNA comprising a segment having a sequence selected from the group consisting of: SEQ ID NOs: 2, 5, 7, 9, 13, 21, 26, 27, 29, 36, 37, 39, 44, 47, 87, 95, 99, 103, 301, 309, 318, 319, 320, 321, 323, 324, 328, 341, 343, 346, 356, 358, 359, 503, 504, 505, 506, 509, 510, 530, 532, 533, 542, 545, 546, 547, 549, 551, 552, 553, 559, 560, 561, 563, 564, 570, 572, 573, 581, 593, 596, 602, 611, 612, 620, 665, 666, 672, 717, 730, 731, 736, 737, 738, 740, 741, 742, 743, 749, 757, 760, 761, 763, 766, 808, 809, 810, 814, 815, 817, 818, 821, 822, 1104, 1105, 1106, 1111, 1113, 1116, 1121, 1123, 1124, 1132, 1133, 1147, 1156, 1157, 1163, 1166, 1187, 1190, 1192, 1195, 1196, 1216, 1217, 1240, 1243, 1251, 1263, 1264, 1265, 1270, 1275, 1279, 1283, 1290, 1308, 1310, 1316, 1318, 1320, 1564, 1565, 1569, 1622, and 1623, or the complement thereof. In some embodiments, the polynucleotide is expressed in a plant. In some embodiments, the polynucleotide is topically provided to the surface of a plant or Lepidopteran pest.

Several embodiments relate to polynucleotides that are designed to modulate expression by inducing regulation or suppression of a Lepidopteran pest target gene. In some embodiments, the polynucleotides are designed to have a nucleotide sequence essentially identical or essentially complementary to the nucleotide sequence of a Lepidopteran pest target gene or cDNA (e.g., The Target Gene Sequences Group) or to the sequence of RNA transcribed from a Lepidopteran pest target gene, which can be coding sequence or non-coding sequence. These effective polynucleotide molecules that modulate expression may be referred to herein as a “polynucleotide”, “polynucleotide trigger”, “trigger”, or “triggers”.

Effective polynucleotides of any size can be used, alone or in combination, in the various methods and compositions described herein. In some embodiments, a single polynucleotide trigger is used to make a composition (e.g., a composition for topical application, or a recombinant DNA construct useful for making a transgenic plant). In other embodiments, a mixture or pool of different polynucleotide triggers is used; in such cases the polynucleotide triggers can be for a single target gene or for multiple target genes.

As used herein, the term “isolated” refers to separating a molecule from other molecules normally associated with it in its native or natural state. The term “isolated” thus may refer to a DNA molecule that has been separated from other DNA molecule(s) which normally are associated with it in its native or natural state. Such a DNA molecule may be present in a recombined state, such as a recombinant DNA molecule. Thus, DNA molecules fused to regulatory or coding sequences with which they are not normally associated, for example as the result of recombinant techniques, are considered isolated, even when integrated as a transgene into the chromosome of a cell or present with other DNA molecules.

As used herein, the term “Target Gene Sequences Group” refers to the group of sequences consisting of SEQ ID NOs: 1-114, 229-361, 495-511; 530-535, 542-822, 1104-1333, and 1564-1623. As used herein, the term “Trigger Sequences Group” refers to the group of sequences consisting of SEQ ID NOs: 115-228, 362-494, 512-529; 536-541, 823-1103, 1334-1563, and 1624-1683.

In various embodiments, the Lepidopteran pest is at least one insect selected from the group consisting of Spodoptera spp and Plutella spp. An example of a Spodoptera species includes S. frugiperda (fall armyworm). An example of a Plutella species includes P. xylostella (diamondback moth). The Lepidopteran pest may be at any stage of development.

Several embodiments relate to a polynucleotide designed to suppress one or more genes (“target genes”). The term “gene” refers to any portion of a nucleic acid that provides for expression of a transcript or encodes a transcript. A “gene” can include, but is not limited to, a promoter region, 5′ untranslated regions, transcript encoding regions that can include intronic regions, 3′ untranslated regions, or combinations of these regions. In some embodiments, the target genes can include coding or non-coding sequence or both. In other embodiments, the target gene has a sequence identical to or complementary to a messenger RNA, e.g., in some embodiments the target gene is a cDNA. In specific embodiments, the polynucleotide is designed to suppress one or more target genes, where each target gene is encoded by a DNA sequence selected from the Target Gene Sequences Group. In various embodiments, the polynucleotide is designed to suppress one or more target genes, where each target gene is encoded by a sequence selected from the Target Gene Sequences Group, and can be designed to suppress multiple target genes from this group, or to target different regions of one or more of these target genes. In an embodiment, the polynucleotide comprises multiple segments of 21 contiguous nucleotides with 100% identity with a fragment of equivalent length of a DNA or target gene having a sequence selected from the Target Gene Sequences Group or the Trigger Sequences Group, or an RNA transcript of any thereof, or the DNA or RNA complement any of the foregoing. In such cases, each segment can be identical or different in size or in sequence, and can be sense or anti-sense relative to the target gene. For example, in one embodiment the polynucleotide comprises multiple segments in tandem or repetitive arrangements, wherein each segment comprises 21 contiguous nucleotides with a sequence of 100% identity with a fragment of equivalent length of a DNA or target gene having a sequence selected from the Target Gene Sequences Group or Trigger Sequences Group, or the DNA or RNA complement of any of the foregoing. In some embodiments, the segments can be from different regions of the target gene, e.g., the segments can correspond to different exon regions of the target gene. In some embodiments, “spacer” nucleotides which do not correspond to a target gene can optionally be used in between or adjacent to the segments.

Other Definitions are provided in the sections below.

### II. Controlling Lepidopteran Infestations by Contacting with a Polynucleotide

Provided herein are methods for controlling a Lepidopteran pest infestation of a plant by contacting the Lepidopteran pest with a polynucleotide comprising at least one segment of 18 or more contiguous nucleotides having about 95% to about 100% identity or complementarity with a corresponding fragment of a DNA or target gene selected from the group consisting of: the Target Gene Sequences Group or the Trigger Sequences Group, or a RNA transcript of any thereof, or the DNA or RNA complement of any of the foregoing. In an embodiment, the method for controlling a Lepidopteran pest infestation of a plant comprises contacting the Lepidopteran pest with a polynucleotide comprising at least 18 contiguous nucleotides with 100% identity with a corresponding fragment of a target gene having a DNA sequence selected from the group consisting of: SEQ ID NOs: 2, 5, 7, 9, 13, 21, 26, 27, 29, 36, 37, 39, 44, 47, 87, 95, 99, 103, 301, 309, 318, 319, 320, 321, 323, 324, 328, 341, 343, 346, 356, 358, 359, 503, 504, 505, 506, 509, 510, 530, 532, 533, 542, 545, 546, 547, 549, 551, 552, 553, 559, 560, 561, 563, 564, 570, 572, 573, 581, 593, 596, 602, 611, 612, 620, 665, 666, 672, 717, 730, 731, 736, 737, 738, 740, 741, 742, 743, 749, 757, 760, 761, 763, 766, 808, 809, 810, 814, 815, 817, 818, 821, 822, 1104, 1105, 1106, 1111, 1113, 1116, 1121, 1123, 1124, 1132, 1133, 1147, 1156, 1157, 1163, 1166, 1187, 1190, 1192, 1195, 1196, 1216, 1217, 1240, 1243, 1251, 1263, 1264, 1265, 1270, 1275, 1279, 1283, 1290, 1308, 1310, 1316, 1318, 1320, 1564, 1565, 1569, 1622, and 1623, or the RNA transcript of any thereof, or the DNA or RNA complement of any of the foregoing. In some embodiments, the polynucleotide is a double-stranded RNA. In some embodiments, the polynucleotide (e.g., double-stranded RNA) is chemically or enzymatically synthesized or is produced by expression in a microorganism or by expression in a plant cell. Embodiments include those in which the polynucleotide is a dsRNA comprising a strand having a sequence selected from the Trigger Sequences Group. Polynucleotides of use in the method can be designed for multiple target genes. Related aspects of the invention include isolated polynucleotides of use in the method and plants having improved Lepidopteran resistance provided by the method. Specific embodiments include those in which the polynucleotide is a dsRNA comprising a sequence selected from the group consisting of SEQ ID NO: 115, 116, 119, 121, 123, 127, 135, 140, 141, 143, 150, 151, 153, 158, 161, 201, 209, 213, 217, 434, 442, 451, 452, 453, 454, 456, 457, 461, 474, 476, 479, 489, 491, 492, 521, 522, 523, 524, 527, 528, 536, 538, 539, 823, 826, 827, 828, 830, 832, 833, 834, 840, 841, 842, 844, 845, 851, 853, 854, 862, 874, 877, 883, 892, 893, 901, 946, 947, 953, 998, 1011, 1012, 1017, 1018, 1019, 1021, 1022, 1023, 1024, 1030, 1038, 1041, 1042, 1044, 1047, 1089, 1090, 1091, 1095, 1096, 1098, 1099, 1102, 1103, 1334, 1335, 1336, 1341, 1343, 1346, 1351, 1353, 1354, 1362, 1363, 1377, 1386, 1387, 1393, 1396, 1417, 1420, 1422, 1425, 1426, 1446, 1447, 1470, 1473, 1481, 1493, 1494, 1495, 1500, 1505, 1509, 1513, 1520, 1538, 1540, 1546, 1548, 1550, 1624, 1625, 1629, 1682, or 1683, or the complement thereof. Other specific embodiments include those in which the polynucleotide is a dsRNA comprising a sequence selected from the group consisting of to SEQ ID NOs: 434, 442, 451, 452, 453, 454, 456, 457, 461, 474, 476, 479, 489, 491, 492, 521, 522, 523, 524, 527, 528, 536, 538 and 539, or a combination thereof.

In some embodiments, the contiguous nucleotides have a sequence of about 95%, about 96%, about 97%, about 98%, about 99%, or about 100% identity with a fragment of equivalent length of a DNA or target gene having a sequence selected from the Target gene sequences group, or any RNA transcript thereof, or the DNA or RNA complement of any of the foregoing. In some embodiments, the contiguous nucleotides are exactly (100%) identical to a fragment of equivalent length of a DNA or target gene having a sequence selected from the Target Gene Sequences Group or the Trigger Sequences Group, or any RNA transcript thereof, or the DNA or RNA complement any of the foregoing. In some embodiments, the polynucleotide has an overall sequence of about 95%, about 96%, about 97%, about 98%, about 99%, or about 100% identity with a fragment of equivalent length of a DNA or target gene having a sequence selected from the Target Gene Sequences Group or the Trigger Sequences Group, or the DNA or RNA complement of any of the foregoing.

In an embodiment, the polynucleotide comprises at least one segment of 21 contiguous nucleotides with 100% identity with the corresponding fragment of a target gene having a DNA sequence selected from the group consisting of: SEQ ID NOs: 2, 5, 7, 9, 13, 21, 26, 27, 29, 36, 37, 39, 44, 47, 87, 95, 99, 103, 301, 309, 318, 319, 320, 321, 323, 324, 328, 341, 343, 346, 356, 358, 359, 503, 504, 505, 506, 509, 510, 530, 532, 533, 542, 545, 546, 547, 549, 551, 552, 553, 559, 560, 561, 563, 564, 570, 572, 573, 581, 593, 596, 602, 611, 612, 620, 665, 666, 672, 717, 730, 731, 736, 737, 738, 740, 741, 742, 743, 749, 757, 760, 761, 763, 766, 808, 809, 810, 814, 815, 817, 818, 821, 822, 1104, 1105, 1106, 1111, 1113, 1116, 1121, 1123, 1124, 1132, 1133, 1147, 1156, 1157, 1163, 1166, 1187, 1190, 1192, 1195, 1196, 1216, 1217, 1240, 1243, 1251, 1263, 1264, 1265, 1270, 1275, 1279, 1283, 1290, 1308, 1310, 1316, 1318, 1320, 1564, 1565, 1569, 1622, and 1623, or any RNA transcript thereof, or the DNA or RNA complement of any of the foregoing. In some embodiments, the polynucleotide comprises “neutral” sequence (sequence having no sequence identity or complementarity to the target gene) in addition to one or more segments of 21 contiguous nucleotides with 100% identity with the corresponding fragment of the target gene, and therefore the polynucleotide as a whole is of much lower overall sequence identity with a target gene.

The total length of the polynucleotide of use in this method can be greater than 18 contiguous nucleotides, and can include nucleotides in addition to the contiguous nucleotides having the sequence of about 95% to about 100% identity with a fragment of equivalent length of a DNA or target gene having a sequence selected from the group consisting of: the Target Gene Sequences Group or the Trigger Sequences Group, or any RNA transcript thereof, or the DNA or RNA complement of any of the foregoing. In other words, the total length of the polynucleotide can be greater than the length of the section or segment of the polynucleotide designed to suppress one or more target genes, where each target gene has a DNA sequence selected from the group consisting of the Target Gene Sequences Group. For example, the polynucleotide can have nucleotides flanking the “active” segment of at least one segment of 18 or more contiguous nucleotides that suppresses the target gene, or include “spacer” nucleotides between active segments, or can have additional nucleotides at the 5′ end, or at the 3′ end, or at both the 5′ and 3′ ends. In an embodiment, the polynucleotide can include additional nucleotides that are not specifically related (having a sequence not complementary or identical to) to the DNA or target gene having a sequence selected from the group consisting of: the Target Gene Sequences Group or the Trigger Sequences Group, or any RNA transcript thereof, or the DNA or RNA complement thereof, e.g., nucleotides that provide stabilizing secondary structure or for convenience in cloning or manufacturing. In an embodiment, the polynucleotide can include additional nucleotides located immediately adjacent to one or more segment of 18 or more contiguous nucleotides with a sequence of about 95% to about 100% identity with a fragment of equivalent length of a sequence selected from the group consisting of: the Target Gene Sequences Group or the Trigger Sequences Group, or any RNA transcript thereof, or the DNA or RNA complement of any of the foregoing. In an embodiment, the polynucleotide comprises one such segment, with an additional 5′ G or an additional 3′ C or both, adjacent to the segment. In another embodiment, the polynucleotide is a double-stranded RNA comprising additional nucleotides to form an overhang, for example, a dsRNA comprising 2 deoxyribonucleotides to form a 3′ overhang. Thus in various embodiments, the nucleotide sequence of the entire polynucleotide is not 100% identical or complementary to a sequence of contiguous nucleotides in the DNA or target gene having a sequence selected the Target Gene Sequences Group, the Trigger Sequences Group, or the DNA complement of any thereof. For example, in some embodiments the polynucleotide comprises at least two segments each of 21 contiguous nucleotides with a sequence of 100% identity with a fragment of a DNA having a sequence selected from the Target Gene Sequences Group or the Trigger Sequences Group, or the DNA complement of any of the foregoing, wherein (1) the at least two segments are separated by one or more spacer nucleotides, or (2) the at least two segments are arranged in an order different from that in which the corresponding fragments occur in the DNA having a sequence selected from the group consisting of: the Target Gene Sequences Group or the Trigger Sequences Group, or the DNA complement of any of the foregoing.

The polynucleotide of use in this method is provided by suitable means known to one in the art. Embodiments include those wherein the polynucleotide is chemically or enzymatically synthesized (e.g., by in vitro transcription, such as transcription using a T7 polymerase or other polymerase), produced by expression in a microorganism or in cell culture (such as plant or insect cells grown in culture), produced by expression in a plant cell, or produced by microbial fermentation.

In some embodiments the polynucleotide of use in this method is provided as an isolated DNA or RNA fragment. In some embodiments the polynucleotide of use in this method is not part of an expression construct and is lacking additional elements such as a promoter or terminator sequences). Such polynucleotides can be relatively short, such as single- or double-stranded polynucleotides of between about 18 to about 300 or between about 50 to about 500 nucleotides (for single-stranded polynucleotides) or between about 18 to about 300 or between about 50 to about 500 base-pairs (for double-stranded polynucleotides). In some embodiments, the polynucleotide is a dsRNA of between about 100 to about 500 base-pairs, such as a dsRNA of the length of any of the dsRNA triggers disclosed in Tables 1A, 1B and 1C. Alternatively the polynucleotide can be provided in more complex constructs, e.g., as part of a recombinant expression construct, or included in a recombinant vector, for example in a recombinant plant virus vector or in a recombinant baculovirus vector. In some embodiments such recombinant expression constructs or vectors are designed to include additional elements, such as expression cassettes for expressing a gene of interest (e.g., an insecticidal protein).

Several embodiments relate to a method for controlling a Lepidopteran pest infestation of a plant comprising contacting the Lepidopteran pest with a polynucleotide comprising at least one segment of 18 or more contiguous nucleotides that is essentially identical or complementary to a fragment of equivalent length of a DNA of a target gene selected from the group consisting of the genes identified in the Target Gene Sequences Group. In some embodiments the polynucleotide comprises a dsRNA with a strand having a sequence selected from the group consisting of the Trigger Sequences Group. In some embodiments, this invention provides a method for controlling a Lepidopteran pest infestation of a plant comprising contacting the Lepidopteran pest with an effective amount of a solution comprising a double-stranded RNA from the Trigger Sequences Group, the solution further comprises one or more components selected from the group consisting of an organosilicone surfactant or a cationic lipid.

In various embodiments of the method, the contacting comprises application to a surface of the Lepidopteran pest of a suitable composition comprising the polynucleotide of use in this method; such a composition can be provided, e.g., as a solid, liquid (including homogeneous mixtures such as solutions and non-homogeneous mixtures such as suspensions, colloids, micelles, and emulsions), powder, suspension, emulsion, spray, encapsulated or micro-encapsulation formulation, in or on microbeads or other carrier particulates, in a film or coating, or on or within a matrix, or as a seed treatment. The contacting can be in the form of a seed treatment, or in the form of treatment of “seed potato” tubers or pieces of tuber (e.g., by soaking, coating, or dusting the seed potato). Suitable binders, inert carriers, surfactants, and the like can optionally be included in the composition, as is known to one skilled in formulation of pesticides and seed treatments. In some embodiments, the contacting comprises providing the polynucleotide in a composition that further comprises one or more components selected from the group consisting of a carrier agent, a surfactant, a cationic lipid (such as that disclosed in Example 18 of U.S. patent application publication 2011/0296556, incorporated by reference herein), an organosilicone, an organosilicone surfactant, a polynucleotide herbicidal molecule, a non-polynucleotide herbicidal molecule, a non-polynucleotide pesticide, a safener, and an insect growth regulator. In some embodiments, the contacting comprises providing the polynucleotide in a composition that further comprises at least one pesticidal agent selected from the group consisting of a patatin, a plant lectin, a phytoecdysteroid, a Bacillus thuringiensis insecticidal protein, a Xenorhabdus insecticidal protein, a Photorhabdus insecticidal protein, a Bacillus laterosporus insecticidal protein, and a Bacillus sphaericus insecticidal protein. In one embodiment the contacting comprises providing the polynucleotide in a composition that can be ingested or otherwise absorbed internally by the Lepidopteran pest.

It is anticipated that the combination of certain polynucleotides of use in this method (e.g., the polynucleotide triggers described in the working Examples) with one or more non-polynucleotide pesticidal agents will result in an enhanced improvement in prevention or control of Lepidopteran pest infestations, when compared to the effect obtained with the polynucleotide alone or the non-polynucleotide pesticidal agent alone. In an embodiment, a composition containing one or more polynucleotides and one or more non-polynucleotide pesticidal agent selected from the group consisting of a patatin, a plant lectin, a phytoecdysteroid, a Bacillus thuringiensis insecticidal protein, a Xenorhabdus insecticidal protein, a Photorhabdus insecticidal protein, a Bacillus laterosporus insecticidal protein, and a Bacillus sphaericus insecticidal protein, is found to effect improved prevention or control of Lepidopteran pest infestations.

### III. Controlling Lepidopteran Infestations by Providing a Dietary Polynucleotide

Another aspect of this invention provides a method for controlling a Lepidopteran pest infestation of a plant comprising providing in the diet of a Lepidopteran pest an agent comprising a polynucleotide having at least one segment of 18 or more contiguous nucleotides with a sequence of about 95% to about 100% identity with a fragment of equivalent length of a DNA having a sequence selected from the group consisting of: The Target Gene Sequences Group, the Trigger Sequences Group, or the DNA complement thereof, wherein the agent functions upon ingestion by the Lepidopteran pest to inhibit a biological function within the Lepidopteran pest thereby controlling infestation by the Lepidopteran pest. The polynucleotide can be longer than the segment or segments it contains, but each polynucleotide segment and corresponding DNA fragment are of equivalent length. Polynucleotides of use in the method can be designed for multiple target genes. Embodiments include those in which the agent comprises a dsRNA comprising a strand having a sequence selected from the Trigger Sequences group, or the complement thereof, or wherein the agent comprises a polynucleotide or RNA encoded by a sequence selected from the group consisting of SEQ ID NOs: SEQ ID NO: 115, 116, 119, 121, 123, 127, 135, 140, 141, 143, 150, 151, 153, 158, 161, 201, 209, 213, 217, 434, 442, 451, 452, 453, 454, 456, 457, 461, 474, 476, 479, 489, 491, 492, 521, 522, 523, 524, 527, 528, 536, 538, 539, 823, 826, 827, 828, 830, 832, 833, 834, 840, 841, 842, 844, 845, 851, 853, 854, 862, 874, 877, 883, 892, 893, 901, 946, 947, 953, 998, 1011, 1012, 1017, 1018, 1019, 1021, 1022, 1023, 1024, 1030, 1038, 1041, 1042, 1044, 1047, 1089, 1090, 1091, 1095, 1096, 1098, 1099, 1102, 1103, 1334, 1335, 1336, 1341, 1343, 1346, 1351, 1353, 1354, 1362, 1363, 1377, 1386, 1387, 1393, 1396, 1417, 1420, 1422, 1425, 1426, 1446, 1447, 1470, 1473, 1481, 1493, 1494, 1495, 1500, 1505, 1509, 1513, 1520, 1538, 1540, 1546, 1548, 1550, 1624, 1625, 1629, 1682, or 1683. In another embodiment, the agent comprises a polynucleotide or RNA encoded by a sequence selected from the group consisting of SEQ ID NOs: 434, 442, 451, 452, 453, 454, 456, 457, 461, 474, 476, 479, 489, 491, 492, 521, 522, 523, 524, 527, 528, 536, 538 and 539, or a combination thereof. In an embodiment, a method for controlling a Lepidopteran pest infestation of a plant comprising providing in the diet of the Lepidopteran pest a polynucleotide comprising a nucleotide sequence that is complementary to at least 18 contiguous nucleotides of a target gene having a nucleotide sequence selected from the group consisting of: SEQ ID NOs: 2, 5, 7, 9, 13, 21, 26, 27, 29, 36, 37, 39, 44, 47, 87, 95, 99, 103, 301, 309, 318, 319, 320, 321, 323, 324, 328, 341, 343, 346, 356, 358, 359, 503, 504, 505, 506, 509, 510, 530, 532, 533, 542, 545, 546, 547, 549, 551, 552, 553, 559, 560, 561, 563, 564, 570, 572, 573, 581, 593, 596, 602, 611, 612, 620, 665, 666, 672, 717, 730, 731, 736, 737, 738, 740, 741, 742, 743, 749, 757, 760, 761, 763, 766, 808, 809, 810, 814, 815, 817, 818, 821, 822, 1104, 1105, 1106, 1111, 1113, 1116, 1121, 1123, 1124, 1132, 1133, 1147, 1156, 1157, 1163, 1166, 1187, 1190, 1192, 1195, 1196, 1216, 1217, 1240, 1243, 1251, 1263, 1264, 1265, 1270, 1275, 1279, 1283, 1290, 1308, 1310, 1316, 1318, 1320, 1564, 1565, 1569, 1622, and 1623, or an RNA transcribed from the target gene is provided. In some embodiments, the polynucleotide is a double-stranded RNA. In some embodiments, the polynucleotide (e.g., double-stranded RNA) is chemically or enzymatically synthesized or is produced by expression in a microorganism or by expression in a plant cell. Related aspects of the invention include isolated polynucleotides of use in the method and plants having improved Lepidopteran resistance provided by the method.

In various embodiments, the agent comprising a polynucleotide comprises a microbial cell or is produced in a microorganism. For example, the agent can include or can be produced in bacteria or yeast cells. In other embodiments the agent comprising a polynucleotide comprises a transgenic plant cell or is produced in a plant cell (for example a plant cell transiently expressing the polynucleotide); such plant cells can be cells in an plant or cells grown in tissue culture or in cell suspension.

In various embodiments, the agent comprising a polynucleotide is provided for dietary uptake by the Lepidopteran pest in a form suitable for ingestion, for example, as a solid, liquid (including homogeneous mixtures such as solutions and non-homogeneous mixtures such as suspensions, colloids, micelles, and emulsions), powder, suspension, emulsion, spray, encapsulated or micro-encapsulation formulation, in or on microbeads or other carrier particulates, in a film or coating, or on or within a matrix, or as a seed treatment. The agent comprising a polynucleotide can be provided for dietary uptake by the Lepidopteran pest by applying the agent to a plant subject to infestation by the Lepidopteran pest or by applying the agent to seed of the plant, for example by spraying, dusting, or coating the plant, or by application of a soil drench, or by providing in an artificial diet. The agent comprising a polynucleotide can be provided for dietary uptake by the Lepidopteran pest in an artificial diet formulated to meet the particular nutritional requirements for maintaining the Lepidopteran pest, wherein the artificial diet is supplemented with some amount of the polynucleotide obtained from a separate source such as chemical synthesis or purified from a microbial fermentation; this embodiment can be useful, e.g., for determining the timing and amounts of effective polynucleotide treatment regimes. In some embodiments the agent comprising a polynucleotide is provided for dietary uptake by the Lepidopteran pest in the form of a plant cell or in plant cell components, or in a microorganism (such as a bacterium or a yeast) or a microbial fermentation product, or in a synthetic or man-made diet. In one embodiment the agent comprising a polynucleotide is provided in the form of bait that is ingested by the Lepidopteran pest. The agent comprising a polynucleotide can be provided for dietary uptake by the Lepidopteran pest in the form of a seed treatment, or in the form of treatment of “seed potato” tubers or pieces of tuber (e.g., by soaking, coating, or dusting the seed potato). Suitable binders, inert carriers, surfactants, and the like can be included in the agent, as is known to one skilled in formulation of pesticides and seed treatments. In some embodiments, the agent comprising a polynucleotide further comprises one or more components selected from the group consisting of a carrier agent, a surfactant, a cationic lipid (such as that disclosed in Example 18 of U.S. patent application publication 2011/0296556, incorporated by reference herein), an organosilicone, an organosilicone surfactant, a polynucleotide herbicidal molecule, a non-polynucleotide herbicidal molecule, a non-polynucleotide pesticide, a safener, and an insect growth regulator. In some embodiments, the agent comprising a polynucleotide further comprises at least one pesticidal agent selected from the group consisting of a patatin, a plant lectin, a phytoecdysteroid, a phytoecdysteroid, a Bacillus thuringiensis insecticidal protein, a Xenorhabdus insecticidal protein, a Photorhabdus insecticidal protein, a Bacillus laterosporus insecticidal protein, and a Bacillus sphaericus insecticidal protein. Other proteins include plant-derived proteins described in Toxins (Basel). 2019 Jul. 1; 11(7). In some embodiments, the agent comprising a polynucleotide comprises at least one implantable formulation selected from the group consisting of a particulate, pellet, or capsule implanted in the plant; in such embodiments the method comprises implanting in the plant the implantable formulation. In some embodiments, the agent comprising a polynucleotide comprises at least one in-furrow formulation selected from the group consisting of a powder, granule, pellet, capsule, spray, or drench, or any other forms suited for applying to a furrow; in such embodiments, the method comprises an in-furrow treatment with the in-furrow formulation. In some embodiments, the method comprises treatment of a solanaceous plant seed, potato tuber, or piece of potato tuber with the agent.

It is anticipated that the combination of certain polynucleotides of use in agents of use in this method (e.g., the polynucleotide triggers described in the working Examples) with one or more non-polynucleotide pesticidal agents will result in an enhanced improvement in prevention or control of Lepidopteran pest infestations, when compared to the effect obtained with the polynucleotide alone or the non-polynucleotide pesticidal agent alone. In an embodiment, a composition containing one or more polynucleotides and one or more non-polynucleotide pesticidal agent selected from the group consisting of a patatin, a plant lectin, a phytoecdysteroid, a Bacillus thuringiensis insecticidal protein, a Xenorhabdus insecticidal protein, a Photorhabdus insecticidal protein, a Bacillus laterosporus insecticidal protein, and a Bacillus sphaericus insecticidal protein, is found to effect improved prevention or control of Lepidopteran pest infestations when provided to the Lepidopteran pest in a diet.

In some embodiments, the polynucleotide used in this method is a dsRNA comprising a segment having a sequence selected from the Trigger sequences group or the complement thereof, or wherein the polynucleotide is encoded by a sequence selected from the group consisting of SEQ ID NO: 115, 116, 119, 121, 123, 127, 135, 140, 141, 143, 150, 151, 153, 158, 161, 201, 209, 213, 217, 434, 442, 451, 452, 453, 454, 456, 457, 461, 474, 476, 479, 489, 491, 492, 521, 522, 523, 524, 527, 528, 536, 538, 539, 823, 826, 827, 828, 830, 832, 833, 834, 840, 841, 842, 844, 845, 851, 853, 854, 862, 874, 877, 883, 892, 893, 901, 946, 947, 953, 998, 1011, 1012, 1017, 1018, 1019, 1021, 1022, 1023, 1024, 1030, 1038, 1041, 1042, 1044, 1047, 1089, 1090, 1091, 1095, 1096, 1098, 1099, 1102, 1103, 1334, 1335, 1336, 1341, 1343, 1346, 1351, 1353, 1354, 1362, 1363, 1377, 1386, 1387, 1393, 1396, 1417, 1420, 1422, 1425, 1426, 1446, 1447, 1470, 1473, 1481, 1493, 1494, 1495, 1500, 1505, 1509, 1513, 1520, 1538, 1540, 1546, 1548, 1550, 1624, 1625, 1629, 1682, or 1683. In another embodiment, the agent comprises a polynucleotide or RNA encoded by a sequence selected from the group consisting of SEQ ID NOs: 434, 442, 451, 452, 453, 454, 456, 457, 461, 474, 476, 479, 489, 491, 492, 521, 522, 523, 524, 527, 528, 536, 538 and 539, or a combination thereof.

In some embodiments, the contiguous nucleotides have a sequence of at least about 95%, about 96%, about 97%, about 98%, about 99%, or about 100% identity with a fragment of equivalent length of a DNA or target gene having a sequence selected from The Target Gene Sequences Group or the Trigger Sequences Group, or any RNA transcript thereof, or the DNA or RNA complement of any of the foregoing. In some embodiments the contiguous nucleotides are exactly (100%) identical to a fragment of equivalent length of a DNA or target gene having a sequence selected from The Target Gene Sequences Group or the Trigger Sequences Group, or any RNA transcript thereof, or the DNA or RNA complement of any of the foregoing. In some embodiments, the polynucleotide has an overall sequence of at least about 95%, about 96%, about 97%, about 98%, about 99%, or about 100% identity with a fragment of equivalent length of a DNA or target gene having a sequence selected from The Target Gene Sequences Group or the Trigger Sequences Group, or any RNA transcript thereof, or the DNA or RNA complement of any of the foregoing. In an embodiment, the polynucleotide comprises at least one segment of 21 contiguous nucleotides with a sequence of 100% identity with the corresponding fragment of a target gene having a DNA sequence selected from the group consisting of: SEQ ID NOs: 2, 5, 7, 9, 13, 21, 26, 27, 29, 36, 37, 39, 44, 47, 87, 95, 99, 103, 301, 309, 318, 319, 320, 321, 323, 324, 328, 341, 343, 346, 356, 358, 359, 503, 504, 505, 506, 509, 510, 530, 532, 533, 542, 545, 546, 547, 549, 551, 552, 553, 559, 560, 561, 563, 564, 570, 572, 573, 581, 593, 596, 602, 611, 612, 620, 665, 666, 672, 717, 730, 731, 736, 737, 738, 740, 741, 742, 743, 749, 757, 760, 761, 763, 766, 808, 809, 810, 814, 815, 817, 818, 821, 822, 1104, 1105, 1106, 1111, 1113, 1116, 1121, 1123, 1124, 1132, 1133, 1147, 1156, 1157, 1163, 1166, 1187, 1190, 1192, 1195, 1196, 1216, 1217, 1240, 1243, 1251, 1263, 1264, 1265, 1270, 1275, 1279, 1283, 1290, 1308, 1310, 1316, 1318, 1320, 1564, 1565, 1569, 1622, and 1623, or the DNA complement thereof; in some embodiments, the polynucleotide comprises “neutral” sequence (having no sequence identity or complementarity to the target gene) in addition to a segment of 21 contiguous nucleotides with 100% identity with the corresponding fragment of the target gene, and therefore the polynucleotide as a whole is of much lower overall sequence identity with a target gene.

The polynucleotide of use in this method is generally designed to suppress one or more genes (“target genes”). In other embodiments, the target gene has a sequence identical to or complementary to a messenger RNA, e.g., in some embodiments the target gene is a cDNA. In specific embodiments, the polynucleotide is designed to suppress one or more target genes, where each target gene has a DNA sequence selected from the group consisting of the Target Gene Sequences Group. In various embodiments, the polynucleotide is designed to suppress one or more target genes, where each target gene has a sequence selected from the group consisting of the Target Gene Sequences Group, and can be designed to suppress multiple target genes from this group, or to target different regions of one or more of these target genes. In an embodiment, the polynucleotide comprises multiple segments of 21 contiguous nucleotides with a sequence of 100% identity with a fragment of equivalent length of a DNA or target gene having a sequence selected from The Target Gene Sequences Group or the Trigger Sequences Group, or any RNA transcript thereof, or the DNA or RNA complement of any of the foregoing. In such cases, each segment can be identical or different in size or in sequence, and can be sense or anti-sense relative to the target gene. For example, in one embodiment the polynucleotide comprises multiple segments in tandem or repetitive arrangements, wherein each segment comprises 21 contiguous nucleotides with a sequence of 100% identity with a fragment of equivalent length of a DNA or target gene having a sequence selected from The Target Gene Sequences Group or the Trigger Sequences Group, or any RNA transcript thereof, or the DNA or RNA complement of any of the foregoing; the segments can be from different regions of the target gene, e.g., the segments can correspond to different exon regions of the target gene, and “spacer” nucleotides which do not correspond to a target gene can optionally be used in between or adjacent to the segments.

The total length of the polynucleotide of use in this method can be greater than 18 contiguous nucleotides, and can include nucleotides in addition to the contiguous nucleotides having the sequence of about 95% to about 100% identity with a fragment of equivalent length of a DNA or target gene having a sequence selected from The Target Gene Sequences Group or the Trigger Sequences Group, or any RNA transcript thereof, or the DNA or RNA complement of any of the foregoing. In other words, the total length of the polynucleotide can be greater than the length of the section or segment of the polynucleotide designed to suppress one or more target genes, where each target gene has a DNA sequence selected from the group consisting of the Target Gene Sequences Group or the Trigger Sequences Group. For example, the polynucleotide can have nucleotides flanking the “active” segment of at least one segment of 18 or more contiguous nucleotides that suppresses the target gene, or include “spacer” nucleotides between active segments, or can have additional nucleotides at the 5′ end, or at the 3′ end, or at both the 5′ and 3′ ends. In an embodiment, the polynucleotide can include additional nucleotides that are not specifically related (having a sequence not complementary or identical to) to the DNA or target gene having a sequence selected from the Target Gene Sequences Group, the Trigger Sequences Group, or the DNA complement of any thereof, e.g., nucleotides that provide stabilizing secondary structure or for convenience in cloning or manufacturing. In an embodiment, the polynucleotide can include additional nucleotides located immediately adjacent to one or more segment of 18 or more contiguous nucleotides with a sequence of about 95% to about 100% identity with a fragment of equivalent length of a DNA or target gene having a sequence selected from the Target Gene Sequences Group, the Trigger Sequences Group, or the DNA complement of any thereof. In an embodiment, the polynucleotide comprises one such segment, with an additional 5′ G or an additional 3′ C or both, adjacent to the segment. In another embodiment, the polynucleotide is a double-stranded RNA comprising additional nucleotides to form an overhang, for example, a dsRNA comprising 2 deoxyribonucleotides to form a 3′ overhang. Thus in various embodiments, the nucleotide sequence of the entire polynucleotide is not 100% identical or complementary to a sequence of contiguous nucleotides in the DNA or target gene having a sequence selected from The Target Gene Sequences Group, the Trigger Sequences Group, or the DNA complement of any thereof. For example, in some embodiments the polynucleotide comprises at least two segments of 21 contiguous nucleotides with a sequence of 100% identity with a fragment of a DNA having a sequence selected from The Target Gene Sequences Group, the Trigger Sequences Group, or the DNA complement of any thereof, wherein (1) the at least two segments are separated by one or more spacer nucleotides, or (2) the at least two segments are arranged in an order different from that in which the corresponding fragments occur in the DNA having a sequence selected from The Target Gene Sequences Group, the Trigger Sequences Group, or the DNA complement of any thereof.

The polynucleotide of use in this method is provided by suitable means known to one in the art. Embodiments include those wherein the polynucleotide is chemically or enzymatically synthesized (e.g., by in vitro transcription, such as transcription using a T7 polymerase or other polymerase), produced by expression in a microorganism or in cell culture (such as plant or insect cells grown in culture), produced by expression in a plant cell, or produced by microbial fermentation.

In some embodiments the polynucleotide of use in this method is provided as an isolated DNA or RNA fragment. In some embodiments the polynucleotide of use in this method is not part of an expression construct and is lacking additional elements such as a promoter or terminator sequences. Such polynucleotides can be relatively short, such as single- or double-stranded polynucleotides of between about 18 to about 300 or between about 50 to about 500 nucleotides (for single-stranded polynucleotides) or between about 18 to about 300 or between about 50 to about 500 base-pairs (for double-stranded polynucleotides). In some embodiments, the polynucleotide is a dsRNA of between about 100 to about 500 base-pairs, such as a dsRNA of the length of any of the dsRNA triggers disclosed in Tables 1A, 1B and 1C. Alternatively the polynucleotide can be provided in more complex constructs, e.g., as part of a recombinant expression construct, or included in a recombinant vector, for example in a recombinant plant virus vector or in a recombinant baculovirus vector. In some embodiments such recombinant expression constructs or vectors are designed to include additional elements, such as expression cassettes for expressing a gene of interest (e.g., an insecticidal protein).

### IV. Controlling Lepidopteran Infestations by Providing a Dietary RNA

Another aspect of this invention provides a method of causing mortality or stunting in larvae of the Lepidopteran pest by providing in the diet of the larvae at least one polynucleotide comprising at least one silencing element comprising at least 18, 19, 20, or 21 contiguous nucleotides that are complementary to a target gene having a nucleotide sequence selected from the Target Gene Sequences Group, or an RNA transcribed from the target gene. In some embodiments, the polynucleotide is a double-stranded RNA. In some embodiments, the polynucleotide (e.g., double-stranded RNA) is chemically or enzymatically synthesized or is produced by expression in a microorganism or by expression in a plant cell. In an embodiment, a method of causing mortality or stunting in Lepidopteran pest larvae comprising providing in the diet of Lepidopteran pest larvae at least one RNA comprising at least one silencing element essentially identical or essentially complementary to a fragment of a target gene sequence of the Lepidopteran pest larvae, wherein the target gene sequence is selected from the Target Gene Sequences Group, and wherein ingestion of the RNA by the Lepidopteran pest larvae results in mortality or stunting in the Lepidopteran pest larvae is provided. A related aspect of this invention is an RNA comprising at least one silencing element, wherein the at least one silencing element is essentially identical or essentially complementary to a fragment of a target gene of the Lepidopteran pest larvae, wherein the target gene sequence is selected from the Target Gene Sequences Group. The RNA can be longer than the silencing element or silencing elements it contains, but each silencing element and corresponding fragment of a target gene sequence are of equivalent length. RNAs of use in the method can be designed for multiple target genes. Embodiments include those in which the RNA comprises a dsRNA with a strand having a sequence selected from the Trigger Sequences Group. In a related aspect, a method of causing mortality or lower fecundity in Lepidopteran pest comprising providing in the diet of Lepidopteran pest at least one RNA comprising at least one silencing element essentially identical or essentially complementary to a fragment of a target gene sequence of the Lepidopteran pest larvae, wherein the target gene sequence is selected from The Target Gene Sequences Group, the Trigger Sequences Group, or the DNA complement of any thereof, and wherein ingestion of the RNA by the Lepidopteran pest results in mortality or lower fecundity in the Lepidopteran pest is provided. Related aspects of the invention include isolated RNAs of use in the method and plants having improved Lepidopteran resistance provided by the method.

In various embodiments, the diet providing the RNA comprises a microbial cell or is produced in a microorganism. For example, the diet providing the RNA can include or can be produced in bacteria or yeast cells. In similar embodiments the diet providing the RNA comprises a transgenic plant cell or is produced in a plant cell (for example a plant cell transiently expressing the polynucleotide); such plant cells can be cells in an plant or cells grown in tissue culture or in cell suspension.

In one embodiment the diet providing the RNA is provided in the form of any plant that is subject to infestation by a Lepidopteran pest, wherein the RNA is contained in or on the plant. Such plants can be stably transgenic plants that express the RNA, or non-transgenic plants that transiently express the RNA or that have been treated with the RNA, e.g., by spraying or coating. Stably transgenic plants generally contain integrated into their genome a recombinant construct that encodes the RNA. Of particular interest are embodiments wherein the plant is a crop plant.

In various embodiments, the diet providing the RNA is provided in a form suitable for ingestion by the Lepidopteran pest, for example, as a solid, liquid (including homogeneous mixtures such as solutions and non-homogeneous mixtures such as suspensions, colloids, micelles, and emulsions), powder, suspension, emulsion, spray, encapsulated or micro-encapsulation formulation, in or on microbeads or other carrier particulates, in a film or coating, or on or within a matrix, or as a seed treatment. The diet providing the RNA can be provided by applying the diet to a plant subject to infestation by the Lepidopteran pest, for example by spraying, dusting, or coating the plant, or by application of a soil drench, or by providing in an artificial diet. In one embodiment the diet providing the recombinant RNA is provided in the form of bait that is ingested by the Lepidopteran pest. The diet providing the RNA can be an artificial diet formulated to meet the particular nutritional requirements for maintaining the Lepidopteran pest, wherein the artificial diet is supplemented with some amount of the RNA obtained from a separate source such as chemical synthesis or purified from a microbial fermentation; this embodiment can be useful, e.g., for determining the timing and amounts of effective polynucleotide treatment regimes. In some embodiments the diet providing the RNA is provided in the form of a plant cell or in plant cell components, or in a microorganism (such as a bacterium or a yeast) or a microbial fermentation product, or in a synthetic diet. In one embodiment the diet providing the RNA is provided in the form of bait that is ingested by the Lepidopteran pest. The diet providing the RNA can be provided in the form of a seed treatment, or in the form of treatment of “seed potato” tubers or pieces of tuber (e.g., by soaking, coating, or dusting the seed potato). Suitable binders, inert carriers, surfactants, and the like can be included in the diet, as is known to one skilled in formulation of pesticides and seed treatments. In some embodiments, the diet providing the RNA further comprises one or more components selected from the group consisting of a carrier agent, a surfactant, a cationic lipid (such as that disclosed in Example 18 of U.S. patent application publication 2011/0296556, incorporated by reference herein), an organosilicone, an organosilicone surfactant, a polynucleotide herbicidal molecule, a non-polynucleotide herbicidal molecule, a non-polynucleotide pesticide, a safener, and an insect growth regulator. In some embodiments, the diet providing the RNA further comprises at least one pesticidal agent selected from the group consisting of a patatin, a plant lectin, a phytoecdysteroid, a Bacillus thuringiensis insecticidal protein, a Xenorhabdus insecticidal protein, a Photorhabdus insecticidal protein, a Bacillus laterosporus insecticidal protein, and a Bacillus sphaericus insecticidal protein. In some embodiments, the diet providing the RNA includes at least one implantable formulation selected from the group consisting of a particulate, pellet, or capsule implanted in the plant; in such embodiments the method comprises implanting in the plant the implantable formulation. In some embodiments, the diet providing the RNA includes at least one in-furrow formulation selected from the group consisting of a powder, granule, pellet, capsule, spray, or drench, or any other forms suited for applying to a furrow; in such embodiments, the method includes an in-furrow treatment with the in-furrow formulation. In some embodiments, the method comprises treatment of a solanaceous plant seed, potato tuber, or piece of potato tuber with the agent.

It is anticipated that the combination of certain RNAs of use in this method (e.g., the dsRNA triggers described in the working Examples) with one or more non-polynucleotide pesticidal agents will result in an enhanced improvement in prevention or control of Lepidopteran pest infestations, when compared to the effect obtained with the RNA alone or the non-polynucleotide pesticidal agent alone. In an embodiment, a composition containing one or more RNAs and one or more non-polynucleotide pesticidal agent selected from the group consisting of a patatin, a plant lectin, a phytoecdysteroid, a Bacillus thuringiensis insecticidal protein, a Xenorhabdus insecticidal protein, a Photorhabdus insecticidal protein, a Bacillus laterosporus insecticidal protein, and a Bacillus sphaericus insecticidal protein, is found to effect improved prevention or control of Lepidopteran pest infestations.

The RNA of use in this method can be single-stranded (ss) or double-stranded (ds). Embodiments of the method include those wherein the RNA is at least one selected from the group consisting of sense single-stranded RNA (ssRNA), anti-sense single-stranded (ssRNA), or double-stranded RNA (dsRNA); a mixture of RNAs of any of these types can be used. In one embodiment a double-stranded DNA/RNA hybrid is used. The RNA can include components other than standard ribonucleotides, e.g., an embodiment is an RNA that comprises terminal deoxyribonucleotides.

The RNA comprises at least one silencing element, wherein the silencing element is essentially identical (as the RNA equivalent) or essentially complementary to a fragment of a target gene of the Lepidopteran pest larvae, wherein the target gene sequence is selected from the Target Gene Sequences Group. In some embodiments, the silencing element has a sequence of at least about 95%, about 96%, about 97%, about 98%, about 99%, or about 100% identity with or complementarity to a fragment of equivalent length of a DNA having a sequence selected from the Target Gene Sequences Group. In some embodiments the silencing element is exactly (100%) identical or exactly (100%) complementary (as the RNA equivalent) to a fragment of equivalent length of a DNA having a sequence selected from the Target Gene Sequences Group, the Trigger Sequences Group, or the DNA complement of any thereof. In some embodiments, the RNA containing the silencing element(s) has an overall sequence of about 95%, about 96%, about 97%, about 98%, about 99%, or about 100% identity with or complementarity to the fragment of a DNA having a sequence selected from the Target Gene Sequences Group.

In some embodiments, the silencing element comprises at least one segment of 18 or more contiguous nucleotides with a sequence of about 95% to about 100% identity with or complementarity to a fragment of equivalent length of the target gene. In some embodiments the silencing element comprises at least one segment of 18 or more contiguous nucleotides with a sequence of about 95% to about 100% identity with or complementarity to a fragment of equivalent length of a DNA having a sequence selected from the Target Gene Sequences Group, or the Trigger Sequences Group, or a DNA complement of any thereof. In some embodiments the silencing element comprises at least one segment of 18 or more contiguous nucleotides, e.g., between 18-24, or between 18-28, or between 20-30, or between 20-50, or between 20-100, or between 50-100, or between 50-500, or between 100-250, or between 100-500, or between 200-1000, or between 500-2000, or even greater. In some embodiments the silencing element comprises more than 18 contiguous nucleotides, e.g., 19, 20, 21, 22, 23, 24, 25, 26, 27, 28, 29, 30, or greater than 30, e.g., at least about 35, at least about 40, at least about 45, at least about 50, at least about 55, at least about 60, at least about 65, at least about 70, at least about 75, at least about 80, at least about 85, at least about 90, at least about 95, at least about 100, at least about 110, at least about 120, at least about 130, at least about 140, at least about 150, at least about 160, at least about 170, at least about 180, at least about 190, at least about 200, at least about 210, at least about 220, at least about 230, at least about 240, at least about 250, at least about 260, at least about 270, at least about 280, at least about 290, at least about 300, at least about 350, at least about 400, at least about 450, at least about 500, or greater than 500 contiguous nucleotides. In particular embodiments, the silencing element comprises at least one segment of at least 18, 19, 20, or 21 contiguous nucleotides with a sequence of 100% identity with a fragment of equivalent length of a DNA or target gene having a sequence selected from the Target Gene Sequences Group, the Trigger Sequences Group, or the DNA complement of any thereof. In particular embodiments, the RNA is a double-stranded nucleic acid (e.g., dsRNA) with one strand comprising at least one segment of at least 18, 19, 20, or 21 contiguous nucleotides with a sequence of 100% identity with a fragment of equivalent length of a DNA or target gene having a sequence selected from the Target Gene Sequences Group, the Trigger Sequences Group, or the DNA complement of any thereof; expressed as base-pairs, such a double-stranded nucleic acid comprises at least one segment of at least 18, 19, 20, or 21 contiguous, perfectly matched base-pairs which correspond to a fragment of equivalent length of a DNA or target gene having a sequence selected from the Target Gene Sequences Group, the Trigger Sequences Group, or the DNA complement of any thereof. In particular embodiments, each silencing element contained in the RNA is of a length greater than that which is typical of naturally occurring regulatory small RNAs, e.g., each segment is at least about 30 contiguous nucleotides (or base-pairs) in length. In some embodiments, the total length of the RNA, or the length of each silencing element contained in the RNA, is less than the total length of the sequence of interest (DNA or target gene having a sequence selected from the Target Gene Sequences Group or Trigger Sequences Group). In some embodiments, the total length of the RNA is between about 50 to about 500 nucleotides (for single-stranded polynucleotides) or base-pairs (for double-stranded polynucleotides). In some embodiments, the RNA is a dsRNA of between about 100 to about 500 base-pairs, such as a dsRNA of the length of any of the dsRNA triggers disclosed in Tables 1A, 1B and 1C. Embodiments include those in which the RNA is a dsRNA comprising a segment having a sequence selected from the group consisting of: SEQ ID NO: 115, 116, 119, 121, 123, 127, 135, 140, 141, 143, 150, 151, 153, 158, 161, 201, 209, 213, 217, 434, 442, 451, 452, 453, 454, 456, 457, 461, 474, 476, 479, 489, 491, 492, 521, 522, 523, 524, 527, 528, 536, 538, 539, 823, 826, 827, 828, 830, 832, 833, 834, 840, 841, 842, 844, 845, 851, 853, 854, 862, 874, 877, 883, 892, 893, 901, 946, 947, 953, 998, 1011, 1012, 1017, 1018, 1019, 1021, 1022, 1023, 1024, 1030, 1038, 1041, 1042, 1044, 1047, 1089, 1090, 1091, 1095, 1096, 1098, 1099, 1102, 1103, 1334, 1335, 1336, 1341, 1343, 1346, 1351, 1353, 1354, 1362, 1363, 1377, 1386, 1387, 1393, 1396, 1417, 1420, 1422, 1425, 1426, 1446, 1447, 1470, 1473, 1481, 1493, 1494, 1495, 1500, 1505, 1509, 1513, 1520, 1538, 1540, 1546, 1548, 1550, 1624, 1625, 1629, 1682, or 1683, or a combination thereof. In another embodiment, the agent comprises a polynucleotide or RNA encoded by a sequence selected from the group consisting of SEQ ID NOs: 434, 442, 451, 452, 453, 454, 456, 457, 461, 474, 476, 479, 489, 491, 492, 521, 522, 523, 524, 527, 528, 536, 538 and 539, or a combination thereof.

The RNA of use in this method is generally designed to suppress one or more genes (“target genes”). In some embodiments, the target genes can include coding or non-coding sequence or both. In other embodiments, the target gene has a sequence identical to or complementary to a messenger RNA, e.g., in some embodiments the target gene is a cDNA. In specific embodiments, the RNA is designed to suppress one or more target genes, where each target gene has a DNA sequence selected from the Target Gene Sequences Group or the Trigger Sequences Group. In various embodiments, the RNA is designed to suppress one or more genes, where each gene has a sequence selected from the Target Gene Sequences Group or the Trigger Sequences Group, and can be designed to suppress multiple genes from this group, or to target different regions of one or more of these genes. In an embodiment, the RNA comprises multiple silencing elements each of which comprises at least one segment of 21 contiguous nucleotides with a sequence of 100% identity with or 100% complementarity to a fragment of equivalent length of a DNA having a sequence selected from the Target Gene Sequences Group, the Trigger Sequences Group, or the DNA complement of any thereof. In such cases, each silencing element can be identical or different in size or in sequence, and can be sense or anti-sense relative to the target gene. For example, in one embodiment the RNA can include multiple silencing elements in tandem or repetitive arrangements, wherein each silencing element comprises at least one segment of 21 contiguous nucleotides with a sequence of 100% identity with or 100% complementarity to a fragment of equivalent length of a DNA having a sequence selected from the Target Gene Sequences Group or Trigger Sequences Group, or a DNA complement of any thereof; the segments can be from different regions of the target gene, e.g., the segments can correspond to different exon regions of the target gene, and “spacer” nucleotides which do not correspond to a target gene can optionally be used in between or adjacent to the segments.

The total length of the RNA can be greater than 18 contiguous nucleotides, and can include nucleotides in addition to the silencing element having a sequence of about 95% to about 100% identity with or complementarity to a fragment of equivalent length of a DNA or target gene having a sequence selected from the Target Gene Sequences Group, the Trigger Sequences Group, or the DNA complement of any thereof. In other words, the total length of the RNA can be greater than the length of the silencing element designed to suppress one or more target genes, where each target gene has a DNA sequence selected from the group consisting of the Target Gene Sequences Group. For example, the RNA can have nucleotides flanking the “active” silencing element of at least one segment of 18 or more contiguous nucleotides that suppresses the target gene, or include “spacer” nucleotides between active silencing elements, or can have additional nucleotides at the 5′ end, or at the 3′ end, or at both the 5′ and 3′ ends. In an embodiment, the RNA comprises additional nucleotides that are not specifically related (having a sequence not complementary or identical to) to the DNA or target gene having a sequence selected from the Target Gene Sequences Group, the Trigger Sequences Group, or the DNA complement of any thereof, e.g., nucleotides that provide stabilizing secondary structure or for convenience in cloning or manufacturing. In an embodiment, the RNA comprises additional nucleotides located immediately adjacent to one or more silencing element of 18 or more contiguous nucleotides with a sequence of about 95% to about 100% identity with or complementarity to a fragment of equivalent length of a DNA or target gene having a sequence selected from the Target Gene Sequences Group, the Trigger Sequences Group, or the DNA complement of any thereof. In an embodiment, the RNA comprises one such silencing element, with an additional 5′ G or an additional 3′ C or both, adjacent to the silencing element. In another embodiment, the RNA is a double-stranded RNA comprising additional nucleotides to form an overhang, for example, a dsRNA comprising 2 deoxyribonucleotides to form a 3′ overhang. Thus in various embodiments, the nucleotide sequence of the entire RNA is not 100% identical or complementary to a fragment of contiguous nucleotides in the DNA or target gene having a sequence selected from the Target Gene Sequences Group, the Trigger Sequences Group, or the DNA complement of any thereof. For example, in some embodiments the RNA comprises at least two silencing elements each of 21 contiguous nucleotides with a sequence of 100% identity with a fragment of a DNA having a sequence selected from the Target Gene Sequences Group, the Trigger Sequences Group, or the DNA complement of any thereof, wherein (1) the at least two silencing elements are separated by one or more spacer nucleotides, or (2) the at least two silencing elements are arranged in an order different from that in which the corresponding fragments occur in the DNA having a sequence selected from the Target Gene Sequences Group, the Trigger Sequences Group, or the DNA complement of any thereof.

In some embodiments the RNA consists of naturally occurring ribonucleotides. In certain embodiments, the RNA comprises components other than ribonucleotides, for example, synthetic RNAs consisting mainly of ribonucleotides but with one or more terminal deoxyribonucleotides or one or more terminal dideoxyribonucleotides. In certain embodiments, the RNA comprises non-canonical nucleotides such as inosine, thiouridine, or pseudouridine. In certain embodiments, the RNA comprises chemically modified nucleotides.

The RNA of use in this method is provided by suitable means known to one in the art. Embodiments include those wherein the RNA is chemically or enzymatically synthesized (e.g., by in vitro transcription, such as transcription using a T7 polymerase or other polymerase), produced by expression in a microorganism or in cell culture (such as plant or insect cells grown in culture), produced by expression in a plant cell, or produced by microbial fermentation.

In some embodiments the RNA is provided as an isolated RNA that is not part of an expression construct and is lacking additional elements such as a promoter or terminator sequences. Such RNAs can be relatively short, such as single- or double-stranded RNAs of between about 18 to about 300 or between about 50 to about 500 nucleotides (for single-stranded RNAs) or between about 18 to about 300 or between about 50 to about 500 base-pairs (for double-stranded RNAs). Alternatively the RNA can be provided in more complex constructs, e.g., as part of a recombinant expression construct, or included in a recombinant vector, for example in a recombinant plant virus vector or in a recombinant baculovirus vector. In some embodiments such recombinant expression constructs or vectors are designed to include additional elements, such as including additional RNA encoding an aptamer or ribozyme or an expression cassette for expressing a gene of interest (e.g., an insecticidal protein).

### V. Methods of Providing Plants Having Improved Resistance to Lepidopteran Infestations, and the Plants, Plant Parts, and Seeds Thus Provided

Several embodiments relate to a method of providing a plant having improved resistance to a Lepidopteran pest infestation comprising providing to the plant at least one polynucleotide comprising at least one segment of 18 or more contiguous nucleotides that is essentially identical or complementary to a fragment of a target gene selected from the group consisting of the genes identified in the Target Gene Sequences Group. In an embodiment, this invention provides a method of providing a plant having improved resistance to a Lepidopteran pest infestation comprising providing to the plant at least one polynucleotide comprising at least one segment that is identical or complementary to at least 18 contiguous nucleotides of a target gene or an RNA transcribed from the target gene, wherein the target gene is selected from the genes identified in the Target Gene Sequences Group or an RNA transcribed from the target gene. Embodiments of sequences that target genes are identified by SEQ ID NO in Tables 1A, 1B and 1C and include sequences that target genes having a sequence selected from the group consisting of the Target Gene Sequences Group, as well as related genes, including orthologues from related insect pest, for example related genes from other Lepidopteran pests. In some embodiments, the polynucleotide (e.g., double-stranded RNA) is chemically or enzymatically synthesized or is produced by expression in a microorganism or by expression in a plant cell. In some embodiments the polynucleotide comprises at least one segment of 18 or more contiguous nucleotides that is essentially identical or complementary to a sequence selected from the Target Gene Sequences Group, the Trigger Sequences Group, or the DNA complement of any thereof. In some embodiments the polynucleotide is a dsRNA with a strand having a sequence selected from the Trigger Sequences Group, or the complement thereof. In some embodiments the polynucleotide comprises a dsRNA with a strand having a sequence selected from the Trigger Sequences Group.

In a related aspect, this invention is directed to the plant having improved resistance to a Lepidopteran pest infestation, provided by expressing in the plant at least one polynucleotide comprising at least one segment of 18 or more contiguous nucleotides that are essentially identical or complementary to a fragment of equivalent length of a target gene selected from the group consisting of the genes identified in the Target Gene Sequences Group, whereby the resulting plant has improved resistance to a Lepidopteran pest infestation when compared to a control plant in which the polynucleotide is not expressed. In a related aspect, this invention is directed to the plant having improved resistance to a Lepidopteran pest infestation, provided by expressing in the plant at least one polynucleotide comprising at least one segment of 18 or more contiguous nucleotides with a sequence of about 95% to about 100% identity with or complementarity to a fragment of a target gene selected from the group consisting of the genes identified in the Target Gene Sequences Group, whereby the resulting plant has improved resistance to a Lepidopteran pest infestation when compared to a control plant in which the polynucleotide is not expressed.

In yet another aspect, this invention is directed to seed or propagatable parts (especially transgenic progeny seed or propagatable parts) produced by the plant having improved resistance to a Lepidopteran pest infestation, as provided by this method. Also contemplated is a commodity product produced by the plant having improved resistance to a Lepidopteran pest infestation, as provided by this method, and a commodity product produced from the transgenic progeny seed or propagatable parts of such a plant.

Another aspect of this invention provides a method of providing a plant having improved resistance to a Lepidopteran pest infestation comprising topically applying to the plant a composition comprising at least one polynucleotide having at least one segment of 18 or more contiguous nucleotides with a sequence of about 95% to about 100% identity with a fragment of a target gene or DNA having a sequence selected from The Target Gene Sequences Group, the Trigger Sequences Group, or the DNA complement of any thereof, in a manner such that the plant treated with the polynucleotide-containing composition exhibits improved resistance to a Lepidopteran pest infestation, relative to an untreated plant. In an embodiment, the at least one polynucleotide comprises at least one segment of 18 or more contiguous nucleotides that are essentially identical to a fragment of equivalent length of a DNA having a sequence selected from the Target Gene Sequences Group, the Trigger Sequences Group, or the DNA complement of any thereof. The polynucleotide can be longer than the segment or segments it contains, but each segment and corresponding fragment of a target gene are of equivalent length. In an embodiment, this invention provides a method of providing a plant having improved resistance to a Lepidopteran pest infestation comprising topically applying to the plant a composition comprising at least one polynucleotide comprising a nucleotide sequence that is complementary to at least 18 contiguous nucleotides of a target gene having a nucleotide sequence selected from the group consisting of: SEQ ID NOs: 2, 5, 7, 9, 13, 21, 26, 27, 29, 36, 37, 39, 44, 47, 87, 95, 99, 103, 301, 309, 318, 319, 320, 321, 323, 324, 328, 341, 343, 346, 356, 358, 359, 503, 504, 505, 506, 509, 510, 530, 532, 533, 542, 545, 546, 547, 549, 551, 552, 553, 559, 560, 561, 563, 564, 570, 572, 573, 581, 593, 596, 602, 611, 612, 620, 665, 666, 672, 717, 730, 731, 736, 737, 738, 740, 741, 742, 743, 749, 757, 760, 761, 763, 766, 808, 809, 810, 814, 815, 817, 818, 821, 822, 1104, 1105, 1106, 1111, 1113, 1116, 1121, 1123, 1124, 1132, 1133, 1147, 1156, 1157, 1163, 1166, 1187, 1190, 1192, 1195, 1196, 1216, 1217, 1240, 1243, 1251, 1263, 1264, 1265, 1270, 1275, 1279, 1283, 1290, 1308, 1310, 1316, 1318, 1320, 1564, 1565, 1569, 1622, and 1623, or an RNA transcribed from the target gene. In an embodiment, this invention provides a method of providing a plant having improved resistance to a Lepidopteran pest infestation comprising topically applying to the plant a composition comprising at least one polynucleotide in a manner such that an effective amount of the polynucleotide is ingested by Lepidopteran pest feeding on the plant, the polynucleotide comprising at least 18 contiguous nucleotides that are complementary to a target gene having a nucleotide sequence selected from the group consisting of: SEQ ID NOs: 2, 5, 7, 9, 13, 21, 26, 27, 29, 36, 37, 39, 44, 47, 87, 95, 99, 103, 301, 309, 318, 319, 320, 321, 323, 324, 328, 341, 343, 346, 356, 358, 359, 503, 504, 505, 506, 509, 510, 530, 532, 533, 542, 545, 546, 547, 549, 551, 552, 553, 559, 560, 561, 563, 564, 570, 572, 573, 581, 593, 596, 602, 611, 612, 620, 665, 666, 672, 717, 730, 731, 736, 737, 738, 740, 741, 742, 743, 749, 757, 760, 761, 763, 766, 808, 809, 810, 814, 815, 817, 818, 821, 822, 1104, 1105, 1106, 1111, 1113, 1116, 1121, 1123, 1124, 1132, 1133, 1147, 1156, 1157, 1163, 1166, 1187, 1190, 1192, 1195, 1196, 1216, 1217, 1240, 1243, 1251, 1263, 1264, 1265, 1270, 1275, 1279, 1283, 1290, 1308, 1310, 1316, 1318, 1320, 1564, 1565, 1569, 1622, and 1623, or an RNA transcribed from the target gene.

Polynucleotides of use in the method can be designed for multiple target genes. Embodiments include those in which the composition comprises a dsRNA with a strand having a sequence selected from the group consisting of the Trigger Sequences Group. Related aspects of the invention include compositions for topical application and isolated polynucleotides of use in the method, and plants having improved Lepidopteran resistance provided by the method.

By “topical application” as used throughout herein is meant application to the surface or exterior of an object, such as the surface or exterior of a plant, such as application to the surfaces of a plant part such as a leaf, stem, flower, fruit, shoot, root, seed, tuber, flowers, anthers, or pollen, or application to an entire plant, or to the above-ground or below-ground portions of a plant. Topical application can be carried out on non-living surfaces, such as application to soil, or to a surface or matrix by which a Lepidopteran pest can come in contact with the polynucleotide. In various embodiments of the method, the composition comprising at least one polynucleotide is topically applied to the plant in a suitable form, e.g., as a solid, liquid (including homogeneous mixtures such as solutions and non-homogeneous mixtures such as suspensions, colloids, micelles, and emulsions), powder, suspension, emulsion, spray, encapsulated or micro-encapsulation formulation, in or on microbeads or other carrier particulates, in a film or coating, or on or within a matrix, or as a seed treatment. In some embodiments of the method, the polynucleotide-containing composition is topically applied to above-ground parts of the plant, e.g., sprayed or dusted onto leaves, stems, and flowering parts of the plant.

Embodiments of the method include topical application of a foliar spray (e.g., spraying a liquid polynucleotide-containing composition on leaves of a solanaceous plant) or a foliar dust (e.g., dusting a crop plant with a polynucleotide-containing composition in the form of a powder or on carrier particulates). In other embodiments, the polynucleotide-containing composition is topically applied to below-ground parts of the plant, such as to the roots, e.g., by means of a soil drench. In other embodiments, the polynucleotide-containing composition is topically applied to a seed that is grown into the plant. The topical application can be in the form of topical treatment of fruits of crop plants or seeds from fruits of crop plants, or in the form of topical treatment of “seed potato” tubers or pieces of tuber (e.g., by soaking, coating, or dusting the seed potato). Suitable binders, inert carriers, surfactants, and the like can optionally be included in the polynucleotide-containing composition, as is known to one skilled in formulation of pesticides and seed treatments.

In some embodiments, the polynucleotide-containing composition is at least one topically implantable formulation selected from the group consisting of a particulate, pellet, or capsule topically implanted in the plant; in such embodiments the method comprises topically implanting in the plant the topically implantable formulation. In some embodiments, the polynucleotide-containing composition is at least one in-furrow formulation selected from the group consisting of a powder, granule, pellet, capsule, spray, or drench, or any other forms suited for topically applying to a furrow; in such embodiments, the method includes an in-furrow treatment with the in-furrow formulation. In one embodiment the polynucleotide-containing composition can be ingested or otherwise absorbed internally by the Lepidopteran pest. For example, the polynucleotide-containing composition can be in the form of bait. In some embodiments, the polynucleotide-containing composition further comprises one or more components selected from the group consisting of a carrier agent, a surfactant, a cationic lipid (such as that disclosed in Example 18 of U.S. patent application publication 2011/0296556, incorporated by reference herein), an organosilicone, an organosilicone surfactant, a polynucleotide herbicidal molecule, a non-polynucleotide herbicidal molecule, a non-polynucleotide pesticide, a safener, and an insect growth regulator. In one embodiment the composition further comprises a nonionic organosilicone surfactant such as SILWET® brand surfactants, e.g., SILWET L-77® brand surfactant having CAS Number 27306-78-1 and EPA Number: CAL.REG.NO. 5905-50073-AA, currently available from Momentive Performance Materials, Albany, N.Y. In some embodiments, the topically applied composition further comprises at least one pesticidal agent selected from the group consisting of a patatin, a plant lectin, a phytoecdysteroid, a Bacillus thuringiensis insecticidal protein, a Xenorhabdus insecticidal protein, a Photorhabdus insecticidal protein, a Bacillus laterosporus insecticidal protein, and a Bacillus sphaericus insecticidal protein. Alternatively such additional components or pesticidal agents can be provided separately, e.g., by separate topical application or by transgenic expression in the plant. Alternatively the plant is topically treated with the polynucleotide-containing composition as well as with a separate (preceding, following, or concurrent) application of a substance that improves the efficacy of the polynucleotide-containing composition. For example, a plant can be sprayed with a first topical application of a solution containing a nonionic organosilicone surfactant such as SILWET® brand surfactants, e.g., SILWET L-77® brand surfactant, followed by a second topical application of the polynucleotide-containing composition, or vice-versa.

It is anticipated that the combination of certain polynucleotides useful in the polynucleotide-containing composition (e.g., the polynucleotide triggers described in the working Examples) with one or more non-polynucleotide pesticidal agents will result in an enhanced improvement in prevention or control of Lepidopteran pest infestations, when compared to the effect obtained with the polynucleotide alone or the non-polynucleotide pesticidal agent alone. In an embodiment, the polynucleotide-containing composition is provided as a transgenic plant expressing one or more polynucleotides and one or more genes encoding a non-polynucleotide pesticidal agent selected from the group consisting of a patatin, a plant lectin, a phytoecdysteroid, a Bacillus thuringiensis insecticidal protein, a Xenorhabdus insecticidal protein, a Photorhabdus insecticidal protein, a Bacillus laterosporus insecticidal protein, and a Bacillus sphaericus insecticidal protein, wherein the transgenic plant is found to exhibit improved resistance to Lepidopteran pest infestations.

The polynucleotide useful in the polynucleotide-containing composition is provided by suitable means known to one in the art. Embodiments include those wherein the polynucleotide is chemically or enzymatically synthesized (e.g., by in vitro transcription, such as transcription using a T7 polymerase or other polymerase), produced by expression in a microorganism or in cell culture (such as plant or insect cells grown in culture), produced by expression in a plant cell, or produced by microbial fermentation.

In many embodiments the polynucleotide useful in the polynucleotide-containing composition is provided as an isolated DNA or RNA fragment. In some embodiments the polynucleotide useful in the polynucleotide-containing composition is not part of an expression construct and is lacking additional elements such as a promoter or terminator sequences). Such polynucleotides can be relatively short, such as single- or double-stranded polynucleotides of between about 18 to about 300 or between about 50 to about 500 nucleotides (for single-stranded polynucleotides) or between about 18 to about 300 or between about 50 to about 500 base-pairs (for double-stranded polynucleotides). In some embodiments, the polynucleotide is a dsRNA of between about 100 to about 500 base-pairs, such as a dsRNA of the length of any of the dsRNA triggers disclosed in Tables 1A, 1B and 1C. Alternatively the polynucleotide can be provided in more complex constructs, e.g., as part of a recombinant expression construct, or included in a recombinant vector, for example in a recombinant plant virus vector or in a recombinant baculovirus vector. Such recombinant expression constructs or vectors can be designed to include additional elements, such as expression cassettes for expressing a gene of interest (e.g., an insecticidal protein).

The polynucleotide useful in the polynucleotide-containing composition has at least one segment of 18 or more contiguous nucleotides with a sequence of about 95% to about 100% identity with a fragment of equivalent length of a DNA having a sequence selected from the Target Gene Sequences Group, the Trigger Sequences Group, or the DNA complement of any thereof. In an embodiment the polynucleotide comprises at least one segment of 18 or more contiguous nucleotides that are essentially identical or complementary to a fragment of equivalent length of a DNA having a sequence selected from Target Gene Sequences Group or the Trigger Sequences Group. In some embodiments, the contiguous nucleotides have a sequence of about 95%, about 96%, about 97%, about 98%, about 99%, or about 100% identity with a fragment of a DNA having a sequence selected from the Target Gene Sequences Group, the Trigger Sequences Group, or the DNA complement of any thereof. In some embodiments the contiguous nucleotides are exactly (100%) identical to a fragment of equivalent length of a DNA having a sequence selected from the Target Gene Sequences Group, the Trigger Sequences Group, or the DNA complement of any thereof. In some embodiments, the polynucleotide has an overall sequence of about 95%, about 96%, about 97%, about 98%, about 99%, or about 100% identity with a fragment of a DNA having a sequence selected from the Target Gene Sequences Group, the Trigger Sequences Group, or the DNA complement thereof.

The polynucleotide useful in the polynucleotide-containing composition comprises at least one segment of 18 or more contiguous nucleotides, e.g., between 18-24, or between 18-28, or between 20-30, or between 20-50, or between 20-100, or between 50-100, or between 50-500, or between 100-250, or between 100-500, or between 200-1000, or between 500-2000, or even greater. In some embodiments the segment comprises more than 18 contiguous nucleotides, e.g., 19, 20, 21, 22, 23, 24, 25, 26, 27, 28, 29, 30, or greater than 30, e.g., at least about 35, at least about 40, at least about 45, at least about 50, at least about 55, at least about 60, at least about 65, at least about 70, at least about 75, at least about 80, at least about 85, at least about 90, at least about 95, at least about 100, at least about 110, at least about 120, at least about 130, at least about 140, at least about 150, at least about 160, at least about 170, at least about 180, at least about 190, at least about 200, at least about 210, at least about 220, at least about 230, at least about 240, at least about 250, at least about 260, at least about 270, at least about 280, at least about 290, at least about 300, at least about 350, at least about 400, at least about 450, at least about 500, or greater than 500 contiguous nucleotides. In particular embodiments, the polynucleotide comprises at least one segment of at least 18, 19, 20, or 21 contiguous nucleotides with a sequence of 100% identity with a fragment of equivalent length of a DNA or target gene having a sequence selected from the Target Gene Sequences Group, the Trigger Sequences Group, or the DNA complement thereof. In particular embodiments, the polynucleotide is a double-stranded nucleic acid (e.g., dsRNA) with one strand comprising at least one segment of at least 18, 19, 20, or 21 contiguous nucleotides with a sequence of 100% identity with a fragment of equivalent length of a DNA or target gene having a sequence selected from the Target Gene Sequences Group, the Trigger Sequences Group, or the DNA complement of any thereof expressed as base-pairs, such a double-stranded nucleic acid comprises at least one segment of at least 18, 19, 20, or 21 contiguous, perfectly matched base-pairs which correspond to a fragment of equivalent length of a DNA or target gene having a sequence selected from the Target Gene Sequences Group, the Trigger Sequences Group, or the DNA complement of any thereof. In particular embodiments, each segment contained in the polynucleotide is of a length greater than that which is typical of naturally occurring regulatory small RNAs, e.g., each segment is at least about 30 contiguous nucleotides (or base-pairs) in length. In some embodiments, the total length of the polynucleotide, or the length of each segment contained in the polynucleotide, is less than the total length of the sequence of interest (DNA or target gene having a sequence selected from the group consisting of the Target Gene Sequences Group). In some embodiments, the total length of the polynucleotide is between about 50 to about 500 nucleotides (for single-stranded polynucleotides) or base-pairs (for double-stranded polynucleotides). In some embodiments, the polynucleotide is a dsRNA of between about 100 to about 500 base-pairs, such as a dsRNA of the length of any of the dsRNA triggers disclosed in Tables 1A, 1B and 1C. In some embodiments, the polynucleotide is dsRNA encoded by a sequence selected from the group consisting of SEQ ID NO: 115, 116, 119, 121, 123, 127, 135, 140, 141, 143, 150, 151, 153, 158, 161, 201, 209, 213, 217, 434, 442, 451, 452, 453, 454, 456, 457, 461, 474, 476, 479, 489, 491, 492, 521, 522, 523, 524, 527, 528, 536, 538, 539, 823, 826, 827, 828, 830, 832, 833, 834, 840, 841, 842, 844, 845, 851, 853, 854, 862, 874, 877, 883, 892, 893, 901, 946, 947, 953, 998, 1011, 1012, 1017, 1018, 1019, 1021, 1022, 1023, 1024, 1030, 1038, 1041, 1042, 1044, 1047, 1089, 1090, 1091, 1095, 1096, 1098, 1099, 1102, 1103, 1334, 1335, 1336, 1341, 1343, 1346, 1351, 1353, 1354, 1362, 1363, 1377, 1386, 1387, 1393, 1396, 1417, 1420, 1422, 1425, 1426, 1446, 1447, 1470, 1473, 1481, 1493, 1494, 1495, 1500, 1505, 1509, 1513, 1520, 1538, 1540, 1546, 1548, 1550, 1624, 1625, 1629, 1682, or 1683, or a combination thereof. In another embodiment, the agent comprises a polynucleotide or RNA encoded by a sequence selected from the group consisting of SEQ ID NOs: 434, 442, 451, 452, 453, 454, 456, 457, 461, 474, 476, 479, 489, 491, 492, 521, 522, 523, 524, 527, 528, 536, 538 and 539, or a combination thereof.

The topically applied polynucleotide is generally designed to suppress one or more genes (“target genes”). Such target genes can include coding or non-coding sequence or both. In specific embodiments, the polynucleotide is designed to suppress one or more target genes, where each target gene has a DNA sequence selected from the group consisting of the Target Gene Sequences Group. In various embodiments, the topically applied polynucleotide is designed to suppress one or more genes, where each gene has a sequence selected from the group consisting of the Target Gene Sequences Group, and can be designed to suppress multiple genes from this group, or to target different regions of one or more of these genes. In an embodiment, the topically applied polynucleotide comprises multiple sections or segments each of which comprises at least one segment of 21 contiguous nucleotides with a sequence of 100% identity with a fragment of equivalent length of a DNA having a sequence selected from the Target Gene Sequences Group, the Trigger Sequences Group, or the DNA complement of any thereof. In such cases, each section can be identical or different in size or in sequence, and can be sense or anti-sense relative to the target gene. For example, in one embodiment the topically applied polynucleotide can include multiple sections in tandem or repetitive arrangements, wherein each section comprises at least one segment of 21 contiguous nucleotides with a sequence of 100% identity with a fragment of equivalent length of a DNA having a sequence selected from the Target Gene Sequences Group.

The total length of the topically applied polynucleotide can be greater than 18 contiguous nucleotides, and can include nucleotides in addition to the contiguous nucleotides having the sequence of about 95% to about 100% identity with a fragment of equivalent length of a DNA having a sequence selected from the Target Gene Sequences Group, the Trigger Sequences Group, or the DNA complement of any thereof. In other words, the total length of the topically applied polynucleotide can be greater than the length of the section or segment of the polynucleotide designed to suppress one or more target genes, where each target gene has a DNA sequence selected from the group consisting of the Target Gene Sequences Group. For example, the topically applied polynucleotide can have nucleotides flanking the “active” segment of at least one segment of 18 or more contiguous nucleotides that suppresses the target gene, or include “spacer” nucleotides between active segments, or can have additional nucleotides at the 5′ end, or at the 3′ end, or at both the 5′ and 3′ ends. In an embodiment, the topically applied polynucleotide comprises additional nucleotides that are not specifically related (having a sequence not complementary or identical to) to the DNA or target gene having a sequence selected from the Target Gene Sequences Group, the Trigger Sequences Group, or the DNA complement of any thereof, e.g., nucleotides that provide stabilizing secondary structure or for convenience in cloning or manufacturing.

In an embodiment, the topically applied polynucleotide comprises additional nucleotides located immediately adjacent to one or more segment of 18 or more contiguous nucleotides with a sequence of about 95% to about 100% identity with or complementarity to a fragment of equivalent length of a DNA or target gene having a sequence selected from the group consisting of the Target Gene Sequences Group. In an embodiment, the topically applied polynucleotide comprises one such segment, with an additional 5′ G or an additional 3′ C or both, adjacent to the segment. In another embodiment, the topically applied polynucleotide is a double-stranded RNA comprising additional nucleotides to form an overhang, for example, a dsRNA comprising 2 deoxyribonucleotides to form a 3′ overhang. Thus, in various embodiments, the nucleotide sequence of the entire topically applied polynucleotide is not 100% identical or complementary to a fragment of contiguous nucleotides in the DNA or target gene having a sequence selected from the Target Gene Sequences Group, the Trigger Sequences Group, or the DNA complement of any thereof. For example, in some embodiments the topically applied polynucleotide comprises at least two segments each of 21 contiguous nucleotides with a sequence of 100% identity with a fragment of a DNA having a sequence selected from the Target Gene Sequences Group, the Trigger Sequences Group, or the DNA complement of any thereof, wherein (1) the at least two segments are separated by one or more spacer nucleotides, or (2) the at least two segments are arranged in an order different from that in which the corresponding fragments occur in the DNA having a sequence selected from the Target Gene Sequences Group, the Trigger Sequences Group, or the DNA complement of any thereof.

In a related aspect, this invention is directed to the plant having improved resistance to a Lepidopteran pest infestation, provided by this method which comprises topically applying to the plant a composition comprising at least one polynucleotide having at least one segment of 18 or more contiguous nucleotides with a sequence of about 95% to about 100% identity with a fragment of equivalent length of a DNA having a sequence selected from the Target Gene Sequences Group, the Trigger Sequences Group, or the DNA complement of any thereof, whereby the plant treated with the polynucleotide composition exhibits improved resistance to a Lepidopteran pest infestation, relative to an untreated plant.

An embodiment is a crop plant having improved resistance to a Lepidopteran pest infestation when compared to a control plant, provided by topically applying to the plant or to a seed grown into the plant (or, where the plant is a potato plant, to a seed potato grown into the potato plant) a dsRNA trigger having a sequence selected from the Trigger Sequences Group, or the complement thereof, or a dsRNA trigger encoded by a sequence selected from the group consisting of SEQ ID NO: 115, 116, 119, 121, 123, 127, 135, 140, 141, 143, 150, 151, 153, 158, 161, 201, 209, 213, 217, 434, 442, 451, 452, 453, 454, 456, 457, 461, 474, 476, 479, 489, 491, 492, 521, 522, 523, 524, 527, 528, 536, 538, 539, 823, 826, 827, 828, 830, 832, 833, 834, 840, 841, 842, 844, 845, 851, 853, 854, 862, 874, 877, 883, 892, 893, 901, 946, 947, 953, 998, 1011, 1012, 1017, 1018, 1019, 1021, 1022, 1023, 1024, 1030, 1038, 1041, 1042, 1044, 1047, 1089, 1090, 1091, 1095, 1096, 1098, 1099, 1102, 1103, 1334, 1335, 1336, 1341, 1343, 1346, 1351, 1353, 1354, 1362, 1363, 1377, 1386, 1387, 1393, 1396, 1417, 1420, 1422, 1425, 1426, 1446, 1447, 1470, 1473, 1481, 1493, 1494, 1495, 1500, 1505, 1509, 1513, 1520, 1538, 1540, 1546, 1548, 1550, 1624, 1625, 1629, 1682, or 1683, or a combination thereof. In another embodiment, the agent comprises a polynucleotide or RNA encoded by a sequence selected from the group consisting of SEQ ID NOs: 434, 442, 451, 452, 453, 454, 456, 457, 461, 474, 476, 479, 489, 491, 492, 521, 522, 523, 524, 527, 528, 536, 538 and 539, or a combination thereof. In yet another aspect, this invention is directed to seed (especially transgenic progeny seed) produced by the plant having improved resistance to a Lepidopteran pest infestation, as provided by this method. Also contemplated is a commodity product produced by the plant having improved resistance to a Lepidopteran pest infestation, as provided by this method, and a commodity product produced from the transgenic progeny seed of such a plant.

### VI. Insecticidal Compositions for Controlling Lepidopteran Pest

Another aspect of this invention provides an insecticidal composition for controlling a Lepidopteran pest comprising an insecticidally effective amount of at least one RNA comprising at least one segment of 18 or more contiguous nucleotides that is essentially identical or complementary to a fragment of a target gene or DNA having a sequence selected from the Target Gene Sequences Group, the Trigger Sequences Group, or the DNA complement of any thereof. In this context “controlling” includes inducement of a physiological or behavioural change in a Lepidopteran pest (adult or larvae) such as, but not limited to, growth stunting, increased mortality, decrease in reproductive capacity, decrease in or cessation of feeding behavior or movement, or decrease in or cessation of metamorphosis stage development. By “insecticidally effective” is meant effective in inducing a physiological or behavioural change in a Lepidopteran pest (adult or larvae) such as, but not limited to, growth stunting, increased mortality, decrease in reproductive capacity or decreased fecundity, decrease in or cessation of feeding behavior or movement, or decrease in or cessation of metamorphosis stage development; in some embodiments, application of an insecticidally effective amount of the RNA to a plant improves the plant's resistance to infestation by a Lepidopteran pest. The RNA can be longer than the segment or segments it contains, but each segment and corresponding fragment of a target gene are of equivalent length. RNAs of use in the method can be designed for multiple target genes. Embodiments include those in which the insecticidal composition comprises an insecticidally effective amount of a polynucleotide comprising at least 18, 19, 20, or 21 contiguous nucleotides that are complementary to a target gene having a nucleotide sequence selected from the Target Genes Sequences Group, or an RNA transcribed from the target gene; or an insecticidally effective amount of at least one polynucleotide comprising at least one silencing element that is complementary to at least 21 contiguous nucleotides of a target gene or an RNA transcribed from the target gene, wherein the target gene has a nucleotide sequence selected from the Target Gene Sequences Group; or an insecticidally effective amount of at least one RNA comprising at least one segment that is identical or complementary to at least 18, 19, 20, or 21 contiguous nucleotides of a target gene having a nucleotide sequence selected from the group consisting of: SEQ ID NOs: 2, 5, 7, 9, 13, 21, 26, 27, 29, 36, 37, 39, 44, 47, 87, 95, 99, 103, 301, 309, 318, 319, 320, 321, 323, 324, 328, 341, 343, 346, 356, 358, 359, 503, 504, 505, 506, 509, 510, 530, 532, 533, 542, 545, 546, 547, 549, 551, 552, 553, 559, 560, 561, 563, 564, 570, 572, 573, 581, 593, 596, 602, 611, 612, 620, 665, 666, 672, 717, 730, 731, 736, 737, 738, 740, 741, 742, 743, 749, 757, 760, 761, 763, 766, 808, 809, 810, 814, 815, 817, 818, 821, 822, 1104, 1105, 1106, 1111, 1113, 1116, 1121, 1123, 1124, 1132, 1133, 1147, 1156, 1157, 1163, 1166, 1187, 1190, 1192, 1195, 1196, 1216, 1217, 1240, 1243, 1251, 1263, 1264, 1265, 1270, 1275, 1279, 1283, 1290, 1308, 1310, 1316, 1318, 1320, 1564, 1565, 1569, 1622, and 1623, or an RNA transcribed from the target gene; or an RNA molecule that causes mortality or stunting of growth in a Lepidopteran pest when ingested or contacted by the Lepidopteran pest, wherein the RNA molecule comprises at least 18, 19, 20, or 21 contiguous nucleotides that are complementary to a target gene having a nucleotide sequence selected from the group consisting of: SEQ ID NOs: 2, 5, 7, 9, 13, 21, 26, 27, 29, 36, 37, 39, 44, 47, 87, 95, 99, 103, 301, 309, 318, 319, 320, 321, 323, 324, 328, 341, 343, 346, 356, 358, 359, 503, 504, 505, 506, 509, 510, 530, 532, 533, 542, 545, 546, 547, 549, 551, 552, 553, 559, 560, 561, 563, 564, 570, 572, 573, 581, 593, 596, 602, 611, 612, 620, 665, 666, 672, 717, 730, 731, 736, 737, 738, 740, 741, 742, 743, 749, 757, 760, 761, 763, 766, 808, 809, 810, 814, 815, 817, 818, 821, 822, 1104, 1105, 1106, 1111, 1113, 1116, 1121, 1123, 1124, 1132, 1133, 1147, 1156, 1157, 1163, 1166, 1187, 1190, 1192, 1195, 1196, 1216, 1217, 1240, 1243, 1251, 1263, 1264, 1265, 1270, 1275, 1279, 1283, 1290, 1308, 1310, 1316, 1318, 1320, 1564, 1565, 1569, 1622, and 1623, or an RNA transcribed from the target gene; or an insecticidal double-stranded RNA molecule that causes mortality or stunting of growth in a Lepidopteran pest when ingested or contacted by the Lepidopteran pest, wherein at least one strand of the insecticidal double-stranded RNA molecule comprises 21 contiguous nucleotides that are complementary to a target gene or an RNA transcribed from the target gene, wherein the target gene has a sequence selected from the group consisting of: SEQ ID NOs:22, 5, 7, 9, 13, 21, 26, 27, 29, 36, 37, 39, 44, 47, 87, 95, 99, 103, 301, 309, 318, 319, 320, 321, 323, 324, 328, 341, 343, 346, 356, 358, 359, 503, 504, 505, 506, 509, 510, 530, 532, 533, 542, 545, 546, 547, 549, 551, 552, 553, 559, 560, 561, 563, 564, 570, 572, 573, 581, 593, 596, 602, 611, 612, 620, 665, 666, 672, 717, 730, 731, 736, 737, 738, 740, 741, 742, 743, 749, 757, 760, 761, 763, 766, 808, 809, 810, 814, 815, 817, 818, 821, 822, 1104, 1105, 1106, 1111, 1113, 1116, 1121, 1123, 1124, 1132, 1133, 1147, 1156, 1157, 1163, 1166, 1187, 1190, 1192, 1195, 1196, 1216, 1217, 1240, 1243, 1251, 1263, 1264, 1265, 1270, 1275, 1279, 1283, 1290, 1308, 1310, 1316, 1318, 1320, 1564, 1565, 1569, 1622, and 1623; or an insecticidally effective amount of at least one double-stranded RNA comprising a sequence selected from the Trigger Sequences Group. In some embodiments, the polynucleotide is a double-stranded RNA. In some embodiments, the polynucleotide (e.g., double-stranded RNA) is chemically or enzymatically synthesized or is produced by expression in a microorganism or by expression in a plant cell. Embodiments include insecticidal compositions comprising a dsRNA having a sequence selected from the Trigger Sequences Group, or in a more specific embodiment, selected from the group consisting of SEQ ID NO: 115, 116, 119, 121, 123, 127, 135, 140, 141, 143, 150, 151, 153, 158, 161, 201, 209, 213, 217, 434, 442, 451, 452, 453, 454, 456, 457, 461, 474, 476, 479, 489, 491, 492, 521, 522, 523, 524, 527, 528, 536, 538, 539, 823, 826, 827, 828, 830, 832, 833, 834, 840, 841, 842, 844, 845, 851, 853, 854, 862, 874, 877, 883, 892, 893, 901, 946, 947, 953, 998, 1011, 1012, 1017, 1018, 1019, 1021, 1022, 1023, 1024, 1030, 1038, 1041, 1042, 1044, 1047, 1089, 1090, 1091, 1095, 1096, 1098, 1099, 1102, 1103, 1334, 1335, 1336, 1341, 1343, 1346, 1351, 1353, 1354, 1362, 1363, 1377, 1386, 1387, 1393, 1396, 1417, 1420, 1422, 1425, 1426, 1446, 1447, 1470, 1473, 1481, 1493, 1494, 1495, 1500, 1505, 1509, 1513, 1520, 1538, 1540, 1546, 1548, 1550, 1624, 1625, 1629, 1682, or 1683, or a combination thereof, or the complement thereof.

In various embodiments, the insecticidal composition for controlling a Lepidopteran pest is in the form of at least one selected from the group consisting of a solid, liquid (including homogeneous mixtures such as solutions and non-homogeneous mixtures such as suspensions, colloids, micelles, and emulsions), powder, suspension, emulsion, spray, encapsulated or micro-encapsulation formulation, in or on microbeads or other carrier particulates, in a film or coating, or on or within a matrix, or as a seed treatment. Suitable binders, inert carriers, surfactants, and the like can optionally be included in the polynucleotide-containing composition, as is known to one skilled in formulation of insecticides and seed treatments. The Lepidopteran pest to be controlled is generally a pest that infests a plant. In some embodiments, the insecticidal composition is at least one implantable formulation selected from the group consisting of a particulate, pellet, or capsule implanted in the plant; in such embodiments the method comprises implanting in the plant the implantable formulation. In some embodiments, the insecticidal composition is at least one in-furrow formulation selected from the group consisting of a powder, granule, pellet, capsule, spray, or drench, or any other forms suited for applying to a furrow; in such embodiments, the method comprises an in-furrow treatment with the in-furrow formulation. In one embodiment the insecticidal composition can be ingested or otherwise absorbed internally by the Lepidopteran pest. For example, the insecticidal composition can be in the form of bait. In some embodiments, the insecticidal composition further comprises one or more components selected from the group consisting of a carrier agent, a surfactant, a cationic lipid (such as that disclosed in Example 18 of U.S. patent application publication 2011/0296556, incorporated by reference herein), an organosilicone, an organosilicone surfactant, a polynucleotide herbicidal molecule, a non-polynucleotide herbicidal molecule, a non-polynucleotide pesticide, a safener, and an insect growth regulator. In one embodiment the insecticidal composition further comprises a nonionic organosilicone surfactant such as SILWET® brand surfactants, e.g., SILWET L-77® brand surfactant having CAS Number 27306-78-1 and EPA Number: CAL.REG.NO. 5905-50073-AA, currently available from Momentive Performance Materials, Albany, N.Y. In some embodiments, the insecticidal composition further comprises at least one pesticidal agent selected from the group consisting of a patatin, a plant lectin, a phytoecdysteroid, a Bacillus thuringiensis insecticidal protein, a Xenorhabdus insecticidal protein, a Photorhabdus insecticidal protein, a Bacillus laterosporus insecticidal protein, and a Bacillus sphaericus insecticidal protein. Alternatively such additional components or pesticidal agents can be provided separately, e.g., by separate topical application or by transgenic expression in the plant. Alternatively the plant is topically treated with the insecticidal composition as well as with a separate (preceding, following, or concurrent) application of a substance that improves the efficacy of the insecticidal composition. For example, a plant can be sprayed with a first topical application of a solution containing a nonionic organosilicone surfactant such as SILWET® brand surfactants, e.g., SILWET L-77® brand surfactant, followed by a second topical application of the insecticidal composition, or vice-versa.

It is anticipated that the combination of certain RNAs of use in this method (e.g., the dsRNA triggers described in the working Examples) with one or more non-polynucleotide pesticidal agents will result in an enhanced improvement in prevention or control of Lepidopteran pest infestations, when compared to the effect obtained with the RNA alone or the non-polynucleotide pesticidal agent alone. In an embodiment, the insecticidal composition contains one or more RNAs and one or more non-polynucleotide pesticidal agent selected from the group consisting of a patatin, a plant lectin, a phytoecdysteroid, a Bacillus thuringiensis insecticidal protein, a Xenorhabdus insecticidal protein, a Photorhabdus insecticidal protein, a Bacillus laterosporus insecticidal protein, and a Bacillus sphaericus insecticidal protein, and is found to effect improved prevention or control of Lepidopteran pest infestations.

In various embodiments, the insecticidal composition comprises a microbial cell or is produced in a microorganism. For example, the insecticidal composition can include or can be produced in bacteria or yeast cells. In similar embodiments the insecticidal composition comprises a transgenic plant cell or is produced in a plant cell (for example a plant cell transiently expressing the polynucleotide); such plant cells can be cells in an plant or cells grown in tissue culture or in cell suspension.

The insecticidal composition can be provided for dietary uptake by the Lepidopteran pest by applying the composition to a plant or surface subject to infestation by the Lepidopteran pest, for example by spraying, dusting, or coating the plant or a seed of the plant or a seed potato, or by application of a soil drench or in-furrow treatment, or by providing in an artificial diet. The insecticidal composition can be provided for dietary uptake by the Lepidopteran pest in an artificial diet formulated to meet the particular nutritional requirements for maintaining the Lepidopteran pest, wherein the artificial diet is supplemented with some amount of the RNA obtained from a separate source such as chemical synthesis or purified from a microbial fermentation; this embodiment can be useful, e.g., for determining the timing and amounts of effective RNA treatment regimes. In some embodiments the insecticidal composition is provided for dietary uptake by the Lepidopteran pest in the form of a plant cell or in plant cell components, or in a microorganism (such as a bacterium or a yeast) or a microbial fermentation product, or in a synthetic diet. In one embodiment the insecticidal composition is provided in the form of bait that is ingested by the Lepidopteran pest. The insecticidal composition can be provided for dietary uptake by the Lepidopteran pest in the form of a seed (or seed potato) treatment.

In one embodiment the insecticidal composition is provided in the form of any plant that is subject to infestation by a Lepidopteran pest, wherein the RNA is contained in or on the plant. Such plants can be stably transgenic plants that express the RNA, or non-transgenic plants that transiently express the RNA or that have been treated with the RNA, e.g., by spraying or coating. Stably transgenic plants generally contain integrated into their genome a recombinant construct that encodes the RNA.

The RNA useful in the insecticidal composition can be single-stranded (ss) or double-stranded (ds). Embodiments include those wherein the RNA is at least one selected from the group consisting of sense single-stranded RNA (ssRNA), anti-sense single-stranded (ssRNA), or double-stranded RNA (dsRNA); a mixture of RNAs of any of these types can be used. In one embodiment a double-stranded DNA/RNA hybrid is used. The RNA can include components other than standard ribonucleotides, e.g., an embodiment is an RNA that comprises terminal deoxyribonucleotides.

The RNA in the insecticidal composition has at least one segment of 18 or more contiguous nucleotides with a sequence of about 95% to about 100% identity with a fragment of equivalent length of a target gene or DNA having a sequence selected from the Target Gene Sequences Group, the Trigger Sequences Group, or the DNA complement of any thereof. In an embodiment the RNA comprises at least one segment of 18 or more contiguous nucleotides that are essentially identical or complementary to a fragment of equivalent length of a DNA having a sequence selected from the Target Gene Sequences Group, the Trigger Sequences Group, or the DNA complement of any thereof. In some embodiments, the contiguous nucleotides have a sequence of about 95%, about 96%, about 97%, about 98%, about 99%, or about 100% identity with a fragment of a DNA having a sequence selected from the Target Gene Sequences Group, the Trigger Sequences Group, or the DNA complement of any thereof. In some embodiments the contiguous nucleotides are exactly (100%) identical to a fragment of equivalent length of a DNA having a sequence selected from the Target Gene Sequences Group, the Trigger Sequences Group, or the DNA complement of any thereof. In some embodiments, the RNA has an overall sequence of about 95%, about 96%, about 97%, about 98%, about 99%, or about 100% identity with a fragment of a DNA having a sequence selected from the Target Gene Sequences Group, the Trigger Sequences Group, or the DNA complement of any thereof.

The RNA in the insecticidal composition comprises at least one segment of 18 or more contiguous nucleotides with a sequence of about 95% to about 100% identity with a fragment of equivalent length of a DNA having a sequence selected from the Target Gene Sequences Group, the Trigger Sequences Group, or the DNA complement of any thereof. In some embodiments the RNA comprises at least one segment of 18 or more contiguous nucleotides, e.g., between 18-24, or between 18-28, or between 20-30, or between 20-50, or between 20-100, or between 50-100, or between 50-500, or between 100-250, or between 100-500, or between 200-1000, or between 500-2000, or even greater. In some embodiments the segment comprises more than 18 contiguous nucleotides, e.g., 19, 20, 21, 22, 23, 24, 25, 26, 27, 28, 29, 30, or greater than 30, e.g., at least about 35, at least about 40, at least about 45, at least about 50, at least about 55, at least about 60, at least about 65, at least about 70, at least about 75, at least about 80, at least about 85, at least about 90, at least about 95, at least about 100, at least about 110, at least about 120, at least about 130, at least about 140, at least about 150, at least about 160, at least about 170, at least about 180, at least about 190, at least about 200, at least about 210, at least about 220, at least about 230, at least about 240, at least about 250, at least about 260, at least about 270, at least about 280, at least about 290, at least about 300, at least about 350, at least about 400, at least about 450, at least about 500, or greater than 500 contiguous nucleotides. In particular embodiments, the RNA comprises at least one segment of at least 18, 19, 20, or 21 contiguous nucleotides with a sequence of 100% identity with a fragment of equivalent length of a DNA or target gene having a sequence selected from the Target Gene Sequences Group, the Trigger Sequences Group, or the DNA complement of any thereof. In particular embodiments, the RNA is a double-stranded nucleic acid (e.g., dsRNA) with one strand comprising at least one segment of at least 18, 19, 20, or 21 contiguous nucleotides with a sequence of 100% identity with a fragment of equivalent length of a DNA or target gene having a sequence selected from the Target Gene Sequences Group, the Trigger Sequences Group, or the DNA complement of any thereof; expressed as base-pairs, such a double-stranded nucleic acid comprises at least one segment of at least 18, 19, 20, or 21 contiguous, perfectly matched base-pairs which correspond to a fragment of equivalent length of a DNA or target gene having a sequence selected from the Target Gene Sequences Group, the Trigger Sequences Group, or the DNA complement of any thereof. In particular embodiments, each segment contained in the RNA is of a length greater than that which is typical of naturally occurring regulatory small RNAs, e.g., each segment is at least about 30 contiguous nucleotides (or base-pairs) in length. In some embodiments, the total length of the RNA, or the length of each segment contained in the RNA, is less than the total length of the sequence of interest (DNA or target gene having a sequence selected from the group consisting of the Target Gene Sequences Group). In some embodiments, the total length of the RNA is between about 50 to about 500 nucleotides (for single-stranded RNAs) or base-pairs (for double-stranded RNAs). In some embodiments, the RNA comprises at least one RNA strand of between about 50 to about 500 nucleotides in length.

The RNA in the insecticidal composition is generally designed to suppress one or more genes (“target genes”). Such target genes can include coding or non-coding sequence or both. In specific embodiments, the RNA is designed to suppress one or more target genes, where each target gene has a DNA sequence selected from the group consisting of the Target Gene Sequences Group. In various embodiments, the RNA is designed to suppress one or more genes, where each gene has a sequence selected from the group consisting of the Target Gene Sequences Group, and can be designed to suppress multiple genes from this group, or to target different regions of one or more of these genes. In an embodiment, the RNA comprises multiple sections or segments each of which comprises at least one segment of 21 contiguous nucleotides with a sequence of 100% identity with a fragment of equivalent length of a DNA having a sequence selected from the Target Gene Sequences Group, the Trigger Sequences Group, or the DNA complement of any thereof. In such cases, each section can be identical or different in size or in sequence, and can be sense or anti-sense relative to the target gene. For example, in one embodiment the RNA can include multiple sections in tandem or repetitive arrangements, wherein each section comprises at least one segment of 21 contiguous nucleotides with a sequence of 100% identity with a fragment of equivalent length of a DNA having a sequence selected from the Target Gene Sequences Group, the Trigger Sequences Group, or the DNA complement of any thereof; the segments can be from different regions of the target gene, e.g., the segments can correspond to different exon regions of the target gene, and “spacer” nucleotides which do not correspond to a target gene can optionally be used in between or adjacent to the segments.

The total length of the RNA in the insecticidal composition can be greater than 18 contiguous nucleotides, and can include nucleotides in addition to the contiguous nucleotides having the sequence of about 95% to about 100% identity with a fragment of equivalent length of a DNA having a sequence selected from the Target Gene Sequences Group, the Trigger Sequences Group, or the DNA complement of any thereof. In other words, the total length of the RNA can be greater than the length of the section or segment of the RNA designed to suppress one or more target genes, where each target gene has a DNA sequence selected from the group consisting of the Target Gene Sequences Group and Trigger Sequences Group. For example, the RNA can have nucleotides flanking the “active” segment of at least one segment of 18 or more contiguous nucleotides that suppresses the target gene, or include “spacer” nucleotides between active segments, or can have additional nucleotides at the 5′ end, or at the 3′ end, or at both the 5′ and 3′ ends. In an embodiment, the RNA comprises additional nucleotides that are not specifically related (having a sequence not complementary or identical to) to the DNA or target gene having a sequence selected from the Target Gene Sequences Group, the Trigger Sequences Group, or the DNA complement of any thereof, e.g., nucleotides that provide stabilizing secondary structure or for convenience in cloning or manufacturing. In an embodiment, the RNA comprises additional nucleotides located immediately adjacent to one or more segment of 18 or more contiguous nucleotides with a sequence of about 95% to about 100% identity with or complementarity to a fragment of equivalent length of a DNA, RNA or target gene having a sequence selected from the Target Gene Sequences Group, the Trigger Sequences Group, or the DNA complement of any thereof. In an embodiment, the RNA comprises one such segment, with an additional 5′ G or an additional 3′ C or both, adjacent to the segment. In another embodiment, the RNA is a double-stranded RNA comprising additional nucleotides to form an overhang, for example, a dsRNA comprising 2 deoxyribonucleotides to form a 3′ overhang. Thus in various embodiments, the nucleotide sequence of the entire RNA is not 100% identical or complementary to a fragment of contiguous nucleotides in the DNA or target gene having a sequence selected from the Target Gene Sequences Group or Trigger Sequences Group. For example, in some embodiments the RNA comprises at least two segments each of 21 contiguous nucleotides with a sequence of 100% identity with a fragment of a DNA having a sequence selected from the Target Gene Sequences Group, the Trigger Sequences Group, or the DNA complement of any thereof, wherein (1) the at least two segments are separated by one or more spacer nucleotides, or (2) the at least two segments are arranged in an order different from that in which the corresponding fragments occur in the DNA having a sequence selected from the Target Gene Sequences Group, the Trigger Sequences Group, or the DNA complement of any thereof.

In various embodiments the RNA in the insecticidal composition consists of naturally occurring ribonucleotides. Embodiments include, for example, synthetic RNAs consisting wholly of ribonucleotides or mainly of ribonucleotides but with one or more terminal deoxyribonucleotides or one or more terminal dideoxyribonucleotides. In certain embodiments, the RNA comprises non-canonical nucleotides such as inosine, thiouridine, or pseudouridine. In certain embodiments, the RNA comprises chemically modified nucleotides. (a) The RNA in the insecticidal composition is provided by suitable means known to one in the art. Embodiments include those wherein the RNA is chemically or enzymatically synthesized (e.g., by in vitro transcription, such as transcription using a T7 polymerase or other polymerase), produced by expression in a microorganism or in cell culture (such as plant or insect cells grown in culture), produced by expression in a plant cell, or produced by microbial fermentation.

In some embodiments the RNA is provided as an isolated RNA that is not part of an expression construct. In some embodiments the RNA is provided as an isolated RNA that is lacking additional elements such as a promoter or terminator sequences. Such RNAs can be relatively short, such as single- or double-stranded RNAs of between about 18 to about 300 or between about 50 to about 500 nucleotides (for single-stranded RNAs) or between about 18 to about 300 or between about 50 to about 500 base-pairs (for double-stranded RNAs). Alternatively the RNA can be provided in more complex constructs, e.g., as part of a recombinant expression construct, or included in a recombinant vector, for example in a recombinant plant virus vector or in a recombinant baculovirus vector. In some embodiments such recombinant expression constructs or vectors are designed to include additional elements, such as including additional RNA encoding an aptamer or ribozyme or an expression cassette for expressing a gene of interest (e.g., an insecticidal protein).

### VII. Methods of Providing Plants Having Improved Resistance to Lepidopteran Pest Infestations, and the Plants and Seeds Thus Provided

Another aspect of this invention is directed to a method of providing a plant having improved resistance to a Lepidopteran pest infestation comprising expressing in the plant at least one polynucleotide comprising at least one segment of 18 or more contiguous nucleotides that is essentially identical or complementary to a fragment of a target gene or DNA having a sequence selected from the Target Gene Sequences Group, the Trigger Sequences Group, or the DNA complement of any thereof, whereby the resulting plant has improved resistance to a Lepidopteran pest when compared to a control plant in which the polynucleotide is not expressed. In an embodiment, the method comprises expressing in the plant at least one polynucleotide comprising at least one segment of 18 or more contiguous nucleotides with a sequence of about 95% to about 100% identity with a fragment of equivalent length of a target gene or DNA having a sequence selected from the Target Gene Sequences Group, the Trigger Sequences Group, or the DNA complement of any thereof. In an embodiment, the invention provides a method of providing a plant having improved resistance to a Lepidopteran pest infestation comprising expressing in the plant at least one polynucleotide comprising at least one segment that is identical or complementary to at least 18, 19, 20, or 21 contiguous nucleotides of a DNA having a sequence selected from the group consisting of: SEQ ID NOs: 2, 5, 7, 9, 13, 21, 26, 27, 29, 36, 37, 39, 44, 47, 87, 95, 99, 103, 301, 309, 318, 319, 320, 321, 323, 324, 328, 341, 343, 346, 356, 358, 359, 503, 504, 505, 506, 509, 510, 530, 532, 533, 542, 545, 546, 547, 549, 551, 552, 553, 559, 560, 561, 563, 564, 570, 572, 573, 581, 593, 596, 602, 611, 612, 620, 665, 666, 672, 717, 730, 731, 736, 737, 738, 740, 741, 742, 743, 749, 757, 760, 761, 763, 766, 808, 809, 810, 814, 815, 817, 818, 821, 822, 1104, 1105, 1106, 1111, 1113, 1116, 1121, 1123, 1124, 1132, 1133, 1147, 1156, 1157, 1163, 1166, 1187, 1190, 1192, 1195, 1196, 1216, 1217, 1240, 1243, 1251, 1263, 1264, 1265, 1270, 1275, 1279, 1283, 1290, 1308, 1310, 1316, 1318, 1320, 1564, 1565, 1569, 1622, and 1623. By “expressing a polynucleotide in the plant” is generally meant “expressing an RNA transcript in the plant”, e.g., expressing in the plant an RNA comprising a ribonucleotide sequence that is anti-sense or essentially complementary to at least a fragment of a target gene or DNA having a sequence selected from the Target Gene Sequences Group, the Trigger Sequences Group, or the DNA complement of any thereof. Embodiments include those in which the polynucleotide expressed in the plant is an RNA comprising at least one segment having a sequence selected from the Trigger Sequences Group, or the complement thereof. However, the polynucleotide expressed in the plant can also be DNA (e.g., a DNA produced in the plant during genome replication), or the RNA encoded by such DNA. Related aspects of the invention include isolated polynucleotides of use in the method and plants having improved Lepidopteran resistance provided by the method.

The method comprises expressing at least one polynucleotide in a plant, wherein the polynucleotide comprises at least one segment of 18 or more contiguous nucleotides that is essentially identical or complementary to a fragment of a target gene or DNA having a sequence selected from the Target Gene Sequences Group, the Trigger Sequences Group, or the DNA complement of any thereof. In some embodiments, a first polynucleotide is provided to a plant in the form of DNA (e.g., in the form of an isolated DNA molecule, or as an expression construct, or as a transformation vector), and the polynucleotide expressed in the plant is a second polynucleotide (e.g., the RNA transcript of the first polynucleotide) in the plant. In an embodiment, the polynucleotide is expressed in the plant by transgenic expression, i.e., by stably integrating the polynucleotide into the plant's genome from where it can be expressed in a cell or cells of the plant. In an embodiment, a first polynucleotide (e.g., a recombinant DNA construct comprising a promoter operably linked to DNA comprising at least one segment of 18 or more contiguous nucleotides that is essentially identical or complementary to a fragment of a target gene or DNA having a sequence selected from the Target Gene Sequences Group, the Trigger Sequences Group, or the DNA complement of any thereof) is stably integrated into the plant's genome from where secondarily produced polynucleotides (e.g., an RNA transcript comprising the transcript of the segment of 18 or more contiguous nucleotides that is essentially identical or complementary to a fragment of a target gene or DNA having a sequence selected from the Target Gene Sequences Group, the Trigger Sequences Group, or the DNA complement of any thereof) are expressed in a cell or cells of the plant. Methods of providing stably transformed plants are provided in the section headed “Making and Using Transgenic Plant Cells and Transgenic Plants”.

In another embodiment the polynucleotide expressed in the plant is expressed by transient expression (i.e., expression not resulting from stable integration of a sequence into the plant's genome). In such embodiments the method can include a step of introducing a polynucleotide (e.g., dsRNA or dsDNA) into the plant by routine techniques known in the art. For example, transient expression can be accomplished by infiltration of a polynucleotide solution using a needle-less syringe into a leaf of a plant.

In some embodiments where the polynucleotide expressed in the plant is expressed by transient expression, a first polynucleotide is provided to a plant in the form of RNA or DNA or both RNA and DNA, and a secondarily produced second polynucleotide is transiently expressed in the plant. In some embodiments, the first polynucleotide is one or more selected from: (a) a single-stranded RNA molecule (ssRNA), (b) a single-stranded RNA molecule that self-hybridizes to form a double-stranded RNA molecule, (c) a double-stranded RNA molecule (dsRNA), (d) a single-stranded DNA molecule (ssDNA), (e) a single-stranded DNA molecule that self-hybridizes to form a double-stranded DNA molecule, (f) a single-stranded DNA molecule comprising a modified Pol III gene that is transcribed to an RNA molecule, (g) a double-stranded DNA molecule (dsDNA), (h) a double-stranded DNA molecule comprising a modified Pol III gene that is transcribed to an RNA molecule, and (i) a double-stranded, hybridized RNA/DNA molecule, or combinations thereof. In specific embodiments, a first polynucleotide is introduced into the plant by topical application to the plant of a polynucleotide-containing composition in a suitable form, e.g., as a solid, liquid (including homogeneous mixtures such as solutions and non-homogeneous mixtures such as suspensions, colloids, micelles, and emulsions), powder, suspension, emulsion, spray, encapsulated or micro-encapsulation formulation, in or on microbeads or other carrier particulates, in a film or coating, or on or within a matrix, or in the form of a treatment of a crop plant seed or treatment of a seed potato. Suitable binders, inert carriers, surfactants, and the like can optionally be included in the composition, as is known to one skilled in formulation of pesticides and seed treatments. In such embodiments, the polynucleotide-containing composition can further include one or more components selected from the group consisting of a carrier agent, a surfactant, a cationic lipid (such as that disclosed in Example 18 of U.S. patent application publication 2011/0296556, incorporated by reference herein), an organosilicone, an organosilicone surfactant, a polynucleotide herbicidal molecule, a non-polynucleotide herbicidal molecule, a non-polynucleotide pesticide, a safener, and an insect growth regulator; in one embodiment the composition further comprises a nonionic organosilicone surfactant such as SILWET® brand surfactants, e.g., SILWET L-77® brand surfactant having CAS Number 27306-78-1 and EPA Number: CAL.REG.NO. 5905-50073-AA, currently available from Momentive Performance Materials, Albany, N.Y. In some embodiments, the topically applied composition further comprises at least one pesticidal agent selected from the group consisting of a patatin, a plant lectin, a phytoecdysteroid, a Bacillus thuringiensis insecticidal protein, a Xenorhabdus insecticidal protein, a Photorhabdus insecticidal protein, a Bacillus laterosporus insecticidal protein, and a Bacillus sphaericus insecticidal protein. Alternatively such additional components or pesticidal agents can be provided separately, e.g., by separate topical application or by transgenic expression in the plant. Alternatively the plant is topically treated with the polynucleotide-containing composition as well as with a separate (preceding, following, or concurrent) application of a substance that improves the efficacy of the polynucleotide-containing composition. For example, a plant can be sprayed with a first topical application of a solution containing a nonionic organosilicone surfactant such as SILWET® brand surfactants, e.g., SILWET L-77® brand surfactant, followed by a second topical application of the polynucleotide-containing composition, or vice-versa.

It is anticipated that the combination of certain polynucleotides of use in this method (e.g., the polynucleotide triggers described in the working Examples) with one or more non-polynucleotide pesticidal agents will result in an enhanced improvement in prevention or control of Lepidopteran pest infestations, when compared to the effect obtained with the polynucleotide alone or the non-polynucleotide pesticidal agent alone. In an embodiment, a transgenic plant expressing at least one polynucleotide comprising at least one segment of 18 or more contiguous nucleotides that is essentially identical or complementary to a fragment of a target gene or DNA having a sequence selected from the Target Gene Sequences Group, the Trigger Sequences Group, or the DNA complement of any thereof (e.g., the polynucleotide triggers described in the working Examples) and one or more genes encoding a non-polynucleotide pesticidal agent selected from the group consisting of a patatin, a plant lectin, a phytoecdysteroid, a Bacillus thuringiensis insecticidal protein, a Xenorhabdus insecticidal protein, a Photorhabdus insecticidal protein, a Bacillus laterosporus insecticidal protein, and a Bacillus sphaericus insecticidal protein, is found to exhibit improved resistance to Lepidopteran pest infestations.

In some embodiments where the polynucleotide expressed in the plant is expressed by transient expression, a first polynucleotide is provided to a plant in the form of RNA or DNA or both RNA and DNA, and a secondarily produced second polynucleotide is transiently expressed in the plant; the site of application of the first polynucleotide need not be the same site where the second polynucleotide is transiently expressed. For example, a first polynucleotide can be provided to a plant by topical application onto a leaf, or by injection into a stem, and the second polynucleotide can be transiently expressed elsewhere in the plant, e.g., in the roots or throughout the plant. In some embodiments of the method, a composition comprising at least one polynucleotide is topically applied to above-ground parts of the plant, e.g., sprayed or dusted onto leaves, stems, and flowering parts of the plant. In other embodiments, a composition comprising at least one polynucleotide is topically applied to below-ground parts of the plant, such as to the roots, e.g., by means of a soil drench. In other embodiments, a composition comprising at least one polynucleotide is topically applied to a seed (or, in the case of potatoes, topically applied to a seed potato) that is grown into the plant having improved resistance to a Lepidopteran pest infestation. In some embodiments the polynucleotide expressed in the plant is RNA, which can be single-stranded (ss) or double-stranded (ds) RNA or a combination of both.

In some embodiments a first polynucleotide (DNA or RNA or both) is provided to a plant and a second polynucleotide having a sequence corresponding (identical or complementary) to the first polynucleotide is subsequently expressed in the plant. In such embodiments the polynucleotide expressed in the plant is an RNA transcript which can be ssRNA or dsRNA or a combination of both. In some embodiments where the polynucleotide is expressed by transient expression, a first polynucleotide is provided to a plant in the form of RNA or DNA or both RNA and DNA, and a secondarily produced second polynucleotide is transiently expressed in the plant; in such embodiments, the first polynucleotide one or more selected from: (a) a single-stranded RNA molecule (ssRNA), (b) a single-stranded RNA molecule that self-hybridizes to form a double-stranded RNA molecule, (c) a double-stranded RNA molecule (dsRNA), (d) a single-stranded DNA molecule (ssDNA), (e) a single-stranded DNA molecule that self-hybridizes to form a double-stranded DNA molecule, (f) a single-stranded DNA molecule comprising a modified Pol III gene that is transcribed to an RNA molecule, (g) a double-stranded DNA molecule (dsDNA), (h) a double-stranded DNA molecule comprising a modified Pol III gene that is transcribed to an RNA molecule, and (i) a double-stranded, hybridized RNA/DNA molecule, or combinations thereof. In such embodiments where the polynucleotide is expressed by transient expression the first polynucleotide can consist of naturally occurring nucleotides, such as those which occur in DNA and RNA. In such embodiments where the polynucleotide is expressed by transient expression the first polynucleotide can be chemically modified, or comprises chemically modified nucleotides. The first polynucleotide is provided by suitable means known to one in the art. Embodiments include those wherein the first polynucleotide is chemically or enzymatically synthesized (e.g., by in vitro transcription, such as transcription using a T7 polymerase or other polymerase), produced by expression in a microorganism or in cell culture (such as plant or insect cells grown in culture), produced by expression in a plant cell, or produced by microbial fermentation. The first polynucleotide can be provided as an RNA or DNA fragment. Alternatively the first polynucleotide can be provided in more complex constructs, e.g., as part of a recombinant expression construct, or included in a recombinant vector, for example in a recombinant plant virus vector or in a recombinant baculovirus vector; such recombinant expression constructs or vectors can be designed to include additional elements, such as expression cassettes for expressing a gene of interest (e.g., an insecticidal protein).

In some embodiments the polynucleotide expressed in the plant is an RNA molecule and can be relatively short, such as single- or double-stranded RNAs of between about 18 to about 300 or between about 50 to about 500 nucleotides (for single-stranded RNAs) or between about 18 to about 300 or between about 50 to about 500 base-pairs (for double-stranded RNAs). Alternatively the polynucleotide can be provided in more complex constructs, e.g., as part of a recombinant expression construct, or included in a recombinant vector, for example in a recombinant plant virus vector or in a recombinant baculovirus vector. In some embodiments such recombinant expression constructs or vectors are designed to include additional elements, such as expression cassettes for expressing a gene of interest (e.g., an insecticidal protein).

The polynucleotide expressed in the plant has at least one segment of 18 or more contiguous nucleotides with a sequence of about 95% to about 100% identity with a fragment of equivalent length of a DNA having a sequence selected from the Target Gene Sequences Group or the Trigger Sequences Group, or the DNA complement of any of the foregoing. In an embodiment the polynucleotide expressed in the plant comprises at least one segment of 18 or more contiguous nucleotides that are essentially identical or complementary to a fragment of equivalent length of a DNA having a sequence selected from the Target Gene Sequences Group, the Trigger Sequences Group, or the DNA complement of any thereof. In some embodiments, the contiguous nucleotides have a sequence of about 95%, about 96%, about 97%, about 98%, about 99%, or about 100% identity with a fragment of a DNA having a sequence selected from the Target Gene Sequences Group or the Trigger Sequences Group, or the DNA complement of any thereof. In some embodiments the contiguous nucleotides are exactly (100%) identical to a fragment of equivalent length of a DNA having a sequence selected from the Target Gene Sequences Group or the Trigger Sequences Group, or the DNA complement of any thereof. In some embodiments, the polynucleotide expressed in the plant has an overall sequence of about 95%, about 96%, about 97%, about 98%, about 99%, or about 100% identity with a fragment of a DNA having a sequence selected from the Target Gene Sequences Group or the Trigger Sequences Group, or the DNA complement of any thereof.

The polynucleotide expressed in the plant is generally designed to suppress one or more genes (“target genes”). Such target genes can include coding or non-coding sequence or both. In specific embodiments, the polynucleotide expressed in the plant is designed to suppress one or more target genes, where each target gene has a DNA sequence selected from the group consisting of the Target Gene Sequences Group. In various embodiments, the polynucleotide expressed in the plant is designed to suppress one or more genes, where each gene has a sequence selected from the group consisting of the Target Gene Sequences Group, and can be designed to suppress multiple genes from this group, or to target different regions of one or more of these genes. In an embodiment, the polynucleotide expressed in the plant comprises multiple sections or segments each of which comprises at least one segment of 21 contiguous nucleotides with a sequence of 100% identity with a fragment of equivalent length of a DNA having a sequence selected from the Target Gene Sequences Group or the Trigger Sequences Group, or the DNA complement of any thereof. In such cases, each section can be identical or different in size or in sequence, and can be sense or anti-sense relative to the target gene. For example, in one embodiment the polynucleotide expressed in the plant can include multiple sections in tandem or repetitive arrangements, wherein each section comprises at least one segment of 21 contiguous nucleotides with a sequence of 100% identity with a fragment of equivalent length of a DNA having a sequence selected from the Target Gene Sequences Group or the DNA complement thereof the segments can be from different regions of the target gene, e.g., the segments can correspond to different exon regions of the target gene, and “spacer” nucleotides which do not correspond to a target gene can optionally be used in between or adjacent to the segments.

The total length of the polynucleotide expressed in the plant can be greater than 18 contiguous nucleotides, and can include nucleotides in addition to the contiguous nucleotides having the sequence of about 95% to about 100% identity with a fragment of equivalent length of a DNA having a sequence selected from the Target Gene Sequences Group or the DNA complement thereof. In other words, the total length of the polynucleotide expressed in the plant can be greater than the length of the section or segment of the polynucleotide designed to suppress one or more target genes, where each target gene has a DNA sequence selected from the group consisting of the Target Gene Sequences Group. For example, the polynucleotide expressed in the plant can have nucleotides flanking the “active” segment of at least one segment of 18 or more contiguous nucleotides that suppresses the target gene, or include “spacer” nucleotides between active segments, or can have additional nucleotides at the 5′ end, or at the 3′ end, or at both the 5′ and 3′ ends. In an embodiment, the polynucleotide expressed in the plant comprises additional nucleotides that are not specifically related (i.e., having a sequence not complementary or identical to) to the DNA or target gene having a sequence selected from the Target Gene Sequences Group or the DNA complement thereof, e.g., nucleotides that provide stabilizing secondary structure or for convenience in cloning or manufacturing. In an embodiment, the polynucleotide expressed in the plant comprises additional nucleotides located immediately adjacent to one or more segment of 18 or more contiguous nucleotides with a sequence of about 95% to about 100% identity with or complementarity to a fragment of equivalent length of a DNA or target gene having a sequence selected from the group consisting of the Target Gene Sequences Group. In an embodiment, the polynucleotide expressed in the plant comprises one such segment, with an additional 5′ G or an additional 3′ C or both, adjacent to the segment. In another embodiment, the polynucleotide expressed in the plant is a double-stranded RNA comprising additional nucleotides to form an overhang, for example, a dsRNA comprising 2 deoxyribonucleotides to form a 3′ overhang. Thus in various embodiments, the nucleotide sequence of the entire polynucleotide expressed in the plant is not 100% identical or complementary to a fragment of contiguous nucleotides in the DNA or target gene having a sequence selected from the Target Gene Sequences Group, the Trigger Sequences Group, or the DNA complement of any thereof. For example, in some embodiments the polynucleotide expressed in the plant comprises at least two segments each of 21 contiguous nucleotides with a sequence of 100% identity with a fragment of a DNA having a sequence selected from the Target Gene Sequences Group, the Trigger Sequences Group, or the DNA complement of any thereof, wherein (1) the at least two segments are separated by one or more spacer nucleotides, or (2) the at least two segments are arranged in an order different from that in which the corresponding fragments occur in the DNA having a sequence selected from the Target Gene Sequences Group, the Trigger Sequences Group, or the DNA complement of any thereof.

In a related aspect, this invention is directed to the plant having improved resistance to a Lepidopteran pest infestation, provided by expressing in the plant at least one polynucleotide comprising at least one segment of 18 or more contiguous nucleotides that are essentially identical or complementary to a fragment of equivalent length of a DNA having a sequence selected from the Target Gene Sequences Group, the Trigger Sequences Group, or the DNA complement of any thereof, whereby the resulting plant has improved resistance to a Lepidopteran pest infestation when compared to a control plant in which the polynucleotide is not expressed. In a related aspect, this invention is directed to the plant having improved resistance to a Lepidopteran pest infestation, provided by expressing in the plant at least one polynucleotide comprising at least one segment of 18 or more contiguous nucleotides with a sequence of about 95% to about 100% identity with a fragment of equivalent length of a DNA having a sequence selected from the Target Gene Sequences Group or the Trigger Sequences Group, or the DNA complement of any thereof, whereby the resulting plant has improved resistance to a Lepidopteran pest infestation when compared to a control plant in which the polynucleotide is not expressed. An embodiment is a crop plant having improved resistance to a Lepidopteran pest infestation when compared to a control plant, provided by expressing in the plant an RNA having a sequence selected from the Trigger Sequences Group, or the complement thereof. In yet another aspect, this invention is directed to seed (especially transgenic progeny seed) produced by the plant having improved resistance to a Lepidopteran pest infestation, as provided by this method. Also contemplated is a commodity product produced by the plant having improved resistance to a Lepidopteran pest infestation, as provided by this method, and a commodity product produced from the transgenic progeny seed of such a plant.

### VIII. Recombinant DNA Constructs for Controlling a Lepidopteran Pest

Another aspect of this invention provides a recombinant DNA construct comprising a heterologous promoter operably linked to a DNA element comprising at least one segment of 18 or more contiguous nucleotides with a sequence of about 95% to about 100% identity with a fragment of a DNA having a sequence selected from the Target Gene Sequences Group, the Trigger Sequences Group, or the DNA complement of any thereof. In some embodiments, the recombinant DNA construct comprises a heterologous promoter operably linked to: (a) DNA comprising a nucleotide sequence that is complementary to at least 18, 19, 20 or 21 contiguous nucleotides of a target gene having a sequence selected from the group consisting of: SEQ ID NOs: 2, 5, 7, 9, 13, 21, 26, 27, 29, 36, 37, 39, 44, 47, 87, 95, 99, 103, 301, 309, 318, 319, 320, 321, 323, 324, 328, 341, 343, 346, 356, 358, 359, 503, 504, 505, 506, 509, 510, 530, 532, 533, 542, 545, 546, 547, 549, 551, 552, 553, 559, 560, 561, 563, 564, 570, 572, 573, 581, 593, 596, 602, 611, 612, 620, 665, 666, 672, 717, 730, 731, 736, 737, 738, 740, 741, 742, 743, 749, 757, 760, 761, 763, 766, 808, 809, 810, 814, 815, 817, 818, 821, 822, 1104, 1105, 1106, 1111, 1113, 1116, 1121, 1123, 1124, 1132, 1133, 1147, 1156, 1157, 1163, 1166, 1187, 1190, 1192, 1195, 1196, 1216, 1217, 1240, 1243, 1251, 1263, 1264, 1265, 1270, 1275, 1279, 1283, 1290, 1308, 1310, 1316, 1318, 1320, 1564, 1565, 1569, 1622, and 1623, or an RNA transcribed from the target gene; or (b) a DNA comprising 18, 19, 20, or 21 or more contiguous nucleotides having 100% identity to a fragment of equivalent length of a DNA having a sequence selected from the group consisting of: SEQ ID NOs: 2, 5, 7, 9, 13, 21, 26, 27, 29, 36, 37, 39, 44, 47, 87, 95, 99, 103, 301, 309, 318, 319, 320, 321, 323, 324, 328, 341, 343, 346, 356, 358, 359, 503, 504, 505, 506, 509, 510, 530, 532, 533, 542, 545, 546, 547, 549, 551, 552, 553, 559, 560, 561, 563, 564, 570, 572, 573, 581, 593, 596, 602, 611, 612, 620, 665, 666, 672, 717, 730, 731, 736, 737, 738, 740, 741, 742, 743, 749, 757, 760, 761, 763, 766, 808, 809, 810, 814, 815, 817, 818, 821, 822, 1104, 1105, 1106, 1111, 1113, 1116, 1121, 1123, 1124, 1132, 1133, 1147, 1156, 1157, 1163, 1166, 1187, 1190, 1192, 1195, 1196, 1216, 1217, 1240, 1243, 1251, 1263, 1264, 1265, 1270, 1275, 1279, 1283, 1290, 1308, 1310, 1316, 1318, 1320, 1564, 1565, 1569, 1622, and 1623, or the DNA complement thereof; or (c) DNA encoding at least one silencing element that is complementary to at least 18, 19, 20, or 21 contiguous nucleotides of a target gene or an RNA transcribed from the target gene, wherein the target gene has a sequence selected from the group consisting of: SEQ ID NOs: 2, 5, 7, 9, 13, 21, 26, 27, 29, 36, 37, 39, 44, 47, 87, 95, 99, 103, 301, 309, 318, 319, 320, 321, 323, 324, 328, 341, 343, 346, 356, 358, 359, 503, 504, 505, 506, 509, 510, 530, 532, 533, 542, 545, 546, 547, 549, 551, 552, 553, 559, 560, 561, 563, 564, 570, 572, 573, 581, 593, 596, 602, 611, 612, 620, 665, 666, 672, 717, 730, 731, 736, 737, 738, 740, 741, 742, 743, 749, 757, 760, 761, 763, 766, 808, 809, 810, 814, 815, 817, 818, 821, 822, 1104, 1105, 1106, 1111, 1113, 1116, 1121, 1123, 1124, 1132, 1133, 1147, 1156, 1157, 1163, 1166, 1187, 1190, 1192, 1195, 1196, 1216, 1217, 1240, 1243, 1251, 1263, 1264, 1265, 1270, 1275, 1279, 1283, 1290, 1308, 1310, 1316, 1318, 1320, 1564, 1565, 1569, 1622, and 1623; or (d) DNA encoding at least one silencing element comprising at least 18, 19, 20 or 21 contiguous nucleotides that are complementary to a target gene selected from the genes in the Target Gene Sequences Group or an RNA transcribed from the target gene; or (e) DNA encoding a RNA comprising at least 18, 19, 20, or 21 contiguous nucleotides that are complementary to a nucleotide sequence selected from the Trigger Sequences Group, or the complement thereof, or an orthologous nucleotide sequence from a Lepidopteran pest, wherein the orthologous nucleotide sequence has at least 95% sequence identity with a nucleotide sequence selected from the Trigger Sequences Group, wherein the percentage sequence identity is calculated over the same length; or (f) DNA encoding a RNA comprising at least one double-stranded RNA region, at least one strand of which comprises at least 18, 19, 20 or 21 contiguous nucleotides that are complementary to a nucleotide sequence selected from the Trigger Sequences Group, or the complement thereof, or an orthologous nucleotide sequence from a Lepidopteran pest, wherein the orthologous nucleotide sequence has at least 95% sequence identity with a nucleotide sequence selected from the Target Gene Sequences Group, the Trigger Sequences Group, or the DNA complement of any thereof, wherein the percentage sequence identity is calculated over the same length; or (g) DNA encoding RNA comprising a nucleotide sequence selected from the Trigger Sequences Group, or the complement thereof. Embodiments include a recombinant DNA construct comprising a heterologous promoter operably linked to a DNA element encoding an RNA having a sequence selected from the group consisting of: SEQ ID NO: 115, 116, 119, 121, 123, 127, 135, 140, 141, 143, 150, 151, 153, 158, 161, 201, 209, 213, 217, 434, 442, 451, 452, 453, 454, 456, 457, 461, 474, 476, 479, 489, 491, 492, 521, 522, 523, 524, 527, 528, 536, 538, 539, 823, 826, 827, 828, 830, 832, 833, 834, 840, 841, 842, 844, 845, 851, 853, 854, 862, 874, 877, 883, 892, 893, 901, 946, 947, 953, 998, 1011, 1012, 1017, 1018, 1019, 1021, 1022, 1023, 1024, 1030, 1038, 1041, 1042, 1044, 1047, 1089, 1090, 1091, 1095, 1096, 1098, 1099, 1102, 1103, 1334, 1335, 1336, 1341, 1343, 1346, 1351, 1353, 1354, 1362, 1363, 1377, 1386, 1387, 1393, 1396, 1417, 1420, 1422, 1425, 1426, 1446, 1447, 1470, 1473, 1481, 1493, 1494, 1495, 1500, 1505, 1509, 1513, 1520, 1538, 1540, 1546, 1548, 1550, 1624, 1625, 1629, 1682, or 1683, or a combination thereof. or the complement thereof.

Embodiments include a recombinant DNA construct comprising a heterologous promoter operably linked to a DNA encoding a dsRNA with a strand having a sequence selected from the Trigger Sequences Group, or a fragment thereof, or a complement of any thereof. The recombinant DNA constructs are useful in providing a plant having improved resistance to a Lepidopteran pest infestation, e.g., by expressing in a plant a transcript of such a recombinant DNA construct. The recombinant DNA constructs are also useful in the manufacture of polynucleotides useful in making compositions that can be applied to a plant, seed, propagatable plant part, soil or field, or surface in need of protection from a Lepidopteran pest infestation. Related aspects of the invention include: compositions comprising the recombinant DNA construct; a plant chromosome or a plastid or a recombinant plant virus vector or a recombinant baculovirus vector comprising the recombinant DNA construct; a transgenic solanaceous plant cell having in its genome the recombinant DNA construct, optionally comprising in its genome DNA encoding at least one pesticidal agent selected from the group consisting of a patatin, a plant lectin, a phytoecdysteroid, a Bacillus thuringiensis insecticidal protein, a Xenorhabdus insecticidal protein, a Photorhabdus insecticidal protein, a Bacillus laterosporus insecticidal protein, and a Bacillus sphaericus insecticidal protein, and a transgenic solanaceous plant including such a transgenic solanaceous plant cell, or a fruit, seed, or propagatable part of the transgenic solanaceous plant; and plants having improved Lepidopteran resistance provided by expression of or treatment with the recombinant DNA construct or the RNA encoded therein.

The recombinant DNA construct comprises a heterologous promoter operably linked to DNA comprising at least one segment of 18 or more contiguous nucleotides with a sequence of about 95% to about 100% identity with a fragment of equivalent length of a DNA having a sequence selected from the Target Gene Sequences Group or the DNA complement thereof. In some embodiments, the segment of 18 or more contiguous nucleotides has a sequence with about 95%, about 96%, about 97%, about 98%, about 99%, or about 100% identity with a fragment of a DNA having a sequence selected from the Target Gene Sequences Group or the DNA complement thereof. In some embodiments the contiguous nucleotides are exactly (100%) identical to a fragment of equivalent length of a DNA having a sequence selected from the Target Gene Sequences Group or the DNA complement thereof. In some embodiments, the DNA has an overall sequence of about 95%, about 96%, about 97%, about 98%, about 99%, or about 100% identity with a DNA having a sequence selected from the Target Gene Sequences Group or the DNA complement thereof.

The recombinant DNA construct therefore comprises a heterologous promoter operably linked to DNA comprising at least one segment of 18 or more contiguous nucleotides designed to suppress expression of a target gene having a sequence selected from the Target Gene Sequences Group or the DNA complement thereof. In some embodiments the DNA comprises at least one segment of 18 or more contiguous nucleotides, e.g., between 18-24, or between 18-28, or between 20-30, or between 20-50, or between 20-100, or between 50-100, or between 50-500, or between 100-250, or between 100-500, or between 200-1000, or between 500-2000, or even greater. In some embodiments the segment comprises more than 18 contiguous nucleotides, e.g., 19, 20, 21, 22, 23, 24, 25, 26, 27, 28, 29, 30, or greater than 30, e.g., at least about 35, at least about 40, at least about 45, at least about 50, at least about 55, at least about 60, at least about 65, at least about 70, at least about 75, at least about 80, at least about 85, at least about 90, at least about 95, at least about 100, at least about 110, at least about 120, at least about 130, at least about 140, at least about 150, at least about 160, at least about 170, at least about 180, at least about 190, at least about 200, at least about 210, at least about 220, at least about 230, at least about 240, at least about 250, at least about 260, at least about 270, at least about 280, at least about 290, at least about 300, at least about 350, at least about 400, at least about 450, at least about 500, or greater than 500 contiguous nucleotides. In particular embodiments, the DNA encodes an RNA containing at least one segment of at least 18, 19, 20, or 21 contiguous nucleotides with a sequence of 100% identity with a fragment of equivalent length of a DNA or target gene having a sequence selected from the Target Gene Sequences Group or the DNA complement thereof. In particular embodiments, the DNA encodes a double-stranded nucleic acid (e.g., dsRNA) with one strand comprising at least one segment of at least 18, 19, 20, or 21 contiguous nucleotides with a sequence of 100% identity with a fragment of equivalent length of a DNA or target gene having a sequence selected from the Target Gene Sequences Group or the DNA complement thereof; expressed as base-pairs, such a double-stranded nucleic acid comprises at least one segment of at least 18, 19, 20, or 21 contiguous, perfectly matched base-pairs which correspond to a fragment of equivalent length of a DNA or target gene having a sequence selected from the Target Gene Sequences Group or the DNA complement thereof. In particular embodiments, each segment contained in the DNA is of a length greater than that which is typical of naturally occurring regulatory small RNAs. In some embodiments, each segment is at least about 30 contiguous nucleotides (or base-pairs) in length. In some embodiments, the total length of the DNA, or the length of each segment contained in the polynucleotide, is less than the total length of the sequence of interest (DNA or target gene having a sequence selected from the group consisting of the Target Gene Sequences Group). In some embodiments, the total length of the DNA is between about 50 to about 500. In some embodiments, the DNA encodes an RNA having a sequence selected from the group consisting of: SEQ ID NO: 115, 116, 119, 121, 123, 127, 135, 140, 141, 143, 150, 151, 153, 158, 161, 201, 209, 213, 217, 434, 442, 451, 452, 453, 454, 456, 457, 461, 474, 476, 479, 489, 491, 492, 521, 522, 523, 524, 527, 528, 536, 538, 539, 823, 826, 827, 828, 830, 832, 833, 834, 840, 841, 842, 844, 845, 851, 853, 854, 862, 874, 877, 883, 892, 893, 901, 946, 947, 953, 998, 1011, 1012, 1017, 1018, 1019, 1021, 1022, 1023, 1024, 1030, 1038, 1041, 1042, 1044, 1047, 1089, 1090, 1091, 1095, 1096, 1098, 1099, 1102, 1103, 1334, 1335, 1336, 1341, 1343, 1346, 1351, 1353, 1354, 1362, 1363, 1377, 1386, 1387, 1393, 1396, 1417, 1420, 1422, 1425, 1426, 1446, 1447, 1470, 1473, 1481, 1493, 1494, 1495, 1500, 1505, 1509, 1513, 1520, 1538, 1540, 1546, 1548, 1550, 1624, 1625, 1629, 1682, or 1683 or a combination thereof, or the complement thereof.

The recombinant DNA construct comprises a heterologous promoter operably linked to DNA generally designed to suppress one or more genes (“target genes”). Such target genes can include coding or non-coding sequence or both. In specific embodiments, the recombinant DNA construct is designed to suppress one or more target genes, where each target gene has a DNA sequence selected from the group consisting of the Target Gene Sequences Group. In various embodiments, the recombinant DNA construct is designed to suppress one or more genes, where each gene has a sequence selected from the group consisting of the Target Gene Sequences Group, and can be designed to suppress multiple genes from this group, or to target different regions of one or more of these genes. In an embodiment, the recombinant DNA construct comprises a heterologous promoter operably linked to multiple sections or segments each of which comprises at least one segment of 21 contiguous nucleotides with a sequence of 100% identity with a fragment of equivalent length of a DNA having a sequence selected from the Target Gene Sequences Group or the DNA complement thereof. In such cases, each section can be identical or different in size or in sequence, and can be sense or anti-sense relative to the target gene. For example, in one embodiment the recombinant DNA construct can include a heterologous promoter operably linked to multiple sections in tandem or repetitive arrangements, wherein each section comprises at least one segment of 21 contiguous nucleotides with a sequence of 100% identity with a fragment of equivalent length of a DNA having a sequence selected from the Target Gene Sequences Group or the DNA complement thereof the segments can be from different regions of the target gene, e.g., the segments can correspond to different exon regions of the target gene, and “spacer” nucleotides which do not correspond to a target gene can optionally be used in between or adjacent to the segments.

The recombinant DNA construct comprises a heterologous promoter operably linked to DNA which can have a total length that is greater than 18 contiguous nucleotides, and can include nucleotides in addition to the segment of at least one segment of 18 or more contiguous nucleotides having the sequence of about 95% to about 100% identity with a fragment of equivalent length of a DNA having a sequence selected from the Target Gene Sequences Group or the DNA complement thereof. In other words, the total length of the DNA can be greater than the length of the segment of the DNA designed to suppress one or more target genes, where each target gene has a DNA sequence selected from the group consisting of the Target Gene Sequences Group. For example, the DNA can have nucleotides flanking the “active” segment of at least one segment of 18 or more contiguous nucleotides that suppresses the target gene, or include “spacer” nucleotides between active segments, or can have additional nucleotides at the 5′ end, or at the 3′ end, or at both the 5′ and 3′ ends. In an embodiment, the heterologous promoter is operably linked to DNA comprising additional nucleotides that are not specifically related (having a sequence not complementary or identical to) to the DNA or target gene having a sequence selected from the Target Gene Sequences Group or the DNA complement thereof, e.g., nucleotides that provide stabilizing secondary structure or for convenience in cloning or manufacturing. In an embodiment, the heterologous promoter is operably linked to DNA comprising additional nucleotides located immediately adjacent to one or more segment of 18 or more contiguous nucleotides with a sequence of about 95% to about 100% identity with or complementarity to a fragment of equivalent length of a DNA or target gene having a sequence selected from the Target Gene Sequences Group, the Trigger Sequences Group, or the DNA complement of any thereof. In an embodiment, the heterologous promoter is operably linked to DNA comprising one such segment, with an additional 5′ G or an additional 3′ C or both, adjacent to the segment. In another embodiment, the heterologous promoter is operably linked to DNA encoding a double-stranded RNA comprising additional nucleotides to form an overhang. Thus in various embodiments, the nucleotide sequence of the entire DNA operably linked to the heterologous promoter is not 100% identical or complementary to a fragment of contiguous nucleotides in the DNA or target gene having a sequence selected from the Target Gene Sequences Group, the Trigger Sequences Group, or the DNA complement of any thereof. For example, in some embodiments the heterologous promoter is operably linked to DNA comprising at least two segments each of 21 contiguous nucleotides with a sequence of 100% identity with a fragment of a DNA having a sequence selected from the Target Gene Sequences Group, the Trigger Sequences Group, or the DNA complement of any thereof, wherein (1) the at least two segments are separated by one or more spacer nucleotides, or (2) the at least two segments are arranged in an order different from that in which the corresponding fragments occur in the DNA having a sequence selected from the Target Gene Sequences Group, the Trigger Sequences Group, or the DNA complement of any thereof.

In recombinant DNA constructs, the heterologous promoter is operably linked to DNA that encodes a transcript that can be single-stranded (ss) or double-stranded (ds) or a combination of both. Embodiments of the method include those wherein the DNA encodes a transcript comprising sense single-stranded RNA (ssRNA), anti-sense ssRNA, or double-stranded RNA (dsRNA), or a combination of any of these.

The recombinant DNA construct is provided by suitable means known to one in the art. Embodiments include those wherein the recombinant DNA construct is synthesized in vitro, produced by expression in a microorganism or in cell culture (such as plant or insect cells grown in culture), produced by expression in a plant cell, or produced by microbial fermentation.

The heterologous promoter of use in recombinant DNA constructs is selected from the group consisting of a promoter functional in a plant, a promoter functional in a prokaryote, a promoter functional in a fungal cell, and a baculovirus promoter. Non-limiting examples of promoters are described in the section headed “Promoters”.

In some embodiments, the recombinant DNA construct comprises a second promoter also operably linked to the DNA. For example, the DNA comprising at least one segment of 18 or more contiguous nucleotides can be flanked by two promoters arranged so that the promoters transcribe in opposite directions and in a convergent manner, yielding opposite-strand transcripts of the DNA that are complementary to and capable of hybridizing with each other to form double-stranded RNA. In one embodiment, the DNA is located between two root-specific promoters, which enable transcription of the DNA in opposite directions, resulting in the formation of dsRNA.

In some embodiments the recombinant DNA construct comprises other DNA elements in addition to the heterologous promoter operably linked to DNA comprising at least one segment of 18 or more contiguous nucleotides with a sequence of about 95% to about 100% identity with a fragment of equivalent length of a DNA having a sequence selected from the Target Gene Sequences Group or the DNA complement thereof. Such DNA elements are known in the art, and include but are not limited to introns, recombinase recognition sites, aptamers or ribozymes, additional and additional expression cassettes for expressing coding sequences (e.g., to express a transgene such as an insecticidal protein or selectable marker) or non-coding sequences (e.g., to express additional suppression elements). Inclusion of one or more recognition sites for binding and cleavage by a small RNA (e.g., by a miRNA or an siRNA that is expressed only in a particular cell or tissue) allows for more precise expression patterns in a plant, wherein the expression of the recombinant DNA construct is suppressed where the small RNA is expressed.

In some embodiments, the recombinant DNA construct is provided in a recombinant vector. By “recombinant vector” is meant a recombinant polynucleotide molecule that is used to transfer genetic information from one cell to another. Embodiments suitable to this invention include, but are not limited to, recombinant plasmids, recombinant cosmids, artificial chromosomes, and recombinant viral vectors such as recombinant plant virus vectors and recombinant baculovirus vectors. Alternative embodiments include recombinant plasmids, recombinant cosmids, artificial chromosomes, and recombinant viral vectors such as recombinant plant virus vectors and recombinant baculovirus vectors comprising the DNA element without the heterologous promoter.

In some embodiments, the recombinant DNA construct is provided in a plant chromosome or plastid, e.g., in a transgenic plant cell or a transgenic plant. Thus, also encompassed by this invention is a transgenic plant cell having in its genome the recombinant DNA construct, as well as a transgenic plant or partially transgenic plant including such a transgenic plant cell. Partially transgenic plants include, e.g., a non-transgenic scion grafted onto a transgenic rootstock including the transgenic plant cell. Embodiments include a transgenic tomato rootstock including the transgenic plant cell. The plant can be any plant that is subject to infestation by a Lepidopteran pest. Of particular interest are embodiments wherein the plant is a crop plant. Embodiments include those wherein the plant is an ungerminated crop plant seed, a crop plant in a vegetative stage, or a crop plant in a reproductive stage. Embodiments include those wherein the plant is a “seed potato”, meaning a potato tuber or piece of potato tuber which can be propagated into new potato plants. In yet another aspect, this invention is directed to seed (especially transgenic progeny seed) produced by the transgenic plant having in its genome a recombinant DNA construct as described herein. Embodiments also encompass a transgenic seed potato having in its genome a recombinant DNA construct as described herein. Also contemplated is a commodity product produced by such a transgenic plant, and a commodity product produced from the transgenic progeny seed of such a transgenic plant.

The recombinant DNA construct can be provided in a composition for topical application to a surface of a plant or of a plant seed, or for topical application to any substrate needing protection from a Lepidopteran pest infestation. Likewise, the recombinant DNA construct can be provided in a composition for topical application to a Lepidopteran pest, or in a composition for ingestion by a Lepidopteran pest. In various embodiments, such compositions containing the recombinant DNA construct are provided in the form of at least one selected from the group consisting of a solid, liquid (including homogeneous mixtures such as solutions and non-homogeneous mixtures such as suspensions, colloids, micelles, and emulsions), powder, suspension, emulsion, spray, encapsulated or micro-encapsulation formulation, in or on microbeads or other carrier particulates, in a film or coating, or on or within a matrix, or as a seed treatment. The topical application can be in the form of topical treatment of fruits of solanaceous plants or seeds from fruits of solanaceous plants, or in the form of topical treatment of “seed potato” tubers or pieces of tuber (e.g., by soaking, coating, or dusting the seed potato). Suitable binders, inert carriers, surfactants, and the like can be included in the composition containing the recombinant DNA construct, as is known to one skilled in formulation of pesticides and seed treatments. In some embodiments, the composition for topical application containing the recombinant DNA construct is at least one topically implantable formulation selected from the group consisting of a particulate, pellet, or capsule topically implanted in the plant; in such embodiments the method comprises topically implanting in the plant the topically implantable formulation. In some embodiments, the composition for topical application containing the recombinant DNA construct is at least one in-furrow formulation selected from the group consisting of a powder, granule, pellet, capsule, spray, or drench, or any other forms suited for topically applying to a furrow; in such embodiments, the method includes an in-furrow treatment with the in-furrow formulation. In one embodiment the composition for topical application containing the recombinant DNA construct can be ingested or otherwise absorbed internally by the Lepidopteran pest. For example, the composition for topical application containing the recombinant DNA construct can be in the form of bait. In some embodiments, the composition containing the recombinant DNA construct further comprises one or more components selected from the group consisting of a carrier agent, a surfactant, a cationic lipid (such as that disclosed in Example 18 of U.S. patent application publication 2011/0296556, incorporated by reference herein), an organosilicone, an organosilicone surfactant, a polynucleotide herbicidal molecule, a non-polynucleotide herbicidal molecule, a non-polynucleotide pesticide, a safener, and an insect growth regulator. In one embodiment the composition containing the recombinant DNA construct further comprises a nonionic organosilicone surfactant such as SILWET® brand surfactants, e.g., SILWET L-77® brand surfactant having CAS Number 27306-78-1 and EPA Number: CAL.REG.NO. 5905-50073-AA, currently available from Momentive Performance Materials, Albany, N.Y. In some embodiments, the composition containing the recombinant DNA construct further comprises at least one pesticidal agent selected from the group consisting of a patatin, a plant lectin, a phytoecdysteroid, a phytoecdysteroid, a Bacillus thuringiensis insecticidal protein, a Xenorhabdus insecticidal protein, a Photorhabdus insecticidal protein, a Bacillus laterosporus insecticidal protein, and a Bacillus sphaericus insecticidal protein.

It is anticipated that the combination of certain recombinant DNA constructs as described herein (e.g., recombinant DNA constructs including the polynucleotide triggers described in the working Examples), whether transgenically expressed or topically applied, with one or more non-polynucleotide pesticidal agents, whether transgenically expressed or topically applied, will result in an enhanced improvement in prevention or control of Lepidopteran pest infestations, when compared to the effect obtained with the recombinant DNA constructs alone or the non-polynucleotide pesticidal agent alone. In an embodiment, a recombinant DNA construct for expressing one or more polynucleotides as well as one or more genes encoding a non-polynucleotide pesticidal agent selected from the group consisting of a patatin, a plant lectin, a phytoecdysteroid, a Bacillus thuringiensis insecticidal protein, a Xenorhabdus insecticidal protein, a Photorhabdus insecticidal protein, a Bacillus laterosporus insecticidal protein, and a Bacillus sphaericus insecticidal protein, is found to provide improved resistance to Lepidopteran pest infestations in plants expressing the recombinant DNA construct. An embodiment relates to a recombinant DNA construct for expressing an RNA comprising a segment having a sequence selected from the Trigger Sequences Group as well as one or more genes encoding a non-polynucleotide pesticidal agent selected from the group consisting of a patatin, a plant lectin, a phytoecdysteroid, a Bacillus thuringiensis insecticidal protein, a Xenorhabdus insecticidal protein, a Photorhabdus insecticidal protein, a Bacillus laterosporus insecticidal protein, and a Bacillus sphaericus insecticidal protein.

The composition containing the recombinant DNA construct can be provided for dietary uptake by a Lepidopteran pest by applying the composition to a plant or surface subject to infestation by the Lepidopteran pest, for example by spraying, dusting, or coating the plant, or by application of a soil drench, or by providing in an artificial diet. The composition containing the recombinant DNA construct can be provided for dietary uptake by a Lepidopteran pest in an artificial diet formulated to meet the particular nutritional requirements for maintaining the Lepidopteran pest, wherein the artificial diet is supplemented with some amount of the recombinant DNA construct obtained from a separate source such as in vitro synthesis or purified from a microbial fermentation or other biological source; this embodiment can be useful, e.g., for determining the timing and amounts of effective treatment regimes. In some embodiments the composition containing the recombinant DNA construct is provided for dietary uptake by the Lepidopteran pest in the form of a plant cell or in plant cell components, or in a microorganism (such as a bacterium or a yeast) or a microbial fermentation product, or in a synthetic diet. In one embodiment the composition containing the recombinant DNA construct is provided in the form of bait that is ingested by the Lepidopteran pest. The composition containing the recombinant DNA construct can be provided for dietary uptake by the Lepidopteran pest in the form of a seed treatment.

In various embodiments, the composition containing the recombinant DNA construct comprises a microbial cell or is produced in a microorganism. For example, the composition for containing the recombinant DNA construct can include or can be produced in bacteria or yeast cells. In similar embodiments the composition containing the recombinant DNA construct comprises a transgenic plant cell or is produced in a plant cell (for example a plant cell transiently expressing the recombinant DNA construct); such plant cells can be cells in an plant or cells grown in tissue culture or in cell suspension.

### IX. Transgenic Plant Cells

Several embodiments relate to transgenic crop plant cells expressing a polynucleotide useful in the methods described herein for suppressing expression of a target gene in a Lepidopteran pest or for controlling a Lepidopteran infestation. In one aspect this invention provides a transgenic solanaceous plant cell having in its genome a recombinant DNA encoding RNA comprising at least one segment of 18 or more contiguous nucleotides with a sequence of about 95% to about 100% identity with a fragment of a DNA having a sequence selected from the Target Gene Sequences Group, the Trigger Sequences Group, or the DNA complement of any thereof. In one aspect this invention provides a transgenic solanaceous plant cell having in its genome a recombinant DNA encoding RNA comprising at least one silencing element essentially identical or essentially complementary to a fragment of a target gene sequence of the Lepidopteran pest larvae, wherein the target gene sequence is selected from the Target Gene Sequences Group, the Trigger Sequences Group, or the DNA complement of any thereof. In one aspect this invention provides a transgenic solanaceous plant cell having in its genome a recombinant DNA encoding RNA that suppresses expression of a target gene in a Lepidopteran pest that contacts or ingests the RNA, wherein the RNA comprises at least one silencing element having at least one segment of 18 or more contiguous nucleotides complementary to a fragment of the target gene, and wherein the target gene is selected from the group consisting of the genes in the Target Gene Sequences Group. A specific embodiment is a transgenic crop plant cell having in its genome a recombinant DNA encoding RNA that suppresses expression of a target gene in a Lepidopteran pest that contacts or ingests the RNA, wherein the RNA comprises at least one silencing element having at least one segment of 18 or more contiguous nucleotides complementary to a fragment of one or more Target Gene Sequences Group. In one aspect this invention provides a transgenic crop plant cell having in its genome a recombinant DNA encoding an RNA having a sequence selected from the Trigger Sequences Group. Such transgenic crop plant cells are useful in providing a transgenic crop plant having improved resistance to a Lepidopteran pest infestation when compared to a control plant lacking such plant cells. The transgenic crop plant cell can an isolated transgenic solanaceous plant cell, or a transgenic solanaceous plant cell grown in culture, or a transgenic cell of any transgenic crop plant that is subject to infestation by a Lepidopteran pest.

In an embodiment, the recombinant DNA is stably integrated into the transgenic crop plant's genome from where it can be expressed in a cell or cells of the transgenic solanaceous plant. Methods of providing stably transformed plants are provided in the section headed “Making and Using Transgenic Plant Cells and Transgenic Plants”.

Several embodiments relate to a transgenic solanaceous plant cell having in its genome a recombinant DNA encoding RNA that suppresses expression of a target gene in a Lepidopteran pest that contacts or ingests the RNA, wherein the RNA comprises at least one silencing element complementary to the target gene, and wherein the target gene sequence is selected from the Target Gene Sequences Group or the complement thereof. In some embodiments, the silencing element comprises at least one 18 or more contiguous nucleotides with a sequence of about 95% to about 100% complementarity to a fragment of equivalent length of a DNA having a sequence selected from the Target Gene Sequences Group, the Trigger Sequences Group, or the DNA complement of any thereof. In some embodiments, the silencing element comprises at least one 18 or more contiguous nucleotides capable of hybridizing in vivo or of hybridizing under physiological conditions (e.g., such as physiological conditions normally found in the cells of a Lepidopteran pest) to a fragment of equivalent length of a DNA having a sequence selected from the Target Gene Sequences Group, the Trigger Sequences Group, or the DNA complement of any thereof. The contiguous nucleotides number at least 18, e.g., between 18-24, or between 18-28, or between 20-30, or between 20-50, or between 20-100, or between 50-100, or between 50-500, or between 100-250, or between 100-500, or between 200-1000, or between 500-2000, or even greater. In some embodiments, the contiguous nucleotides number more than 18, e.g., 19, 20, 21, 22, 23, 24, 25, 26, 27, 28, 29, 30, or greater than 30, e.g., at least about 35, at least about 40, at least about 45, at least about 50, at least about 55, at least about 60, at least about 65, at least about 70, at least about 75, at least about 80, at least about 85, at least about 90, at least about 95, at least about 100, at least about 110, at least about 120, at least about 130, at least about 140, at least about 150, at least about 160, at least about 170, at least about 180, at least about 190, at least about 200, at least about 210, at least about 220, at least about 230, at least about 240, at least about 250, at least about 260, at least about 270, at least about 280, at least about 290, at least about 300, at least about 350, at least about 400, at least about 450, at least about 500, or greater than 500 contiguous nucleotides. In particular embodiments, the silencing element comprises at least one segment of at least 18, 19, 20, or 21 contiguous nucleotides with a sequence of 100% identity with a fragment of equivalent length of a DNA or target gene having a sequence selected from the Target Gene Sequences Group or the Trigger Sequences Group, or the DNA complement thereof. In particular embodiments, the RNA is a double-stranded nucleic acid (e.g., dsRNA) with one strand comprising at least one segment of at least 18, 19, 20, or 21 contiguous nucleotides with a sequence of 100% identity with a fragment of equivalent length of a DNA or target gene having a sequence selected from the Target Gene Sequences Group or the Trigger Sequences Group, or the DNA complement thereof; expressed as base-pairs, such a double-stranded nucleic acid comprises at least one segment of at least 18, 19, 20, or 21 contiguous, perfectly matched base-pairs which correspond to a fragment of equivalent length of a DNA or target gene having a sequence selected from the Target Gene Sequences Group or the Trigger Sequences Group, or the DNA complement thereof. In particular embodiments, each silencing element contained in the RNA is of a length greater than that which is typical of naturally occurring regulatory small RNAs. In some embodiments, each segment is at least about 30 contiguous nucleotides (or base-pairs) in length. In particular embodiments, the RNA is between about 50 to about 500 nucleotides in length. In particular embodiments, the RNA has a sequence selected from the Trigger Sequences Group.

In some embodiments, the transgenic crop plant cell is further capable expressing additional heterologous DNA sequences. In an embodiment, the transgenic solanaceous plant cell has a genome that further comprises recombinant DNA encoding at least one pesticidal agent selected from the group consisting of a patatin, a plant lectin, a phytoecdysteroid, a phytoecdysteroid, a Bacillus thuringiensis insecticidal protein, a Xenorhabdus insecticidal protein, a Photorhabdus insecticidal protein, a Bacillus laterosporus insecticidal protein, and a Bacillus sphaericus insecticidal protein. In particular embodiments, the transgenic crop plant cell has stably integrated in its genome (i) recombinant DNA encoding at least one RNA with a sequence selected from the Trigger Sequences Group and (ii) DNA encoding at least one pesticidal agent selected from the group consisting of a patatin, a plant lectin, a phytoecdysteroid, a phytoecdysteroid, a Bacillus thuringiensis insecticidal protein, a Xenorhabdus insecticidal protein, a Photorhabdus insecticidal protein, a Bacillus laterosporus insecticidal protein, and a Bacillus sphaericus insecticidal protein.

In a related aspect, this invention is directed to a transgenic crop plant including the transgenic crop plant cell, a commodity product produced from the transgenic crop plant, and transgenic progeny solanaceous plant seed or transgenic propagatable part of the transgenic solanaceous plant. Also contemplated is a commodity product produced by the transgenic crop plant, and a commodity product produced from the transgenic progeny seed of such a transgenic crop plant.

### X. Methods of Selecting Target Genes

Another aspect of this invention provides a method of non-random selection of target genes for RNAi-mediated silencing. In an embodiment, the method provides a subset of target genes that are present in single- or low-copy-number (non-repetitive and non-redundant) in a particular genome. Such target genes can be genes from a plant genome or genes from an animal genome. In some embodiments, the target genes are genes of an invertebrate pest, e.g. an invertebrate pest of a plant or an invertebrate pest of a vertebrate. In some embodiments, the target genes are genes of an insect pest of a plant or a nematode pest of a plant. In some embodiments, the target genes are genes of a Lepidopteran pest. Further aspects include manufacturing a polynucleotide (e.g., an ssRNA or dsRNA trigger, such as the dsRNA triggers described in the working Examples, or a recombinant DNA construct useful for making transgenic plants) based on target genes for RNAi-mediated silencing selected by any of the methods described herein.

In an embodiment, the method comprises the step of identifying single- or low-copy-number genes in the chosen genome, or alternatively to identify single- or low-copy-number genes in an orthologous database from related organisms to predict which genes will be single/low copy in the chosen organism. Low-copy genes, and in particular single-copy genes, are selected as targets for RNAi-mediated silencing. In one embodiment, the identification of single- or low-copy-number genes is carried out by sequence comparison between a set of genes from a first species and a set of genes from a second species, wherein the set of genes from a second species have been identified as single- or low-copy-number in the second species. In one embodiment, the identification of single- or low-copy-number genes is carried out by applying an algorithm performed by a computer to a set of genes from a first species to identify a subset of single- or low-copy-number genes in the set of genes from the first species, then comparing a set of genes from a second species to the subset of single- or low-copy-number genes from the first species to identify corresponding single- or low-copy-number genes from the second species. The single- or low-copy-number genes from the second species are useful as target genes for RNAi-mediated silencing; the sequences of these target genes are used for designing polynucleotides (e.g., an ssRNA or dsRNA trigger, such as the dsRNA triggers described in the working Examples, or recombinant DNA constructs for making transgenic plants) and methods of use thereof for preventing or controlling infestations by the second species. Another embodiment relates to identifying genes which are vital to the organism's survival by searching databases containing such information for model species orthologous to the target organism.

Embodiments of the method include a further step of estimating nucleotide diversity for low/single-copy genes in a population of the chosen organism and selecting those low-/single-copy genes that further have the lowest nucleotide diversity. Low-/single-copy genes that further have low nucleotide diversity are selected as targets for RNAi-mediated silencing. Another embodiment relates to identifying genes which are vital to the organism's survival by searching databases containing such information for model species orthologous to the target organism.

Embodiments of the method include a further step of comparing the ratio of synonymous (Kₛ) to nonsynonymous (Kₐ) nucleotide changes as an estimate of functional or evolutionary constraint. In an embodiment, the method comprises the step of selecting genes where Kₛ is at least equal to or greater than Kₐ. In an embodiment, the method comprises the step of selecting genes where Kₐ>>Kₐ.

A related aspect of this invention is a set of target genes for RNAi-mediated silencing identified from a genome by any of the gene selection methods described herein. An embodiment relates to a set of target genes for RNAi-mediated silencing selected from a genome by identifying single- or low-copy-number target genes from a larger set of genes from that genome. One embodiment relates to a set of target genes for RNAi-mediated silencing selected from an invertebrate genome by identifying single- or low-copy-number target genes from a larger set of genes from that invertebrate genome. A specific embodiment relates to a set of target genes for RNAi-mediated silencing in a Lepidopteran pest selected from a Lepidopteran genome by identifying single- or low-copy-number target genes from a larger set of genes from that Lepidopteran genome. A specific embodiment relates to a set of target genes for RNAi-mediated silencing in a Lepidopteran pest selected from a Lepidopteran genome by identifying single- or low-copy-number target genes from a larger set of genes from that Lepidopteran genome.

Another embodiment relates to a set of target genes for RNAi-mediated silencing selected from a genome by estimating nucleotide diversity for a given set of genes in a population of individuals of the pest having that genome, and selecting those genes that have the lowest nucleotide diversity. One embodiment relates to a set of target genes for RNAi-mediated silencing selected from an invertebrate genome by estimating nucleotide diversity for a given set of genes in a population of individuals of the invertebrate having that genome, and selecting those genes that have the lowest nucleotide diversity. Another embodiment relates to a set of target genes for RNAi-mediated silencing selected from an invertebrate genome by estimating nucleotide diversity for low-/single-copy genes in a population of individuals of the invertebrate having that genome, and selecting those low-/single-copy genes that further have the lowest nucleotide diversity.

Another embodiment relates to a set of target genes for RNAi-mediated silencing selected from a genome by comparing the ratio of synonymous (Kₛ) to nonsynonymous (Kₐ) nucleotide changes in genes of that genome and selecting genes where Kₛ is at least equal to or greater than Kₐ. In an embodiment, the set of target genes for RNAi-mediated silencing are genes where Kₛ is at least equal to or greater than Kₐ. In an embodiment, the set of target genes for RNAi-mediated silencing are genes where Kₛ>>Kₐ. An embodiment relates to a set of target genes for RNAi-mediated silencing selected from an invertebrate genome and where Kₛ>>Kₐ for the selected genes.

A further aspect of this invention are polyclonal or monoclonal antibodies that bind a protein encoded by a sequence or a fragment of a sequence selected from the group consisting of the Target Gene Sequences Group and polyclonal or monoclonal antibodies that bind a protein encoded by a sequence or a fragment of a sequence selected from the Trigger Sequences Group, or the complement thereof; such antibodies are made by routine methods as known to one of ordinary skill in the art, for example using routine protocols as described in “Antibody Methods and Protocols” (Proetzel and Ebersbach, editors, 2012, Humana Press, New York) or “Making and Using Antibodies” (Howard and Kaser, editors, 2006, CRC Press, Boca Raton).

### XI. Selection of Effective Polynucleotides by “Tiling”

Polynucleotides of use in the embodiments described herein need not be of the full length of a target gene, and in many embodiments are much shorter than the target gene. An example of a technique that is useful for selecting effective polynucleotides is “tiling”, or evaluation of polynucleotides corresponding to adjacent or partially overlapping segments of a target gene.

In some embodiments, effective polynucleotide triggers can be identified by “tiling” gene targets in selected length fragments, e.g., fragments of 200-300 nucleotides in length, with partially overlapping regions, e.g., of about 25 nucleotides, along the length of the target gene. In some embodiments, polynucleotide trigger sequences are designed to correspond to (have a nucleotide identity or complementarity with) regions that are unique to the target gene. In some embodiments, the selected region of the target gene can include coding sequence or non-coding sequence (e.g., promoter regions, 3′ untranslated regions, introns and the like) or a combination of both.

Where it is of interest to design a target effective in suppressing multiple target genes, the multiple target gene sequences are aligned and polynucleotide triggers are designed to correspond to regions with high sequence homology in common among the multiple targets. Conversely, where it is of interest to design a target effective in selectively suppressing one among multiple target sequences, the multiple target gene sequences are aligned and polynucleotide triggers designed to correspond to regions with no or low sequence homology in common among the multiple targets.

### XII. Thermodynamic Considerations in Selection of Effective Polynucleotides

In some embodiments, polynucleotide triggers can be designed or their sequence optimised using thermodynamic considerations. For example, polynucleotide triggers can be selected based on the thermodynamics controlling hybridization between one nucleic acid strand (e.g., a polynucleotide trigger or an individual siRNA) and another (e.g., a target gene transcript).

Methods and algorithms to predict nucleotide sequences that may be effective at RNAi-mediated silencing of a target gene are known in the art. Non-limiting examples of such methods and algorithms include “i-score”, described by Ichihara et al. (2007) Nucleic Acids Res., 35(18): 123e; “Oligowalk”, publicly available at rna.urmc.rochester.edu/servers/oligowalk and described by Lu et al. (2008) Nucleic Acids Res., 36:W104-108; and “Reynolds score”, described by Khovorova et al. (2004) Nature Biotechnol., 22:326-330, and “si-Fi”, described by Luck et al. (2019) Front. Plant Sci. publicly available at http://www.snowformatics.com/si-fi.html; and “SnapDragon”, described by Hu et al. (2016) Nucleic Acids Res., 45: D672-D678, publicly available at https://www.flyrnai.org/snapdragon; and “E-RNAi”, described by Horn et al. (2010) Nucleic Acids Res., 38: W332-W339, publicly available at https://www.dkfz.de/signaling/e-rnai3/.

### XIII. Permitted Mismatches

“Essentially identical” or “essentially complementary”, as used herein, means that a polynucleotide (or at least one strand of a double-stranded polynucleotide) has sufficient identity or complementarity to the target gene or to the RNA transcribed from a target gene (e.g., the transcript) to suppress expression of a target gene (e.g., to effect a reduction in levels or activity of the target gene transcript and/or encoded protein). Polynucleotides as described herein need not have 100 percent identity or complementarity to a target gene or sequence or to the RNA transcribed from a target gene to suppress expression of the target gene (e.g., to effect a reduction in levels or activity of the target gene transcript or encoded protein, or to provide control of a Lepidopteran pest). In some embodiments, the polynucleotide or a portion thereof is designed to be essentially identical to, or essentially complementary to, a sequence of at least 18 or 19 contiguous nucleotides in either the target gene or the RNA transcribed from the target gene. In some embodiments, the polynucleotide or a portion thereof is designed to be 100% identical to, or 100% complementary to, one or more sequences of 21 contiguous nucleotides in either the target gene or the RNA transcribed from the target gene. In certain embodiments, an “essentially identical” polynucleotide has 100 percent sequence identity or at least about 83, 84, 85, 86, 87, 88, 89, 90, 91, 92, 93, 94, 95, 96, 97, 98, or 99 percent sequence identity when compared to the sequence of 18 or more contiguous nucleotides in either the endogenous target gene or to an RNA transcribed from the target gene. In certain embodiments, an “essentially complementary” polynucleotide has 100 percent sequence complementarity or at least about 83, 84, 85, 86, 87, 88, 89, 90, 91, 92, 93, 94, 95, 96, 97, 98, or 99 percent sequence complementarity when compared to the sequence of 18 or more contiguous nucleotides in either the target gene or RNA transcribed from the target gene.

Sequence identity: The term “sequence identity” or “identity,” as used herein in the context of two polynucleotides or polypeptides, refers to the residues in the sequences of the two molecules that are the same when aligned for maximum correspondence over a specified comparison window.

As used herein, the term “percentage of sequence identity” may refer to the value determined by comparing two optimally aligned sequences (e.g., nucleic acid sequences or polypeptide sequences) of a molecule over a comparison window, wherein the portion of the sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleotide or amino acid residue occurs in both sequences to yield the number of matched positions, dividing the number of matched positions by the total number of positions in the comparison window, and multiplying the result by 100 to yield the percentage of sequence identity. A sequence that is identical at every position in comparison to a reference sequence is said to be 100% identical to the reference sequence, and vice-versa. The term “about” with respect to a numerical value of a sequence length means the stated value with a +/−variance of up to 1-5 percent. For example, about 30 contiguous nucleotides means a range of 27-33 contiguous nucleotides, or any range in between. The term “about” with respect to a numerical value of percentage of sequence identity means the stated percentage value with a +/−variance of up to 1-3 percent rounded to the nearest integer. For example, about 90% sequence identity means a range of 87-93%. However, the percentage of sequence identity cannot exceed 100 percent. Thus, about 98% sequence identity means a range of 95-100%.

Polynucleotides containing mismatches to the target gene or transcript can be used in certain embodiments of the compositions and methods described herein. In some embodiments, the polynucleotide includes at least 18 or at least 19 or at least 21 contiguous nucleotides that are essentially identical or essentially complementary to a segment of equivalent length in the target gene or target gene's transcript. In certain embodiments, a polynucleotide of 18, 19, 20, or 21 or more contiguous nucleotides that is essentially identical or essentially complementary to a segment of equivalent length in the target gene or target gene's transcript can have 1 or 2 mismatches to the target gene or transcript (i.e., 1 or 2 mismatches between the polynucleotide's 21 contiguous nucleotides and the segment of equivalent length in the target gene or target gene's transcript). In certain embodiments, a polynucleotide of about 50, 100, 150, 200, 250, 300, 350 or more nucleotides that contains a contiguous 18, 19, 20, or 21 or more nucleotide span of identity or complementarity to a segment of equivalent length in the target gene or target gene's transcript can have 1 or 2 or more mismatches to the target gene or transcript.

In designing polynucleotides with mismatches to an endogenous target gene or to an RNA transcribed from the target gene, mismatches of certain types and at certain positions that are more likely to be tolerated can be used. In certain embodiments, mismatches formed between adenine and cytosine or guanosine and uracil residues are used as described by Du et al. (2005) Nucleic Acids Res., 33:1671-1677. In some embodiments, mismatches in 19 base-pair overlap regions are located at the low tolerance positions 5, 7, 8 or 11 (from the 5′ end of a 19-nucleotide target), at medium tolerance positions 3, 4, and 12-17 (from the 5′ end of a 19-nucleotide target), and/or at the high tolerance positions at either end of the region of complementarity, i.e., positions 1, 2, 18, and 19 (from the 5′ end of a 19-nucleotide target) as described by Du et al. (2005) Nucleic Acids Res., 33:1671-1677. Tolerated mismatches can be empirically determined in routine assays, e.g., in in vitro dietary assays on Lepidopteran pest larvae.

### XIV. Embedding Silencing Elements in Neutral Sequence

In some embodiments, a silencing element comprising a sequence corresponding to the target gene and which is responsible for an observed suppression of the target gene is embedded in “neutral” sequence, i.e., inserted into additional nucleotides that have no sequence identity or complementarity to the target gene. Neutral sequence can be desirable, e.g., to increase the overall length of a polynucleotide. For example, it can be desirable for a polynucleotide to be of a particular size for reasons of stability, cost-effectiveness in manufacturing, or biological activity. In some embodiments, neutral sequence is also useful in forming the loop in a hairpin trigger or as a spacer between trigger regions.

It has been reported that in another coleopteran species, Diabrotica virgifera, dsRNAs greater than or equal to approximately 60 base-pairs (bp) are required for biological activity in artificial diet bioassays; see Bolognesi et al. (2012) PLoS ONE 7(10): e47534. doi:10.1371/journal.pone.0047534. Thus, in one embodiment, a 21-base-pair dsRNA silencing element corresponding to a target gene in the Target Gene Sequences Group and found to provide control of a Lepidopteran infestation is embedded in neutral sequence of an additional 39 base pairs, thus forming a polynucleotide of about 60 base pairs. In some embodiments, the dsRNA trigger includes neutral sequence of between about 60 to about 500, or between 100 to about 450 base-pairs, in which is embedded at least one segment of 21 contiguous nucleotides with a sequence of 100% identity or 100% complementarity with a fragment of equivalent length of a target gene having a sequence selected from the Target Gene Sequences Group. In another embodiment, a single 21-base-pair silencing element with a sequence of 100% identity or 100% complementarity with a fragment of equivalent length of a target gene is found to be efficacious when embedded in larger sections of neutral sequence, e.g., where the total polynucleotide length is from about 60 to about 300 base pairs. In embodiments where the polynucleotide includes regions of neutral sequence, the polynucleotide will have relatively low overall sequence identity in comparison to the target gene; for example, a dsRNA with an overall length of 210 base-pairs, containing a single 21-base-pair trigger (of 100% identity or complementarity to a 21-nucleotide fragment of a target gene) embedded in an additional 189 base-pairs of neutral sequence, will have an overall sequence identity with the target gene of about 10%.

### XV. Insecticidal Double-Stranded RNA Molecules

Another aspect of this invention provides an insecticidal double-stranded RNA molecule that causes mortality or stunting of growth in a Lepidopteran pest when ingested or contacted by the Lepidopteran pest, wherein the insecticidal double-stranded RNA molecule comprises at least one segment of 18 or more contiguous nucleotides that is essentially identical or essentially complementary to a segment of equivalent length of a target gene or DNA (cDNA) having a sequence selected from The Target Gene Sequences Group. In some embodiments, the insecticidal double-stranded RNA molecule is between about 50 to about 500 base-pairs in length. In some embodiments, the insecticidal double-stranded RNA molecule comprises at least one segment of at least 30 contiguous nucleotides in length. In some embodiments, the insecticidal double-stranded RNA molecule comprises multiple segments of 18 or more contiguous nucleotides that are essentially identical or essentially complementary to a segment of equivalent length of a target gene or DNA (cDNA) having a sequence selected from The Target Gene Sequences Group, wherein the segments are from different regions of the target gene (e.g., the segments can correspond to different exon regions of the target gene, and “spacer” nucleotides which do not correspond to a target gene can optionally be used in between or adjacent to the segments), or are from different target genes. In some embodiments, the insecticidal double-stranded RNA molecule comprises multiple segments of 18 or more contiguous nucleotides that are essentially identical or essentially complementary to a segment of equivalent length of a target gene or DNA (cDNA) having a sequence selected from The Target Gene Sequences Group, wherein the segments are from different regions of the target gene and are arranged in the insecticidal double-stranded RNA molecule in an order different from the order in which the segments naturally occur in the target gene. In some embodiments, the insecticidal double-stranded RNA molecule comprises multiple segments each of 21 contiguous nucleotides with a sequence of 100% identity or 100% complementary to a segment of equivalent length of a target gene or DNA (cDNA) having a sequence selected from The Target Gene Sequences Group, wherein the segments are from different regions of the target gene and are arranged in the insecticidal double-stranded RNA molecule in an order different from the order in which the segments naturally occur in the target gene. In some embodiments, the insecticidal double-stranded RNA molecule comprises one strand comprising a sequence selected from the Trigger Sequences Group, or the complement thereof. The insecticidal double-stranded RNA molecule can be topically applied to a plant to control or prevent infestation by a Lepidopteran pest. The insecticidal double-stranded RNA molecule can be provided in a form suitable for ingestion or direct contact by a Lepidopteran pest, e.g., in the form of a spray or powder or bait. Other methods and suitable compositions for providing the insecticidal double-stranded RNA molecule are similar to those described in the preceding paragraphs for other aspects of this invention.

Several embodiments relate to a tank mixture comprising one or more insecticidal polynucleotides and water or other solvent, optionally including a cationic lipid or an organosilicone surfactant or both. Embodiments include tank mixture formulations of the polynucleotide and optionally at least one pesticidal agent selected from the group consisting of a patatin, a plant lectin, a phytoecdysteroid, a Bacillus thuringiensis insecticidal protein, a Xenorhabdus insecticidal protein, a Photorhabdus insecticidal protein, a Bacillus laterosporus insecticidal protein, and a Bacillus sphaericus insecticidal protein. Embodiments of such compositions include those where one or more insecticidal polynucleotides are provided in a living or dead microorganism such as a bacterium or fungal or yeast cell, or provided as a microbial fermentation product, or provided in a living or dead plant cell, or provided as a synthetic recombinant polynucleotide. In an embodiment the composition includes a non-pathogenic strain of a microorganism that contains a polynucleotide as described herein; ingestion or intake of the microorganism results in stunting or mortality of the Lepidopteran pest; non-limiting examples of suitable microorganisms include E. coli, B. thuringiensis, Pseudomonas sp., Photorhabdus sp., Xenorhabdus sp., Serratia entomophila and related Serratia sp., B. sphaericus, B. cereus, B. laterosporus, B. popilliae, Clostridium bifermentans and other Clostridium species, or other spore-forming gram-positive bacteria. In an embodiment, the composition includes a plant virus vector comprising a polynucleotide as described herein; feeding by a Lepidopteran pest on a plant treated with the plant virus vector results in stunting or mortality of the Lepidopteran pest. In an embodiment, the composition includes a baculovirus vector including a polynucleotide as described herein; ingestion or intake of the vector results in stunting or mortality of the Lepidopteran pest. In an embodiment, a polynucleotide as described herein is encapsulated in a synthetic matrix such as a polymer or attached to particulates and topically applied to the surface of a plant; feeding by a Lepidopteran pest on the topically treated plant results in stunting or mortality of the Lepidopteran pest. In an embodiment, a polynucleotide as described herein is provided in the form of a plant cell (e.g., a transgenic solanaceous plant cell of this invention) expressing the polynucleotide; ingestion of the plant cell or contents of the plant cell by a Lepidopteran pest results in stunting or mortality of the Lepidopteran pest.

In some embodiments, one or more polynucleotides as described herein are provided with appropriate stickers and wetters required for efficient foliar coverage as well as UV protectants to protect polynucleotides such as dsRNAs from UV damage. Such additives are commonly used in the bioinsecticide industry and are known to those skilled in the art. Compositions for soil application can include granular formulations that serve as bait for Lepidopteran pest larvae. In some embodiments, one or more polynucleotides as described herein are further provided with a carrier agent, a surfactant, a cationic lipid (such as that disclosed in Example 18 of U.S. patent application publication 2011/0296556, incorporated by reference herein), an organosilicone, an organosilicone surfactant, a polynucleotide herbicidal molecule, a non-polynucleotide herbicidal molecule, a non-polynucleotide pesticide, a safener, and an insect growth regulator. In some embodiments, the composition further includes at least one pesticidal agent selected from the group consisting of a patatin, a plant lectin, a phytoecdysteroid, a Bacillus thuringiensis insecticidal protein, a Xenorhabdus insecticidal protein, a Photorhabdus insecticidal protein, a Bacillus laterosporus insecticidal protein, and a Bacillus sphaericus insecticidal protein.

Such compositions are applied in any convenient manner, e.g., by spraying or dusting the Lepidopteran pest directly, or spraying or dusting a plant or environment wherein prevention or control of infestation by that Lepidopteran pest is desired, or by applying a coating to a surface of a plant, or by applying a coating to a seed (or seed potato) in preparation for the seed's planting, or by applying a soil drench around roots of a plant for which prevention or control of infestation by that Lepidopteran pest is desired.

An effective amount of a polynucleotide as described herein is an amount sufficient to provide control of the Lepidopteran pest, or to prevent infestation by the Lepidopteran pest; determination of effective amounts of a polynucleotide are made using routine assays. While there is no upper limit on the concentrations and dosages of an insecticidal polynucleotide that can be useful in the methods and compositions provided herein, lower effective concentrations and dosages will generally be sought for efficiency and economy. Non-limiting embodiments of effective amounts of a polynucleotide include a range from about 10 nanograms per milliliter to about 100 micrograms per milliliter of a polynucleotide in a liquid form sprayed on a plant, or from about 10 milligrams per acre to about 100 grams per acre of polynucleotide applied to a field of plants, or from about 0.001 to about 0.1 microgram per milliliter of polynucleotide in an artificial diet for feeding the Lepidopteran pest. Where polynucleotides as described herein are topically applied to a plant, the concentrations can be adjusted in consideration of the volume of spray or treatment applied to plant leaves or other plant part surfaces, such as flower petals, stems, tubers, fruit, anthers, pollen, leaves, roots, or seeds. In one embodiment, a useful treatment for herbaceous plants using 25-mer polynucleotides as described herein is about 1 nanomole (nmol) of polynucleotides per plant, for example, from about 0.05 to 1 nmol polynucleotides per plant. Other embodiments for herbaceous plants include useful ranges of about 0.05 to about 100 nmol, or about 0.1 to about 20 nmol, or about 1 nmol to about 10 nmol of polynucleotides per plant. In certain embodiments, about 40 to about 50 nmol of a ssDNA polynucleotide are applied. In certain embodiments, about 0.5 nmol to about 2 nmol of a dsRNA is applied. In certain embodiments, a composition containing about 0.5 to about 2.0 milligrams per milliliter, or about 0.14 milligrams per milliliter of a dsRNA or an ssDNA (21-mer) is applied. In certain embodiments, a composition of about 0.5 to about 1.5 milligrams per milliliter of a dsRNA polynucleotide of this invention of about 50 to about 200 or more nucleotides is applied. In certain embodiments, about 1 nmol to about 5 nmol of a dsRNA of this invention is applied to a plant. In certain embodiments, the polynucleotide composition as topically applied to the plant contains at least one polynucleotide of this invention at a concentration of about 0.01 to about 10 milligrams per milliliter, or about 0.05 to about 2 milligrams per milliliter, or about 0.1 to about 2 milligrams per milliliter. Very large plants, trees, or vines can require correspondingly larger amounts of polynucleotides. When using long dsRNA molecules of this invention that can be processed into multiple oligonucleotides (e.g., multiple triggers encoded by a single recombinant DNA molecule of this invention), lower concentrations can be used. Non-limiting examples of effective polynucleotide treatment regimes include a treatment of between about 0.1 to about 1 nmol of polynucleotide molecule per plant, or between about 1 nmol to about 10 nmol of polynucleotide molecule per plant, or between about 10 nmol to about 100 nmol of polynucleotide molecule per plant.

In some embodiments, one or more polynucleotides is provided with a “transfer agent”, which is an agent that enables a topically applied polynucleotide to enter the cells of an organism. Such transfer agents can be incorporated as part of a composition comprising a polynucleotide as described herein, or can be applied prior to, contemporaneously with, or following application of the polynucleotide. In some embodiments, a transfer agent is an agent that improves the uptake of a polynucleotide of this invention by a Lepidopteran pest. In some embodiments, a transfer agent is an agent that conditions the surface of plant tissue, e.g., seeds, leaves, stems, roots, flowers, or fruits, to permeation by a polynucleotide into plant cells. In some embodiments, the transfer agent enables a pathway for a polynucleotide through cuticle wax barriers, stomata, and/or cell wall or membrane barriers into plant cells.

Suitable transfer agents include agents that increase permeability of the exterior of the organism or that increase permeability of cells of the organism to polynucleotides. Suitable transfer agents include a chemical agent, or a physical agent, or combinations thereof. Chemical agents for conditioning or transfer include (a) surfactants, (b) an organic solvent or an aqueous solution or aqueous mixtures of organic solvents, (c) oxidizing agents, (d) acids, (e) bases, (f) oils, (g) enzymes, or any combination thereof. In some embodiments, application of a polynucleotide and a transfer agent optionally includes an incubation step, a neutralization step (e.g., to neutralize an acid, base, or oxidizing agent, or to inactivate an enzyme), a rinsing step, or combinations thereof. Suitable transfer agents can be in the form of an emulsion, a reverse emulsion, a liposome, or other micellar-like composition, or can cause the polynucleotide to take the form of an emulsion, a reverse emulsion, a liposome, or other micellar-like composition. Embodiments of transfer agents include counter-ions or other molecules that are known to associate with nucleic acid molecules, e.g., inorganic ammonium ions, alkyl ammonium ions, lithium ions, polyamines such as spermine, spermidine, or putrescine, and other cations. Embodiments of transfer agents include organic solvents such as DMSO, DMF, pyridine, N-pyrrolidine, hexamethylphosphoramide, acetonitrile, dioxane, polypropylene glycol, or other solvents miscible with water or that dissolve phosphonucleotides in non-aqueous systems (such as is used in synthetic reactions). Embodiments of transfer agents include naturally derived or synthetic oils with or without surfactants or emulsifiers, e.g., plant-sourced oils, crop oils (such as those listed in the 9^{th} Compendium of Herbicide Adjuvants, publicly available on-line at herbicide.adjuvants.com), paraffinic oils, polyol fatty acid esters, or oils with short-chain molecules modified with amides or polyamines such as polyethyleneimine or N-pyrrolidine.

Embodiments of transfer agents include organosilicone preparations. For example, a suitable transfer agent is an organosilicone preparation that is commercially available as SILWET L-77® brand surfactant having CAS Number 27306-78-1 and EPA Number: CAL.REG.NO. 5905-50073-AA, and currently available from Momentive Performance Materials, Albany, N.Y. One embodiment includes a composition that comprises a polynucleotide and a transfer agent including an organosilicone preparation such as Silwet L-77 in the range of about 0.015 to about 2 percent by weight (wt percent) (e.g., about 0.01, 0.015, 0.02, 0.025, 0.03, 0.035, 0.04, 0.045, 0.05, 0.055, 0.06, 0.065, 0.07, 0.075, 0.08, 0.085, 0.09, 0.095, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.5 wt percent). One embodiment includes a composition that comprises a polynucleotide of this invention and a transfer agent including SILWET L-77® brand surfactant in the range of about 0.3 to about 1 percent by weight (wt percent) or about 0.5 to about 1%, by weight (wt percent).

Organosilicone compounds useful as transfer agents for use in this invention include, but are not limited to, compounds that include: (a) a trisiloxane head group that is covalently linked to, (b) an alkyl linker including, but not limited to, an n-propyl linker, that is covalently linked to, (c) a polyglycol chain, that is covalently linked to, (d) a terminal group. Trisiloxane head groups of such organosilicone compounds include, but are not limited to, heptamethyltrisiloxane. Alkyl linkers can include, but are not limited to, an n-propyl linker. Polyglycol chains include, but are not limited to, polyethylene glycol or polypropylene glycol. Polyglycol chains can comprise a mixture that provides an average chain length “n” of about “7.5”. In certain embodiments, the average chain length “n” can vary from about 5 to about 14. Terminal groups can include, but are not limited to, alkyl groups such as a methyl group. Organosilicone compounds useful as transfer agents include, but are not limited to, trisiloxane ethoxylate surfactants or polyalkylene oxide modified heptamethyl trisiloxane. An example of a transfer agent for use in this invention is Compound I:

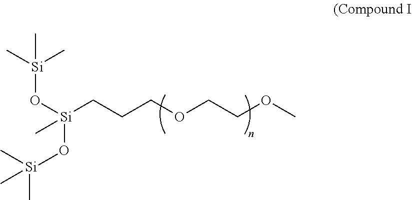

polyalkyleneoxide heptamethyltrisiloxane, average n=7.5).
Organosilicone compounds useful as transfer agents are used, e.g., as freshly made concentrations in the range of about 0.015 to about 2 percent by weight (wt percent) (e.g., about 0.01, 0.015, 0.02, 0.025, 0.03, 0.035, 0.04, 0.045, 0.05, 0.055, 0.06, 0.065, 0.07, 0.075, 0.08, 0.085, 0.09, 0.095, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.5 wt percent).

Embodiments of transfer agents include one or more salts such as ammonium chloride, tetrabutylphosphonium bromide, and ammonium sulfate, provided in or used with a composition including a polynucleotide. In some embodiments, ammonium chloride, tetrabutylphosphonium bromide, and/or ammonium sulfate are used at a concentration of about 0.5% to about 5% (w/v), or about 1% to about 3% (w/v), or about 2% (w/v). In certain embodiments, the composition including a polynucleotide includes an ammonium salt at a concentration greater or equal to 300 millimolar. In certain embodiments, the composition including a polynucleotide includes an organosilicone transfer agent in a concentration of about 0.015 to about 2 percent by weight (wt percent) as well as ammonium sulfate at concentrations from about 80 to about 1200 mM or about 150 mM to about 600 mM.

Embodiments of transfer agents include a phosphate salt. Phosphate salts useful in a composition including a polynucleotide include, but are not limited to, calcium, magnesium, potassium, or sodium phosphate salts. In certain embodiments, a composition including a polynucleotide includes a phosphate salt at a concentration of at least about 5 millimolar, at least about 10 millimolar, or at least about 20 millimolar. In certain embodiments, a composition including a polynucleotide a phosphate salt in a range of about 1 mM to about 25 mM or in a range of about 5 mM to about 25 mM. In certain embodiments, the composition including a polynucleotide sodium phosphate at a concentration of at least about 5 millimolar, at least about 10 millimolar, or at least about 20 millimolar. In certain embodiments, a composition including a polynucleotide includes sodium phosphate at a concentration of about 5 millimolar, about 10 millimolar, or about 20 millimolar. In certain embodiments, a composition including a polynucleotide includes a sodium phosphate salt in a range of about 1 mM to about 25 mM or in a range of about 5 mM to about 25 mM. In certain embodiments, a composition including a polynucleotide includes a sodium phosphate salt in a range of about 10 mM to about 160 mM or in a range of about 20 mM to about 40 mM. In certain embodiments, a composition including a polynucleotide includes a sodium phosphate buffer at a pH of about 6.8.

Embodiments of transfer agents include surfactants and/or effective molecules contained therein. Surfactants and/or effective molecules contained therein include, but are not limited to, sodium or lithium salts of fatty acids (such as tallow or tallowamines or phospholipids) and organosilicone surfactants. In certain embodiments, a composition including a polynucleotide is formulated with counter-ions or other molecules that are known to associate with nucleic acid molecules. Non-limiting examples include, tetraalkyl ammonium ions, trialkyl ammonium ions, sulfonium ions, lithium ions, and polyamines such as spermine, spermidine, or putrescine. In certain embodiments, a composition including a polynucleotide is formulated with a non-polynucleotide herbicide e.g., glyphosate, auxin-like benzoic acid herbicides including dicamba, chloramben, and TBA, glufosinate, auxin-like herbicides including phenoxy carboxylic acid herbicide, pyridine carboxylic acid herbicide, quinoline carboxylic acid herbicide, pyrimidine carboxylic acid herbicide, and benazolin-ethyl herbicide, sulfonylureas, imidazolinones, bromoxynil, delapon, cyclohezanedione, protoporphyrinogen oxidase inhibitors, and 4-hydroxyphenyl-pyruvate-dioxygenase inhibiting herbicides. In certain embodiments, a composition including a polynucleotide is formulated with a non-polynucleotide pesticide, e.g., a patatin, a plant lectin, a phytoecdysteroid, a Bacillus thuringiensis insecticidal protein, a Xenorhabdus insecticidal protein, a Photorhabdus insecticidal protein, a Bacillus laterosporus insecticidal protein, and a Bacillus sphaericus insecticidal protein. In some embodiments, a composition including a polynucleotide and a non-polynucleotide pesticide provides enhanced improvement in prevention or control of Lepidopteran pest infestations, when compared to the effect obtained with the polynucleotide alone or the non-polynucleotide pesticide alone. In some embodiments, a composition comprising a double-stranded RNA with a strand having a sequence selected from the Trigger Sequences Group is combined with a non-polynucleotide pesticide (e.g., a patatin, a plant lectin, a phytoecdysteroid, a Bacillus thuringiensis insecticidal protein, a Xenorhabdus insecticidal protein, a Photorhabdus insecticidal protein, a Bacillus laterosporus insecticidal protein, and a Bacillus sphaericus insecticidal protein), wherein the combination is found to effect improved prevention or control of Lepidopteran pest infestations, when compared to the effect obtained with the double-stranded RNA alone or the non-polynucleotide pesticide alone.

### XVI. Related Techniques

Embodiments of the polynucleotides and nucleic acid molecules as described herein can include additional elements, such as promoters, small RNA recognition sites, aptamers or ribozymes, additional and additional expression cassettes for expressing coding sequences (e.g., to express a transgene such as an insecticidal protein or selectable marker) or non-coding sequences (e.g., to express additional suppression elements). For example, an aspect of this invention provides a recombinant DNA construct comprising a heterologous promoter operably linked to DNA comprising at least one segment of 18 or more contiguous nucleotides with a sequence of about 95% to about 100% identity with a fragment of equivalent length of a DNA having a sequence selected from the Target Gene Sequences Group or the Trigger Sequences Group, or the DNA complement thereof. Another aspect of the invention provides a recombinant DNA construct comprising a heterologous promoter operably linked to DNA encoding an RNA hairpin having an anti-sense region having a sequence, or a fragment of a sequence, selected from the group selected from the Trigger Sequences Group. In another embodiment, a recombinant DNA construct comprising a promoter operably linked to DNA encoding: (a) an RNA silencing element for suppressing a target gene selected from the Target Gene Sequences Group, and (b) an aptamer, is stably integrated into the plant's genome from where RNA transcripts including the RNA aptamer and the RNA silencing element are expressed in cells of the plant; the aptamer serves to guide the RNA silencing element to a desired location in the cell. In another embodiment, inclusion of one or more recognition sites for binding and cleavage by a small RNA (e.g., by a miRNA or an siRNA that is expressed only in a particular cell or tissue) allows for more precise expression patterns in a plant, wherein the expression of the recombinant DNA construct is suppressed where the small RNA is expressed. Such additional elements are described below.

### XVII. Promoters

Promoters of use in the invention are functional in the cell in which the construct is intended to be transcribed. Generally these promoters are heterologous promoters, as used in recombinant constructs, i.e., they are not in nature found to be operably linked to the other nucleic elements used in the constructs described herein. In various embodiments, the promoter is selected from the group consisting of a constitutive promoter, a spatially specific promoter, a temporally specific promoter, a developmentally specific promoter, and an inducible promoter. In many embodiments the promoter is a promoter functional in a plant, for example, a pol II promoter, a pol III promoter, a pol IV promoter, or a pol V promoter.

Non-constitutive promoters suitable for use with the recombinant DNA constructs of this invention include spatially specific promoters, temporally specific promoters, and inducible promoters. Spatially specific promoters can include organelle-, cell-, tissue-, or organ-specific promoters (e.g., a plastid-specific, a root-specific, a pollen-specific, or a seed-specific promoter for expression in plastids, roots, pollen, or seeds, respectively). In many cases a seed-specific, embryo-specific, aleurone-specific, or endosperm-specific promoter is especially useful. Temporally specific promoters can include promoters that tend to promote expression during certain developmental stages in a plant's growth cycle, or during different times of day or night, or at different seasons in a year. Inducible promoters include promoters induced by chemicals or by environmental conditions such as, but not limited to, biotic or abiotic stress (e.g., water deficit or drought, heat, cold, high or low nutrient or salt levels, high or low light levels, or pest or pathogen infection). MicroRNA promoters are useful, especially those having a temporally specific, spatially specific, or inducible expression pattern; examples of miRNA promoters, as well as methods for identifying miRNA promoters having specific expression patterns, are provided in U.S. Patent Application Publications 2006/0200878, 2007/0199095, and 2007/0300329, which are specifically incorporated herein by reference. An expression-specific promoter can also include promoters that are generally constitutively expressed but at differing degrees or “strengths” of expression, including promoters commonly regarded as “strong promoters” or as “weak promoters”.

Promoters of particular interest include the following examples: an opaline synthase promoter isolated from T-DNA of Agrobacterium; a cauliflower mosaic virus 35S promoter; enhanced promoter elements or chimeric promoter elements such as an enhanced cauliflower mosaic virus (CaMV) 35S promoter linked to an enhancer element (an intron from heat shock protein 70 of Zea mays); root specific promoters such as those disclosed in U.S. Pat. Nos. 5,837,848; 6,437,217 and 6,426,446; a maize L3 oleosin promoter disclosed in U.S. Pat. No. 6,433,252; a promoter for a plant nuclear gene encoding a plastid-localized aldolase disclosed in U.S. Patent Application Publication 2004/0216189; cold-inducible promoters disclosed in U.S. Pat. No. 6,084,089; salt-inducible promoters disclosed in U.S. Pat. No. 6,140,078; light-inducible promoters disclosed in U.S. Pat. No. 6,294,714; pathogen-inducible promoters disclosed in U.S. Pat. No. 6,252,138; and water deficit-inducible promoters disclosed in U.S. Patent Application Publication 2004/0123347 A1. All of the above-described patents and patent publications disclosing promoters and their use, especially in recombinant DNA constructs functional in plants are incorporated herein by reference.

Plant vascular- or phloem-specific promoters of interest include a roIC or roIA promoter of Agrobacterium rhizogenes, a promoter of a Agrobacterium tumefaciens T-DNA gene 5, the rice sucrose synthase RSs1 gene promoter, a Commelina yellow mottle badnavirus promoter, a coconut foliar decay virus promoter, a rice tungro bacilliform virus promoter, the promoter of a pea glutamine synthase GS3A gene, a invCD111 and invCD141 promoters of a potato invertase genes, a promoter isolated from Arabidopsis shown to have phloem-specific expression in tobacco by Kertbundit et al. (1991) Proc. Natl. Acad. Sci. USA., 88:5212-5216, a VAHOX1 promoter region, a pea cell wall invertase gene promoter, an acid invertase gene promoter from carrot, a promoter of a sulfate transporter gene Sultr1; 3, a promoter of a plant sucrose synthase gene, and a promoter of a plant sucrose transporter gene.

Promoters suitable for use with a recombinant DNA construct or polynucleotide of this invention include polymerase II (“pol II”) promoters and polymerase III (“pol III”) promoters. RNA polymerase II transcribes structural or catalytic RNAs that are usually shorter than 400 nucleotides in length, and recognizes a simple run of T residues as a termination signal; it has been used to transcribe siRNA duplexes (see, e.g., Lu et al. (2004) Nucleic Acids Res., 32:e171). Pol II promoters are therefore in certain embodiments where a short RNA transcript is to be produced from a recombinant DNA construct of this invention. In one embodiment, the recombinant DNA construct comprises a pol II promoter to express an RNA transcript flanked by self-cleaving ribozyme sequences (e.g., self-cleaving hammerhead ribozymes), resulting in a processed RNA, such as a single-stranded RNA that binds to the transcript of the Lepidopteran target gene, with defined 5′ and 3′ ends, free of potentially interfering flanking sequences. An alternative approach uses pol III promoters to generate transcripts with relatively defined 5′ and 3′ ends, i.e., to transcribe an RNA with minimal 5′ and 3′ flanking sequences. In some embodiments, Pol III promoters (e.g., U6 or H1 promoters) are for adding a short AT-rich transcription termination site that results in 2 base-pair overhangs (UU) in the transcribed RNA; this is useful, e.g., for expression of siRNA-type constructs. Use of pol III promoters for driving expression of siRNA constructs has been reported; see van de Wetering et al. (2003) EMBO Rep., 4: 609-615, and Tuschl (2002) Nature Biotechnol., 20: 446-448. Baculovirus promoters such as baculovirus polyhedrin and p10 promoters are known in the art and commercially available; see, e.g., Invitrogen's “Guide to Baculovirus Expression Vector Systems (BEVS) and Insect Cell Culture Techniques”, 2002 (Life Technologies, Carlsbad, Calif.) and F. J. Haines et al. “Baculovirus Expression Vectors”, undated (Oxford Expression Technologies, Oxford, UK).

The promoter element can include nucleic acid sequences that are not naturally occurring promoters or promoter elements or homologues thereof but that can regulate expression of a gene. Examples of such “gene independent” regulatory sequences include naturally occurring or artificially designed RNA sequences that include a ligand-binding region or aptamer (see “Aptamers”, below) and a regulatory region (which can be cis-acting). See, for example, Isaacs et al. (2004) Nat. Biotechnol., 22:841-847, Bayer and Smolke (2005) Nature Biotechnol., 23:337-343, Mandal and Breaker (2004) Nature Rev. Mol. Cell Biol., 5:451-463, Davidson and Ellington (2005) Trends Biotechnol., 23:109-112, Winkler et al. (2002) Nature, 419:952-956, Sudarsan et al. (2003) RNA, 9:644-647, and Mandal and Breaker (2004) Nature Struct. Mol. Biol., 11:29-35. Such “riboregulators” could be selected or designed for specific spatial or temporal specificity, for example, to regulate translation of DNA that encodes a silencing element for suppressing a Lepidopteran target gene only in the presence (or absence) of a given concentration of the appropriate ligand. One example is a riboregulator that is responsive to an endogenous ligand (e.g., jasmonic acid or salicylic acid) produced by the plant when under stress (e.g., abiotic stress such as water, temperature, or nutrient stress, or biotic stress such as attach by pests or pathogens); under stress, the level of endogenous ligand increases to a level sufficient for the riboregulator to begin transcription of the DNA that encodes a silencing element for suppressing a Lepidopteran target gene.

### XVIII. Recombinase Sites

In some embodiments, the recombinant DNA construct or polynucleotide of this invention comprises DNA encoding one or more site-specific recombinase recognition sites. In one embodiment, the recombinant DNA construct comprises at least a pair of loxP sites, wherein site-specific recombination of DNA between the loxP sites is mediated by a Cre recombinase. The position and relative orientation of the loxP sites is selected to achieve the desired recombination; for example, when the loxP sites are in the same orientation, the DNA between the loxP sites is excised in circular form. In another embodiment, the recombinant DNA construct comprises DNA encoding one loxP site; in the presence of Cre recombinase and another DNA with a loxP site, the two DNAs are recombined.

### XIX. Aptamers

In some embodiments, the recombinant DNA construct or polynucleotide of this invention comprises DNA that is processed to an RNA aptamer, that is, an RNA that binds to a ligand through binding mechanism that is not primarily based on Watson-Crick base-pairing (in contrast, for example, to the base-pairing that occurs between complementary, anti-parallel nucleic acid strands to form a double-stranded nucleic acid structure). See, for example, Ellington and Szostak (1990) Nature, 346:818-822. Examples of aptamers can be found, for example, in the public Aptamer Database, available on line at aptamer.icmb.utexas.edu (Lee et al. (2004) Nucleic Acids Res., 32(1):D95-100). Aptamers useful in the invention can, however, be monovalent (binding a single ligand) or multivalent (binding more than one individual ligand, e.g., binding one unit of two or more different ligands).

Ligands useful in the invention include any molecule (or part of a molecule) that can be recognized and be bound by a nucleic acid secondary structure by a mechanism not primarily based on Watson-Crick base pairing. In this way, the recognition and binding of ligand and aptamer is analogous to that of antigen and antibody, or of biological effector and receptor. Ligands can include single molecules (or part of a molecule), or a combination of two or more molecules (or parts of a molecule), and can include one or more macromolecular complexes (e.g., polymers, lipid bilayers, liposomes, cellular membranes or other cellular structures, or cell surfaces). Examples of specific ligands include vitamins such as coenzyme B12 and thiamine pyrophosphate, flavin mononucleotide, guanine, adenosine, S-adenosylmethionine, S-adenosylhomocysteine, coenzyme A, lysine, tyrosine, dopamine, glucosamine-6-phosphate, caffeine, theophylline, antibiotics such as chloramphenicol and neomycin, herbicides such as glyphosate and dicamba, proteins including viral or phage coat proteins and invertebrate epidermal or digestive tract surface proteins, and RNAs including viral RNA, transfer-RNAs (t-RNAs), ribosomal RNA (rRNA), and RNA polymerases such as RNA-dependent RNA polymerase (RdRP). One class of RNA aptamers useful in the invention are “thermoswitches” that do not bind a ligand but are thermally responsive, that is to say, the aptamer's conformation is determined by temperature; see, for example, Box 3 in Mandal and Breaker (2004) Nature Rev. Mol. Cell Biol., 5:451-463.

### XX. Transgene Transcription Units

In some embodiments, the recombinant DNA construct or polynucleotide of this invention comprises a transgene transcription unit. A transgene transcription unit comprises DNA sequence encoding a gene of interest, e.g., a natural protein or a heterologous protein. A gene of interest can be any coding or non-coding sequence from any species (including, but not limited to, non-eukaryotes such as bacteria, and viruses; fungi, protists, plants, invertebrates, and vertebrates. Particular genes of interest are genes encoding at least one pesticidal agent selected from the group consisting of a patatin, a plant lectin, a phytoecdysteroid, a phytoecdysteroid, a Bacillus thuringiensis insecticidal protein, a Xenorhabdus insecticidal protein, a Photorhabdus insecticidal protein, a Bacillus laterosporus insecticidal protein, and a Bacillus sphaericus insecticidal protein. The transgene transcription unit can further include 5′ or 3′ sequence or both as required for transcription of the transgene.

### XXI. Introns

In some embodiments, the recombinant DNA construct or polynucleotide of this invention comprises DNA encoding a spliceable intron. By “intron” is generally meant a segment of DNA (or the RNA transcribed from such a segment) that is located between exons (protein-encoding segments of the DNA or corresponding transcribed RNA), wherein, during maturation of the messenger RNA, the intron present is enzymatically “spliced out” or removed from the RNA strand by a cleavage/ligation process that occurs in the nucleus in eukaryotes. The term “intron” is also applied to non-coding DNA sequences that are transcribed to RNA segments that can be spliced out of a maturing RNA transcript, but are not introns found between protein-coding exons. One example of these are spliceable sequences that that have the ability to enhance expression in plants (in some cases, especially in monocots) of a downstream coding sequence; these spliceable sequences are naturally located in the 5′ untranslated region of some plant genes, as well as in some viral genes (e.g., the tobacco mosaic virus 5′ leader sequence or “omega” leader described as enhancing expression in plant genes by Gallie and Walbot (1992) Nucleic Acids Res., 20:4631-4638). These spliceable sequences or “expression-enhancing introns” can be artificially inserted in the 5′ untranslated region of a plant gene between the promoter but before any protein-coding exons. Examples of such expression-enhancing introns include, but are not limited to, a maize alcohol dehydrogenase (Zm-Adh1), a maize Bronze-1 expression-enhancing intron, a rice actin 1 (Os-Act1) intron, a Shrunken-1 (Sh-1) intron, a maize sucrose synthase intron, a heat shock protein 18 (hsp18) intron, and an 82 kilodalton heat shock protein (hsp82) intron. U.S. Pat. Nos. 5,593,874 and 5,859,347, specifically incorporated by reference herein, describe methods of improving recombinant DNA constructs for use in plants by inclusion of an expression-enhancing intron derived from the 70 kilodalton maize heat shock protein (hsp70) in the non-translated leader positioned 3′ from the gene promoter and 5′ from the first protein-coding exon.

### XXII. Ribozymes

In some embodiments, the recombinant DNA construct or polynucleotide of this invention comprises DNA encoding one or more ribozymes. Ribozymes of particular interest include a self-cleaving ribozyme, a hammerhead ribozyme, or a hairpin ribozyme. In one embodiment, the recombinant DNA construct comprises DNA encoding one or more ribozymes that serve to cleave the transcribed RNA to provide defined segments of RNA, such as silencing elements for suppressing a Lepidopteran target gene.

### XXIII. Gene Suppression Elements

In some embodiments, the recombinant DNA construct or polynucleotide of this invention comprises DNA encoding additional gene suppression element for suppressing a target gene other than a Lepidopteran target gene. The target gene to be suppressed can include coding or non-coding sequence or both.

Suitable gene suppression elements are described in detail in U.S. Patent Application Publication 2006/0200878, which disclosure is specifically incorporated herein by reference, and include one or more of:
(a) DNA that comprises at least one anti-sense DNA segment that is anti-sense to at least one segment of the gene to be suppressed;
(b) DNA that comprises multiple copies of at least one anti-sense DNA segment that is anti-sense to at least one segment of the gene to be suppressed;
(c) DNA that comprises at least one sense DNA segment that is at least one segment of the gene to be suppressed;
(d) DNA that comprises multiple copies of at least one sense DNA segment that is at least one segment of the gene to be suppressed;
(e) DNA that transcribes to RNA for suppressing the gene to be suppressed by forming double-stranded RNA and comprises at least one anti-sense DNA segment that is anti-sense to at least one segment of the gene to be suppressed and at least one sense DNA segment that is at least one segment of the gene to be suppressed;
(f) DNA that transcribes to RNA for suppressing the gene to be suppressed by forming a single double-stranded RNA and comprises multiple serial anti-sense DNA segments that are anti-sense to at least one segment of the gene to be suppressed and multiple serial sense DNA segments that are at least one segment of the gene to be suppressed;
(g) DNA that transcribes to RNA for suppressing the gene to be suppressed by forming multiple double strands of RNA and comprises multiple anti-sense DNA segments that are anti-sense to at least one segment of the gene to be suppressed and multiple sense DNA segments that are at least one segment of the gene to be suppressed, and wherein the multiple anti-sense DNA segments and the multiple sense DNA segments are arranged in a series of inverted repeats;
(h) DNA that comprises nucleotides derived from a plant miRNA;
(i) DNA that comprises nucleotides of a siRNA;
(j) DNA that transcribes to an RNA aptamer capable of binding to a ligand; and
(k) DNA that transcribes to an RNA aptamer capable of binding to a ligand, and DNA that transcribes to regulatory RNA capable of regulating expression of the gene to be suppressed, wherein the regulation is dependent on the conformation of the regulatory RNA, and the conformation of the regulatory RNA is allosterically affected by the binding state of the RNA aptamer.

In some embodiments, an intron is used to deliver a gene suppression element in the absence of any protein-coding exons (coding sequence). In one example, an intron, such as an expression-enhancing intron, is interrupted by embedding within the intron a gene suppression element, wherein, upon transcription, the gene suppression element is excised from the intron. Thus, protein-coding exons are not required to provide the gene suppressing function of the recombinant DNA constructs disclosed herein.

### XXIV. Transcription Regulatory Elements

In some embodiments, the recombinant DNA construct or polynucleotide of this invention comprises DNA encoding a transcription regulatory element. Transcription regulatory elements include elements that regulate the expression level of the recombinant DNA construct of this invention (relative to its expression in the absence of such regulatory elements). Examples of suitable transcription regulatory elements include riboswitches (cis- or trans-acting), transcript stabilizing sequences, and miRNA recognition sites, as described in detail in U.S. Patent Application Publication 2006/0200878, specifically incorporated herein by reference.

### XXIV. Making and Using Transgenic Plant Cells and Transgenic Plants

Transformation of a plant can include any of several well-known methods and compositions. Suitable methods for plant transformation include virtually any method by which DNA can be introduced into a cell. One method of plant transformation is microprojectile bombardment, for example, as illustrated in U.S. Pat. No. 5,015,580 (soybean), U.S. Pat. No. 5,538,880 (maize), U.S. Pat. No. 5,550,318 (maize), U.S. Pat. No. 5,914,451 (soybean), U.S. Pat. No. 6,153,812 (wheat), U.S. Pat. No. 6,160,208 (maize), U.S. Pat. No. 6,288,312 (rice), U.S. Pat. No. 6,365,807 (rice), and U.S. Pat. No. 6,399,861 (maize), and U.S. Pat. No. 6,403,865 (maize), all of which are incorporated by reference for enabling the production of transgenic plants.

Another useful method of plant transformation is Agrobacterium-mediated transformation by means of Agrobacterium containing a binary Ti plasmid system, wherein the Agrobacterium carries a first Ti plasmid and a second, chimeric plasmid containing at least one T-DNA border of a wild-type Ti plasmid, a promoter functional in the transformed plant cell and operably linked to a polynucleotide or recombinant DNA construct of this invention. See, for example, the binary system described in U.S. Pat. No. 5,159,135, incorporated by reference. Also see De Framond (1983) Biotechnology, 1:262-269; and Hoekema et al., (1983) Nature, 303:179. In such a binary system, the smaller plasmid, containing the T-DNA border or borders, can be conveniently constructed and manipulated in a suitable alternative host, such as E. coli, and then transferred into Agrobacterium.

Detailed procedures for Agrobacterium-mediated transformation of plants, especially crop plants, include procedures disclosed in U.S. Pat. Nos. 5,004,863, 5,159,135, and 5,518,908 (cotton); U.S. Pat. Nos. 5,416,011, 5,569,834, 5,824,877 and 6,384,301 (soybean); U.S. Pat. Nos. 5,591,616 and 5,981,840 (maize); U.S. Pat. No. 5,463,174 (brassicas including canola), U.S. Pat. No. 7,026,528 (wheat), and U.S. Pat. No. 6,329,571 (rice), and in U.S. Patent Application Publications 2004/0244075 (maize) and 2001/0042257 A1 (sugar beet), all of which are specifically incorporated by reference for enabling the production of transgenic plants. U. S. Patent Application Publication 2011/0296555 discloses in Example 5 the transformation vectors (including the vector sequences) and detailed protocols for transforming maize, soybean, canola, cotton, and sugarcane) and is specifically incorporated by reference for enabling the production of transgenic plants. Similar methods have been reported for many plant species, both dicots and monocots, including, among others, peanut (Cheng et al. (1996) Plant Cell Rep., 15: 653); asparagus (Bytebier et al. (1987) Proc. Natl. Acad. Sci. U.S.A., 84:5345); barley (Wan and Lemaux (1994) Plant Physiol., 104:37); rice (Toriyama et al. (1988) Bio/Technology, 6:10; Zhang et al. (1988) Plant Cell Rep., 7:379; wheat (Vasil et al. (1992) Bio/Technology, 10:667; Becker et al. (1994) Plant J., 5:299), alfalfa (Masoud et al. (1996) Transgen. Res., 5:313); and tomato (Sun et al. (2006) Plant Cell Physiol., 47:426-431). See also a description of vectors, transformation methods, and production of transformed Arabidopsis thaliana plants where transcription factors are constitutively expressed by a CaMV35S promoter, in U. S. Patent Application Publication 2003/0167537 A1, incorporated by reference. Transformation methods specifically useful for solanaceous plants are well known in the art. See, for example, publicly described transformation methods for tomato (Sharma et al. (2009), J. Biosci., 34:423-433), eggplant (Arpaia et al. (1997) Theor. Appl. Genet., 95:329-334), potato (Bannerjee et al. (2006) Plant Sci., 170:732-738; Chakravarty et al. (2007) Amer. J. Potato Res., 84:301-311; S. Millam “Agrobacterium-mediated transformation of potato.” Chapter 19 (pp. 257-270), “Transgenic Crops of the World: Essential Protocols”, Ian S. Curtis (editor), Springer, 2004), and peppers (Li et al. (2003) Plant Cell Reports, 21: 785-788). Stably transgenic potato, tomato, and eggplant have been commercially introduced in various regions; see, e. g., K. Redenbaugh et al. “Safety Assessment of Genetically Engineered Fruits and Vegetables: A Case Study of the FLAVR SAVR Tomato”, CRC Press, Boca Raton, 1992, and the extensive publicly available documentation of commercial genetically modified crops in the GM Crop Database; see: CERA. (2012). GM Crop Database. Center for Environmental Risk Assessment (CERA), ILSI Research Foundation, Washington D.C., available electronically at cera-gmc.org/?action=gm_crop_database. Various methods of transformation of other plant species are well known in the art, see, for example, the encyclopedic reference, “Compendium of Transgenic Crop Plants”, edited by Chittaranjan Kole and Timothy C. Hall, Blackwell Publishing Ltd., 2008; ISBN 978-1-405-16924-0 (available electronically at mrw.interscience.wiley.com/emrw/9781405181099/hpt/toc), which describes transformation procedures for cereals and forage grasses (rice, maize, wheat, barley, oat, sorghum, pearl millet, finger millet, cool-season forage grasses, and bahiagrass), oilseed crops (soybean, oilseed brassicas, sunflower, peanut, flax, sesame, and safflower), legume grains and forages (common bean, cowpea, pea, faba bean, lentil, tepary bean, Asiatic beans, pigeonpea, vetch, chickpea, lupin, alfalfa, and clovers), temperate fruits and nuts (apple, pear, peach, plums, berry crops, cherries, grapes, olive, almond, and Persian walnut), tropical and subtropical fruits and nuts (citrus, grapefruit, banana and plantain, pineapple, papaya, mango, avocado, kiwifruit, passionfruit, and persimmon), vegetable crops (tomato, eggplant, peppers, vegetable brassicas, radish, carrot, cucurbits, alliums, asparagus, and leafy vegetables), sugar, tuber, and fiber crops (sugarcane, sugar beet, stevia, potato, sweet potato, cassava, and cotton), plantation crops, ornamentals, and turf grasses (tobacco, coffee, cocoa, tea, rubber tree, medicinal plants, ornamentals, and turf grasses), and forest tree species.

Transformation methods to provide transgenic plant cells and transgenic plants containing stably integrated recombinant DNA are preferably practiced in tissue culture on media and in a controlled environment. “Media” refers to the numerous nutrient mixtures that are used to grow cells in vitro, that is, outside of the intact living organism. Recipient cell targets include, but are not limited to, meristem cells, callus, immature embryos or parts of embryos, and gametic cells such as microspores, pollen, sperm, and egg cells. Any cell from which a fertile plant can be regenerated is contemplated as a useful recipient cell for practice of this invention. Callus can be initiated from various tissue sources, including, but not limited to, immature embryos or parts of embryos, seedling apical meristems, microspores, and the like. Those cells which are capable of proliferating as callus can serve as recipient cells for genetic transformation. Practical transformation methods and materials for making transgenic plants of this invention (e.g., various media and recipient target cells, transformation of immature embryos, and subsequent regeneration of fertile transgenic plants) are disclosed, for example, in U.S. Pat. Nos. 6,194,636 and 6,232,526 and U.S. Patent Application Publication 2004/0216189, which are specifically incorporated by reference.

In general transformation practice, DNA is introduced into only a small percentage of target cells in any one transformation experiment. Marker genes are generally used to provide an efficient system for identification of those cells that are stably transformed by receiving and integrating a transgenic DNA construct into their genomes. Preferred marker genes provide selective markers which confer resistance to a selective agent, such as an antibiotic or herbicide. Any of the antibiotics or herbicides to which a plant cell is resistant can be a useful agent for selection. Potentially transformed cells are exposed to the selective agent. In the population of surviving cells will be those cells where, generally, the resistance-conferring gene is integrated and expressed at sufficient levels to permit cell survival. Cells can be tested further to confirm stable integration of the recombinant DNA. Commonly used selective marker genes include those conferring resistance to antibiotics such as kanamycin or paromomycin (nptII), hygromycin B (aph IV) and gentamycin (aac3 and aacC4) or resistance to herbicides such as glufosinate (bar or pat) and glyphosate (EPSPS). Examples of useful selective marker genes and selection agents are illustrated in U.S. Pat. Nos. 5,550,318, 5,633,435, 5,780,708, and 6,118,047, all of which are specifically incorporated by reference. Screenable markers or reporters, such as markers that provide an ability to visually identify transformants can also be employed. Examples of useful screenable markers include, for example, a gene expressing a protein that produces a detectable color by acting on a chromogenic substrate (e.g., beta glucuronidase (GUS) (uidA) or luciferase (luc)) or that itself is detectable, such as green fluorescent protein (GFP) (gfp) or an immunogenic molecule. Those of skill in the art will recognize that many other useful markers or reporters are available for use.

Detecting or measuring transcription of a recombinant DNA construct in a transgenic plant cell can be achieved by any suitable method, including protein detection methods (e.g., western blots, ELISAs, and other immunochemical methods), measurements of enzymatic activity, or nucleic acid detection methods (e.g., Southern blots, northern blots, PCR, RT-PCR, fluorescent in situ hybridization).

Other suitable methods for detecting or measuring transcription in a plant cell of a recombinant polynucleotide of this invention targeting a Lepidopteran pest target gene include measurement of any other trait that is a direct or proxy indication of the level of expression of the target gene in the Lepidopteran pest, relative to the level of expression observed in the absence of the recombinant polynucleotide, e.g., growth rates, mortality rates, or reproductive or recruitment rates of the Lepidopteran pest, or measurements of injury (e.g., root injury) or yield loss in a plant or field of plants infested by the Lepidopteran pest. In general, suitable methods for detecting or measuring transcription in a plant cell of a recombinant polynucleotide of interest include, e.g., gross or microscopic morphological traits, growth rates, yield, reproductive or recruitment rates, resistance to pests or pathogens, or resistance to biotic or abiotic stress (e.g., water deficit stress, salt stress, nutrient stress, heat or cold stress). Such methods can use direct measurements of a phenotypic trait or proxy assays (e.g., in plants, these assays include plant part assays such as leaf or root assays to determine tolerance of abiotic stress). Such methods include direct measurements of resistance to an invertebrate pest or pathogen (e.g., damage to plant tissues) or proxy assays (e.g., plant yield assays, or bioassays such as the Western corn rootworm (Diabrotica virgifera virgifera LeConte) larval bioassay described in International Patent Application Publication WO2005/110068 A2 and U.S. Patent Application Publication US 2006/0021087 A1, specifically incorporated by reference, or the soybean cyst nematode bioassay described by Steeves et al. (2006) Funct. Plant Biol., 33:991-999, wherein cysts per plant, cysts per gram root, eggs per plant, eggs per gram root, and eggs per cyst are measured, or the Colorado potato beetle (Lepidopteran decemlineata) bioassay described herein in the working Examples.

The recombinant DNA constructs of this invention can be stacked with other recombinant DNA for imparting additional traits (e.g., in the case of transformed plants, traits including herbicide resistance, pest resistance, cold germination tolerance, water deficit tolerance, and the like) for example, by expressing or suppressing other genes. Constructs for coordinated decrease and increase of gene expression are disclosed in U.S. Patent Application Publication 2004/0126845 A1, specifically incorporated by reference.

Seeds of fertile transgenic plants can be harvested and used to grow progeny generations, including hybrid generations, of transgenic plants of this invention that include the recombinant DNA construct in their genome. Thus, in addition to direct transformation of a plant with a recombinant DNA construct of this invention, transgenic plants of this invention can be prepared by crossing a first plant having the recombinant DNA with a second plant lacking the construct. For example, the recombinant DNA can be introduced into a plant line that is amenable to transformation to produce a transgenic plant, which can be crossed with a second plant line to introgress the recombinant DNA into the resulting progeny. A transgenic plant of this invention can be crossed with a plant line having other recombinant DNA that confers one or more additional trait(s) (such as, but not limited to, herbicide resistance, pest or disease resistance, environmental stress resistance, modified nutrient content, and yield improvement) to produce progeny plants having recombinant DNA that confers both the desired target sequence expression behavior and the additional trait(s).

In such breeding for combining traits the transgenic plant donating the additional trait can be a male line (pollinator) and the transgenic plant carrying the base traits can be the female line. The progeny of this cross segregate such that some of the plant will carry the DNA for both parental traits and some will carry DNA for one parental trait; such plants can be identified by markers associated with parental recombinant DNA Progeny plants carrying DNA for both parental traits can be crossed back into the female parent line multiple times, e.g., usually 6 to 8 generations, to produce a homozygous progeny plant with substantially the same genotype as one original transgenic parental line as well as the recombinant DNA of the other transgenic parental line.

Yet another aspect of this invention is a transgenic plant grown from the transgenic seed (or in the case of potatoes, a transgenic seed potato) of this invention. This invention contemplates transgenic plants grown directly from transgenic seed containing the recombinant DNA as well as progeny generations of plants, including inbred or hybrid plant lines, made by crossing a transgenic plant grown directly from transgenic seed to a second plant not grown from the same transgenic seed. Crossing can include, for example, the following steps:
(a) plant seeds of the first parent plant (e.g., non-transgenic or a transgenic) and a second parent plant that is transgenic according to the invention;
(b) grow the seeds of the first and second parent plants into plants that bear flowers;
(c) pollinate a flower from the first parent with pollen from the second parent; and
(d) harvest seeds produced on the parent plant bearing the fertilized flower.

It is often desirable to introgress recombinant DNA into elite varieties, e.g., by backcrossing, to transfer a specific desirable trait from one source to an inbred or other plant that lacks that trait. This can be accomplished, for example, by first crossing a superior inbred (“A”) (recurrent parent) to a donor inbred (“B”) (non-recurrent parent), which carries the appropriate gene(s) for the trait in question, for example, a construct prepared in accordance with the current invention. The progeny of this cross first are selected in the resultant progeny for the desired trait to be transferred from the non-recurrent parent “B”, and then the selected progeny are mated back to the superior recurrent parent “A”. After five or more backcross generations with selection for the desired trait, the progeny can be essentially hemizygous for loci controlling the characteristic being transferred, but are like the superior parent for most or almost all other genes. The last backcross generation would be selfed to give progeny which are pure breeding for the gene(s) being transferred, e.g., one or more transformation events.

Through a series of breeding manipulations, a selected DNA construct can be moved from one line into an entirely different line without the need for further recombinant manipulation. One can thus produce inbred plants which are true breeding for one or more DNA constructs. By crossing different inbred plants, one can produce a large number of different hybrids with different combinations of DNA constructs. In this way, plants can be produced which have the desirable agronomic properties frequently associated with hybrids (“hybrid vigor”), as well as the desirable characteristics imparted by one or more DNA constructs.

In certain transgenic plant cells and transgenic plants of this invention, it is sometimes desirable to concurrently express a gene of interest while also modulating expression of a Lepidopteran target gene. Thus, in some embodiments, the transgenic plant contains recombinant DNA further comprising a gene expression element for expressing at least one gene of interest, and transcription of the recombinant DNA construct of this invention is effected with concurrent transcription of the gene expression element.

In some embodiments, the recombinant DNA constructs of this invention can be transcribed in any plant cell or tissue or in a whole plant of any developmental stage. Transgenic plants can be derived from any monocot or dicot plant, such as, but not limited to, plants of commercial or agricultural interest, such as crop plants (especially crop plants used for human food or animal feed), wood- or pulp-producing trees, vegetable plants, fruit plants, and ornamental plants. Examples of plants of interest include grain crop plants (such as wheat, oat, barley, maize, rye, triticale, rice, millet, sorghum, quinoa, amaranth, and buckwheat); forage crop plants (such as forage grasses and forage dicots including alfalfa, vetch, clover, and the like); oilseed crop plants (such as cotton, safflower, sunflower, soybean, canola, rapeseed, flax, peanuts, and oil palm); tree nuts (such as walnut, cashew, hazelnut, pecan, almond, and the like); sugarcane, coconut, date palm, olive, sugarbeet, tea, and coffee; wood- or pulp-producing trees; vegetable crop plants such as legumes (for example, beans, peas, lentils, alfalfa, peanut), lettuce, asparagus, artichoke, celery, carrot, radish, the brassicas (for example, cabbages, kales, mustards, and other leafy brassicas, broccoli, cauliflower, Brussels sprouts, turnip, kohlrabi), edible cucurbits (for example, cucumbers, melons, summer squashes, winter squashes), edible alliums (for example, onions, garlic, leeks, shallots, chives), edible members of the Solanaceae (for example, tomatoes, eggplants, potatoes, peppers, groundcherries), and edible members of the Chenopodiaceae (for example, beet, chard, spinach, quinoa, amaranth); fruit crop plants such as apple, pear, citrus fruits (for example, orange, lime, lemon, grapefruit, and others), stone fruits (for example, apricot, peach, plum, nectarine), banana, pineapple, grape, kiwifruit, papaya, avocado, and berries; plants grown for biomass or biofuel (for example, Miscanthus grasses, switchgrass, jatropha, oil palm, eukaryotic microalgae such as Botryococcus braunii, Chlorella spp., and Dunaliella spp., and eukaryotic macroalgae such as Gracilaria spp., and Sargassum spp.); and ornamental plants including ornamental flowering plants, ornamental trees and shrubs, ornamental groundcovers, and ornamental grasses.

This invention also provides commodity products produced from a transgenic plant cell, plant, or seed of this invention, including, but not limited to, harvested leaves, roots, shoots, tubers, stems, fruits, seeds, or other parts of a plant, meals, oils, extracts, fermentation or digestion products, crushed or whole grains or seeds of a plant, or any food or non-food product including such commodity products produced from a transgenic plant cell, plant, or seed of this invention. The detection of one or more of nucleic acid sequences of the recombinant DNA constructs of this invention in one or more commodity or commodity products contemplated herein is de facto evidence that the commodity or commodity product contains or is derived from a transgenic plant cell, plant, or seed of this invention.

Generally a transgenic plant having in its genome a recombinant DNA construct of this invention exhibits increased resistance to a Lepidopteran pest infestation. In various embodiments, for example, where the transgenic plant expresses a recombinant DNA construct of this invention that is stacked with other recombinant DNA for imparting additional traits, the transgenic plant has at least one additional altered trait, relative to a plant lacking the recombinant DNA construct, selected from the group of traits consisting of:
(a) improved abiotic stress tolerance;
(b) improved biotic stress tolerance;
(c) modified primary metabolite composition;
(d) modified secondary metabolite composition;
(e) modified trace element, carotenoid, or vitamin composition;
(f) improved yield;
(g) improved ability to use nitrogen, phosphate, or other nutrients;
(h) modified agronomic characteristics;
(i) modified growth or reproductive characteristics; and
(j) improved harvest, storage, or processing quality.

In some embodiments, the transgenic plant is characterized by: improved tolerance of abiotic stress (e.g., tolerance of water deficit or drought, heat, cold, non-optimal nutrient or salt levels, non-optimal light levels) or of biotic stress (e.g., crowding, allelopathy, or wounding); by a modified primary metabolite (e.g., fatty acid, oil, amino acid, protein, sugar, or carbohydrate) composition; a modified secondary metabolite (e.g., alkaloids, terpenoids, polyketides, non-ribosomal peptides, and secondary metabolites of mixed biosynthetic origin) composition; a modified trace element (e.g., iron, zinc), carotenoid (e.g., beta-carotene, lycopene, lutein, zeaxanthin, or other carotenoids and xanthophylls), or vitamin (e.g., tocopherols) composition; improved yield (e.g., improved yield under non-stress conditions or improved yield under biotic or abiotic stress); improved ability to use nitrogen, phosphate, or other nutrients; modified agronomic characteristics (e.g., delayed ripening; delayed senescence; earlier or later maturity; improved shade tolerance; improved resistance to root or stalk lodging; improved resistance to “green snap” of stems; modified photoperiod response); modified growth or reproductive characteristics (e.g., intentional dwarfing; intentional male sterility, useful, e.g., in improved hybridization procedures; improved vegetative growth rate; improved germination; improved male or female fertility); improved harvest, storage, or processing quality (e.g., improved resistance to pests during storage, improved resistance to breakage, improved appeal to consumers); or any combination of these traits.

In another embodiment, transgenic seed, or seed produced by the transgenic plant, has modified primary metabolite (e.g., fatty acid, oil, amino acid, protein, sugar, or carbohydrate) composition, a modified secondary metabolite composition, a modified trace element, carotenoid, or vitamin composition, an improved harvest, storage, or processing quality, or a combination of these. In another embodiment, it can be desirable to change levels of native components of the transgenic plant or seed of a transgenic plant, for example, to decrease levels of an allergenic protein or glycoprotein or of a toxic metabolite.

Generally, screening a population of transgenic plants each regenerated from a transgenic plant cell is performed to identify transgenic plant cells that develop into transgenic plants having the desired trait. The transgenic plants are assayed to detect an enhanced trait, e.g., enhanced water use efficiency, enhanced cold tolerance, increased yield, enhanced nitrogen use efficiency, enhanced seed protein, and enhanced seed oil. Screening methods include direct screening for the trait in a greenhouse or field trial or screening for a surrogate trait. Such analyses are directed to detecting changes in the chemical composition, biomass, physiological properties, or morphology of the plant. Changes in chemical compositions such as nutritional composition of grain are detected by analysis of the seed composition and content of protein, free amino acids, oil, free fatty acids, starch, tocopherols, or other nutrients. Changes in growth or biomass characteristics are detected by measuring plant height, stem diameter, internode length, root and shoot dry weights, and (for grain-producing plants such as maize, rice, or wheat) ear or seed head length and diameter. Changes in physiological properties are identified by evaluating responses to stress conditions, e.g., assays under imposed stress conditions such as water deficit, nitrogen or phosphate deficiency, cold or hot growing conditions, pathogen or insect attack, light deficiency, or increased plant density. Other selection properties include days to flowering, days to pollen shed, days to fruit maturation, fruit or tuber quality or amount produced, days to silking in maize, leaf extension rate, chlorophyll content, leaf temperature, stand, seedling vigor, internode length, plant height, leaf number, leaf area, tillering, brace roots, staying green, stalk lodging, root lodging, plant health, fertility, green snap, and pest resistance. In addition, phenotypic characteristics of harvested fruit, seeds, or tubers can be evaluated; for example, in tomato and eggplant this can include the total number or weight of fruit harvested or the color, acidity, sugar content, or flavor of such fruit, and in potato this can include the number or total weight of tubers harvested and the quality of such tubers.

The following Examples are presented for the purposes of illustration and should not be construed as limitations.

### EXAMPLES

### Identification of Target Genes

Target genes used for RNAi were identified by analyzing the target Lepidopteran pest's transcriptome for genes with desired nucleotide expression profiles at product-relevant tags and single- and low-copy numbers using publicly available data. Secondary confirmations on these were performed using literature searches and public databases such as Database of Essential Genes (DEG), iBeetle, Flybase, Lepbase etc. to assess their importance as RNAi targets. These genes were then nominated for design of dsRNA triggers

### Design of Efficacious Trigger Sequences

The identified target genes were then processed by a proprietary algorithm to be partitioned into all possible 18-25 bp long segments. These segments were then matched by the Burrows Wheeler Aligner (BWA) tool to the specific target genes' RNA sequence obtained from the respective publicly available transcriptome. These matching segments were then parsed to identify one or more subsequences of the target gene. Efficacious dsRNA triggers were then designed by combining then inputting these subsequences into proprietary algorithms and a variety of public dsRNA design tools such as Snapdragon, E-RNAi and SiFi21. Specificity of the designed triggers was then checked using BLAST and proprietary code for matches to species that were not targeted, and any hits from this analysis were re-designed using the methodology above.

TABLE 1A

| | Accession in Model | | | |
|---|---|---|---|---|
| Genes Targeted | Species | Target Species | Trigger ID | SEQ ID NO |
| PGRP | XM_011556718.1 | Plutella xylostella | GS144 | 115 |
| VATpaseE | NM_001305532.1 | Plutella xylostella | GS146 | 116 |
| ABCE | XM_011562286.1 | Plutella xylostella | GS148 | 117 |
| TH | NM_001305520.1 | Plutella xylostella | GS150 | 118 |
| Hemolin | FJ687752.1 | Plutella xylostella | GS152 | 119 |
| TH | MF440319.1 | Spodoptera | GS262 | 120 |
| | | frugiperda | | |
| Hemolin | XP_022819117.1 | [symbol] | GS263 | 121 |
| | | [symbol] | | |
| vATPasesubE | XP_030031453.1 | Spodoptera | GS264 | 122 |
| | | frugiperda | | |
| ABCE | XP_022815393.1 | [symbol] | GS265 | 123 |
| | | [symbol] | | |
| ABCE | XP_022815393.1 | Spodoptera | GS266 | 124 |
| | | frugiperda | | |
| IAP | AFA43941.1 | Spodoptera | GS267 | 125 |
| | | frugiperda | | |
| PGRP | XM_011556718.1 | Plutella xylostella | GS268 | 126 |
| vATPaseE | NM_001305532.1 | Plutella xylostella | GS269 | 127 |
| ABCE | XM_011562286.1 | Plutella xylostella | GS270 | 128 |
| TH | NM_001305520.1 | Plutella xylostella | GS271 | 129 |
| Hemolin | FJ687752.1 | Plutella xylostella | GS272 | 130 |
| IAP | AFA43941.1 | Plutella xylostella | GS273 | 131 |
| Proteasome beta | XM_022979686.1 | Spodoptera | GS297 | 132 |
| | | frugiperda | | |
| Rpll 140 | XM_022978887.1 | Spodoptera | GS298 | 133 |
| | | frugiperda | | |
| Rop (Ras opposite) | XM_022974274.1 | Spodoptera | GS299 | 134 |
| | | frugiperda | | |
| Voltage-gated potassium | XM_022966217.1 | [symbol] | GS300 | 135 |
| channel | | [symbol] | | |
| Rieske | XM_022965227.1 | Spodoptera | GS301 | 136 |
| | | frugiperda | | |
| Duox | XM_011560542.1 | Spodoptera | GS302 | 137 |
| | | frugiperda | | |
| Domeless | Px009358 | Spodoptera | GS303 | 138 |
| | | frugiperda | | |
| PPO | Px002274 | Spodoptera | GS304 | 139 |
| | | frugiperda | | |
| TAK1 | Px002003 | [symbol] | GS305 | 140 |
| | | [symbol] | | |
| Dred | Px000411 | [symbol] | GS306 | 141 |
| | | [symbol] | | |
| FADD | Px011635 | Spodoptera | GS307 | 142 |
| | | frugiperda | | |
| COPI beta′ coatomer | XM_011567740.1 | [symbol] | GS309 | 143 |
| | | [symbol] | | |
| Cactus | XM_011555277.1 | Spodoptera | GS311 | 144 |
| | | frugiperda | | |
| IKK-β | Px005490 | Spodoptera | GS313 | 145 |
| | | frugiperda | | |
| Tube | Px003725 | Spodoptera | GS314 | 146 |
| | | frugiperda | | |
| Imd | JQ710735.1 | Spodoptera | GS315 | 147 |
| | | frugiperda | | |
| PBAN | AY173075.1 | Spodoptera | GS316 | 148 |
| | | frugiperda | | |
| beta-1,3-glucan recognition | AY135522.1 | Spodoptera | GS317 | 149 |
| protein 2 | | frugiperda | | |
| Dred | Px000411 | Plutella xylostella | GS318 | 150 |
| TAK1 | Px002003 | Plutella xylostella | GS319 | 151 |
| PPO | Px002274 | Plutella xylostella | GS320 | 152 |
| Domeless | Px009358 | Plutella xylostella | GS321 | 153 |
| Duox | XM_011560542.1 | Plutella xylostella | GS322 | 154 |
| Rieske | XM_022965227.1 | Plutella xylostella | GS323 | 155 |
| Voltage-gated potassium | XM_022966217.1 | Plutella xylostella | GS324 | 156 |
| channel | | | | |
| Rop (Ras opposite) | XM_022974274.1 | Plutella xylostella | GS325 | 157 |
| Rpll 140 | XM_022978887.1 | Plutella xylostella | GS326 | 158 |
| Proteasome beta | XM_022979686.1 | Plutella xylostella | GS327 | 159 |
| beta-1,3-glucan recognition | AY135522.1 | Plutella xylostella | GS328 | 160 |
| protein 2 | | | | |
| PBAN | AY173075.1 | Plutella xylostella | GS329 | 161 |
| Imd | JQ710735.1 | Plutella xylostella | GS330 | 162 |
| Tube | Px003725 | Plutella xylostella | GS331 | 163 |
| IKK-β | Px005490 | Plutella xylostella | GS332 | 164 |
| FADD | Px011635 | Plutella xylostella | GS333 | 165 |
| ATF-2 | XM_011549568.1 | Plutella xylostella | GS334 | 166 |
| Cactus | XM_011555277.1 | Plutella xylostella | GS335 | 167 |
| p38 | XM_011563266.1 | Plutella xylostella | GS336 | 168 |
| COPI beta′ coatomer | XM_011567740.1 | Plutella xylostella | GS337 | 169 |
| Hopscotch | XM_011569785.1 | Plutella xylostella | GS338 | 170 |
| dorsal | AIA24466.1 | Spodoptera | GS440 | 171 |
| | | frugiperda | | |
| Kenny | dme:Dmel_CG16910 | Spodoptera | GS441 | 172 |
| | | frugiperda | | |
| Ankyrin | dme:Dmel_CG1651 | Spodoptera | GS442 | 173 |
| | | frugiperda | | |
| caspar | dme:Dmel_CG8400 | Spodoptera | GS443 | 174 |
| | | frugiperda | | |
| duf | TC002914 | Spodoptera | GS444 | 175 |
| | | frugiperda | | |
| htl | TC004713 | Spodoptera | GS445 | 176 |
| | | frugiperda | | |
| axed | NP_001356975.1 | Spodoptera | GS446 | 177 |
| | | frugiperda | | |
| Rbm24 | TC001720 | Spodoptera | GS447 | 178 |
| | | frugiperda | | |
| pirk | dme:Dmel_CG15678 | Spodoptera | GS448 | 179 |
| | | frugiperda | | |
| bendless | dme:Dmel_CG18319 | Spodoptera | GS449 | 180 |
| | | frugiperda | | |
| shadow | QCX08945.1 | Spodoptera | GS450 | 181 |
| | | frugiperda | | |
| ABCH1 | AKC96438.1 | Spodoptera | GS451 | 182 |
| | | frugiperda | | |
| Dumpy | NP_001260042.1 | Spodoptera | GS452 | 183 |
| | | frugiperda | | |
| dorsal | AIA24466.1 | Plutella xylostella | GS453 | 184 |
| Kenny | dme:Dmel_CG16910 | Plutella xylostella | GS454 | 185 |
| Ankyrin | dme:Dmel_CG1651 | Plutella xylostella | GS455 | 186 |
| caspar | dme:Dmel_CG8400 | Plutella xylostella | GS456 | 187 |
| duf | TC002914 | Plutella xylostella | GS457 | 188 |
| htl | TC004713 | Plutella xylostella | GS458 | 189 |
| axed | NP_001356975.1 | Plutella xylostella | GS459 | 190 |
| Rbm24 | TC001720 | Plutella xylostella | GS460 | 191 |
| pirk | dme:Dmel_CG15678 | Plutella xylostella | GS461 | 192 |
| bendless | dme:Dmel_CG18319 | Plutella xylostella | GS462 | 193 |
| shadow | QCX08945.1 | Plutella xylostella | GS463 | 194 |
| ABCH1 | AKC96438.1 | Plutella xylostella | GS464 | 195 |
| Dumpy | NP_001260042.1 | Plutella xylostella | GS465 | 196 |
| AK (arginine kinase) | AIY55683.1 | Spodoptera | GS466 | 197 |
| | | frugiperda | | |
| VHDL (very high density | XP_021195805.1 | Spodoptera | GS467 | 198 |
| lipoprotein) | | frugiperda | | |
| AK (arginine kinase) | AIY55683.1 | Plutella xylostella | GS468 | 199 |
| VHDL (very high density | XP_021195805.1 | Plutella xylostella | GS469 | 200 |
| lipoprotein) | | | | |
| diuretic hormone 31 | NP_523514.1 | [symbol] | GS470 | 201 |
| | | [symbol] | | |
| diuretic hormone 31 | NP_523514.1 | Plutella xylostella | GS471 | 202 |
| pre-mRNA splicing factor | XP_022820559.1 | Spodoptera | GS472 | 203 |
| | | frugiperda | | |
| PRGP-LE | dme:Dmel_CG8995 | Spodoptera | GS473 | 204 |
| | | frugiperda | | |
| COP I gamma | TC011806 | Spodoptera | GS474 | 205 |
| | | frugiperda | | |
| COP I zeta | NP_648910.1 | Spodoptera | GS475 | 206 |
| | | frugiperda | | |
| COP I beta prime | NP_524836.2 | Spodoptera | GS476 | 207 |
| | | frugiperda | | |
| COP I alpha | NP_477395.1 | Spodoptera | GS477 | 208 |
| | | frugiperda | | |
| COP I delta | NP_652012.1 | [symbol] | GS478 | 209 |
| | | [symbol] | | |
| COP I epsilon | NP_609037.1 | Spodoptera | GS479 | 210 |
| | | frugiperda | | |
| verm (vermiform) | TC014101 | Spodoptera | GS480 | 211 |
| | | frugiperda | | |
| kr-h1 (kruppel homolog 1) | TC006419 | Spodoptera | GS481 | 212 |
| | | frugiperda | | |
| Calcium-activated potassium | AAA28651.1 | [symbol] | GS482 | 213 |
| channel | | [symbol] | | |
| InR2 (insulin receptor 2) | ARD07922.1 | Spodoptera | GS483 | 214 |
| | | frugiperda | | |
| tubulin 2 | XP_021194879.1 | Spodoptera | GS484 | 215 |
| | | frugiperda | | |
| pre-mRNA splicing factor | XP_022820559.1 | Plutella xylostella | GS485 | 216 |
| COP I gamma | TC011806 | Plutella xylostella | GS486 | 217 |
| COP I zeta | NP_648910.1 | Plutella xylostella | GS487 | 218 |
| COP I beta prime | NP_524836.2 | Plutella xylostella | GS488 | 219 |
| COP I alpha | NP_477395.1 | Plutella xylostella | GS489 | 220 |
| COP I delta | NP_652012.1 | Plutella xylostella | GS490 | 221 |
| COP I epsilon | NP_609037.1 | Plutella xylostella | GS491 | 222 |
| verm (vermiform) | TC014101 | Plutella xylostella | GS492 | 223 |
| kr-h1 (kruppel homolog 1) | TC006419 | Plutella xylostella | GS493 | 224 |
| Calcium-activated potassium | AAA28651.1 | Plutella xylostella | GS494 | 225 |
| channel | | | | |
| InR2 (insulin receptor 2) | ARD07922.1 | Plutella xylostella | GS495 | 226 |
| tubulin 2 | XP_021194879.1 | Plutella xylostella | GS496 | 227 |
| PRGP-LE | dme:Dmel_CG8995 | Plutella xylostella | GS497 | 228 |

TABLE 1B

| | | Accession in Model | | |
|---|---|---|---|---|
| Target_species | Genes Targeted | Species | Universal_ID | SEQ ID NO. |
| Spodoptera | sd | TC032219 | GS921 | 362 |
| frugiperda | | | | |
| Spodoptera | Rfabg - Retinoid- and fatty acid-binding | TC034740 | GS985 | 363 |
| frugiperda | glycoprotein | | | |
| Spodoptera | Rpb7 | TC014109 | GS922 | 364 |
| frugiperda | | | | |
| Spodoptera | calypso | TC009963 | GS924 | 365 |
| frugiperda | | | | |
| Spodoptera | Rbm24 | TC001720 | GS917 | 366 |
| frugiperda | | | | |
| Spodoptera | twisted bristles roughened eye | TC000179 | GS919 | 367 |
| frugiperda | | | | |
| Spodoptera | surfeit 4 | TC000161 | GS923 | 368 |
| frugiperda | | | | |
| Spodoptera | Rpn7 regulatory particle non-ATPase 7 | TC006375 | GS925 | 369 |
| frugiperda | | | | |
| Spodoptera | how - held out wings | TC000827 | GS986 | 370 |
| frugiperda | | | | |
| Spodoptera | Phenylalanyl-tRNA synthetase beta subunit | TC032227 | GS926 | 371 |
| frugiperda | | | | |
| Spodoptera | twi | TC014598 | GS918 | 372 |
| frugiperda | | | | |
| Spodoptera | mbc | TC012454 | GS920 | 373 |
| frugiperda | | | | |
| Spodoptera | psc - posterior sex combs | TC005276 | GS868 | 374 |
| frugiperda | | | | |
| Spodoptera | Upf1 | TC000192 | GS872 | 375 |
| frugiperda | | | | |
| Spodoptera | sr | TC004846 | GS870 | 376 |
| frugiperda | | | | |
| Spodoptera | Fit1- Fit2 | TC010123 | GS867 | 377 |
| frugiperda | | | | |
| Spodoptera | RRM domain-containing protein | TC010637 | GS869 | 378 |
| frugiperda | | | | |
| Spodoptera | Tc croc | TC002813 | GS866 | 379 |
| frugiperda | | | | |
| Spodoptera | Protein kinase | TC000040 | GS877 | 380 |
| frugiperda | | | | |
| Spodoptera | Arp3 | TC002963 | GS876 | 381 |
| frugiperda | | | | |
| Spodoptera | Actin related protein 2 | TC000144 | GS878 | 382 |
| frugiperda | | | | |
| Spodoptera | TAR DNA-binding protein 43-like Protein | TC006055 | GS865 | 383 |
| frugiperda | | | | |
| Spodoptera | wb | TC014773 | GS884 | 384 |
| frugiperda | | | | |
| Spodoptera | TC004745 | TC004745 | GS987 | 385 |
| frugiperda | | | | |
| Spodoptera | ara | TC031040 | GS873 | 386 |
| frugiperda | | | | |
| Spodoptera | Mef2 | TC010850 | GS885 | 387 |
| frugiperda | | | | |
| Spodoptera | FA elongase | TC010977 | GS883 | 388 |
| frugiperda | | | | |
| Spodoptera | Uncharacterized protein | TC010693 | GS875 | 389 |
| frugiperda | | | | |
| Spodoptera | Translocase of inner mitochondrial | TC000047 | GS880 | 390 |
| frugiperda | membrane 23 | | | |
| Spodoptera | Pax2 | TC003570 | GS879 | 391 |
| frugiperda | | | | |
| Spodoptera | Vrp1 | TC012341 | GS871 | 392 |
| frugiperda | | | | |
| Spodoptera | dre4-CG1828 | TC014294 | GS882 | 393 |
| frugiperda | | | | |
| Spodoptera | Dhx15 | TC000481 | GS881 | 394 |
| frugiperda | | | | |
| Spodoptera | CG40470 (nearest homolog) | TC000165 | GS874 | 395 |
| frugiperda | | | | |
| Spodoptera | Rpl40 | FBgn0003941 | GS932 | 396 |
| frugiperda | | | | |
| Spodoptera | Rps21 | FBgn0015521 | GS942 | 397 |
| frugiperda | | | | |
| Spodoptera | RpL3 | FBgn0020910 | GS943 | 398 |
| frugiperda | | | | |
| Spodoptera | Vps28 | FBgn0021814 | GS944 | 399 |
| frugiperda | | | | |
| Spodoptera | Sec23A | FBgn0262125 | GS933 | 400 |
| frugiperda | | | | |
| Spodoptera | Sec6 | FBgn0266671 | GS934 | 401 |
| frugiperda | | | | |
| Spodoptera | vATPase-Vha26 | FBgn0283535 | GS945 | 402 |
| frugiperda | | | | |
| Spodoptera | Vps16A | FBgn0285911 | GS946 | 403 |
| frugiperda | | | | |
| Spodoptera | UEV1a | Dmel_CG10640 | GS935 | 404 |
| frugiperda | | | | |
| Spodoptera | relish | Dmel_CG11992 | GS936 | 405 |
| frugiperda | | | | |
| Spodoptera | diptericin | Dmel_CG12763 | GS979 | 406 |
| frugiperda | | | | |
| Spodoptera | spirit | Dmel_CG2056 | GS973 | 407 |
| frugiperda | | | | |
| Spodoptera | sphynx | Dmel_CG32382 | GS981 | 408 |
| frugiperda | | | | |
| Spodoptera | grass | Dmel_CG5896 | GS976 | 409 |
| frugiperda | | | | |
| Spodoptera | pelle | Dmel_CG5974 | GS974 | 410 |
| frugiperda | | | | |
| Spodoptera | DIF | Dmel_CG6667 | GS977 | 411 |
| frugiperda | | | | |
| Spodoptera | Effete | Dmel_CG7425 | GS980 | 412 |
| frugiperda | | | | |
| Spodoptera | spheroide | Dmel_CG9675 | GS968 | 413 |
| frugiperda | | | | |
| Spodoptera | Toll | Dmel_CG5490 | GS975 | 414 |
| frugiperda | | | | |
| Spodoptera | Tab2 | Dmel_CG7417 | GS966 | 415 |
| frugiperda | | | | |
| Spodoptera | caudal | NM_001273712 | GS971 | 416 |
| frugiperda | | | | |
| Spodoptera | methonine-rich storage protein (hexamerin | JF798635 | GS970 | 417 |
| frugiperda | homolog) | | | |
| Spodoptera | Insulin receptor 1 | KX507134 | GS978 | 418 |
| frugiperda | | | | |
| Spodoptera | Sh - Shaker | TC003580 | GS965 | 419 |
| frugiperda | | | | |
| Spodoptera | SK - small conductance calcium-activated | TC014196 | GS967 | 420 |
| frugiperda | potassium channel | | | |
| Spodoptera | chitinase1 | JQ653040 | GS982 | 421 |
| frugiperda | | | | |
| Spodoptera | ATPase | JQ653046 | GS972 | 422 |
| frugiperda | | | | |
| Spodoptera | Vacuolar ATPase A | KM591219 | GS969 | 423 |
| frugiperda | | | | |
| Spodoptera | methionine_rich_storage_protein | ABX55887.1 | GS1046 | 424 |
| frugiperda | | | | |
| Spodoptera | SK_gene | XP_008193517.1 | GS1026 | 425 |
| frugiperda | | | | |
| Spodoptera | troponin | AYD60125.1 | GS1048 | 426 |
| frugiperda | | | | |
| Spodoptera | P102 | AIE56154.1 | GS1045 | 427 |
| frugiperda | | | | |
| Spodoptera | vATPase | ADN84935.1 | GS1036 | 428 |
| frugiperda | | | | |
| Spodoptera | chitinase | AAS18266.1 | GS1044 | 429 |
| frugiperda | | | | |
| Plutella xylostella | sd | TC032219 | GS907 | 430 |
| Plutella xylostella | Rpb7 | TC014109 | GS910 | 431 |
| Plutella xylostella | calypso | TC009963 | GS914 | 432 |
| Plutella xylostella | Rbm24 | TC001720 | GS913 | 433 |
| [symbol] | twisted bristles roughened eye | TC000179 | GS909 | 434 |
| Plutella xylostella | surfeit 4 | TC000161 | GS912 | 435 |
| Plutella xylostella | Rpn7 | TC006375 | GS915 | 436 |
| Plutella xylostella | how | TC000827 | GS911 | 437 |
| Plutella xylostella | Phenylalanyl-tRNA synthetase beta subunit | TC032227 | GS916 | 438 |
| Plutella xylostella | twi | TC014598 | GS908 | 439 |
| Plutella xylostella | mbc | TC012454 | GS983 | 440 |
| Plutella xylostella | psc - posterior sex combs | TC005276 | GS899 | 441 |
| [symbol] | Upf1 | TC000192 | GS900 | 442 |
| Plutella xylostella | sr | TC004846 | GS894 | 443 |
| Plutella xylostella | Fit1-2 | TC010123 | GS897 | 444 |
| Plutella xylostella | RRM domain-containing protein | TC010637 | GS903 | 445 |
| Plutella xylostella | Tc croc | TC002813 | GS906 | 446 |
| Plutella xylostella | Protein kinase | TC000040 | GS889 | 447 |
| Plutella xylostella | Arp3 | TC002963 | GS904 | 448 |
| Plutella xylostella | Arp2 | TC000144 | GS895 | 449 |
| Plutella xylostella | TAR DNA-binding protein 43-like Protein | TC006055 | GS890 | 450 |
| [symbol] | wb | TC014773 | GS898 | 451 |
| [symbol] | Unknown | TC004745 | GS984 | 452 |
| [symbol] | ara | TC031040 | GS887 | 453 |
| [symbol] | Mef2 | TC010850 | GS901 | 454 |
| Plutella xylostella | FA elongase | TC010977 | GS902 | 455 |
| [symbol] | Uncharacterized protein | TC010693 | GS892 | 456 |
| [symbol] | Translocase of inner mitochondrial | TC000047 | GS905 | 457 |
| | membrane 23 | | | |
| Plutella xylostella | Pax2 | TC003570 | GS891 | 458 |
| Plutella xylostella | Vrp1 | TC012341 | GS893 | 459 |
| Plutella xylostella | dre4-CG1828 | TC014294 | GS886 | 460 |
| [symbol] | Dhx15 | TC000481 | GS896 | 461 |
| Plutella xylostella | CG40470 | TC000165 | GS888 | 462 |
| Plutella xylostella | Rpl40 | FBgn0003941 | GS927 | 463 |
| Plutella xylostella | Rps21 | FBgn0015521 | GS937 | 464 |
| Plutella xylostella | RpL3 | FBgn0020910 | GS938 | 465 |
| Plutella xylostella | Vps28 | FBgn0021814 | GS939 | 466 |
| Plutella xylostella | Sec23A | FBgn0262125 | GS928 | 467 |
| Plutella xylostella | Sec6 | FBgn0266671 | GS929 | 468 |
| Plutella xylostella | vATPase-Vha26 | FBgn0283535 | GS940 | 469 |
| Plutella xylostella | Vps16A | FBgn0285911 | GS941 | 470 |
| Plutella xylostella | UEV1a | Dmel_CG10640 | GS930 | 471 |
| Plutella xylostella | relish | Dmel_CG11992 | GS931 | 472 |
| Plutella xylostella | diptericin | Dmel_CG12763 | GS963 | 473 |
| [symbol] | spirit | Dmel_{—}CG2056 | GS964 | 474 |
| Plutella xylostella | sphynx | Dmel_CG32382 | GS948 | 475 |
| [symbol] | grass | Dmel_{—}CG5896 | GS957 | 476 |
| Plutella xylostella | pelle | Dmel_CG5974 | GS953 | 477 |
| Plutella xylostella | DIF | Dmel_CG6667 | GS951 | 478 |
| [symbol] | Effete | Dmel_{—}CG7425 | GS958 | 479 |
| Plutella xylostella | spheroide | Dmel_CG9675 | GS960 | 480 |
| Plutella xylostella | Toll | Dmel_CG5490 | GS952 | 481 |
| Plutella xylostella | Tab2 | Dmel_CG7417 | GS961 | 482 |
| Plutella xylostella | caudal | NM_001273712 | GS955 | 483 |
| Plutella xylostella | methonine-rich_storage_protein | JF798635 | GS949 | 484 |
| Plutella xylostella | Insulin_receptor_1 | KX507134 | GS947 | 485 |
| Plutella xylostella | Sh - Shaker | TC003580 | GS950 | 486 |
| Plutella xylostella | SK - small conductance calcium-activated | TC014196 | GS962 | 487 |
| | potassium channel | | | |
| Plutella xylostella | chitinase1 | JQ653040 | GS954 | 488 |
| [symbol] | ATPase | JQ653046 | GS959 | 489 |
| Plutella xylostella | Vacuolar ATPase A | KM591219 | GS956 | 490 |
| [symbol] | methionine_{—}rich_{—}storage_{—}protein | ABX55887.1 | GS1009 | 491 |
| | (hexamerin homolog) | | | |
| [symbol] | chitinase | AAS18266.1 | GS1001 | 492 |
| Plutella xylostella | troponin | AYD60125.1 | GS1249 | 493 |
| Plutella xylostella | P102 | AIE56154.1_P102 | GS1256 | 494 |
| Plutella xylostella | Sodium/potassium-transporting ATPase | AT1B_ARTSF | GS989 | 512 |
| | subunit alpha-B | | | |
| Plutella xylostella | Lac | LACH_DROME | GS990 | 513 |
| Plutella xylostella | lov | LOV_DROME | GS991 | 514 |
| Plutella xylostella | jar | MYS9_DROME | GS992 | 516 |
| Plutella xylostella | Clk | CLOCK_DROME | GS993 | 517 |
| Plutella xylostella | lap | PICAL_DROME | GS994 | 518 |
| Plutella xylostella | Atu | ATU_DROME | GS995 | 519 |
| Plutella xylostella | LUBEL | LUBEL_DROME | GS996 | 520 |
| [symbol] | XNP | ATRX_{—}DROME | GS997 | 521 |
| [symbol] | Wnt4 | WNT4_{—}DROME | GS998 | 522 |
| [symbol] | Wdr24 | WDR24_{—}DROME | GS999 | 523 |
| [symbol] | HSP70B2 | HSP74_{—}ANOAL | GS1002 | 524 |
| Plutella xylostella | PAN1 | PAN1_PICST | GS1003 | 525 |
| Plutella xylostella | Aplip1 | JIP1_DROME | GS1005 | 526 |
| [symbol] | Taf5 | TAF5_{—}MOUSE | GS1006 | 527 |
| [symbol] | hb | HUNB_{—}MANSE | GS1007 | 528 |
| Plutella xylostella | Fur1 | FUR11_ DROME | GS1008 | 529 |
| [symbol] | Protein Wnt-4 | WNT4_{—}DROME | GS998 | 536 |
| Plutella xylostella | Protein dachsous | DS_DROME | GS1000 | 537 |
| [symbol] | CycE | CCNE_{—}DROME | GS1004 | 538 |
| [symbol] | Slc22a3 | S22A3_{—}RAT | GS1010 | 539 |
| Plutella xylostella | V-type proton ATPase subunit D | VATD_MANSE | GS1011 | 540 |
| Plutella xylostella | Eip74EF | E74EB_DROME | GS1070 | 541 |

TABLE 1C

| | Target | | | Trigger |
|---|---|---|---|---|
| Universal | SEQ ID | | | SEQ ID |
| ID | NO. | Genes Targeted | Target Species | NO. |
| GS1713 | 542 | CHCH2_MOUSE | Plutella xylostella | 823 |
| GS1714 | 543 | LAMA2_HUMAN | Plutella xylostella | 824 |
| GS1715 | 544 | RLR73_PLAVT | Plutella xylostella | 825 |
| GS1716 | 545 | KGP25_DROME | Plutella xylostella | 826 |
| GS1717 | 546 | MDM12_CLAL4 | Plutella xylostella | 827 |
| GS1718 | 547 | DCLK_DROER | Plutella xylostella | 828 |
| GS1719 | 548 | PAR16_HUMAN | Plutella xylostella | 829 |
| GS1720 | 549 | RLMD_LEGPL | Plutella xylostella | 830 |
| GS1721 | 550 | AMGO1_HUMAN | Plutella xylostella | 831 |
| GS1722 | 551 | LRC23_MOUSE | Plutella xylostella | 832 |
| GS1723 | 552 | ANLN_DROME | Plutella xylostella | 833 |
| GS1724 | 553 | FACR1_ARATH | Plutella xylostella | 834 |
| GS1725 | 554 | CDC14_EMENI | Plutella xylostella | 835 |
| GS1726 | 555 | CBPB1_CANLF | Plutella xylostella | 836 |
| GS1727 | 556 | GXCDD_DICDI | Plutella xylostella | 837 |
| GS1729 | 557 | MUC18_MOUSE | Plutella xylostella | 838 |
| GS1730 | 558 | DVR1_STRPU | Plutella xylostella | 839 |
| GS1732 | 559 | BLAB4_ELIME | Plutella xylostella | 840 |
| GS1733 | 560 | TI110_ARATH | Plutella xylostella | 841 |
| GS1734 | 561 | TRM1_ELHVK | Plutella xylostella | 842 |
| GS1736 | 562 | TEKT3_RAT | Plutella xylostella | 843 |
| GS1737 | 563 | TOLB_PELUB | Plutella xylostella | 844 |
| GS1738 | 564 | ATPE_STAA1 | Plutella xylostella | 845 |
| GS1739 | 565 | LUXS_HALH3 | Plutella xylostella | 846 |
| GS1740 | 566 | HSPB1_POELU | Plutella xylostella | 847 |
| GS1741 | 567 | UBP21_SCHPO | Plutella xylostella | 848 |
| GS1742 | 568 | MTCH2_HUMAN | Plutella xylostella | 849 |
| GS1744 | 569 | KIN82_YEAST | Plutella xylostella | 850 |
| GS1745 | 570 | SAHH_ACIAC | Plutella xylostella | 851 |
| GS1746 | 571 | PI16_HUMAN | Plutella xylostella | 852 |
| GS1747 | 572 | GLMM_CELJU | Plutella xylostella | 853 |
| GS1748 | 573 | TBA1_NEUCR | Plutella xylostella | 854 |
| GS1749 | 574 | EIF3A_MAGO7 | Plutella xylostella | 855 |
| GS1750 | 575 | TEN3_DANRE | Plutella xylostella | 856 |
| GS1751 | 576 | FBP1_STRPU | Plutella xylostella | 857 |
| GS1752 | 577 | TBA_COLOR | Plutella xylostella | 858 |
| GS1753 | 578 | NDUB9_MOUSE | Plutella xylostella | 859 |
| GS1755 | 579 | SYT_MYXXD | Plutella xylostella | 860 |
| GS1756 | 580 | POL3_DROME | Plutella xylostella | 861 |
| GS1757 | 581 | CCC1_ORYSJ | Plutella xylostella | 862 |
| GS1758 | 582 | PTR6_ARATH | Plutella xylostella | 863 |
| GS1760 | 583 | DFP_BOMMO | Plutella xylostella | 864 |
| GS1761 | 584 | PXDN_HUMAN | Plutella xylostella | 865 |
| GS1762 | 585 | GYL1_YEAST | Plutella xylostella | 866 |
| GS1765 | 586 | ATPB_BACV8 | Plutella xylostella | 867 |
| GS1766 | 587 | L_MMVR | Plutella xylostella | 868 |
| GS1767 | 588 | SPAN_SHIFL | Plutella xylostella | 869 |
| GS1768 | 589 | SUZ2_DROME | Plutella xylostella | 870 |
| GS1769 | 590 | BROMI_DANRE | Plutella xylostella | 871 |
| GS1770 | 591 | DPOL_ADE12 | Plutella xylostella | 872 |
| GS1771 | 592 | UBE2S_XENTR | Plutella xylostella | 873 |
| GS1772 | 593 | CAD23_HUMAN | Plutella xylostella | 874 |
| GS1773 | 594 | SHO1_ASPOR | Plutella xylostella | 875 |
| GS1774 | 595 | RL15_LACP7 | Plutella xylostella | 876 |
| GS1775 | 596 | HMDH1_YEAST | Plutella xylostella | 877 |
| GS1777 | 597 | FMT_ACTSZ | Plutella xylostella | 878 |
| GS1783 | 598 | PBCB_SCHGR | Plutella xylostella | 879 |
| GS1784 | 599 | FA12_MOUSE | Plutella xylostella | 880 |
| GS1785 | 600 | TRPC2_MOUSE | Plutella xylostella | 881 |
| GS1786 | 601 | RL18_EXISA | Plutella xylostella | 882 |
| GS1787 | 602 | HGFA_MOUSE | Plutella xylostella | 883 |
| GS1788 | 603 | ARHGC_HUMAN | Plutella xylostella | 884 |
| GS1790 | 604 | YCIT_ECOLI | Plutella xylostella | 885 |
| GS1791 | 605 | CU19_LOCMI | Plutella xylostella | 886 |
| GS1793 | 606 | ADDL_OPITP | Plutella xylostella | 887 |
| GS1550 | 607 | TX11A_ETHRU | Plutella xylostella | 888 |
| GS1551 | 608 | SYN_DROME | Plutella xylostella | 889 |
| GS1555 | 609 | KAD2_ANOGA | Plutella xylostella | 890 |
| GS1556 | 610 | 60A_DROVI | Plutella xylostella | 891 |
| GS1557 | 611 | PCAT_DROME | Plutella xylostella | 892 |
| GS1559 | 612 | NACH_DROME | Plutella xylostella | 893 |
| GS1561 | 613 | IPYR_DROME | Plutella xylostella | 894 |
| GS1562 | 614 | TRH_DROME | Plutella xylostella | 895 |
| GS1563 | 615 | IF4A3_DROME | Plutella xylostella | 896 |
| GS1568 | 616 | FUR1C_DROME | Plutella xylostella | 897 |
| GS1569 | 617 | GCSH_DROME | Plutella xylostella | 898 |
| GS1571 | 618 | UBA5_BOMMO | Plutella xylostella | 899 |
| GS1573 | 619 | HSP7E_DROME | Plutella xylostella | 900 |
| GS1574 | 620 | COLT_DROME | Plutella xylostella | 901 |
| GS1575 | 621 | SUH_DROME | Plutella xylostella | 902 |
| GS1579 | 622 | PSD11_DROME | Plutella xylostella | 903 |
| GS1580 | 623 | MO2B1_AEDAE | Plutella xylostella | 904 |
| GS1581 | 624 | NOP14_DROME | Plutella xylostella | 905 |
| GS1583 | 625 | S22A7_MOUSE | Plutella xylostella | 906 |
| GS1585 | 626 | TITIN_DROME | Plutella xylostella | 907 |
| GS1586 | 627 | KLHDB_AEDAE | Plutella xylostella | 908 |
| GS1587 | 628 | CU66_HYACE | Plutella xylostella | 909 |
| GS1588 | 629 | hexamerine | Plutella xylostella | 910 |
| GS1591 | 630 | SY65_DROME | Plutella xylostella | 911 |
| GS1596 | 631 | TUD_DROME | Plutella xylostella | 912 |
| GS1599 | 632 | CUD5_LOCMI | Plutella xylostella | 913 |
| GS1601 | 633 | VP13D_DROME | Plutella xylostella | 914 |
| GS1604 | 634 | RFC2_DROME | Plutella xylostella | 915 |
| GS1606 | 635 | CPSF2_DROME | Plutella xylostella | 916 |
| GS1608 | 636 | METL_DROME | Plutella xylostella | 917 |
| GS1609 | 637 | SYDE_DROME | Plutella xylostella | 918 |
| GS1610 | 638 | C12B2_DROME | Plutella xylostella | 919 |
| GS1612 | 639 | RRF2M_DROVI | Plutella xylostella | 920 |
| GS1615 | 640 | NNRD_AEDAE | Plutella xylostella | 921 |
| GS1616 | 641 | 7LESS_DROVI | Plutella xylostella | 922 |
| GS1621 | 642 | CUO8_BLACR | Plutella xylostella | 923 |
| GS1623 | 643 | POLY_DROME | Plutella xylostella | 924 |
| GS1628 | 644 | PKHF1_DROME | Plutella xylostella | 925 |
| GS1629 | 645 | NPRL2_DROME | Plutella xylostella | 926 |
| GS1758 | 646 | PTR6_ARATH | Plutella xylostella | 927 |
| GS1764 | 647 | CARL2_HUMAN | Plutella xylostella | 928 |
| GS1776 | 648 | ODO2_HAEIN | Plutella xylostella | 929 |
| GS1789 | 649 | R213B_DANRE | Plutella xylostella | 930 |
| GS1941 | 650 | MBF1_YEAST | Plutella xylostella | 931 |
| GS1942 | 651 | SKEL2_DROME | Plutella xylostella | 932 |
| GS1943 | 652 | SKEL2_DROME | Plutella xylostella | 933 |
| GS1944 | 653 | SPA3M_RAT | Plutella xylostella | 934 |
| GS1945 | 654 | PCX1_RAT | Plutella xylostella | 935 |
| GS1946 | 655 | CL14D_ANOGA | Plutella xylostella | 936 |
| GS1947 | 656 | CL14D_ANOGA | Plutella xylostella | 937 |
| GS1951 | 657 | LENG9_MOUSE | Plutella xylostella | 938 |
| GS1952 | 658 | GLSA_SACD2 | Plutella xylostella | 939 |
| GS1953 | 659 | S46A3_CHICK | Plutella xylostella | 940 |
| GS1954 | 660 | SMAP2_CHICK | Plutella xylostella | 941 |
| GS1955 | 661 | POLN_AHEV | Plutella xylostella | 942 |
| GS1956 | 662 | ARH_MOUSE | Plutella xylostella | 943 |
| GS1958 | 663 | CG057_HUMAN | Plutella xylostella | 944 |
| GS1959 | 664 | RELA_HAEIN | Plutella xylostella | 945 |
| GS1960 | 665 | QUEC_NITOC | Plutella xylostella | 946 |
| GS1961 | 666 | CDPKG_ARATH | Plutella xylostella | 947 |
| GS1966 | 667 | RS20_PARPJ | Plutella xylostella | 948 |
| GS1967 | 668 | SCND3_HUMAN | Plutella xylostella | 949 |
| GS1970 | 669 | MILT_DROME | Plutella xylostella | 950 |
| GS1971 | 670 | RGA1_SCHPO | Plutella xylostella | 951 |
| GS1972 | 671 | HSCA_SHEON | Plutella xylostella | 952 |
| GS1973 | 672 | DMD_CHICK | Plutella xylostella | 953 |
| GS1976 | 673 | PLH33_FORAG | Plutella xylostella | 954 |
| GS1977 | 674 | HACD3_DANRE | Plutella xylostella | 955 |
| GS1978 | 675 | CU27_MANSE | Plutella xylostella | 956 |
| GS1979 | 676 | ATM_ASHGO | Plutella xylostella | 957 |
| GS1980 | 677 | DS_DROME | Plutella xylostella | 958 |
| GS1981 | 678 | LITD1_HUMAN | Plutella xylostella | 959 |
| GS1982 | 679 | NRF6_CAEEL | Plutella xylostella | 960 |
| GS1983 | 680 | VPC46_MYCTU | Plutella xylostella | 961 |
| GS1984 | 681 | DNAK_KORVE | Plutella xylostella | 962 |
| GS1985 | 682 | RPOA_ALIF1 | Plutella xylostella | 963 |
| GS1986 | 683 | Y855_MYCLE | Plutella xylostella | 964 |
| GS1990 | 684 | Y855_MYCLE | Plutella xylostella | 965 |
| GS1991 | 685 | SPNA_DICDI | Plutella xylostella | 966 |
| GS1995 | 686 | ATPD_AERS4 | Plutella xylostella | 967 |
| GS1996 | 687 | CFA47_HUMAN | Plutella xylostella | 968 |
| GS1998 | 688 | YEBS_ECO57 | Plutella xylostella | 969 |
| GS2000 | 689 | CHI10_DROME | Plutella xylostella | 970 |
| GS2001 | 690 | PIPE_DROME | Plutella xylostella | 971 |
| GS2002 | 691 | RTXE_DROME | Plutella xylostella | 972 |
| GS2003 | 692 | SRS2L_ARATH | Plutella xylostella | 973 |
| GS2005 | 693 | RTJK_DROME | Plutella xylostella | 974 |
| GS2009 | 694 | APOA_MACMU | Plutella xylostella | 975 |
| GS2010 | 695 | YSH1_CANAL | Plutella xylostella | 976 |
| GS2012 | 696 | SMYD4_PONAB | Plutella xylostella | 977 |
| GS2013 | 697 | ECM5_YEAST | Plutella xylostella | 978 |
| GS2014 | 698 | PRMA_HERA2 | Plutella xylostella | 979 |
| GS2015 | 699 | RS3A_LACBS | Plutella xylostella | 980 |
| GS2016 | 700 | PIPE_DROME | Plutella xylostella | 981 |
| GS2017 | 701 | MURB_STACT | Plutella xylostella | 982 |
| GS2018 | 702 | CU27_MANSE | Plutella xylostella | 983 |
| GS2019 | 703 | LEF11_NPVAH | Plutella xylostella | 984 |
| GS2020 | 704 | CLIP1_CHICK | Plutella xylostella | 985 |
| GS2021 | 705 | YLJ5_CAEEL | Plutella xylostella | 986 |
| GS2022 | 706 | ABCG5_HUMAN | Plutella xylostella | 987 |
| GS2024 | 707 | DUSP3_DICDI | Plutella xylostella | 988 |
| GS2025 | 708 | SAR1_TOBAC | Plutella xylostella | 989 |
| GS2026 | 709 | PRS2_METJA | Plutella xylostella | 990 |
| GS2027 | 710 | SYI_PROMA | Plutella xylostella | 991 |
| GS2029 | 711 | LCA5_HUMAN | Plutella xylostella | 992 |
| GS2030 | 712 | RSMA_CHLP8 | Plutella xylostella | 993 |
| GS2032 | 713 | GRRE1_HUMAN | Plutella xylostella | 994 |
| GS2033 | 714 | PGAP6_HUMAN | Plutella xylostella | 995 |
| GS2034 | 715 | ARGC_METTH | Plutella xylostella | 996 |
| GS2036 | 716 | ITA5_MOUSE | Plutella xylostella | 997 |
| GS2038 | 717 | ERV41_SCHPO | Plutella xylostella | 998 |
| GS2039 | 718 | SYC_MYCMO | Plutella xylostella | 999 |
| GS2040 | 719 | NCAM2_MOUSE | Plutella xylostella | 1000 |
| GS2041 | 720 | RX_DROME | Plutella xylostella | 1001 |
| GS2043 | 721 | HEM1_DELLE | Plutella xylostella | 1002 |
| GS2045 | 722 | MON1_YEAST | Plutella xylostella | 1003 |
| GS2046 | 723 | KPR5_SCHPO | Plutella xylostella | 1004 |
| GS2049 | 724 | YSH1_YEAST | Plutella xylostella | 1005 |
| GS2050 | 725 | TULP4_MOUSE | Plutella xylostella | 1006 |
| GS2051 | 726 | CU03_LONON | Plutella xylostella | 1007 |
| GS2053 | 727 | DRC10_MOUSE | Plutella xylostella | 1008 |
| GS2054 | 728 | TSCOT_CANLF | Plutella xylostella | 1009 |
| GS2055 | 729 | TSCOT_MOUSE | Plutella xylostella | 1010 |
| GS2057 | 730 | PGBM_HUMAN | Plutella xylostella | 1011 |
| GS2058 | 731 | Z354A_HUMAN | Plutella xylostella | 1012 |
| GS2059 | 732 | CU27_MANSE | Plutella xylostella | 1013 |
| GS2060 | 733 | CU03_LONON | Plutella xylostella | 1014 |
| GS2061 | 734 | CLP15_ANOGA | Plutella xylostella | 1015 |
| GS2062 | 735 | AMI_XENLA | Plutella xylostella | 1016 |
| GS2064 | 736 | RTBS_DROME | Plutella xylostella | 1017 |
| GS2065 | 737 | K1C14_RAT | Plutella xylostella | 1018 |
| GS2066 | 738 | SAXO2_HUMAN | Plutella xylostella | 1019 |
| GS2067 | 739 | FUT9_BOVIN | Plutella xylostella | 1020 |
| GS2069 | 740 | NRF6_CAEEL | Plutella xylostella | 1021 |
| GS2071 | 741 | ICCR_DROME | Plutella xylostella | 1022 |
| GS2072 | 742 | INDY1_DROME | Plutella xylostella | 1023 |
| GS2074 | 743 | UREND_METTH | Plutella xylostella | 1024 |
| GS2078 | 744 | Y028_PYRCJ | Plutella xylostella | 1025 |
| GS2083 | 745 | PIPE_DROME | Plutella xylostella | 1026 |
| GS2084 | 746 | NS1BP_XENLA | Plutella xylostella | 1027 |
| GS2085 | 747 | BEST1_HUMAN | Plutella xylostella | 1028 |
| GS2086 | 748 | PABP_SCHPO | Plutella xylostella | 1029 |
| GS2087 | 749 | KCAB2_HUMAN | Plutella xylostella | 1030 |
| GS2088 | 750 | CUD8_SCHGR | Plutella xylostella | 1031 |
| GS2089 | 751 | DPOE_ASPFU | Plutella xylostella | 1032 |
| GS2090 | 752 | ABCA2_HUMAN | Plutella xylostella | 1033 |
| GS2092 | 753 | AMT52_ALTAL | Plutella xylostella | 1034 |
| GS2094 | 754 | NIFN_NOSS1 | Plutella xylostella | 1035 |
| GS2095 | 755 | SPZ4_DROME | Plutella xylostella | 1036 |
| GS2096 | 756 | CU14_MANSE | Plutella xylostella | 1037 |
| GS2097 | 757 | OACYL_MOUSE | Plutella xylostella | 1038 |
| GS2098 | 758 | HCN1_RABIT | Plutella xylostella | 1039 |
| GS2100 | 759 | COPB_ENTHA | Plutella xylostella | 1040 |
| GS2102 | 760 | SYDND_PARUW | Plutella xylostella | 1041 |
| GS2105 | 761 | MTP2_NEIGO | Plutella xylostella | 1042 |
| GS2106 | 762 | 5HT2B_HUMAN | Plutella xylostella | 1043 |
| GS1728 | 763 | XM_011551654 | Plutella xylostella | 1044 |
| GS1731 | 764 | XM_011571056 | Plutella xylostella | 1045 |
| GS1735 | 765 | XM_038118719 | Plutella xylostella | 1046 |
| GS1743 | 766 | MG571541 | Plutella xylostella | 1047 |
| GS1754 | 767 | XM_038117076 | Plutella xylostella | 1048 |
| GS1759 | 768 | XM_011563718 | Plutella xylostella | 1049 |
| GS1763 | 769 | NA | Plutella xylostella | 1050 |
| GS1778 | 770 | XM_038112923 | Plutella xylostella | 1051 |
| GS1780 | 771 | XM_038106157 | Plutella xylostella | 1052 |
| GS1781 | 772 | LN594483 | Plutella xylostella | 1053 |
| GS1782 | 773 | XR_005253948 | Plutella xylostella | 1054 |
| GS1792 | 774 | XM_038108360 | Plutella xylostella | 1055 |
| GS1795 | 775 | XM_011565794 | Plutella xylostella | 1056 |
| GS1948 | 776 | XM_038113609 | Plutella xylostella | 1057 |
| GS1949 | 777 | XM_038105622 | Plutella xylostella | 1058 |
| GS1950 | 778 | NA | Plutella xylostella | 1059 |
| GS1957 | 779 | LN590690 | Plutella xylostella | 1060 |
| GS1962 | 780 | XM_038108184 | Plutella xylostella | 1061 |
| GS1963 | 781 | XM_011565785 | Plutella xylostella | 1062 |
| GS1964 | 782 | NA | Plutella xylostella | 1063 |
| GS1965 | 783 | XM_038110733 | Plutella xylostella | 1064 |
| GS1968 | 784 | HG992030 | Plutella xylostella | 1065 |
| GS1969 | 785 | XM_038119701 | Plutella xylostella | 1066 |
| GS1974 | 786 | XM_038109269 | Plutella xylostella | 1067 |
| GS1975 | 787 | XM_038105771 | Plutella xylostella | 1068 |
| GS1988 | 788 | NA | Plutella xylostella | 1069 |
| GS1989 | 789 | KY965816 | Plutella xylostella | 1070 |
| GS1992 | 790 | XM_038114699 | Plutella xylostella | 1071 |
| GS1994 | 791 | XM_038106632 | Plutella xylostella | 1072 |
| GS1997 | 792 | FR997851 | Plutella xylostella | 1073 |
| GS2004 | 793 | XM_038112617 | Plutella xylostella | 1074 |
| GS2006 | 794 | XM_038114699 | Plutella xylostella | 1075 |
| GS2007 | 795 | LR990146 | Plutella xylostella | 1076 |
| GS2008 | 796 | LN590690 | Plutella xylostella | 1077 |
| GS2023 | 797 | XM_011571033 | Plutella xylostella | 1078 |
| GS2028 | 798 | LR994581 | Plutella xylostella | 1079 |
| GS2031 | 799 | XM_038120499 | Plutella xylostella | 1080 |
| GS2035 | 800 | XM_038121414 | Plutella xylostella | 1081 |
| GS2037 | 801 | XM_038122197 | Plutella xylostella | 1082 |
| GS2042 | 802 | XM_011552302 | Plutella xylostella | 1083 |
| GS2044 | 803 | XM_038111095 | Plutella xylostella | 1084 |
| GS2047 | 804 | XM_011556613 | Plutella xylostella | 1085 |
| GS2048 | 805 | XM_038110436 | Plutella xylostella | 1086 |
| GS2052 | 806 | NA | Plutella xylostella | 1087 |
| GS2056 | 807 | XM_011550987 | Plutella xylostella | 1088 |
| GS2063 | 808 | LN590703 | Plutella xylostella | 1089 |
| GS2068 | 809 | LN590687 | Plutella xylostella | 1090 |
| GS2070 | 810 | NA | Plutella xylostella | 1091 |
| GS2073 | 811 | XM_038114293 | Plutella xylostella | 1092 |
| GS2075 | 812 | XM_011552302 | Plutella xylostella | 1093 |
| GS2076 | 813 | XR_005254795 | Plutella xylostella | 1094 |
| GS2077 | 814 | XM_038116680 | Plutella xylostella | 1095 |
| GS2079 | 815 | XR_005254795 | Plutella xylostella | 1096 |
| GS2080 | 816 | NA | Plutella xylostella | 1097 |
| GS2081 | 817 | LN596718 | Plutella xylostella | 1098 |
| GS2082 | 818 | XM_038107108 | Plutella xylostella | 1099 |
| GS2091 | 819 | XM_038121040 | Plutella xylostella | 1100 |
| GS2093 | 820 | XM_011554829 | Plutella xylostella | 1101 |
| GS2103 | 821 | XM_038120174 | Plutella xylostella | 1102 |
| GS2104 | 822 | XM_038113068 | Plutella xylostella | 1103 |
| GS1240 | 1104 | RU17_DICDI | Plutella xylostella | 1334 |
| GS1241 | 1105 | CSW_DROME | Plutella xylostella | 1335 |
| GS1243 | 1106 | PHM_DROME | Plutella xylostella | 1336 |
| GS1244 | 1107 | BDL_DROME | Plutella xylostella | 1337 |
| GS1245 | 1108 | NFX1_HUMAN | Plutella xylostella | 1338 |
| GS1246 | 1109 | WECH_DROME | Plutella xylostella | 1339 |
| GS1247 | 1110 | ENA_DROME | Plutella xylostella | 1340 |
| GS1248 | 1111 | TCPD_CAEEL | Plutella xylostella | 1341 |
| GS1250 | 1112 | HSP74_DROME | Plutella xylostella | 1342 |
| GS1251 | 1113 | SEM2A_DROME | Plutella xylostella | 1343 |
| GS1252 | 1114 | E75_GALME | Plutella xylostella | 1344 |
| GS1253 | 1115 | DCTN2_ANOGA | Plutella xylostella | 1345 |
| GS1254 | 1116 | VATB_HELVI | Plutella xylostella | 1346 |
| GS1255 | 1117 | RRF2M_DROGR | Plutella xylostella | 1347 |
| GS1258 | 1118 | OSA_DROME | Plutella xylostella | 1348 |
| GS1259 | 1119 | BAB2_DROME | Plutella xylostella | 1349 |
| GS1260 | 1120 | MDR2_CRIGR | Plutella xylostella | 1350 |
| GS1261 | 1121 | PROS_DROME | Plutella xylostella | 1351 |
| GS1262 | 1122 | RAD54_DROMO | Plutella xylostella | 1352 |
| GS1263 | 1123 | SEPT1_DROME | Plutella xylostella | 1353 |
| GS1265 | 1124 | ACSA_DROME | Plutella xylostella | 1354 |
| GS1270 | 1125 | RAD54_DROWI | Plutella xylostella | 1355 |
| GS1271 | 1126 | MCM7_DROME | Plutella xylostella | 1356 |
| GS1272 | 1127 | EF1A_SPOFR | Plutella xylostella | 1357 |
| GS1273 | 1128 | PAL1_DROME | Plutella xylostella | 1358 |
| GS1274 | 1129 | E75_METEN | Plutella xylostella | 1359 |
| GS1275 | 1130 | DJC25_DROME | Plutella xylostella | 1360 |
| GS1277 | 1131 | RPA2_DROME | Plutella xylostella | 1361 |
| GS1278 | 1132 | YAA4_YEAST | Plutella xylostella | 1362 |
| GS1281 | 1133 | EGON_DROME | Plutella xylostella | 1363 |
| GS1282 | 1134 | CPO_DROME | Plutella xylostella | 1364 |
| GS1288 | 1135 | POK_DROME | Plutella xylostella | 1365 |
| GS1290 | 1136 | KI26L_DROME | Plutella xylostella | 1366 |
| GS1291 | 1137 | DRONC_DROME | Plutella xylostella | 1367 |
| GS1293 | 1138 | GIL_DROME | Plutella xylostella | 1368 |
| GS1294 | 1139 | DDC_DROLE | Plutella xylostella | 1369 |
| GS1296 | 1140 | SPAST_DROGR | Plutella xylostella | 1370 |
| GS1297 | 1141 | SUV3_DROME | Plutella xylostella | 1371 |
| GS1298 | 1142 | RDGC_DROME | Plutella xylostella | 1372 |
| GS1300 | 1143 | 7UP1_DROME | Plutella xylostella | 1373 |
| GS1301 | 1144 | DCTN2_AEDAE | Plutella xylostella | 1374 |
| GS1302 | 1145 | HIRA_DROME | Plutella xylostella | 1375 |
| GS1305 | 1146 | RPP30_MOUSE | Plutella xylostella | 1376 |
| GS1306 | 1147 | MES4_DROME | Plutella xylostella | 1377 |
| GS1307 | 1148 | RIM2_DROME | Plutella xylostella | 1378 |
| GS1308 | 1149 | KL98A_DROME | Plutella xylostella | 1379 |
| GS1310 | 1150 | FAS3_DROME | Plutella xylostella | 1380 |
| GS1311 | 1151 | ITPR_DROME | Plutella xylostella | 1381 |
| GS1312 | 1152 | CNC_DROME | Plutella xylostella | 1382 |
| GS1314 | 1153 | LOLA3_DROME | Plutella xylostella | 1383 |
| GS1316 | 1154 | HSP83_BOMMO | Plutella xylostella | 1384 |
| GS1327 | 1155 | ES8L2_HUMAN | Plutella xylostella | 1385 |
| GS1329 | 1156 | BRAT_DROME | Plutella xylostella | 1386 |
| GS1333 | 1157 | UBIA1_DROME | Plutella xylostella | 1387 |
| GS1335 | 1158 | DDX41_DROME | Plutella xylostella | 1388 |
| GS1338 | 1159 | DDX1_DROME | Plutella xylostella | 1389 |
| GS1344 | 1160 | THOC6_DROME | Plutella xylostella | 1390 |
| GS1345 | 1161 | ARL8_DROME | Plutella xylostella | 1391 |
| GS1352 | 1162 | DRE2_AEDAE | Plutella xylostella | 1392 |
| GS1354 | 1163 | HMDH_AGRIP | Plutella xylostella | 1393 |
| GS1356 | 1164 | TSN7_PONPY | Plutella xylostella | 1394 |
| GS1359 | 1165 | TRE12_DROSI | Plutella xylostella | 1395 |
| GS1360 | 1166 | RHEB_DROME | Plutella xylostella | 1396 |
| GS1362 | 1167 | ORB2_DROME | Plutella xylostella | 1397 |
| GS1363 | 1168 | PATJ_DROME | Plutella xylostella | 1398 |
| GS1364 | 1169 | NU154_DROME | Plutella xylostella | 1399 |
| GS1365 | 1170 | TTF2_DROME | Plutella xylostella | 1400 |
| GS1366 | 1171 | INO80_DROME | Plutella xylostella | 1401 |
| GS1367 | 1172 | RHOL_DROME | Plutella xylostella | 1402 |
| GS1368 | 1173 | MCM5_DROME | Plutella xylostella | 1403 |
| GS1369 | 1174 | WDR48_AEDAE | Plutella xylostella | 1404 |
| GS1370 | 1175 | RRF2M_DROWI | Plutella xylostella | 1405 |
| GS1371 | 1176 | VATD1_DROME | Plutella xylostella | 1406 |
| GS1372 | 1177 | EF1A_BOMMO | Plutella xylostella | 1407 |
| GS1373 | 1178 | CHD1_BOMMO | Plutella xylostella | 1408 |
| GS1374 | 1179 | DLISH_DROME | Plutella xylostella | 1409 |
| GS1375 | 1180 | DCA10_DROME | Plutella xylostella | 1410 |
| GS1376 | 1181 | PP2B2_DROME | Plutella xylostella | 1411 |
| GS1377 | 1182 | MMD4_DROME | Plutella xylostella | 1412 |
| GS1378 | 1183 | HR3_GALME | Plutella xylostella | 1413 |
| GS1379 | 1184 | PUM2_HUMAN | Plutella xylostella | 1414 |
| GS1380 | 1185 | CAD99_DROME | Plutella xylostella | 1415 |
| GS1381 | 1186 | ATNA_DROME | Plutella xylostella | 1416 |
| GS1382 | 1187 | PFKA_DROME | Plutella xylostella | 1417 |
| GS1383 | 1188 | ETS4_DROME | Plutella xylostella | 1418 |
| GS1384 | 1189 | TID_DROVI | Plutella xylostella | 1419 |
| GS1385 | 1190 | ORCT_DROME | Plutella xylostella | 1420 |
| GS1386 | 1191 | SAS10_RAT | Plutella xylostella | 1421 |
| GS1387 | 1192 | MY31D_DROME | Plutella xylostella | 1422 |
| GS1388 | 1193 | SIMA_DROME | Plutella xylostella | 1423 |
| GS1389 | 1194 | ARI2_DROME | Plutella xylostella | 1424 |
| GS1390 | 1195 | TCAB1_DROME | Plutella xylostella | 1425 |
| GS1391 | 1196 | BLM_DROME | Plutella xylostella | 1426 |
| GS1392 | 1197 | NDUBA_BOMMO | Plutella xylostella | 1427 |
| GS1393 | 1198 | IF2H_RAT | Plutella xylostella | 1428 |
| GS1394 | 1199 | LIS1_DROGR | Plutella xylostella | 1429 |
| GS1395 | 1200 | YMEL1_DROME | Plutella xylostella | 1430 |
| GS1396 | 1201 | LOLA2_DROME | Plutella xylostella | 1431 |
| GS1397 | 1202 | RBP2_BOVIN | Plutella xylostella | 1432 |
| GS1398 | 1203 | SYEP_DROME | Plutella xylostella | 1433 |
| GS1399 | 1204 | PCNA_BOMMO | Plutella xylostella | 1434 |
| GS1400 | 1205 | RS12_DROME | Plutella xylostella | 1435 |
| GS1401 | 1206 | RDX_DROME | Plutella xylostella | 1436 |
| GS1402 | 1207 | AP3D_DROME | Plutella xylostella | 1437 |
| GS1403 | 1208 | MCM3_DROME | Plutella xylostella | 1438 |
| GS1404 | 1209 | FBXW7_DROME | Plutella xylostella | 1439 |
| GS1409 | 1210 | NAAT1_DROMO | Plutella xylostella | 1440 |
| GS1410 | 1211 | CISY2_AEDAE | Plutella xylostella | 1441 |
| GS1411 | 1212 | U430_DROME | Plutella xylostella | 1442 |
| GS1412 | 1213 | ASAH2_MOUSE | Plutella xylostella | 1443 |
| GS1413 | 1214 | CCNB3_DROME | Plutella xylostella | 1444 |
| GS1414 | 1215 | TAF5_HUMAN | Plutella xylostella | 1445 |
| GS1415 | 1216 | HMCS1_BLAGE | Plutella xylostella | 1446 |
| GS1416 | 1217 | COG6_DROME | Plutella xylostella | 1447 |
| GS1417 | 1218 | UNC80_DROME | Plutella xylostella | 1448 |
| GS1418 | 1219 | TRET1_CULQU | Plutella xylostella | 1449 |
| GS1419 | 1220 | SYAM_DROME | Plutella xylostella | 1450 |
| GS1420 | 1221 | PGP2L_DROME | Plutella xylostella | 1451 |
| GS1421 | 1222 | PGSC2_DROSI | Plutella xylostella | 1452 |
| GS1422 | 1223 | AMY1_DROAN | Plutella xylostella | 1453 |
| GS1423 | 1224 | RAS1_DROYA | Plutella xylostella | 1454 |
| GS1424 | 1225 | MLC1_DROVI | Plutella xylostella | 1455 |
| GS1425 | 1226 | 1433E_DROME | Plutella xylostella | 1456 |
| GS1426 | 1227 | MAD_DROME | Plutella xylostella | 1457 |
| GS1427 | 1228 | GOBP2_EPIPO | Plutella xylostella | 1458 |
| GS1428 | 1229 | DOPR1_DROME | Plutella xylostella | 1459 |
| GS1429 | 1230 | SPR_DROME | Plutella xylostella | 1460 |
| GS1430 | 1231 | GS1_DROME | Plutella xylostella | 1461 |
| GS1431 | 1232 | CU21_LOCMI | Plutella xylostella | 1462 |
| GS1432 | 1233 | ADT_DROME | Plutella xylostella | 1463 |
| GS1433 | 1234 | TTC14_DROME | Plutella xylostella | 1464 |
| GS1434 | 1235 | NOI_DROME | Plutella xylostella | 1465 |
| GS1435 | 1236 | RT06_DROME | Plutella xylostella | 1466 |
| GS1436 | 1237 | NAF1_DROME | Plutella xylostella | 1467 |
| GS1437 | 1238 | ASH1_DROME | Plutella xylostella | 1468 |
| GS1438 | 1239 | EF1A2_DROME | Plutella xylostella | 1469 |
| GS1439 | 1240 | GTR1_DROME | Plutella xylostella | 1470 |
| GS1440 | 1241 | YELL_DROER | Plutella xylostella | 1471 |
| GS1441 | 1242 | EXT2_BOVIN | Plutella xylostella | 1472 |
| GS1442 | 1243 | RL11_SPOFR | Plutella xylostella | 1473 |
| GS1443 | 1244 | KDM5_DROME | Plutella xylostella | 1474 |
| GS1444 | 1245 | ESTE_MYZPE | Plutella xylostella | 1475 |
| GS1445 | 1246 | L2GL_DROPS | Plutella xylostella | 1476 |
| GS1446 | 1247 | DHGL_DROME | Plutella xylostella | 1477 |
| GS1447 | 1248 | MRJP1_APIME | Plutella xylostella | 1478 |
| GS1448 | 1249 | NCD_DROME | Plutella xylostella | 1479 |
| GS1449 | 1250 | ICYA_MANSE | Plutella xylostella | 1480 |
| GS1450 | 1251 | MUL1_DROME | Plutella xylostella | 1481 |
| GS1452 | 1252 | NOG1_DROME | Plutella xylostella | 1482 |
| GS1453 | 1253 | YELL_DROME | Plutella xylostella | 1483 |
| GS1454 | 1254 | RBGPR_DROME | Plutella xylostella | 1484 |
| GS1455 | 1255 | ASPP_AEDAE | Plutella xylostella | 1485 |
| GS1456 | 1256 | HTRA2_DROME | Plutella xylostella | 1486 |
| GS1458 | 1257 | RIR1_DROME | Plutella xylostella | 1487 |
| GS1459 | 1258 | PLXB_DROME | Plutella xylostella | 1488 |
| GS1460 | 1259 | CMC_DROME | Plutella xylostella | 1489 |
| GS1461 | 1260 | PROML_DROME | Plutella xylostella | 1490 |
| GS1462 | 1261 | CANB2_DROME | Plutella xylostella | 1491 |
| GS1463 | 1262 | CASP8_DROPS | Plutella xylostella | 1492 |
| GS1464 | 1263 | MED14_AEDAE | Plutella xylostella | 1493 |
| GS1465 | 1264 | GR43A_DROME | Plutella xylostella | 1494 |
| GS1466 | 1265 | POMT2_DROPS | Plutella xylostella | 1495 |
| GS1467 | 1266 | NPY6R_MOUSE | Plutella xylostella | 1496 |
| GS1468 | 1267 | FICD_CULQU | Plutella xylostella | 1497 |
| GS1469 | 1268 | 1433Z_BOMMO | Plutella xylostella | 1498 |
| GS1470 | 1269 | GPR9_AMPAM | Plutella xylostella | 1499 |
| GS1471 | 1270 | PA2_BOMPE | Plutella xylostella | 1500 |
| GS1472 | 1271 | BRC4_DROME | Plutella xylostella | 1501 |
| GS1473 | 1272 | IF4E_DROME | Plutella xylostella | 1502 |
| GS1474 | 1273 | PAL2_DROME | Plutella xylostella | 1503 |
| GS1475 | 1274 | S39AD_DROME | Plutella xylostella | 1504 |
| GS1476 | 1275 | MADD_DROME | Plutella xylostella | 1505 |
| GS1477 | 1276 | RABL3_DANRE | Plutella xylostella | 1506 |
| GS1478 | 1277 | BRM_DROME | Plutella xylostella | 1507 |
| GS1479 | 1278 | DIA_DROME | Plutella xylostella | 1508 |
| GS1480 | 1279 | 41_DROME | Plutella xylostella | 1509 |
| GS1481 | 1280 | CUD3_SCHGR | Plutella xylostella | 1510 |
| GS1482 | 1281 | CDC42_AEDAE | Plutella xylostella | 1511 |
| GS1483 | 1282 | SPZ_DROME | Plutella xylostella | 1512 |
| GS1484 | 1283 | FYV1_DROME | Plutella xylostella | 1513 |
| GS1485 | 1284 | ELG_DROME | Plutella xylostella | 1514 |
| GS1486 | 1285 | IMPL2_DROME | Plutella xylostella | 1515 |
| GS1488 | 1286 | WDR1_DROME | Plutella xylostella | 1516 |
| GS1489 | 1287 | PRS6B_MANSE | Plutella xylostella | 1517 |
| GS1490 | 1288 | TTKA_DROME | Plutella xylostella | 1518 |
| GS1491 | 1289 | IF2G_DROME | Plutella xylostella | 1519 |
| GS1547 | 1290 | DHE2_ACHKL | Plutella xylostella | 1520 |
| GS1548 | 1291 | SPS1_DROME | Plutella xylostella | 1521 |
| GS1549 | 1292 | PP2A_DROME | Plutella xylostella | 1522 |
| GS1552 | 1293 | MTTF_DROME | Plutella xylostella | 1523 |
| GS1553 | 1294 | CAZ_DROME | Plutella xylostella | 1524 |
| GS1554 | 1295 | HDAC1_DROME | Plutella xylostella | 1525 |
| GS1558 | 1296 | TERA_DROME | Plutella xylostella | 1526 |
| GS1560 | 1297 | EXT2_DROME | Plutella xylostella | 1527 |
| GS1564 | 1298 | PRIC1_ANOGA | Plutella xylostella | 1528 |
| GS1565 | 1299 | DGK2_DROME | Plutella xylostella | 1529 |
| GS1566 | 1300 | TCPD_OCHTR | Plutella xylostella | 1530 |
| GS1567 | 1301 | RL18_SPOFR | Plutella xylostella | 1531 |
| GS1570 | 1302 | PYRG_DROME | Plutella xylostella | 1532 |
| GS1572 | 1303 | WHITE_DROME | Plutella xylostella | 1533 |
| GS1576 | 1304 | MFRN1_HUMAN | Plutella xylostella | 1534 |
| GS1577 | 1305 | PRS8_MANSE | Plutella xylostella | 1535 |
| GS1578 | 1306 | DDX49_DROME | Plutella xylostella | 1536 |
| GS1582 | 1307 | ADA17_DROME | Plutella xylostella | 1537 |
| GS1584 | 1308 | ESN_DROPS | Plutella xylostella | 1538 |
| GS1589 | 1309 | GLYR1_AEDAE | Plutella xylostella | 1539 |
| GS1590 | 1310 | CP301_DROME | Plutella xylostella | 1540 |
| GS1592 | 1311 | NARF_AEDAE | Plutella xylostella | 1541 |
| GS1593 | 1312 | COQ4_BOMMO | Plutella xylostella | 1542 |
| GS1594 | 1313 | CP4S3_DROME | Plutella xylostella | 1543 |
| GS1595 | 1314 | WLS_DROVI | Plutella xylostella | 1544 |
| GS1597 | 1315 | SWS_DROME | Plutella xylostella | 1545 |
| GS1598 | 1316 | EVI5_DROME | Plutella xylostella | 1546 |
| GS1600 | 1317 | H1_DROME | Plutella xylostella | 1547 |
| GS1602 | 1318 | EXO1_DROME | Plutella xylostella | 1548 |
| GS1603 | 1319 | Y2678_METMA | Plutella xylostella | 1549 |
| GS1605 | 1320 | PH4H_DROME | Plutella xylostella | 1550 |
| GS1607 | 1321 | CARME_DROME | Plutella xylostella | 1551 |
| GS1611 | 1322 | C12C1_DROME | Plutella xylostella | 1552 |
| GS1613 | 1323 | NOSL_BOMMO | Plutella xylostella | 1553 |
| GS1614 | 1324 | BXA3_SAMCY | Plutella xylostella | 1554 |
| GS1617 | 1325 | GATA_CULQU | Plutella xylostella | 1555 |
| GS1618 | 1326 | OPS2_MANSE | Plutella xylostella | 1556 |
| GS1619 | 1327 | RL24_PLUXY | Plutella xylostella | 1557 |
| GS1620 | 1328 | DCLK_DROSI | Plutella xylostella | 1558 |
| GS1622 | 1329 | CUBN_DROME | Plutella xylostella | 1559 |
| GS1624 | 1330 | PTTH_BOMMO | Plutella xylostella | 1560 |
| GS1625 | 1331 | MED15_AEDAE | Plutella xylostella | 1561 |
| GS1626 | 1332 | ERCC3_DROME | Plutella xylostella | 1562 |
| GS1627 | 1333 | ECE_LOCMI | Plutella xylostella | 1563 |
| GS1249 | 1564 | P36188 | Plutella xylostella | 1624 |
| GS1256 | 1565 | A1L237 | Plutella xylostella | 1625 |
| GS1728 | 1566 | XM_011551654 | Plutella xylostella | 1626 |
| GS1731 | 1567 | XM_038110302 | Plutella xylostella | 1627 |
| GS1735 | 1568 | XM_038118719 | Plutella xylostella | 1628 |
| GS1743 | 1569 | MG571541 | Plutella xylostella | 1629 |
| GS1754 | 1570 | XM_038117076 | Plutella xylostella | 1630 |
| GS1759 | 1571 | XM_011563718 | Plutella xylostella | 1631 |
| GS1763 | 1572 | NA | Plutella xylostella | 1632 |
| GS1778 | 1573 | XM_038112923 | Plutella xylostella | 1633 |
| GS1780 | 1574 | XM_038106157 | Plutella xylostella | 1634 |
| GS1781 | 1575 | XM_038111383 | Plutella xylostella | 1635 |
| GS1782 | 1576 | XR_005253948 | Plutella xylostella | 1636 |
| GS1792 | 1577 | XM_038108360 | Plutella xylostella | 1637 |
| GS1795 | 1578 | XM_011565794 | Plutella xylostella | 1638 |
| GS1948 | 1579 | XM_038113609 | Plutella xylostella | 1639 |
| GS1949 | 1580 | XM_038105623 | Plutella xylostella | 1640 |
| GS1950 | 1581 | NA | Plutella xylostella | 1641 |
| GS1957 | 1582 | LN590690 | Plutella xylostella | 1642 |
| GS1962 | 1583 | XM_038108184 | Plutella xylostella | 1643 |
| GS1963 | 1584 | XM_011565785 | Plutella xylostella | 1644 |
| GS1964 | 1585 | NA | Plutella xylostella | 1645 |
| GS1965 | 1586 | XM_038110733 | Plutella xylostella | 1646 |
| GS1968 | 1587 | HG992030 | Plutella xylostella | 1647 |
| GS1969 | 1588 | XM_038119701 | Plutella xylostella | 1648 |
| GS1974 | 1589 | XM_038109269 | Plutella xylostella | 1649 |
| GS1975 | 1590 | XM_038105771 | Plutella xylostella | 1650 |
| GS1988 | 1591 | NA | Plutella xylostella | 1651 |
| GS1989 | 1592 | KY965816 | Plutella xylostella | 1652 |
| GS1992 | 1593 | XM_038114699 | Plutella xylostella | 1653 |
| GS1994 | 1594 | XM_038106632 | Plutella xylostella | 1654 |
| GS1997 | 1595 | MG571541 | Plutella xylostella | 1655 |
| GS2006 | 1596 | XM_038114699 | Plutella xylostella | 1656 |
| GS2008 | 1597 | LN590690 | Plutella xylostella | 1657 |
| GS2023 | 1598 | XM_011571033 | Plutella xylostella | 1658 |
| GS2028 | 1599 | LR994581 | Plutella xylostella | 1659 |
| GS2031 | 1600 | XM_038120499 | Plutella xylostella | 1660 |
| GS2035 | 1601 | XM_038121414 | Plutella xylostella | 1661 |
| GS2037 | 1602 | MG571541 | Plutella xylostella | 1662 |
| GS2042 | 1603 | XM_011552302 | Plutella xylostella | 1663 |
| GS2044 | 1604 | XM_038111095 | Plutella xylostella | 1664 |
| GS2047 | 1605 | XM_011556613 | Plutella xylostella | 1665 |
| GS2048 | 1606 | XM_038121712 | Plutella xylostella | 1666 |
| GS2052 | 1607 | CP020383 | Plutella xylostella | 1667 |
| GS2056 | 1608 | XM_011550987 | Plutella xylostella | 1668 |
| GS2063 | 1609 | LN594646 | Plutella xylostella | 1669 |
| GS2068 | 1610 | LN596087 | Plutella xylostella | 1670 |
| GS2070 | 1611 | NA | Plutella xylostella | 1671 |
| GS2073 | 1612 | XM_038114293 | Plutella xylostella | 1672 |
| GS2075 | 1613 | XM_011552302 | Plutella xylostella | 1673 |
| GS2076 | 1614 | XR_005254795 | Plutella xylostella | 1674 |
| GS2077 | 1615 | XM_038116680 | Plutella xylostella | 1675 |
| GS2079 | 1616 | XR_005254795 | Plutella xylostella | 1676 |
| GS2080 | 1617 | NA | Plutella xylostella | 1677 |
| GS2081 | 1618 | XM_038107769 | Plutella xylostella | 1678 |
| GS2082 | 1619 | XM_038107108 | Plutella xylostella | 1679 |
| GS2091 | 1620 | XM_038121040 | Plutella xylostella | 1680 |
| GS2093 | 1621 | XM_011554829 | Plutella xylostella | 1681 |
| GS2103 | 1622 | XM_038120174 | Plutella xylostella | 1682 |
| GS2104 | 1623 | XM_038113068 | Plutella xylostella | 1683 |

### Example 1: Identification of RNAi Lethal Genes in P. xylostella

### Primary Screening Bioassays.

dsRNA molecules were tested for insecticidal activity against P. xylostella in artificial diet-based bioassays. Briefly, artificial diet containing unformulated (e.g. naked dsRNA) or formulated (see for example section VI. supra) dsRNA were placed in a 47-mm Petri dish. Five to seven late L1 instar DBM larvae were added per Petri dish using a fine pencil brush. Each treatment contained 5 replicates. Surviving insects 2 days after assay initiation were considered the total number of insects in the test and this number was used for calculating mortality. Freshly treated diet was added to each Petri dish on day 7, and thereafter untreated diet was added as needed. The larvae were observed for mortality at day 9. Observations were continued for up to 14 days, until larvae developed to pupae. Mortality was corrected though the Henderson-Tilton formula as reported in Tables 2 and 3. Bioassays were scored as follows:

% mortality

| 0 | 1-14 | 15-30 | >30 |
|---|---|---|---|
| − | + | ++ | +++ |

The scoring data is provided in Tables 2 and 3 below. Each of the dsRNA molecules in Tables 2 and 3 are identified by their GS number which correlate to the Trigger IDs and SEQ ID NOs provided in Table 1A. The underlined/italicized SEQ ID NOs represent those sequences having a score of ++ or +++ per the data shown in Tables 2 and 3.

TABLE 2

| | Corrected |
|---|---|
| | Mortality |
| ID | D 9 |
| GS144 | + |
| GS146 | ++ |
| GS148 | + |
| GS150 | + |
| GS152 | ++ |
| GS268 | + |
| GS269 | +++ |
| GS270 | + |
| GS271 | + |
| GS272 | + |
| GS273 | + |
| GS318 | ++ |
| GS319 | ++ |
| GS320 | + |
| GS321 | +++ |
| GS322 | + |
| GS323 | + |
| GS324 | + |
| GS325 | + |
| GS326 | ++ |
| GS327 | − |
| GS328 | + |
| GS329 | ++ |
| GS330 | + |
| GS331 | + |
| GS332 | + |
| GS333 | + |
| GS334 | + |
| GS335 | + |
| GS336 | + |
| GS337 | + |
| GS338 | + |

TABLE 3

| | Corrected |
|---|---|
| | Mortality |
| ID | D9 |
| GS453 | − |
| GS454 | − |
| GS455 | − |
| GS456 | − |
| GS457 | − |
| GS458 | + |
| GS459 | − |
| GS460 | − |
| GS461 | − |
| GS462 | − |
| GS463 | + |
| GS464 | − |
| GS465 | + |
| GS468 | + |
| GS469 | − |
| GS471 | − |
| GS485 | − |
| GS486 | +++ |
| GS487 | − |
| GS488 | − |
| GS489 | − |
| GS490 | − |
| GS491 | − |
| GS492 | − |
| GS493 | − |
| GS494 | − |
| GS495 | − |
| GS496 | − |
| GS497 | − |

### Example 2: Identification of RNAi Lethal Genes in S. frugiperda

### Primary Screening Bioassays.

dsRNA molecules were tested for insecticidal activity against S. frugiperda in artificial diet-based bioassays. Briefly, artificial diet containing unformulated or formulated dsRNA were placed in in 24-well plates. One fall armyworm neonate was placed per well using a fine pencil brush. Plates were covered with a commercially available translucent seal. Each treatment contained 16-24 replicates which were randomized in multiple 24-well plates. Insects were re-treated with fresh diet incorporated with dsRNA on day 5. The insects are observed for mortality and stunting (development arrested at L3) for up to 12 days. dsRNA designed to target green fluorescent protein and water were used as negative controls. Bioassays were scored as follows:

% mortality

| 0 | 1-14 | 15-30 | >30 |
|---|---|---|---|
| − | + | ++ | +++ |

The scoring data is provided in Tables 4 and 5 below. Each of the dsRNA molecules in Tables 4 and 5 are identified by their GS number which correlate to the SEQ ID NO provided in Table 1A. The bolded SEQ ID NOs in Table 1A represent those sequences having a score of ++ or +++.

TABLE 4

| ID | Mortality |
|---|---|
| GS262 | − |
| GS263 | +++ |
| GS264 | − |
| GS265 | ++ |
| GS266 | − |
| GS267 | − |
| GS297 | − |
| GS298 | − |
| GS299 | − |
| GS300 | ++ |
| GS301 | − |
| GS302 | − |
| GS303 | − |
| GS304 | − |
| GS305 | +++ |
| GS306 | ++ |
| GS307 | − |
| GS309 | +++ |
| GS311 | − |
| GS313 | − |
| GS314 | − |
| GS315 | − |
| GS316 | − |
| GS317 | − |

TABLE 5

| ID | Mortality |
|---|---|
| GS440 | − |
| GS441 | − |
| GS442 | − |
| GS443 | − |
| GS444 | − |
| GS445 | − |
| GS446 | − |
| GS447 | − |
| GS448 | − |
| GS449 | − |
| GS450 | − |
| GS451 | − |
| GS452 | − |
| GS466 | − |
| GS467 | − |
| GS470 | +++ |
| GS472 | − |
| GS473 | − |
| GS474 | − |
| GS475 | − |
| GS476 | − |
| GS477 | − |
| GS478 | +++ |
| GS479 | − |
| GS480 | − |
| GS481 | − |
| GS482 | +++ |
| GS483 | − |
| GS484 | − |

### Example 3: Identification of RNAi Lethal Genes in P. xylostella

dsRNA molecules were tested for insecticidal activity against P. xylostella in artificial diet-based bioassays. Briefly, artificial diet plugs were incorporated with 50 uL of naked dsRNA at 1 mg/mL. The plugs were placed each in a 47-mm Petri dish. 5 late L1 stage larvae were placed per Petri dish. Fresh untreated diet was added on day 7. Alive insects after 3 days of dsRNA exposure are the total number of insects used for mortality calculations. The insects were monitored for mortality and development up to 13 days.

The scoring data is provided in Tables 6 and 7 below. Mortality was adjusted by the Henderson Tilton's formula and corrected mortality is below. Each of the dsRNA molecules in Tables 6 and 7 are identified by their GS number which correlate to the SEQ ID NO provided in Table 1B. The bolded SEQ ID NOs represent those sequences having a score of ++ or +++.

% mortality

| 0 | 1-14 | 15-30 | >30 |
|---|---|---|---|
| − | + | ++ | +++ |

TABLE 6

| | Corrected |
|---|---|
| ID | Mortality D13 |
| GS889 | + |
| GS891 | + |
| GS895 | + |
| GS897 | + |
| GS898 | ++ |
| GS899 | + |
| GS901 | ++ |
| GS904 | − |
| GS908 | + |
| GS912 | − |
| GS914 | + |
| GS928 | + |
| GS937 | − |
| GS941 | − |
| GS947 | + |
| GS951 | + |
| GS953 | + |
| GS954 | + |
| GS956 | + |
| GS957 | ++ |
| GS958 | ++ |
| GS959 | +++ |
| GS960 | + |
| GS961 | + |
| GS962 | + |
| GS963 | + |
| GS964 | ++ |
| GS984 | ++ |
| GS1000 | + |
| GS1004 | ++ |
| GS1009 | ++ |
| GS1010 | ++ |
| GS1011 | − |
| GS1070 | + |

TABLE 7

| | Corrected |
|---|---|
| ID | Mortality |
| GS907 | − |
| GS909 | +++ |
| GS910 | − |
| GS911 | + |
| GS913 | + |
| GS915 | − |
| GS916 | − |
| GS927 | − |
| GS929 | − |
| GS930 | − |
| GS931 | − |
| GS938 | + |
| GS939 | + |
| GS940 | − |
| GS989 | ++ |
| GS990 | + |
| GS991 | ++ |
| GS992 | ++ |
| GS993 | ++ |
| GS994 | ++ |
| GS995 | ++ |
| GS996 | +++ |
| GS997 | ++ |
| GS998 | +++ |
| GS999 | ++ |
| GS1001 | +++ |
| GS1002 | ++ |
| GS1003 | + |
| GS1005 | + |
| GS1006 | +++ |
| GS1007 | +++ |
| GS1008 | + |
| GS886 | − |
| GS887 | +++ |
| GS888 | − |
| GS890 | − |
| GS892 | +++ |
| GS893 | − |
| GS894 | − |
| GS896 | +++ |
| GS900 | ++ |
| GS902 | − |
| GS903 | − |
| GS905 | ++ |
| GS906 | + |
| GS948 | − |
| GS949 | − |
| GS950 | − |
| GS952 | − |
| GS955 | − |
| GS983 | − |

### Example 4: Identification of RNAi Lethal Genes in P. xylostella

dsRNA molecules were tested for insecticidal activity against P. xylostella in artificial diet-based bioassays. Briefly, artificial diet plugs were incorporated with 50 uL of naked dsRNA at 1 mg/mL. The plugs were placed each in a 47-mm Petri dish. 5 late L1 larvae were placed per Petri dish. Fresh untreated diet was added on day 7. Alive insects after 3 days of dsRNA exposure were the total number of insects used for mortality calculations. The insects were monitored for mortality and development up to 14 days. The scoring data is provided in Table 9 below. Mortality was adjusted by the Henderson Tilton's formula and corrected mortality is below. Each of the dsRNA molecules in Table 9 are identified by their GS number which correlate to the SEQ ID NO provided in Table 1C. The bolded SEQ ID NOs in Table 1C represent those sequences having a score of ++ or +++, and each of such sequences may be used individually in the claimed methods or compositions.

% mortality

| 0 | 1-14 | 15-30 | >30 |
|---|---|---|---|
| − | + | ++ | +++ |

TABLE 8

| | | Corrected |
|---|---|---|
| | Mortality | Mortality |
| ID | D14 | D14 |
| GS1240 | +++ | ++ |
| GS1241 | ++ | ++ |
| GS1243 | +++ | ++ |
| GS1244 | ++ | + |
| GS1245 | ++ | − |
| GS1246 | ++ | + |
| GS1247 | ++ | − |
| GS1248 | +++ | ++ |
| GS1249 | +++ | ++ |
| GS1250 | ++ | + |
| GS1251 | +++ | ++ |
| GS1252 | ++ | + |
| GS1253 | ++ | + |
| GS1254 | +++ | ++ |
| GS1255 | ++ | + |
| GS1256 | +++ | ++ |
| GS1258 | ++ | + |
| GS1259 | ++ | + |
| GS1260 | ++ | + |
| GS1261 | +++ | ++ |
| GS1262 | +++ | + |
| GS1263 | +++ | ++ |
| GS1265 | +++ | +++ |
| GS1270 | ++ | + |
| GS1271 | + | + |
| GS1272 | ++ | + |
| GS1273 | ++ | + |
| GS1274 | ++ | − |
| GS1275 | + | − |
| GS1277 | ++ | + |
| GS1278 | +++ | ++ |
| GS1281 | +++ | ++ |
| GS1282 | ++ | + |
| GS1288 | ++ | + |
| GS1290 | ++ | − |
| GS1291 | ++ | + |
| GS1293 | ++ | + |
| GS1294 | ++ | + |
| GS1296 | ++ | − |
| GS1297 | ++ | − |
| GS1298 | ++ | + |
| GS1300 | ++ | − |
| GS1301 | ++ | − |
| GS1302 | + | − |
| GS1305 | + | − |
| GS1306 | +++ | ++ |
| GS1307 | + | − |
| GS1308 | ++ | + |
| GS1310 | + | − |
| GS1311 | + | − |
| GS1312 | ++ | + |
| GS1314 | ++ | + |
| GS1316 | + | − |
| GS1327 | − | − |
| GS1329 | ++ | ++ |
| GS1333 | ++ | ++ |
| GS1335 | + | + |
| GS1338 | + | − |
| GS1344 | + | + |
| GS1345 | + | + |
| GS1352 | + | − |
| GS1354 | +++ | ++ |
| GS1356 | + | + |
| GS1359 | ++ | + |
| GS1360 | ++ | ++ |
| GS1362 | + | + |
| GS1363 | − | − |
| GS1364 | ++ | + |
| GS1365 | ++ | + |
| GS1366 | + | − |
| GS1367 | + | − |
| GS1368 | ++ | + |
| GS1369 | ++ | + |
| GS1370 | + | − |
| GS1371 | ++ | + |
| GS1372 | ++ | + |
| GS1373 | ++ | + |
| GS1374 | + | − |
| GS1375 | ++ | + |
| GS1376 | ++ | + |
| GS1377 | ++ | + |
| GS1378 | + | − |
| GS1379 | ++ | + |
| GS1380 | ++ | + |
| GS1381 | ++ | + |
| GS1382 | ++ | ++ |
| GS1383 | ++ | + |
| GS1384 | + | − |
| GS1385 | +++ | ++ |
| GS1386 | + | − |
| GS1387 | ++ | ++ |
| GS1388 | ++ | + |
| GS1389 | + | − |
| GS1390 | +++ | ++ |
| GS1391 | +++ | ++ |
| GS1392 | ++ | + |
| GS1393 | + | − |
| GS1394 | ++ | + |
| GS1395 | + | − |
| GS1396 | ++ | + |
| GS1397 | + | − |
| GS1398 | + | − |
| GS1399 | + | − |
| GS1400 | + | − |
| GS1401 | ++ | + |
| GS1402 | + | − |
| GS1403 | ++ | + |
| GS1404 | ++ | + |
| GS1419 | − | − |
| GS1425 | + | − |
| GS1431 | + | − |
| GS1437 | + | − |
| GS1443 | − | − |
| GS1449 | − | − |
| GS1455 | − | − |
| GS1456 | + | + |
| GS1461 | + | + |
| GS1462 | + | + |
| GS1467 | + | + |
| GS1468 | + | − |
| GS1473 | + | + |
| GS1474 | + | + |
| GS1475 | + | − |
| GS1479 | + | − |
| GS1480 | ++ | ++ |
| GS1485 | + | + |
| GS1486 | + | + |
| GS1491 | + | + |
| GS1409 | + | + |
| GS1410 | + | + |
| GS1411 | + | + |
| GS1412 | − | − |
| GS1413 | + | + |
| GS1414 | − | − |
| GS1415 | ++ | ++ |
| GS1416 | ++ | ++ |
| GS1417 | + | + |
| GS1418 | + | + |
| GS1420 | + | + |
| GS1421 | + | + |
| GS1422 | + | + |
| GS1423 | + | + |
| GS1424 | + | + |
| GS1426 | + | + |
| GS1427 | + | + |
| GS1428 | + | + |
| GS1429 | + | + |
| GS1430 | + | + |
| GS1432 | + | + |
| GS1433 | + | + |
| GS1434 | + | + |
| GS1435 | + | + |
| GS1436 | + | + |
| GS1438 | + | + |
| GS1439 | ++ | ++ |
| GS1440 | + | + |
| GS1441 | + | + |
| GS1442 | ++ | ++ |
| GS1444 | + | + |
| GS1445 | + | + |
| GS1446 | − | − |
| GS1447 | + | + |
| GS1448 | + | + |
| GS1450 | ++ | ++ |
| GS1452 | + | + |
| GS1453 | − | − |
| GS1454 | + | + |
| GS1458 | + | + |
| GS1459 | + | + |
| GS1460 | + | + |
| GS1463 | + | + |
| GS1464 | +++ | +++ |
| GS1465 | ++ | ++ |
| GS1466 | ++ | ++ |
| GS1469 | + | + |
| GS1470 | + | + |
| GS1471 | +++ | +++ |
| GS1472 | + | + |
| GS1476 | ++ | ++ |
| GS1477 | + | + |
| GS1478 | − | − |
| GS1481 | + | + |
| GS1482 | + | + |
| GS1483 | + | + |
| GS1484 | ++ | ++ |
| GS1488 | + | + |
| GS1489 | + | + |
| GS1490 | − | − |
| GS1547 | ++ | ++ |
| GS1548 | + | + |
| GS1549 | + | + |
| GS1552 | − | − |
| GS1553 | + | + |
| GS1554 | + | + |
| GS1558 | − | − |
| GS1560 | + | + |
| GS1564 | + | + |
| GS1565 | + | + |
| GS1566 | + | + |
| GS1567 | + | + |
| GS1570 | − | − |
| GS1572 | − | − |
| GS1576 | + | + |
| GS1577 | + | + |
| GS1578 | + | + |
| GS1582 | + | + |
| GS1584 | ++ | ++ |
| GS1589 | + | + |
| GS1590 | ++ | ++ |
| GS1592 | + | + |
| GS1593 | + | + |
| GS1594 | + | + |
| GS1595 | − | − |
| GS1597 | + | + |
| GS1598 | ++ | ++ |
| GS1600 | + | + |
| GS1602 | ++ | ++ |
| GS1603 | − | − |
| GS1605 | ++ | ++ |
| GS1607 | + | + |
| GS1611 | + | + |
| GS1613 | + | + |
| GS1614 | + | + |
| GS1617 | + | + |
| GS1618 | + | + |
| GS1619 | + | + |
| GS1620 | + | + |
| GS1622 | + | + |
| GS1624 | + | + |
| GS1625 | + | + |
| GS1626 | + | + |
| GS1627 | + | + |
| GS1713 | +++ | + |
| GS1714 | ++ | + |
| GS1715 | + | − |
| GS1716 | +++ | +++ |
| GS1717 | +++ | +++ |
| GS1718 | +++ | ++ |
| GS1719 | ++ | − |
| GS1720 | ++ | ++ |
| GS1721 | ++ | + |
| GS1722 | +++ | ++ |
| GS1723 | +++ | ++ |
| GS1724 | +++ | +++ |
| GS1725 | ++ | + |
| GS1726 | ++ | + |
| GS1727 | ++ | + |
| GS1728 | +++ | ++ |
| GS1729 | ++ | + |
| GS1730 | ++ | − |
| GS1731 | ++ | − |
| GS1732 | +++ | +++ |
| GS1733 | +++ | +++ |
| GS1734 | +++ | ++ |
| GS1735 | ++ | + |
| GS1736 | + | − |
| GS1737 | +++ | ++ |
| GS1738 | +++ | +++ |
| GS1739 | ++ | + |
| GS1740 | + | − |
| GS1741 | ++ | − |
| GS1742 | ++ | + |
| GS1743 | +++ | +++ |
| GS1744 | ++ | + |
| GS1745 | +++ | ++ |
| GS1746 | ++ | + |
| GS1747 | +++ | ++ |
| GS1748 | +++ | +++ |
| GS1749 | ++ | + |
| GS1750 | + | − |
| GS1751 | + | − |
| GS1752 | + | − |
| GS1753 | ++ | + |
| GS1754 | ++ | + |
| GS1755 | ++ | + |
| GS1756 | + | − |
| GS1757 | ++ | ++ |
| GS1758 | + | − |
| GS1759 | + | − |
| GS1760 | ++ | + |
| GS1761 | + | − |
| GS1762 | ++ | + |
| GS1763 | + | + |
| GS1765 | + | − |
| GS1766 | + | + |
| GS1767 | ++ | + |
| GS1768 | ++ | + |
| GS1769 | ++ | + |
| GS1770 | ++ | + |
| GS1771 | ++ | + |
| GS1772 | +++ | +++ |
| GS1773 | ++ | + |
| GS1774 | ++ | + |
| GS1775 | ++ | ++ |
| GS1777 | + | + |
| GS1778 | ++ | + |
| GS1780 | ++ | + |
| GS1781 | ++ | + |
| GS1782 | ++ | + |
| GS1783 | + | − |
| GS1784 | + | + |
| GS1785 | + | − |
| GS1786 | + | − |
| GS1787 | ++ | ++ |
| GS1788 | + | − |
| GS1790 | + | − |
| GS1791 | ++ | + |
| GS1791 | + | − |
| GS1792 | ++ | + |
| GS1793 | + | − |
| GS1550 | ++ | + |
| GS1551 | + | − |
| GS1555 | + | − |
| GS1556 | ++ | + |
| GS1557 | ++ | ++ |
| GS1559 | ++ | ++ |
| GS1561 | + | − |
| GS1562 | ++ | + |
| GS1563 | ++ | + |
| GS1568 | + | + |
| GS1569 | ++ | + |
| GS1571 | + | + |
| GS1573 | ++ | + |
| GS1574 | ++ | ++ |
| GS1575 | ++ | + |
| GS1579 | ++ | + |
| GS1580 | + | − |
| GS1581 | ++ | + |
| GS1583 | − | − |
| GS1585 | + | − |
| GS1586 | + | − |
| GS1587 | + | − |
| GS1588 | + | − |
| GS1591 | + | − |
| GS1596 | − | − |
| GS1599 | + | − |
| GS1601 | + | − |
| GS1604 | + | + |
| GS1606 | + | − |
| GS1608 | − | − |
| GS1609 | − | − |
| GS1610 | + | − |
| GS1612 | + | − |
| GS1615 | + | − |
| GS1616 | + | − |
| GS1621 | + | − |
| GS1623 | + | − |
| GS1628 | − | − |
| GS1629 | + | − |
| GS1758 | + | − |
| GS1764 | + | − |
| GS1776 | ++ | + |
| GS1789 | − | − |
| GS1795 | + | − |
| GS1941 | ++ | − |
| GS1942 | + | − |
| GS1943 | + | + |
| GS1944 | ++ | − |
| GS1945 | ++ | − |
| GS1946 | ++ | + |
| GS1947 | + | + |
| GS1948 | ++ | + |
| GS1949 | ++ | + |
| GS1950 | + | + |
| GS1951 | ++ | + |
| GS1952 | + | + |
| GS1953 | ++ | + |
| GS1954 | ++ | − |
| GS1955 | ++ | + |
| GS1956 | ++ | + |
| GS1957 | + | + |
| GS1958 | ++ | − |
| GS1959 | − | + |
| GS1960 | + | ++ |
| GS1961 | + | ++ |
| GS1962 | + | + |
| GS1963 | + | − |
| GS1964 | + | − |
| GS1965 | − | − |
| GS1966 | + | + |
| GS1967 | + | + |
| GS1968 | + | + |
| GS1969 | + | − |
| GS1970 | − | + |
| GS1971 | − | + |
| GS1972 | + | + |
| GS1973 | + | ++ |
| GS1974 | + | − |
| GS1975 | + | − |
| GS1976 | + | − |
| GS1977 | + | − |
| GS1978 | − | + |
| GS1979 | + | − |
| GS1980 | + | + |
| GS1981 | + | − |
| GS1982 | ++ | − |
| GS1983 | ++ | − |
| GS1984 | ++ | − |
| GS1985 | + | − |
| GS1986 | + | − |
| GS1988 | ++ | − |
| GS1989 | + | − |
| GS1990 | + | − |
| GS1991 | ++ | + |
| GS1992 | ++ | − |
| GS1994 | + | − |
| GS1995 | + | − |
| GS1996 | ++ | − |
| GS1997 | + | − |
| GS1998 | + | − |
| GS2000 | ++ | + |
| GS2001 | + | − |
| GS2002 | + | − |
| GS2003 | + | − |
| GS2004 | ++ | − |
| GS2005 | ++ | − |
| GS2006 | + | − |
| GS2007 | + | − |
| GS2008 | ++ | − |
| GS2009 | ++ | − |
| GS2010 | ++ | + |
| GS2012 | + | − |
| GS2013 | + | − |
| GS2014 | + | − |
| GS2015 | + | − |
| GS2016 | ++ | − |
| GS2017 | + | − |
| GS2018 | + | − |
| GS2019 | ++ | − |
| GS2020 | + | − |
| GS2021 | ++ | − |
| GS2022 | ++ | + |
| GS2023 | + | − |
| GS2024 | + | − |
| GS2025 | ++ | − |
| GS2026 | + | − |
| GS2027 | ++ | − |
| GS2028 | +++ | + |
| GS2029 | + | − |
| GS2030 | ++ | − |
| GS2031 | ++ | − |
| GS2032 | ++ | − |
| GS2033 | ++ | − |
| GS2034 | ++ | − |
| GS2035 | + | − |
| GS2036 | ++ | − |
| GS2037 | +++ | + |
| GS2038 | +++ | ++ |
| GS2039 | ++ | − |
| GS2040 | ++ | − |
| GS2041 | ++ | + |
| GS2042 | +++ | + |
| GS2043 | ++ | − |
| GS2044 | ++ | − |
| GS2045 | +++ | + |
| GS2046 | +++ | + |
| GS2047 | ++ | − |
| GS2048 | +++ | + |
| GS2049 | +++ | + |
| GS2050 | ++ | − |
| GS2051 | ++ | − |
| GS2052 | ++ | − |
| GS2053 | ++ | − |
| GS2054 | ++ | − |
| GS2055 | ++ | − |
| GS2056 | +++ | + |
| GS2057 | +++ | ++ |
| GS2058 | +++ | ++ |
| GS2059 | +++ | + |
| GS2060 | ++ | − |
| GS2061 | ++ | + |
| GS2062 | ++ | − |
| GS2063 | ++ | ++ |
| GS2064 | ++ | ++ |
| GS2065 | +++ | ++ |
| GS2066 | +++ | ++ |
| GS2067 | ++ | + |
| GS2068 | +++ | +++ |
| GS2069 | +++ | +++ |
| GS2070 | ++ | ++ |
| GS2071 | +++ | +++ |
| GS2072 | ++ | ++ |
| GS2073 | ++ | + |
| GS2074 | +++ | +++ |
| GS2075 | ++ | + |
| GS2076 | ++ | + |
| GS2077 | +++ | +++ |
| GS2078 | ++ | + |
| GS2079 | ++ | ++ |
| GS2080 | ++ | + |
| GS2081 | ++ | ++ |
| GS2082 | +++ | +++ |
| GS2083 | ++ | + |
| GS2084 | + | − |
| GS2085 | ++ | + |
| GS2086 | + | + |
| GS2087 | +++ | +++ |
| GS2088 | ++ | + |
| GS2089 | + | + |
| GS2090 | + | + |
| GS2091 | ++ | + |
| GS2092 | ++ | + |
| GS2093 | ++ | + |
| GS2094 | ++ | + |
| GS2095 | + | − |
| GS2096 | ++ | + |
| GS2097 | ++ | ++ |
| GS2098 | ++ | + |
| GS2100 | ++ | + |
| GS2102 | ++ | ++ |
| GS2103 | ++ | ++ |
| GS2104 | +++ | +++ |
| GS2105 | +++ | ++ |
| GS2106 | ++ | + |

### Example 5: Microinjection Study of dsRNA in P. xylostella

### DBM Injection Protocol

The scoring data is provided in Table 9 below. Each of the dsRNA molecules in Table 1C are identified by their GS number which correlate to the SEQ ID NO provided in Tables 1A-1C. Certain sequences showing >30% mortality in the diet assays described above were tested by microinjection for further identification of effective sequences, with results showing Table 9 below. P. xylostella larvae were injected with dsRNA of the identified sequences in a similar injection bioassay to that described in Knorr et al. (2018) Scientific Reports 8:2061 at 11 “Gene silencing in Tribolium castaneum as a tool for the targeted identification of candidate RNAi targets in crop pests” with species-appropriate feed. Results are shown in Table 9 with a score of ++ for 50%-63% mortality and +++ for >/=64% mortality.

TABLE 9

| | | Exposure Method |
|---|---|---|
| | | Microinjection |
| | | Estimated |
| | | dsRNA exposure |
| | | 0.05 ug/ |
| | | insect |
| | | ++ >50% |
| | | +++ >64% |
| Wave 1 | GS144 | ++ |
| | GS146 | ++ |
| | GS269 | ++ |
| | GS318 | +++ |
| | GS319 | +++ |
| | GS321 | ++ |
| | GS329 | +++ |
| | GS486 | ++ |
| Wave 2.1 | GS901 | − |
| | GS947 | − |
| | GS953 | − |
| | GS954 | − |
| | GS957 | − |
| | GS958 | +++ |
| | GS959 | − |
| | GS963 | ++ |
| | GS964 | − |
| | GS984 | − |
| | GS961 | +++ |
| Wave 2.2 | GS1265 | − |
| | GS1254 | − |
| | GS1256 | − |
| | GS1261 | ++ |
| | GS1263 | − |
| | GS1281 | − |
| Wave 3 | GS889 | +++ |
| | GS912 | +++ |
| | GS1333 | ++ |
| | GS1373 | ++ |
| | GS1374 | ++ |
| | GS1380 | ++ |
| | GS1464 | − |
| | GS1590 | +++ |
| | GS1594 | +++ |
| | GS1602 | +++ |
| | GS1611 | +++ |
| | GS1618 | − |
| Control | GS134 | − |

What is claimed is:

1. A method for controlling a Lepidopteran pest infestation of a plant comprising:
(a) contacting said Lepidopteran pest with at least one polynucleotide comprising a nucleotide sequence that is complementary to at least 18, 19, 20, 21, 25, 50, 100, 150 or 200 contiguous nucleotides, or complementary to a nucleotide sequence having at least 90 percent, 95 percent or 98 percent identity with said contiguous nucleotides, of a target gene having a nucleotide sequence selected from the group consisting of: SEQ ID NOs 1-114, 229-361, 495-511; 530-535, 542-822, 1104-1333, and 1564-1623, or an RNA transcribed from said target gene; or
(b) providing in the diet of said Lepidopteran pest at least one polynucleotide comprising a nucleotide sequence that is complementary to at least 18, 19, 20, 21, 25, 50, 100, 150 or 200 contiguous nucleotides, or complementary to a nucleotide sequence having at least 90 percent, 95 percent or 98 percent identity with said contiguous nucleotides, of a target gene having a nucleotide sequence selected from the group consisting of: SEQ ID NOs: 1-114, 229-361, 495-511; 530-535, 542-822, 1104-1333, and 1564-1623, or an RNA transcribed from said target gene; or
(c) causing mortality or stunting in larvae of said Lepidopteran pest by providing in the diet of said larvae at least one polynucleotide comprising at least one silencing element comprising at least 18, 19, 20, 21, or 25 contiguous nucleotides that are complementary to a target gene having a nucleotide sequence selected from the group consisting of: SEQ ID NOs: 1-114, 229-361, 495-511; 530-535, 542-822, 1104-1333, and 1564-1623, or an RNA transcribed from said target gene; or
(d) topically applying to said plant a composition comprising at least one polynucleotide comprising a nucleotide sequence that is complementary to at least 18, 19, 20, 21, 25, 50, 100, 150 or 200 contiguous nucleotides, or complementary to a nucleotide sequence having at least 90 percent, 95 percent or 98 percent identity with said contiguous nucleotides, of a target gene having a nucleotide sequence selected from the group consisting of: SEQ ID NOs: 1-114, 229-361, 495-511; 530-535, 542-822, 1104-1333, and 1564-1623, or an RNA transcribed from said target gene; or
(e) topically applying to said plant a composition comprising at least one polynucleotide in a manner such that an effective amount of said polynucleotide is ingested by Lepidopteran species feeding on said plant, said at least one polynucleotide comprising a nucleotide sequence that is complementary to at least 18, 19, 20, 21, 25, 50, 100, 150 or 200 contiguous nucleotides, or complementary to a nucleotide sequence having at least 90 percent, 95 percent or 98 percent identity with said contiguous nucleotides, of a target gene having a nucleotide sequence selected from the group consisting of: SEQ ID NOs: 1-114, 229-361, 495-511; 530-535, 542-822, 1104-1333, and 1564-1623, or an RNA transcribed from said target gene; or
(f) expressing in said plant at least one polynucleotide comprising at least one segment that is essentially identical or complementary to at least 18, 19, 20, 21, 25, 50, 100, 150 or 200 contiguous nucleotides, or essentially identical or complementary to a nucleotide sequence having at least 90 percent, 95 percent or 98 percent identity with said contiguous nucleotides, of a DNA having a sequence selected from the group consisting of: SEQ ID NOs: 1-114, 229-361, 495-511; 530-535, 542-822, 1104-1333, and 1564-1623; or
(g) contacting said Lepidopteran pest with an effective amount of at least one double-stranded RNA, one strand of which is complementary to at least 18, 19, 20, 21, 25, 50, 100, 150 or 200 contiguous nucleotides, or complementary to a nucleotide sequence having at least 90 percent, 95 percent or 98 percent identity with said contiguous nucleotides, of a sequence selected from the group consisting of SEQ ID NOs: 115-228, 362-494, 512-529; 536-541, 823-1103, 1334-1563, and 1624-1683.

2. The method of claim 1, wherein said at least one polynucleotide is a double-stranded RNA.

3. The method of claim 2, wherein said double-stranded RNA is chemically or enzymatically synthesized or is produced by expression in a microorganism or by expression in a plant cell.

4. The method of claim 2, wherein said double-stranded RNA comprises one strand complementary to at least about 50 contiguous nucleotides, at least about 100 nucleotides, at least about 150 nucleotides or at least about 200 contiguous nucleotides, or complementary to a nucleotide sequence having at least 90 percent, 95 percent or 98 percent identity of said contiguous nucleotides, of a sequence selected from the group consisting of SEQ ID NOs: 115, 116, 119, 121, 123, 127, 135, 140, 141, 143, 150, 151, 153, 158, 161, 201, 209, 213, 217, 434, 442, 451, 452, 453, 454, 456, 457, 461, 474, 476, 479, 489, 491, 492, 521, 522, 523, 524, 527, 528, 536, 538, 539, 823, 826, 827, 828, 830, 832, 833, 834, 840, 841, 842, 844, 845, 851, 853, 854, 862, 874, 877, 883, 892, 893, 901, 946, 947, 953, 998, 1011, 1012, 1017, 1018, 1019, 1021, 1022, 1023, 1024, 1030, 1038, 1041, 1042, 1044, 1047, 1089, 1090, 1091, 1095, 1096, 1098, 1099, 1102, 1103, 1334, 1335, 1336, 1341, 1343, 1346, 1351, 1353, 1354, 1362, 1363, 1377, 1386, 1387, 1393, 1396, 1417, 1420, 1422, 1425, 1426, 1446, 1447, 1470, 1473, 1481, 1493, 1494, 1495, 1500, 1505, 1509, 1513, 1520, 1538, 1540, 1546, 1548, 1550, 1624, 1625, 1629, 1682, or 1683.

5. The method of claim 1, wherein said method comprises topically applying to said plant a composition comprising at least one polynucleotide comprising a nucleotide sequence that is complementary to at least 18, 19, 20, 21, 25, 50, 100, 150, or 200 contiguous nucleotides, or complementary to a nucleotide sequence having at least 90 percent, 95 percent or 98 percent identity of said contiguous nucleotides, of a target gene or an RNA transcribed from said target gene, wherein said target gene has a nucleotide sequence selected from the group consisting of: SEQ ID NOs: 2, 5, 7, 9, 13, 21, 26, 27, 29, 36, 37, 39, 44, 47, 87, 95, 99, 103, 301, 309, 318, 319, 320, 321, 323, 324, 328, 341, 343, 346, 356, 358, 359, 503, 504, 505, 506, 509, 510, 530, 532, 533, 542, 545, 546, 547, 549, 551, 552, 553, 559, 560, 561, 563, 564, 570, 572, 573, 581, 593, 596, 602, 611, 612, 620, 665, 666, 672, 717, 730, 731, 736, 737, 738, 740, 741, 742, 743, 749, 757, 760, 761, 763, 766, 808, 809, 810, 814, 815, 817, 818, 821, 822, 1104, 1105, 1106, 1111, 1113, 1116, 1121, 1123, 1124, 1132, 1133, 1147, 1156, 1157, 1163, 1166, 1187, 1190, 1192, 1195, 1196, 1216, 1217, 1240, 1243, 1251, 1263, 1264, 1265, 1270, 1275, 1279, 1283, 1290, 1308, 1310, 1316, 1318, 1320, 1564, 1565, 1569, 1622, and 1623; and, optionally, wherein said composition further comprises one or more components selected from the group consisting of a carrier agent, a surfactant, a cationic lipid, an organosilicone, an organosilicone surfactant, a polynucleotide herbicidal molecule, a nonpolynucleotide herbicidal molecule, a non-polynucleotide pesticide, a safener, and an insect growth regulator.

6. The method of claim 1, wherein said method comprises contacting said Lepidopteran pest with an effective amount of a solution comprising a double-stranded RNA, wherein at least one strand of the double-stranded RNA is complementary to at least 18, 19, 20, 21, 25, 50, 100, 150, or 200 contiguous nucleotides, or complementary to a nucleotide sequence having at least 90 percent, 95 percent or 98 percent identity of said contiguous nucleotides, of a gene that having a sequence selected from the group consisting of SEQ ID NOs: 2, 5, 7, 9, 13, 21, 26, 27, 29, 36, 37, 39, 44, 47, 87, 95, 99, 103, 301, 309, 318, 319, 320, 321, 323, 324, 328, 341, 343, 346, 356, 358, 359, 503, 504, 505, 506, 509, 510, 530, 532, 533, 542, 545, 546, 547, 549, 551, 552, 553, 559, 560, 561, 563, 564, 570, 572, 573, 581, 593, 596, 602, 611, 612, 620, 665, 666, 672, 717, 730, 731, 736, 737, 738, 740, 741, 742, 743, 749, 757, 760, 761, 763, 766, 808, 809, 810, 814, 815, 817, 818, 821, 822, 1104, 1105, 1106, 1111, 1113, 1116, 1121, 1123, 1124, 1132, 1133, 1147, 1156, 1157, 1163, 1166, 1187, 1190, 1192, 1195, 1196, 1216, 1217, 1240, 1243, 1251, 1263, 1264, 1265, 1270, 1275, 1279, 1283, 1290, 1308, 1310, 1316, 1318, 1320, 1564, 1565, 1569, 1622, and 1623, and wherein RNA interference is induced and Lepidopteran pest mortality occurs.

7. The method of claim 6, wherein said solution further comprises one or more components selected from the group consisting of an organosilicone surfactant or a cationic lipid.

8. The method of claim 1, wherein the target gene of (a), (b), (c), (d) or (e) or the DNA of (f) has a sequence selected from the group consisting of SEQ ID NOs. 7, 9, 21, 26, 27, 29, 87, 95, or 99, or wherein the double-stranded RNA of (g) comprises one strand complementary to at least about 50 contiguous nucleotides, at least about 100 nucleotides, at least about 150 nucleotides or at least about 200 contiguous nucleotides of a sequence selected from the group consisting of SEQ ID NOs: 121, 123, 135, 140, 141, 143, 201, 209, and 213, or a sequence having a least 90 percent, at least 95 percent or 98 percent identity to the selected sequence or to a fragment thereof of equivalent length to the one strand.

9. The method of claim 1, wherein said Lepidopteran pest is selected from the group consisting of: Spodoptera frugiperda and Plutella xylostella.

10. The method of any of claim 1-4, 6 or 7, wherein the at least one double stranded RNA comprises one strand complementary to at least about 50 contiguous nucleotides, at least about 100 nucleotides, at least about 150 nucleotides or at least about 200 contiguous nucleotides of a sequence selected from the group consisting of SEQ ID NOs: 115-228, 362-494, 512-529; 536-541, 823-1103, 1334-1563, and 1624-1683, or a sequence having a least 90 percent, at least 95 percent or 98 percent identity to the selected sequence or to a fragment thereof of equivalent length to the one strand.

11. The method of claim 10, wherein the one strand comprises a sequence complementary to at least about 100 contiguous nucleotides of a sequence selected from the group.

12. The method of any of claim 1-4, 6 or 7, wherein the double stranded RNA comprises at least one sequence selected from the group consisting of SEQ ID NOs: 115-228, 362-494, 512-529; 536-541, 823-1103, 1334-1563, and 1624-1683, or a fragment thereof of at least about 50 contiguous nucleotides, at least about 100 nucleotides, at least about 150 nucleotides or at least about 200 contiguous nucleotides, or a sequence having at least 90 percent, at least 95 percent or 98 percent identity to the double stranded RNA or a fragment thereof.

13. The method of claim 10 or 11, wherein the at least one double stranded RNA comprises one strand complementary to at least about 50 contiguous nucleotides, at least about 100 nucleotides, at least about 150 nucleotides or at least about 200 contiguous nucleotides of a sequence selected from the group consisting of SEQ ID NOs: 115, 116, 119, 127, 150, 151, 153, 158, 161, 217, 434, 442, 451, 452, 453, 454, 456, 457, 461, 474, 476, 479, 489, 491, 492, 521, 522, 523, 524, 527, 528, 536, 538 and 539, 823, 826, 827, 828, 830, 832, 833, 834, 840, 841, 842, 844, 845, 851, 853, 854, 862, 874, 877, 883, 892, 893, 901, 946, 947, 953, 998, 1011, 1012, 1017, 1018, 1019, 1021, 1022, 1023, 1024, 1030, 1038, 1041, 1042, 1044, 1047, 1089, 1090, 1091, 1095, 1096, 1098, 1099, 1102, 1103, 1334, 1335, 1336, 1341, 1343, 1346, 1351, 1353, 1354, 1362, 1363, 1377, 1386, 1387, 1393, 1396, 1417, 1420, 1422, 1425, 1426, 1446, 1447, 1470, 1473, 1481, 1493, 1494, 1495, 1500, 1505, 1509, 1513, 1520, 1538, 1540, 1546, 1548, 1550, 1624, 1625, 1629, 1682, and 1683, or a sequence having a least 90 percent, at least 95 percent or 98 percent identity to the selected sequence or to a fragment thereof of equivalent length to the one strand.

14. The method of claim 1, wherein the target gene of (a), (b), (c), (d), or (e), or the DNA of (f) has a sequence selected from the group consisting of SEQ ID NOs. 2, 5, 13, 36, 37, 39, 44, 47, 103, 301, 309, 318, 319, 320, 321, 323, 324, 328, 341, 343, 346, 356, 358, 359, 503, 504, 505, 506, 509, 510, 530, 532, 533, 542, 545, 546, 547, 549, 551, 552, 553, 559, 560, 561, 563, 564, 570, 572, 573, 581, 593, 596, 602, 611, 612, 620, 665, 666, 672, 717, 730, 731, 736, 737, 738, 740, 741, 742, 743, 749, 757, 760, 761, 763, 766, 808, 809, 810, 814, 815, 817, 818, 821, 822, 1104, 1105, 1106, 1111, 1113, 1116, 1121, 1123, 1124, 1132, 1133, 1147, 1156, 1157, 1163, 1166, 1187, 1190, 1192, 1195, 1196, 1216, 1217, 1240, 1243, 1251, 1263, 1264, 1265, 1270, 1275, 1279, 1283, 1290, 1308, 1310, 1316, 1318, 1320, 1564, 1565, 1569, 1622, and 1623,

15. The method of claim 13 or 14, wherein the Lepidopteran pest is Plutella xylostella.

16. A plant having improved resistance to a Lepidopteran pest infestation, provided by the method of claim 1, or a fruit, seed, or propagatable part of said plant.

17. The plant of claim 16, wherein said plant is a crop plant.

18. An insecticidal composition for controlling a Lepidopteran pest, comprising:
(a) an insecticidally effective amount of at least one polynucleotide comprising a nucleotide sequence that is complementary to at least 18, 19, 20, 21, 25, 50, 100, 150 or 200 contiguous nucleotides, or complementary to a nucleotide sequence having at least 90 percent, 95 percent or 98 percent identity with said contiguous nucleotides, of a target gene having a nucleotide sequence selected from the group consisting of: SEQ ID NOs 1-114, 229-361, 495-511; 530-535, 542-822, 1104-1333, and 1564-1623, or an RNA transcribed from said target gene; or
(b) an insecticidally effective amount of at least one polynucleotide comprising at least one silencing element that is complementary to at least 18, 19, 20, 21 or 25 contiguous nucleotides of a target gene or an RNA transcribed from said target gene, wherein said target gene has a nucleotide sequence selected from the group consisting of: SEQ ID NOs: 1-114, 229-361, 495-511; 530-535, 542-822, 1104-1333, and 1564-1623; or
(c) an insecticidally effective amount of at least one RNA comprising at least one segment that is identical or complementary to at least 18 contiguous nucleotides of a target gene having a nucleotide sequence selected from the group consisting of: SEQ ID NOs: 1-114, 229-361, 495-511; 530-535, 542-822, 1104-1333, and 1564-1623, or an RNA transcribed from said target gene; or
(d) an RNA molecule that causes mortality or stunting of growth in a Lepidopteran pest when ingested or contacted by said Lepidopteran pest, wherein said RNA molecule comprises a nucleotide sequence that is complementary to at least 18, 19, 20, 21, 25, 50, 100, 150 or 200 contiguous nucleotides, or complementary to a nucleotide sequence having at least 90 percent, 95 percent or 98 percent identity of said contiguous nucleotides, of a target gene having a nucleotide sequence selected from the group consisting of: SEQ ID NOs: 1-114, 229-361, 495-511; 530-535, 542-822, 1104-1333, and 1564-1623, or an RNA transcribed from said target gene; or
(e) an insecticidal double-stranded RNA molecule that causes mortality or stunting of growth in a Lepidopteran pest when ingested or contacted by said Lepidopteran pest, wherein at least one strand of said insecticidal double-stranded RNA molecule is complementary to at least 18, 19, 20, 21, 25, 50, 100, 150 or 200 contiguous nucleotides, or complementary to a nucleotide sequence having at least 90 percent, 95 percent or 98 percent identity of said contiguous nucleotides, of a target gene or an RNA transcribed from said target gene, wherein said target gene has a sequence selected from the group consisting of: SEQ ID NOs: 1-114, 229-361, 495-511; 530-535, 542-822, 1104-1333, and 1564-1623; or
(f) an insecticidally effective amount of at least one double-stranded RNA comprising, one strand of which is complementary to at least 18, 19, 20, 21, 25, 50, 100, 150 or 200 contiguous nucleotides, or complementary to a nucleotide sequence having at least 90 percent, 95 percent or 98 percent identity of said contiguous nucleotides, of a sequence selected from the group consisting of: SEQ ID NOs: 115-228, 362-494, 512-529; 536-541, 823-1103, 1334-1563, and 1624-1683.

19. The insecticidal composition of claim 18, wherein the double stranded RNA molecule of (e) or double stranded RNA of (f) comprises one strand complementary to at least about 50 contiguous nucleotides, at least about 100 nucleotides, at least about 150 nucleotides or at least about 200 contiguous nucleotides of a sequence selected from the group consisting of SEQ ID NOs: 115-228, 362-494, 512-529; 536-541, 823-1103, 1334-1563, and 1624-1683, or a sequence having a least 90 percent, at least 95 percent or 98 percent identity to the selected sequence or to a fragment thereof of equivalent length to the one strand.

20. The insecticidal composition of claim 19, wherein the one strand comprises a sequence complementary to at least about 100 contiguous nucleotides of a sequence selected from the group.

21. The insecticidal composition of claim 18, wherein the double stranded RNA comprises at least one sequence selected from the group consisting of SEQ ID NOs: SEQ ID NOs: 115-228, 362-494, 512-529; 536-541, 823-1103, 1334-1563, and 1624-1683, or a fragment thereof of at least about 50 contiguous nucleotides, at least about 100 nucleotides, at least about 150 nucleotides or at least about 200 contiguous nucleotides, or a sequence having at least 90 percent, at least 95 percent or 98 percent identity to the double stranded RNA or a fragment thereof.

22. The insecticidal composition of any of claims 19-21, wherein the group is SEQ ID NOs: 115, 116, 119, 127, 150, 151, 153, 158, 161, 217, 434, 442, 451, 452, 453, 454, 456, 457, 461, 474, 476, 479, 489, 491, 492, 521, 522, 523, 524, 527, 528, 536, 538, 539, 823, 826, 827, 828, 830, 832, 833, 834, 840, 841, 842, 844, 845, 851, 853, 854, 862, 874, 877, 883, 892, 893, 901, 946, 947, 953, 998, 1011, 1012, 1017, 1018, 1019, 1021, 1022, 1023, 1024, 1030, 1038, 1041, 1042, 1044, 1047, 1089, 1090, 1091, 1095, 1096, 1098, 1099, 1102, 1103, 1334, 1335, 1336, 1341, 1343, 1346, 1351, 1353, 1354, 1362, 1363, 1377, 1386, 1387, 1393, 1396, 1417, 1420, 1422, 1425, 1426, 1446, 1447, 1470, 1473, 1481, 1493, 1494, 1495, 1500, 1505, 1509, 1513, 1520, 1538, 1540, 1546, 1548, 1550, 1624, 1625, 1629, 1682, and 1683.

23. The insecticidal composition of claim 18, wherein the group in (a), (b), (c), (d) and (e) is 2, 5, 13, 36, 37, 39, 44, 47, 103, 301, 309, 318, 319, 320, 321, 323, 324, 328, 341, 343, 346, 356, 358, 359, 503, 504, 505, 506, 509, 510, 530, 532, 533, 542, 545, 546, 547, 549, 551, 552, 553, 559, 560, 561, 563, 564, 570, 572, 573, 581, 593, 596, 602, 611, 612, 620, 665, 666, 672, 717, 730, 731, 736, 737, 738, 740, 741, 742, 743, 749, 757, 760, 761, 763, 766, 808, 809, 810, 814, 815, 817, 818, 821, 822, 1104, 1105, 1106, 1111, 1113, 1116, 1121, 1123, 1124, 1132, 1133, 1147, 1156, 1157, 1163, 1166, 1187, 1190, 1192, 1195, 1196, 1216, 1217, 1240, 1243, 1251, 1263, 1264, 1265, 1270, 1275, 1279, 1283, 1290, 1308, 1310, 1316, 1318, 1320, 1564, 1565, 1569, 1622, and 1623.

24. The insecticidal composition of claims 22 and 23, wherein the Lepidopteran pest is Plutella xylostella.

25. The insecticidal composition of any of claims 18-24, wherein said insecticidal composition is in the form of at least one selected from the group consisting of a solid, liquid, powder, suspension, emulsion, spray, encapsulation, microbeads, carrier particulates, film, matrix, seed treatment, soil drench, implantable formulation, and in-furrow formulation.

26. The insecticidal composition of any of claims 18-25, further comprising at least one component selected from the group consisting of a carrier agent, a surfactant, a cationic lipid, an organosilicone, an organosilicone surfactant, a polynucleotide herbicidal molecule, a non-polynucleotide herbicidal molecule, a non-polynucleotide pesticide, a safener, and an insect growth regulator.

27. The insecticidal composition of any of claims 18-26, wherein said insecticidal composition comprises an insecticidal double-stranded RNA molecule that causes mortality or stunting of growth in a Lepidopteran pest when ingested or contacted by said Lepidopteran pest, wherein said insecticidal double-stranded RNA molecule comprises at least one segment that is complementary to 21 contiguous nucleotides of a DNA having a sequence selected from the group consisting of: SEQ ID NOs: 1-114, 229-361, 495-511; 530-535, 542-822, 1104-1333, and 1564-1623, or an RNA transcribed from said DNA, and wherein said double-stranded RNA molecule is at least 50 base-pairs in length or is between about 100 to about 500 base-pairs in length.

28. A recombinant DNA construct comprising a heterologous promoter operably linked to:
(a) DNA comprising a nucleotide sequence that is complementary to at least 18, 19, 20, 21, 25, 50, 100, 150, or 200 contiguous nucleotides, or complementary to a nucleotide sequence having at least 90 percent, 95 percent or 98 percent identity of said contiguous nucleotides, of a target gene having a sequence selected from the group consisting of: SEQ ID NOs: 1-114, 229-361, 495-511; 530-535, 542-822, 542-822, 1104-1333, and 1564-1623, or an RNA transcribed from said target gene; or
(b) a DNA comprising 18 or more contiguous nucleotides having 100% identity to a fragment of equivalent length of a DNA having a sequence selected from the group consisting of: SEQ ID NOs: 1-114, 229-361, 495-511; 530-535, 542-822, 542-822, 1104-1333, and 1564-1623, or the DNA complement thereof; or
(c) DNA encoding at least one silencing element that is complementary to at least 18 contiguous nucleotides of a target gene or an RNA transcribed from said target gene, wherein said target gene has a sequence selected from the group consisting of: SEQ ID NOs: 1-114, 229-361, 495-511; 530-535, 542-822, 542-822, 1104-1333, and 1564-1623; or
(d) DNA encoding a RNA comprising a sequence selected from the group consisting of SEQ ID NOs: 115-228, 362-494, 512-529; 536-541, 823-1103, 1334-1563, and 1624-1683.

29. A plant chromosome or a plastid or a recombinant plant virus vector or a recombinant baculovirus vector comprising the recombinant DNA construct of claim 16.

30. A transgenic crop plant cell having in its genome the recombinant DNA construct of claim 28.

31. The transgenic crop plant cell of claim 30, wherein said transgenic crop plant cell further has in its genome DNA encoding at least one pesticidal agent selected from the group consisting of a patatin, a plant lectin, a phytoecdysteroid, a Bacillus thuringiensis insecticidal protein, a Xenorhabdus insecticidal protein, a Photorhabdus insecticidal protein, a Bacillus laterosporus insecticidal protein, and a Bacillus sphaericus insecticidal protein.

32. A transgenic crop plant comprising the transgenic solanaceous plant cell of claim 30, or a fruit, seed, or propagatable part of said transgenic crop plant.

33. A method for producing a polynucleotide for use in Lepidopteran pest control, the method comprising:
(a) incubating in a reaction mixture cellular ribonucleic acid (RNA) and a ribonuclease and producing 5′ nucleoside monophosphates (5′ NMPs);
(b) eliminating the ribonuclease; and
(c) incubating in the reaction mixture, or in a second reaction mixture, the 5′ NMPs, a polyphosphate kinase, a polyphosphate, a polymerase, and a deoxyribonucleic acid (DNA) template having at least 80% identity to SEQ ID NO: 2, 5, 7, 9, 13, 21, 26, 27, 29, 36, 37, 39, 44, 47, 87, 95, 99, 103, 301, 309, 318, 319, 320, 321, 323, 324, 328, 341, 343, 346, 356, 358, 359, 503, 504, 505, 506, 509, 510, 530, 532, 533, 542, 545, 546, 547, 549, 551, 552, 553, 559, 560, 561, 563, 564, 570, 572, 573, 581, 593, 596, 602, 611, 612, 620, 665, 666, 672, 717, 730, 731, 736, 737, 738, 740, 741, 742, 743, 749, 757, 760, 761, 763, 766, 808, 809, 810, 814, 815, 817, 818, 821, 822, 1104, 1105, 1106, 1111, 1113, 1116, 1121, 1123, 1124, 1132, 1133, 1147, 1156, 1157, 1163, 1166, 1187, 1190, 1192, 1195, 1196, 1216, 1217, 1240, 1243, 1251, 1263, 1264, 1265, 1270, 1275, 1279, 1283, 1290, 1308, 1310, 1316, 1318, 1320, 1564, 1565, 1569, 1622, and 1623, or encoding an RNA sequence that comprises a segment that comprises at least 18 contiguous nucleotides, wherein the segment has at least 90% identity to a segment of a sequence of SEQ ID NOs: 115, 116, 119, 127, 150, 151, 153, 158, 161, 217, 434, 442, 451, 452, 453, 454, 456, 457, 461, 474, 476, 479, 489, 491, 492, 521, 522, 523, 524, 527, 528, 536, 538, 539, 823, 826, 827, 828, 830, 832, 833, 834, 840, 841, 842, 844, 845, 851, 853, 854, 862, 874, 877, 883, 892, 893, 901, 946, 947, 953, 998, 1011, 1012, 1017, 1018, 1019, 1021, 1022, 1023, 1024, 1030, 1038, 1041, 1042, 1044, 1047, 1089, 1090, 1091, 1095, 1096, 1098, 1099, 1102, 1103, 1334, 1335, 1336, 1341, 1343, 1346, 1351, 1353, 1354, 1362, 1363, 1377, 1386, 1387, 1393, 1396, 1417, 1420, 1422, 1425, 1426, 1446, 1447, 1470, 1473, 1481, 1493, 1494, 1495, 1500, 1505, 1509, 1513, 1520, 1538, 1540, 1546, 1548, 1550, 1624, 1625, 1629, 1682, or 1683, and producing the RNA of interest, optionally wherein the reaction mixture of step (c) further comprises a nucleoside kinase, a NMP kinase, and/or a NDP kinase.

34. The method of claim 1, wherein the at least 18 contiguous nucleotides recited in (a)-(g) is at least 18, 19, 20, or 21 contiguous nucleotides.

35. The method of claim 1, wherein said method comprises topically applying to said plant a composition comprising at least one polynucleotide in a manner such that an effective amount of said polynucleotide is ingested by Lepidopteran pests feeding on said plant, said polynucleotide comprising a nucleotide sequence that is complementary to at least 18, 19, 20, or 21 contiguous nucleotides of a target gene having a nucleotide sequence selected from the group consisting of: SEQ ID NOs: 1-114, 229-361, 495-511; 530-535, 542-822, 1104-1333, and 1564-1623, or an RNA transcribed from said target gene; wherein said Lepidopteran pest is Plutella xylostella; and wherein said target gene has the sequence of SEQ ID NO: 2, 5, 13, 36, 37, 39, 44, 47, 103, 301, 309, 318, 319, 320, 321, 323, 324, 328, 341, 343, 346, 356, 358, 359, 503, 504, 505, 506, 509, 510, 530, 532, 533, 542, 545, 546, 547, 549, 551, 552, 553, 559, 560, 561, 563, 564, 570, 572, 573, 581, 593, 596, 602, 611, 612, 620, 665, 666, 672, 717, 730, 731, 736, 737, 738, 740, 741, 742, 743, 749, 757, 760, 761, 763, 766, 808, 809, 810, 814, 815, 817, 818, 821, 822, 1104, 1105, 1106, 1111, 1113, 1116, 1121, 1123, 1124, 1132, 1133, 1147, 1156, 1157, 1163, 1166, 1187, 1190, 1192, 1195, 1196, 1216, 1217, 1240, 1243, 1251, 1263, 1264, 1265, 1270, 1275, 1279, 1283, 1290, 1308, 1310, 1316, 1318, 1320, 1564, 1565, 1569, 1622, or 1623, or wherein said polynucleotide is a double-stranded RNA having a strand with a sequence selected from the group consisting of SEQ ID NO: 115, 116, 119, 127, 150, 151, 153, 158, 161, 217, 434, 442, 451, 452, 453, 454, 456, 457, 461, 474, 476, 479, 489, 491, 492, 521, 522, 523, 524, 527, 528, 536, 538, 539, 823, 826, 827, 828, 830, 832, 833, 834, 840, 841, 842, 844, 845, 851, 853, 854, 862, 874, 877, 883, 892, 893, 901, 946, 947, 953, 998, 1011, 1012, 1017, 1018, 1019, 1021, 1022, 1023, 1024, 1030, 1038, 1041, 1042, 1044, 1047, 1089, 1090, 1091, 1095, 1096, 1098, 1099, 1102, 1103, 1334, 1335, 1336, 1341, 1343, 1346, 1351, 1353, 1354, 1362, 1363, 1377, 1386, 1387, 1393, 1396, 1417, 1420, 1422, 1425, 1426, 1446, 1447, 1470, 1473, 1481, 1493, 1494, 1495, 1500, 1505, 1509, 1513, 1520, 1538, 1540, 1546, 1548, 1550, 1624, 1625, 1629, 1682, or 1683.

35. An insecticidal composition for controlling a Lepidopteran pest, comprising an insecticidal double-stranded RNA molecule that causes mortality or stunting of growth in a Lepidopteran pest when ingested or contacted by said Lepidopteran pest, wherein at least one strand of said insecticidal double-stranded RNA molecule comprises at least 18, 19, 20, 21, 25, 50, 100, 150 or 200 contiguous nucleotides that are complementary to a sequence selected from the group consisting of: SEQ ID NOs: 115-228, 362-494, 512-529; 536-541, 823-1103, 1334-1563, and 1624-1683, or a sequence having a least 90 percent, at least 95 percent or 98 percent identity to the selected sequence or to a fragment thereof of equivalent length to the at least one strand.

36. The insecticidal composition of claim 35, wherein the group is SEQ ID NOs: 115, 116, 119, 127, 150, 151, 153, 158, 161, 217, 434, 442, 451, 452, 453, 454, 456, 457, 461, 474, 476, 479, 489, 491, 492, 521, 522, 523, 524, 527, 528, 536, 538, 539, 823, 826, 827, 828, 830, 832, 833, 834, 840, 841, 842, 844, 845, 851, 853, 854, 862, 874, 877, 883, 892, 893, 901, 946, 947, 953, 998, 1011, 1012, 1017, 1018, 1019, 1021, 1022, 1023, 1024, 1030, 1038, 1041, 1042, 1044, 1047, 1089, 1090, 1091, 1095, 1096, 1098, 1099, 1102, 1103, 1334, 1335, 1336, 1341, 1343, 1346, 1351, 1353, 1354, 1362, 1363, 1377, 1386, 1387, 1393, 1396, 1417, 1420, 1422, 1425, 1426, 1446, 1447, 1470, 1473, 1481, 1493, 1494, 1495, 1500, 1505, 1509, 1513, 1520, 1538, 1540, 1546, 1548, 1550, 1624, 1625, 1629, 1682, and 1683, or a sequence having a least 90 percent, at least 95 percent or 98 percent identity to the selected sequence or to a fragment thereof of equivalent length to the at least one strand

37. The insecticidal composition of claim 35, wherein the double stranded RNA comprises one strand complementary to at least about 50 contiguous nucleotides, at least about 100 nucleotides, at least about 150 nucleotides or at least about 200 contiguous nucleotides of a sequence selected from the group consisting of SEQ ID NOs: 115-228, 362-494, 512-529; 536-541, 823-1103, 1334-1563, and 1624-1683, or a sequence having a least 90 percent, at least 95 percent or 98 percent identity to the selected sequence or to a fragment thereof of equivalent length to the one strand.

38. The insecticidal composition of claim 18, wherein the group in (a), (b), (c), (d) and (e) is SEQ ID NOs: 1, 2, 13, 36, 37, 39, 47, 103, 302, 340, 346, 349, 1121, 1157, 1178, 1179, 1185, 1310, 1313, 1318, and 1322.

39. The insecticidal composition of claim 18, wherein the group in (f) is SEQ ID NOs 115, 116, 127, 150, 151, 153, 161, 217, 435, 473, 479, 482, 1351, 1387, 1408, 1409, 1415, 1540, 1543, 1548, and 1552.
